(19) United States
(12) Patent Application Publication
(10) Pub. No.: US 2021/0071080 A1
(43) Pub. Date: Mar. 11, 2021

(54) COMPOUND, LIQUID CRYSTAL COMPOSITION AND LIQUID CRYSTAL DISPLAY DEVICE

(71) Applicants: JNC CORPORATION, Tokyo (JP); JNC PETROCHEMICAL CORPORATION, Tokyo (JP)

(72) Inventors: Tomohiro YANO, Chiba (JP); Fumitaka KONDO, Chiba (JP)

(73) Assignees: JNC CORPORATION, Tokyo (JP); JNC PETROCHEMICAL CORPORATION, Tokyo (JP)

(21) Appl. No.: 16/962,223

(22) PCT Filed: Dec. 13, 2018
(86) PCT No.: PCT/JP2018/045882

§ 371 Date: Jul. 14, 2020

(30) Foreign Application Priority Data
Feb. 7, 2018 (JP) 2018-020435

(51) Int. Cl. C09K 19/12; C09K 19/20; C09K 19/16; C09K 19/34; C09K 19/32

(52) U.S. Cl. CPC C09K 19/12; C09K 19/20; C09K 2019/3027; C09K 19/3402; C09K 19/32; C09K 19/16

(57) ABSTRACT

Provided is a compound represented by formula (1) described below.

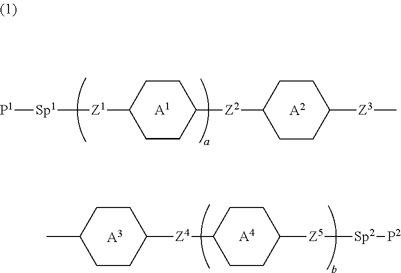

In formula (1), a and b are independently 0, 1 or 2, and expressions: 0≤a+b≤3 hold, ring A¹, ring A², ring A³ and ring A⁴ are independently 1,4-cyclohexylene, for example, Z¹, Z², Z³, Z⁴ and Z³ are independently a single bond, alkylene having 1 to 10 carbons, or the like, Sp¹ and Sp² are independently a single bond, alkylene having 1 to 10 carbons, or the like, and P¹ and P² are independently a specific polymerizable group.

### TECHNICAL FIELD

The invention relates to a compound, a liquid crystal composition and a liquid crystal display device. More specifically, the invention relates to a polymerizable polar compound that have a plurality of kinds of polymerizable groups in one molecule, a liquid crystal composition that has positive or negative dielectric anisotropy and contains the compound, and a liquid crystal display device including the composition.

### BACKGROUND ART

In a liquid crystal display device, a classification based on an operating mode for liquid crystal molecules includes a phase change (PC) mode, a twisted nematic (TN) mode, a super twisted nematic (STN) mode, an electrically controlled birefringence (ECB) mode, an optically compensated bend (OCB) mode, an in-plane switching (IPS) mode, a vertical alignment (VA) mode, a fringe field switching (FFS) mode and a field-induced photo-reactive alignment (FPA) mode. A classification based on a driving mode in the device includes a passive matrix (PM) and an active matrix (AM). The PM is classified into static, multiplex and so forth, and the AM is classified into a thin film transistor (TFT), a metal insulator metal (MIM) and so forth. The TFT is further classified into amorphous silicon and polycrystal silicon. The latter is classified into a high temperature type and a low temperature type based on a production process. A classification based on a light source includes a reflective type utilizing natural light, a transmissive type utilizing backlight, and a transflective type utilizing both the natural light and the backlight.

The liquid crystal display device includes a liquid crystal composition having a nematic phase. The composition has suitable characteristics. An AM device having good characteristics can be obtained by improving characteristics of the composition. Table 1 below summarizes a relationship between two characteristics. The characteristics of the composition will be further described based on a commercially available AM device. A temperature range of the nematic phase relates to a temperature range in which the device can be used. A preferred maximum temperature of the nematic phase is about 70° C. or higher, and a preferred minimum temperature of the nematic phase is about −10° C. or lower. Viscosity of the composition relates to a response time in the device. A short response time is preferred for displaying moving images on the device. A shorter response time even by one millisecond is desirable. Accordingly, small viscosity in the composition is preferred. Small viscosity at low temperature is further preferred.

TABLE 1
Characteristics of composition and AM device

| No. | Characteristics of composition | Characteristics of AM device |
|---|---|---|
| 1 | Wide temperature range | Wide usable temperature range |
| | of a nematic phase | |
| 2 | Small viscosity¹⁾ | Short response time |
| 3 | Suitable optical anisotropy | Large contrast ratio |
| 4 | Large positive or negative | Low threshold voltage and |
| | dielectric anisotropy | small electric power consumption |
| | | Large contrast ratio |
| 5 | Large specific resistance | Large voltage holding ratio and |
| | | large contrast ratio |
| 6 | High stability to ultraviolet | Long service life |
| | light and heat | |
| 7 | Large elastic constant | Large contrast ratio and |
| | | short response time |

¹⁾A composition can be injected into a liquid crystal display device in a short time.

Optical anisotropy of the composition relates to a contrast ratio in the device. According to a mode of the device, large optical anisotropy or small optical anisotropy, more specifically, suitable optical anisotropy is required. A product (Δn×d) of the optical anisotropy (Δn) of the composition and a cell gap in the device is designed so as to maximize the contrast ratio. A suitable value of the product depends on a type of the operating mode. The value is about 0.45 micrometer in a device having a mode such as the TN mode. The value is in the range of about 0.30 micrometer to about 0.40 micrometer in a device having the VA mode, and in the range of about 0.20 micrometer to about 0.30 micrometer in a device having the IPS mode or the FFS mode. In the above case, a composition having large optical anisotropy is preferred for a device having a small cell gap. Large dielectric anisotropy in the composition contributes to low threshold voltage, small electric power consumption and a large contrast ratio in the device. Accordingly, large positive or negative dielectric anisotropy is preferred. Large specific resistance in the composition contributes to a large voltage holding ratio and the large contrast ratio in the device. Accordingly, a composition having large specific resistance at room temperature and also at a temperature close to the maximum temperature of the nematic phase in an initial stage is preferred. The composition having large specific resistance at room temperature and also at a temperature close to the maximum temperature of the nematic phase even after the device has been used for a long period of time is preferred. Stability of the composition to ultraviolet light and heat relates to a service life of the device. In the case where the stability is high, the device has a long service life. Such characteristics are preferred for an AM device for use in a liquid crystal projector, a liquid crystal television and so forth.

A composition having positive dielectric anisotropy is used in an AM device having the TN mode. A composition having negative dielectric anisotropy is used in an AM device having the VA mode. In an AM device having the IPS mode or the FFS mode, a composition having positive or negative dielectric anisotropy is used.

In an AM device having a polymer sustained alignment (PSA) mode, a composition having positive or negative dielectric anisotropy is used. In a liquid crystal display device having the polymer sustained alignment (PSA) mode, a liquid crystal composition containing a polymer is used. First, a composition to which a small amount of a polymerizable compound is added is injected into the device. Next, the composition is irradiated with ultraviolet light while voltage is applied between substrates of the device. The polymerizable compound is polymerized to form a network structure of the polymer in the composition. In the composition, alignment of liquid crystal molecules can be controlled by the polymer, and therefore the response time in the device is shortened and also image persistence is improved. Such an effect of the polymer can be expected for a device having the mode such as the TN mode, the ECB mode, the OCB mode, the IPS mode, the VA mode, the FFS mode and the FPA mode.

A method for controlling alignment of liquid crystals using, in place of an alignment film of polyimide or the like, a low molecular weight compound having a cinnamate group, polyvinyl cinnamate, a low molecular weight compound having a chalcone structure, a low molecular weight compound having an azobenzene structure or dendrimer has been reported (Patent literature Nos. 1, 2 or 3). In the method in Patent literature No. 1, 2 or 3, first, the low molecular weight compound or the polymer is dissolved in the liquid crystal composition as an additive. Next, the additive is phase-separated to form a thin film composed of the low molecular weight compound or the polymer on a substrate. Finally, the substrate is irradiated with linearly polarized light at a temperature higher than the maximum temperature of the liquid crystal composition. When dimerization or isomerization in the low molecular weight compound or the polymer is caused by this linearly polarized light, the molecules are arranged in a fixed direction. In the method, a device having a horizontal alignment mode such as the IPS mode and the FFS mode and a device having a vertical alignment mode such as the VA mode can be produced by selecting a kind of low molecular weight compound or polymer. In the method, importance is on easily causing dissolution of the low molecular weight compound or the polymer therein at a temperature higher than the maximum temperature of the liquid crystal composition, and on easily causing phase separation of the compound from the liquid crystal composition when the temperature is returned to room temperature. However, securement of compatibility between the low molecular weight compound or the polymer and the liquid crystal composition is difficult.

So far, as a compound to allow horizontal alignment of the liquid crystal molecules in the liquid crystal display device having no alignment film, a compound having a methacrylate group at a terminal ([Formula 2]) has been described in Patent literature No. 2, and a compound having an acrylate group at a terminal [14] and so forth have been described in Patent literature No. 3. However, in the compounds, ability to allow horizontal alignment of the liquid crystal molecules is not sufficient. Moreover, the polymerizable group used for replacement is only one kind.

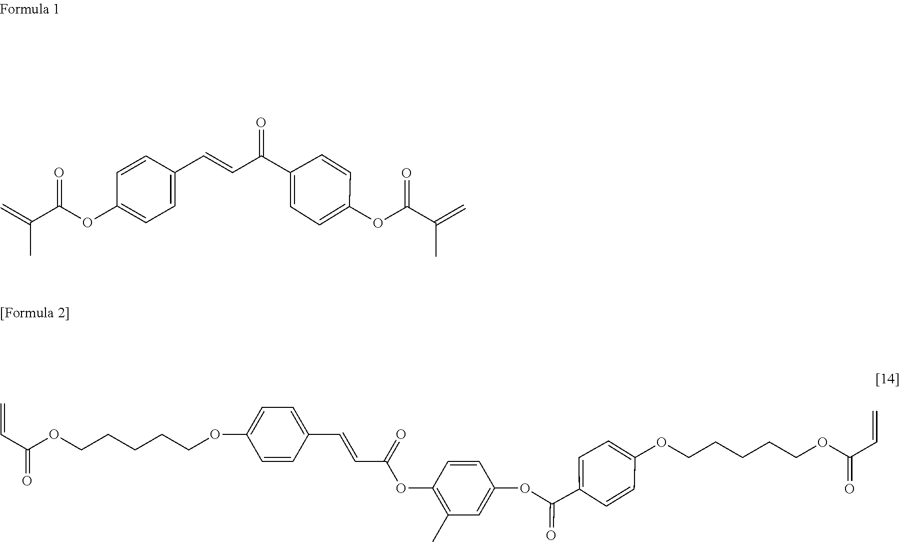 [SMILES: C=C(C)C(=O)Oc1ccc(/C=C/C(=O)c2ccc(OC(=O)C(=C)C)cc2)cc1 ; C=CC(=O)OCCCCCOc1ccc(/C=C/C(=O)Oc2ccc(OC(=O)c3ccc(OCCCCCOC(=O)C=C)cc3)cc2C)cc1]

### CITATION LIST

### Patent Literature

Patent literature No. 1: WO 2015/146369 A.

Patent literature No. 2: WO 2017/057162 A.

Patent literature No. 3: WO 2017/102068 A.

### SUMMARY OF INVENTION

### Technical Problem

A first object of the invention is to provide a compound having at least one of characteristics such as chemically high stability, high ability to allow horizontal alignment of liquid crystal molecules, high alignability in a wide addition concentration range, suitable reactivity, and high solubility in a liquid crystal composition, in which a large voltage holding ratio is expected when the compound is used in a liquid crystal display device. A second object is to provide a liquid crystal composition that contains the compound, and satisfies at least one of characteristics such as high maximum temperature of a nematic phase, low minimum temperature of the nematic phase, small viscosity, suitable optical anisotropy, large positive or negative dielectric anisotropy, large specific resistance, high stability to ultraviolet light, high stability to heat and a large elastic constant. A third object is to provide a liquid crystal display device that includes the composition, and has at least one of characteristics such as a wide temperature range in which the device can be used, a short response time, a high voltage holding ratio, low threshold voltage, a large contrast ratio and a long service life, in which, when a polar compound forms a film inside the device by irradiating the composition with ultraviolet light, the film has at least one of characteristics such as suitable hardness, low permeability of a component in contact therewith, high weather resistance and a suitable volume resistance value.

### Solution to Problem

The present inventors have found that a compound represented by formula (1) described below can solve the problem described above, and have completed the invention.

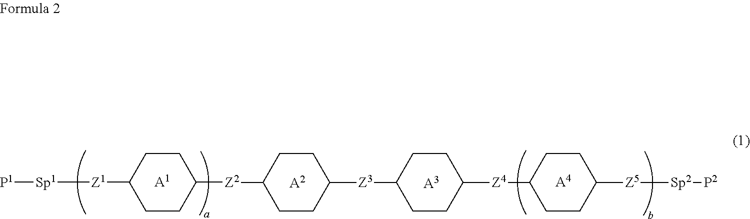

(A symbol in the formula will be described later.).

### Advantageous Effects of Invention

A first advantage of the invention is to provide a compound having at least one of chemically high stability, high ability to allow horizontal alignment of liquid crystal molecules, high alignability in a wide addition concentration range, suitable reactivity, and high solubility in a liquid crystal composition, in which a large voltage holding ratio is expected when the compound is used in a liquid crystal display device. A second advantage is to provide a liquid crystal composition that contains the compound, and satisfies at least one of characteristics such as high maximum temperature of a nematic phase, low minimum temperature of the nematic phase, small viscosity, suitable optical anisotropy, large positive or negative dielectric anisotropy, large specific resistance, high stability to ultraviolet light, high stability to heat and a large elastic constant. A third advantage is to provide a liquid crystal display device that includes the composition, and has at least one of characteristics such as a wide temperature range in which the device can be used, a short response time, a high voltage holding ratio, low threshold voltage, a large contrast ratio and a long service life, in which, when a polar compound forms a film inside the device by irradiating the composition with ultraviolet light, the film has at least one of characteristics such as suitable hardness, low permeability with a component in contact therewith, high weather resistance and a suitable volume resistance value. A step of forming an alignment film becomes unnecessary by utilizing the liquid crystal composition containing the compound of the invention, and therefore the liquid crystal display device in which production cost is reduced can be obtained.

### DESCRIPTION OF EMBODIMENTS

Usage of terms herein is as described below. Terms “liquid crystal composition” and “liquid crystal display device” may be occasionally abbreviated as “composition” and “device,” respectively. “Liquid crystal display device” is a generic term for a liquid crystal display panel and a liquid crystal display module. “Liquid crystal compound” is a generic term for a compound having a liquid crystal phase such as a nematic phase and a smectic phase, and a compound having no liquid crystal phase but to be mixed with the composition for the purpose of adjusting characteristics such as a temperature range of the nematic phase, viscosity and dielectric anisotropy. The compound has a six-membered ring such as 1,4-cyclohexylene and 1,4-phenylene, and has rod-like molecular structure. “Polymerizable compound” is a compound to be added for the purpose of forming a polymer in the composition. “Polar compound” supports a polar group to interact with a substrate surface, thereby causing arrangement of liquid crystal molecules.

The liquid crystal composition is prepared by mixing a plurality of liquid crystal compounds. A proportion (content) of the liquid crystal compounds is expressed in terms of weight percent (% by weight) based on the weight of the liquid crystal composition. An additive such as an optically active compound, an antioxidant, an ultraviolet light absorber, a dye, an antifoaming agent, the polymerizable compound, a polymerization initiator, a polymerization inhibitor and a polar compound is added to the liquid crystal composition when necessary. A proportion (amount of addition) of the additive is expressed in terms of weight percent (% by weight) based on the weight of the liquid crystal composition in a manner similar to the proportion of the liquid crystal compound. Weight parts per million (ppm) may be occasionally used. A proportion of the polymerization initiator and the polymerization inhibitor is exceptionally expressed based on the weight of the polymerizable compound.

A compound represented by formula (1) may be occasionally abbreviated as “compound (1).” Compound (1) means one compound, a mixture of two compounds or a mixture of three or more compounds represented by formula (1). A same rule applies also to at least one compound selected from the group of compounds represented by formula (2), or the like. Symbol B¹, C¹, F or the like surrounded by a hexagonal shape corresponds to ring B¹, ring C¹, ring F or the like, respectively. The hexagonal shape represents a six-membered ring such as a cyclohexane ring and a benzene ring, or a fused ring such as a naphthalene ring. An oblique line crossing the hexagonal shape represents that arbitrary hydrogen on the ring may be replaced by a group such as -Sp¹-P¹. A subscript such as e represents the number of groups used for replacement. When the subscript is 0 (zero), no such replacement exists.

A symbol of terminal group R¹¹ is used in a plurality of component compounds. In the compounds, two groups represented by two of arbitrary R¹¹ may be identical or different. For example, in one case, R¹¹ of compound (2) is ethyl and R¹¹ of compound (3) is ethyl. In another case, R¹¹ of compound (2) is ethyl and R¹¹ of compound (3) is propyl. A same rule applies also to a symbol of any other terminal group, a ring, a bonding group or the like. In formula (8), when i is 2, two of ring D¹ exists. In the compound, two groups represented by two of ring D¹ may be identical or different. A same rule applies also to two of arbitrary ring D¹ when i is larger than 2. A same rule applies also to a symbol of any other ring, a bonding group or the like.

An expression “at least one piece of ‘A’” means that the number of ‘A’ is arbitrary. An expression “at least one piece of ‘A’ may be replaced by ‘B’” means that, when the number of ‘A’ is 1, a position of ‘A’ is arbitrary, and also when the number of ‘A’ is 2 or more, positions thereof can be selected without restriction. A same rule applies also to an expression “at least one piece of ‘A’ is replaced by ‘B’.” An expression “at least one piece of A may be replaced by B, C or D” includes a case where at least one piece of A is replaced by B, a case where at least one piece of A is replaced by C, and a case where at least one piece of A is replaced by D, and also a case where a plurality of pieces of A are replaced by at least two pieces of B, C and D. For example, “alkyl in which at least one piece of —CH₂— (or —CH₂CH₂—) may be replaced by —O— (or —CH═CH—)” includes alkyl, alkenyl, alkoxy, alkoxyalkyl, alkoxyalkenyl and alkenyloxyalkyl. In addition, a case where two pieces of consecutive —CH₂— are replaced by —O— to form —O—O— is not preferred. In alkyl or the like, a case where —CH₂— of a methyl part (—CH₂—H) is replaced by —O— to form —O—H is not preferred, either.

Halogen means fluorine, chlorine, bromine or iodine. Preferred halogen is fluorine or chlorine. Further preferred halogen is fluorine. Alkyl is straight-chain alkyl or branched-chain alkyl, and includes no cyclic alkyl. In general, straight-chain alkyl is preferred to branched-chain alkyl. A same rule applies also to a terminal group such as alkoxy and alkenyl. With regard to a configuration of 1,4-cyclohexylene, trans is preferred to cis for increasing the maximum temperature of the nematic phase. Then, 2-fluoro-1,4-phenylene means two divalent groups described below. In a chemical formula, fluorine may be leftward (L) or rightward (R). A same rule applies also to an asymmetrical divalent group formed by eliminating two hydrogens from a ring, such as tetrahydropyran-2,5-diyl.

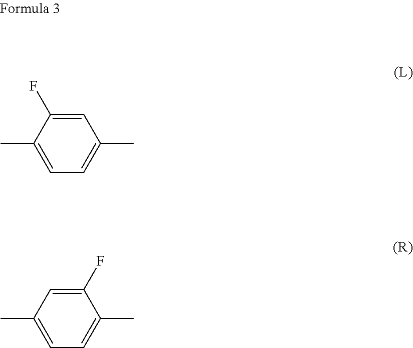

The invention includes items described below.

Item 1. A compound, represented by formula (1):

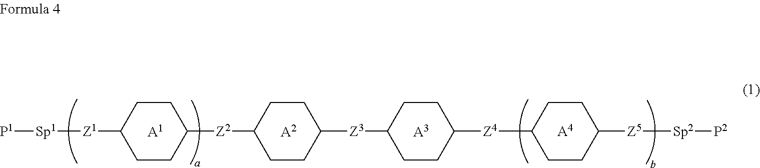

wherein, in formula (1),

a and b are independently 0, 1 or 2, and expressions: 0≤a+b≤3 hold,

ring A¹, ring A², ring A³ and ring A⁴ are independently 1,4-cyclohexylene, 1,4-cyclohexenylene, 1,4-phenylene, naphthalene-2,6-diyl, decahydronaphthalene-2,6-diyl, 1,2,3,4-tetrahydronaphthalene-2,6-diyl, tetrahydropyran-2,5-diyl, 1,3-dioxane-2,5-diyl, pyrimidine-2,5-diyl, pyridine-2,5-diyl, fluorene-2,7-diyl, phenanthrene-2,7-diyl, anthracene-2,6-diyl, perhydrocyclopenta[a]phenanthrene-3,17-diyl or 2,3,4,7,8,9,10,11,12,13,14,15,16,17-tetradecahydrocyclopenta[a]phenanthrene-3,17-diyl, and in the rings, at least one hydrogen may be replaced by fluorine, chlorine, alkyl having 1 to 12 carbons, alkenyl having 2 to 12 carbons, alkoxy having 1 to 11 carbons, alkenyloxy having 2 to 11 carbons, -Sp¹-P¹ or -Sp²-P², and in the groups, at least one hydrogen may be replaced by fluorine or chlorine, and when a is 2, two of ring A¹ may be different, and when b is 2, two of ring A⁴ may be different;

Z¹, Z², Z³, Z⁴ and Z⁵ are independently a single bond or alkylene having 1 to 10 carbons, and in the alkylene, at least one piece of —CH₂— may be replaced by —O—, —CO—, —COO—, —OCO— or —OCOO—, and at least one piece of —(CH₂)₂— may be replaced by —CH═CH— or —C≡C— and in the groups, at least one hydrogen may be replaced by halogen, in which at least one in Z², Z³ or Z⁴ is —COO—, —OCO—, —CH═CHCOO—, —OCOCH═CH—, —CH═CH—, —CH═CHCO— or —COCH═CH—, and when a is 2, two of Z¹ may be different, and two of Z⁵ may be different;

Sp¹ and Sp² are independently a single bond or alkylene having 1 to 10 carbons, and in the alkylene, at least one piece of —CH₂— may be replaced by —O—, —CO—, —COO—, —OCO— or —OCOO—, and at least one piece of —(CH₂)₂— may be replaced by —CH═CH— or —C≡C—, and in the groups, at least one hydrogen may be replaced by halogen, and when a plurality of Sp¹ or Sp² are present in a structure, the plurality each may be different; and

P¹ and P² are independently a group represented by any one of formula (1b) to formula (1h), and when a plurality of P¹ or P² are present a structure, the plurality each may be different, in which a case where all of P¹ and P² have an identical structure is excluded;

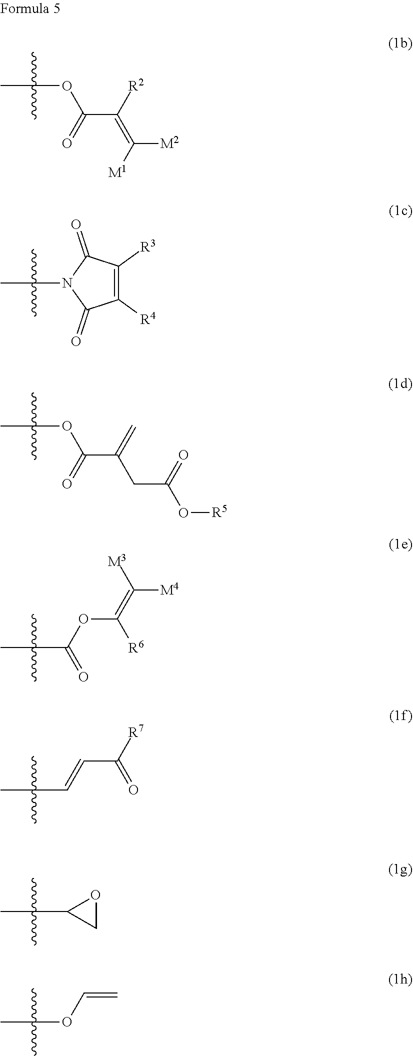

wherein, in formula (1b) to formula (1h),

M¹, M², M³ and M⁴ are independently hydrogen, halogen, alkyl having 1 to 5 carbons, or alkyl having 1 to 5 carbons in which at least one hydrogen is replaced by halogen;

R² is hydrogen, halogen or alkyl having 1 to 5 carbons, and in the alkyl, at least one hydrogen may be replaced by halogen, and at least one piece of —CH₂— may be replaced by —O—; and

R³, R⁴, R⁵, R⁶ and R⁷ are independently hydrogen or alkyl having 1 to 15 carbons, and in the alkyl, at least one piece of —CH₂— may be replaced by —O— or —S—, and at least one piece of —(CH₂)₂— may be replaced by —CH═CH— or —C≡C—, and in the groups, at least one hydrogen may be replaced by halogen.

Item 2. The compound according to item 1, wherein, in formula (1),

a and b are independently 0, 1 or 2, and expressions 0≤a+b≤2 hold;

ring A¹, ring A², ring A³ and ring A⁴ are independently 1,4-cyclohexylene, 1,4-cyclohexenylene, 1,4-phenylene, naphthalene-2,6-diyl, decahydronaphthalene-2,6-diyl, 1,2,3,4-tetrahydronaphthalene-2,6-diyl, tetrahydropyran-2,5-diyl, 1,3-dioxane-2,5-diyl, pyrimidine-2,5-diyl, pyridine-2,5-diyl, fluorene-2,7-diyl, phenanthrene-2,7-diyl, anthracene-2,6-diyl, perhydrocyclopenta[a]phenanthrene-3,17-diyl or 2,3,4,7,8,9,10,11,12,13,14,15,16,17-tetradecahydrocyclopenta[a]phenanthrene-3,17-diyl, and in the rings, at least one hydrogen may be replaced by fluorine, chlorine, alkyl having 1 to 12 carbons, alkenyl having 2 to 12 carbons, alkoxy having 1 to 11 carbons, alkenyloxy having 2 to 11 carbons, -Sp¹-P¹ or -Sp²-P², and in the groups, at least one hydrogen may be replaced by fluorine or chlorine, and when a is 2, two of ring A¹ may be different, and when b is 2, two of ring A⁴ may be different;

Z¹, Z², Z³, Z⁴ and Z⁵ are independently a single bond, —(CH₂)₂—, —CH═CH—, —C≡C—, —COO—, —OCO—, —CF₂O—, —OCF₂—, —CH₂O—, —OCH₂—, —CF═CF—, —CH═CHCOO—, —OCOCH═CH—, —CH═CHCO— or —COCH═CH—, in which at least one in Z², Z³ or Z⁴ is —COO—, —OCO—, —CH═CHCOO—, —OCOCH═CH—, —CH═CH—, —CH═CHCO— or —COCH═CH—, and when a is 2, two of Z¹ may be different, and two of Z⁵ may be different;

Sp¹ and Sp² are independently a single bond or alkylene having 1 to 10 carbons, and in the alkylene, at least one piece of —CH₂— may be replaced by —O—, —COO— or —OCO—, and at least one piece of —(CH₂)₂— may be replaced by —CH═CH—, and in the groups, at least one hydrogen may be replaced by fluorine or chlorine, and when a plurality of Sp¹ or Sp² are present in a structure, the plurality each may be different; and

P¹ and P² are independently a group represented by any one of formula (1b) to formula (1h), and when a plurality of P¹ or P² are present in a structure, the plurality each may be different, in which a case where all of P¹ and P² have an identical structure is excluded;

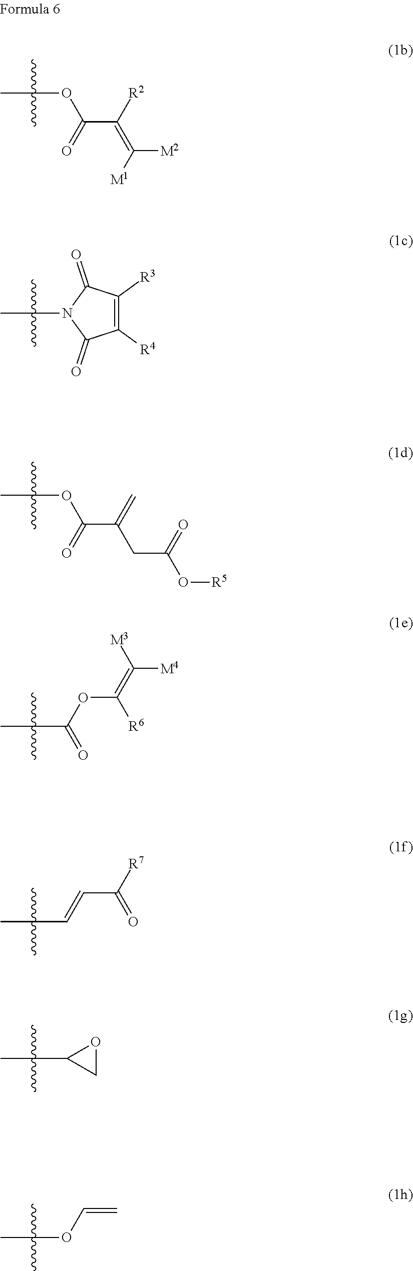

wherein, in formula (1b) to formula (1h),

M¹, M², M³ and M⁴ are independently hydrogen, halogen, alkyl having 1 to 5 carbons, or alkyl having 1 to 5 carbons in which at least one hydrogen is replaced by halogen;

R² is hydrogen, halogen or alkyl having 1 to 5 carbons, and in the alkyl, at least one hydrogen may be replaced by halogen, and at least one piece of —CH₂— may be replaced by —O—; and

R³, R⁴, R⁵, R⁶ and R⁷ are independently hydrogen or alkyl having 1 to 15 carbons, and in the alkyl, at least one piece of —CH₂— may be replaced by —O— or —S—, and at least one piece of —(CH₂)₂— may be replaced by —CH═CH— or —C≡C—, and in the groups, at least one hydrogen may be replaced by halogen.

Item 3. The compound according to item 1 or 2, represented by any one of formula (1-1) to formula (1-3):

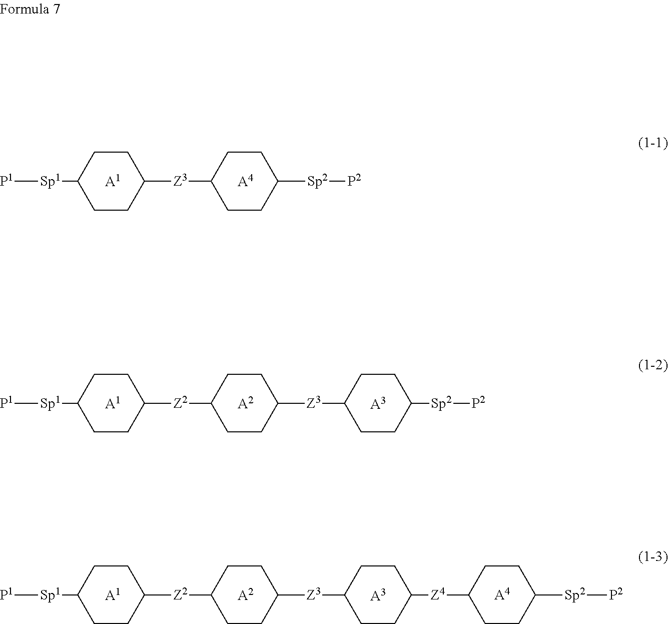

wherein, in formula (1-1) to formula (1-3),

ring A¹, ring A², ring A³ and ring A⁴ are independently 1,4-cyclohexylene, 1,4-phenylene, naphthalene-2,6-diyl, pyrimidine-2,5-diyl, pyridine-2,5-diyl, fluorene-2,7-diyl, phenanthrene-2,7-diyl or anthracene-2,6-diyl, and in the rings, at least one hydrogen may be replaced by fluorine, chlorine, alkyl having 1 to 12 carbons, alkenyl having 2 to 12 carbons, alkoxy having 1 to 11 carbons, alkenyloxy having 2 to 11 carbons, -Sp¹-P¹ or -Sp²-P², and in the groups, at least one hydrogen may be replaced by fluorine or chlorine;

Z², Z³ and Z⁴ are independently a single bond, —(CH₂)₂—, —CH═CH—, —C≡C—, —COO—, —OCO—, —CF₂O—, —OCF₂—, —CH₂O—, —OCH₂—, —CF═CF—, —CH═CHCOO—, —OCOCH═CH—, —CH═CHCO— or —COCH═CH—, in which at least one in Z², Z³ and Z⁴ is —COO—, —OCO—, —CH═CHCOO—, —OCOCH═CH—, —CH═CH—, —CH═CHCO— or —COCH═CH—;

Sp¹ and Sp² are independently a single bond or alkylene having 1 to 10 carbons, and in the alkylene, at least one piece of —CH₂— may be replaced by —O—, —COO—, —OCOO— or —OCO—, and at least one piece of —(CH₂)₂— may be replaced by —CH═CH—, and in the groups, at least one hydrogen may be replaced by fluorine or chlorine, and when a plurality of Sp¹ or Sp² are present in a structure, the plurality each may be different; and

P¹ and P² are independently a group represented by any one of formula (1b) to formula (1h), and when a plurality of P¹ or P² are present in a structure, the plurality each may be different, in which a case where all of P¹ and P² have an identical structure is excluded;

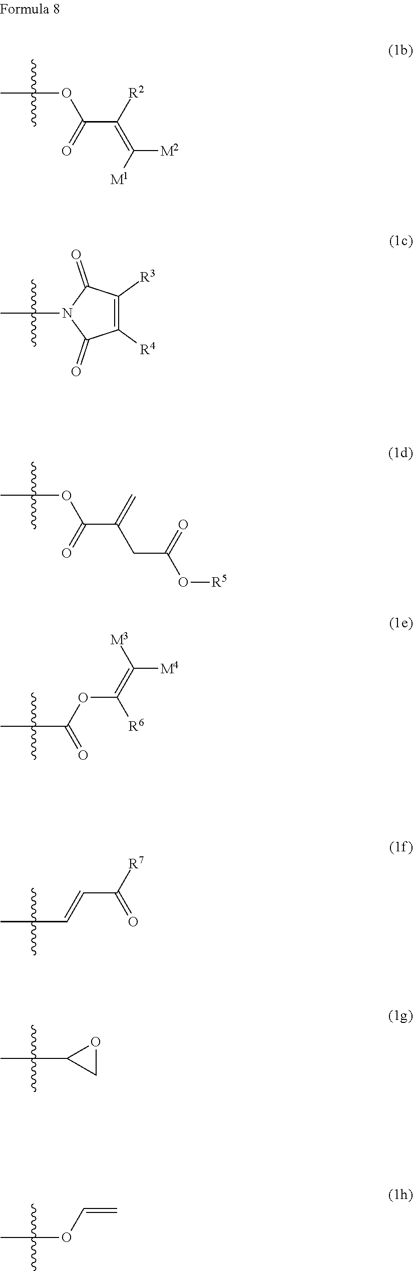

wherein

M¹, M², M³ and M⁴ are independently hydrogen, halogen, alkyl having 1 to 5 carbons, or alkyl having 1 to 5 carbons in which at least one hydrogen is replaced by halogen;

R² is hydrogen, halogen or alkyl having 1 to 5 carbons, and in the alkyl, at least one hydrogen may be replaced by halogen, and at least one piece of —CH₂— may be replaced by and

R³, R⁴, R⁵, R⁶ and R⁷ are independently hydrogen or alkyl having 1 to 15 carbons, and in the alkyl, at least one piece of —CH₂— may be replaced by —O— or —S—, and at least one piece of —(CH₂)₂— may be replaced by —CH═CH— or —C≡C—, and in the groups, at least one hydrogen may be replaced by halogen.

Item 4. The compound according to item 3, wherein, in formula (1-1), formula (1-2) and formula (1-3),

ring A¹, ring A², ring A³ and ring A⁴ are independently 1,4-cyclohexylene, 1,4-phenylene or fluorene-2,7-diyl, and in the rings, at least one hydrogen may be replaced by fluorine, chlorine, alkyl having 1 to 12 carbons, alkenyl having 2 to 12 carbons, alkoxy having 1 to 11 carbons, alkenyloxy having 2 to 11 carbons, -Sp¹-P¹ or -Sp²-P²;

Z², Z³ and Z⁴ are independently a single bond, —(CH₂)₂—, —CH═CH—, —C≡C—, —COO—, —OCO—, —CH═CHCOO—, —OCOCH═CH—, —CH═CHCO— or —COCH═CH—, in which at least one in Z², Z³ and Z⁴ is —COO—, —OCO—, —CH═CHCOO—, —OCOCH═CH—, —CH═CH—, —CH═CHCO— or —COCH═CH—;

Sp¹ and Sp² are independently a single bond or alkylene having 1 to 10 carbons, and in the alkylene, at least one piece of —CH₂— may be replaced by —O—, —COO—, —OCOO— or —OCO—, and at least one piece of —(CH₂)₂— may be replaced by —CH═CH—, and when a plurality of Sp¹ or Sp² are present in a structure, the plurality each may be different; and

P¹ and P² are independently a group represented by any one of formula (1b), formula (1c), formula (1d) or formula (1e), and when a plurality of P¹ or P² are present in a structure, the plurality each may be different, in which a case where all of P¹ and P² have an identical structure is excluded, and a case where P¹ and P² are in a combination of only acrylate or methacrylate is excluded;

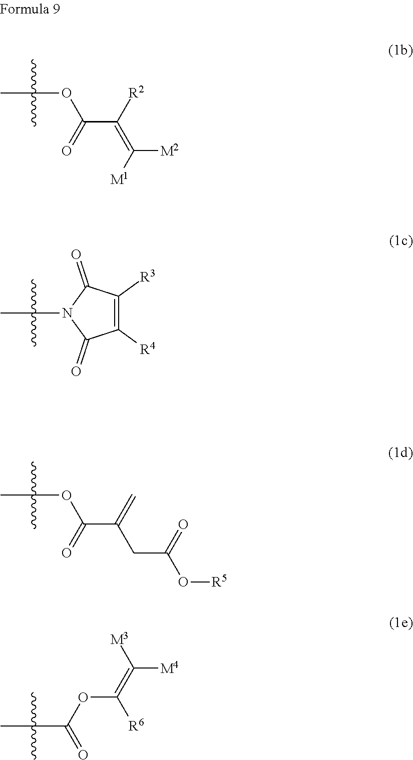

wherein

M¹, M², M³ and M⁴ are independently hydrogen, halogen, alkyl having 1 to 5 carbons, or alkyl having 1 to 5 carbons in which at least one hydrogen is replaced by halogen; and

in formula (1b) to formula (1e),

R² is hydrogen, halogen or alkyl having 1 to 5 carbons, and in the alkyl, at least one hydrogen may be replaced by halogen, and at least one piece of —CH₂— may be replaced by —O—; and

R³, R⁴, R⁵ and R⁶ are independently hydrogen or alkyl having 1 to 15 carbons, and in the alkyl, at least one piece of —CH₂— may be replaced by —O— or —S—, and at least one piece of —(CH₂)₂— may be replaced by —CH═CH— or —C≡C—, and in the groups, at least one hydrogen may be replaced by halogen.

Item 5. The compound according to item 4, wherein, in the compound represented by formula (1-1), formula (1-2) or formula (1-3), any one of Z², Z³ or Z⁴ is —COO— or —OCO—.

Item 6. The compound according to claim 4, wherein, in the compound represented by formula (1-1), formula (1-2) or formula (1-3), any one of Z², Z³ or Z⁴ is —CH═CHCOO—, —OCOCH═CH—, —CH═CH—, —CH═CHCO— or —COCH═CH—.

Item 7. The compound according to any of items 1 to 4, represented by formula (1-A):

Formula 10

P¹-Sp¹-Y-Sp²-P² (1-A)

wherein

P¹ and P² are independently a group represented by formula (1b-1), (1b-2), (1b-3), (1b-4), (1b-5), (1c-1), (1d-1), (1d-2) or (1e-1), in which a case where all of P¹ and P² have an identical structure is excluded, and a case where P¹ and P² are in a combination of only formulas (1b-1) and (1b-2) is excluded;

Sp¹ and Sp² are independently a single bond or alkylene having 1 to 10 carbons, and in the alkylene, at least one piece of —CH₂— may be replaced by —O—, —COO—, —OCOO— or —OCO—, and at least one piece of —(CH₂)₂— may be replaced by —CH═CH—;

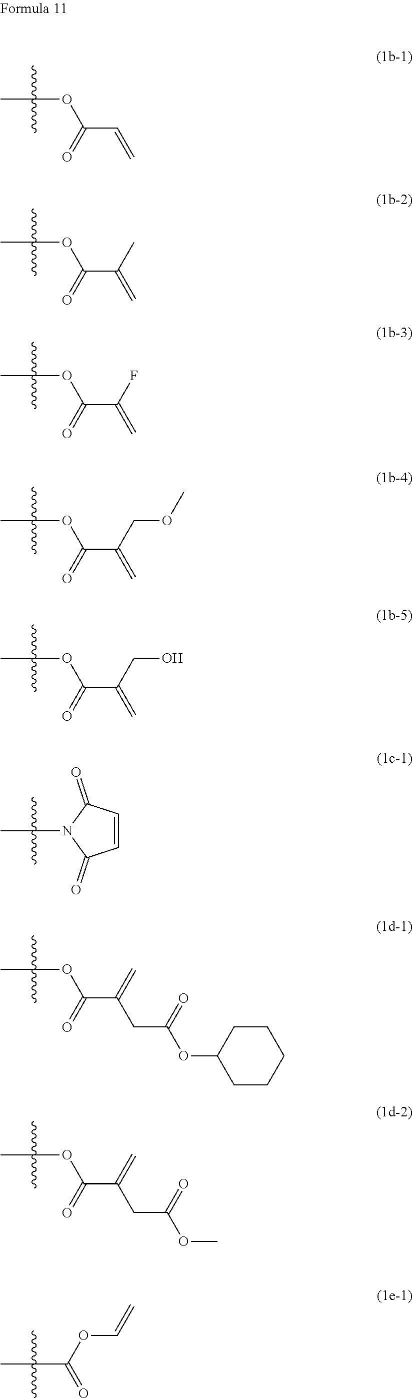

wherein

Y is a group represented by any one of formulas (MES-1-01) to (MES-1-10);

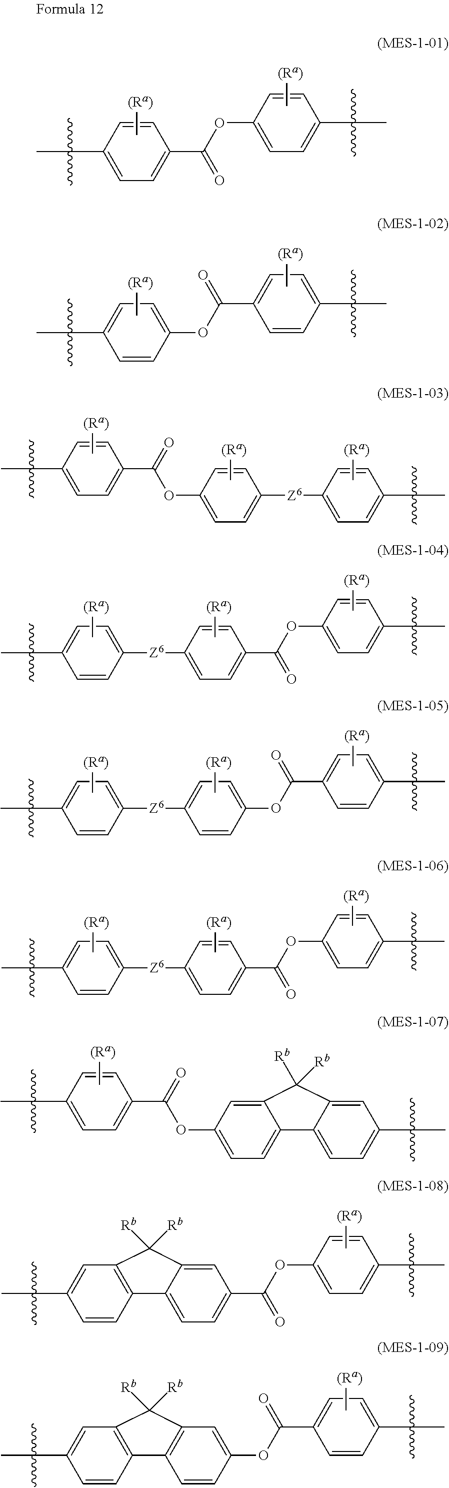

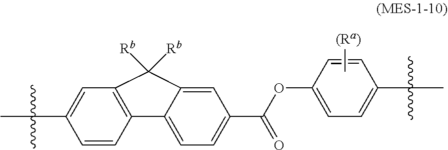

wherein

R^{a} is independently fluorine, chlorine, methyl or ethyl;

R^{b} is independently hydrogen, fluorine, methyl or ethyl;

Z⁶ is independently a single bond or —C≡C—; and

a representation formed by connecting 1,4-phenylene with (R^{a}) by a straight line as shown below in the formulas indicates 1,4-phenylene in which one or two hydrogens may be replaced by R^{a}:

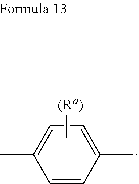

Item 8. The compound according to any one of items 1 to 4, represented by formula (1-A):

Formula 14

P¹-SP¹-Y-Sp²-P² (1-A)

wherein

P¹ and P² are independently a group represented by formula (1b-1), (1b-2), (1b-3), (1b-4), (1b-5), (1c-1), (1d-1), (1d-2) or (1e-1), in which a case where P¹ and P² have an identical structure is excluded, and a case where P¹ and P² are in a combination of only formulas (1b-1) and (1b-2) is excluded;

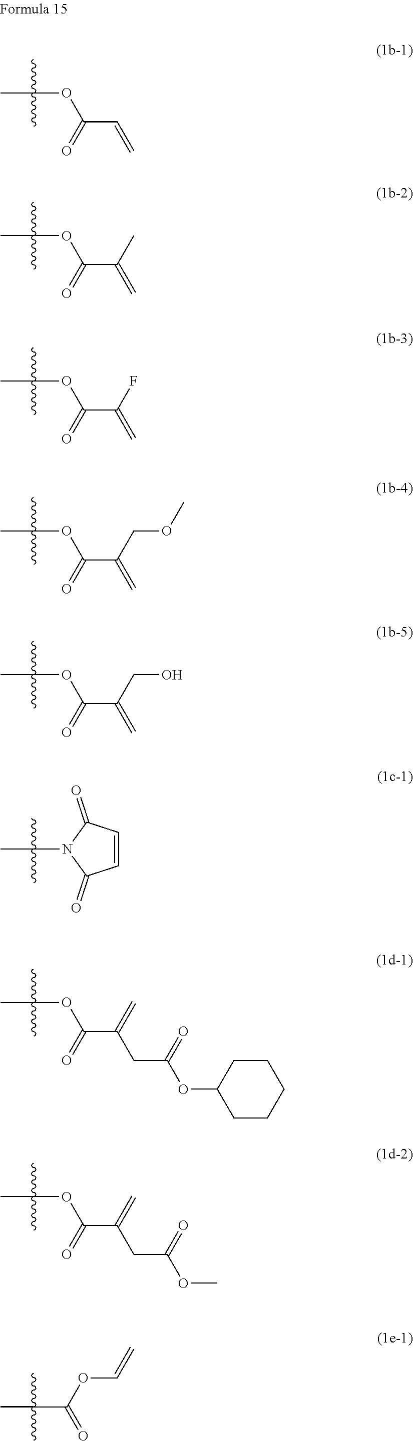

wherein

Sp¹ and Sp² are independently a single bond or alkylene having 1 to 10 carbons, and in the alkylene, at least one piece of —CH₂— may be replaced by —O—, —COO—, —OCOO— or —OCO—, and at least one piece of —(CH₂)₂— may be replaced by —CH═CH—; and

Y is a group represented by any one of formulas (MES-2-01) to (MES-2-15);

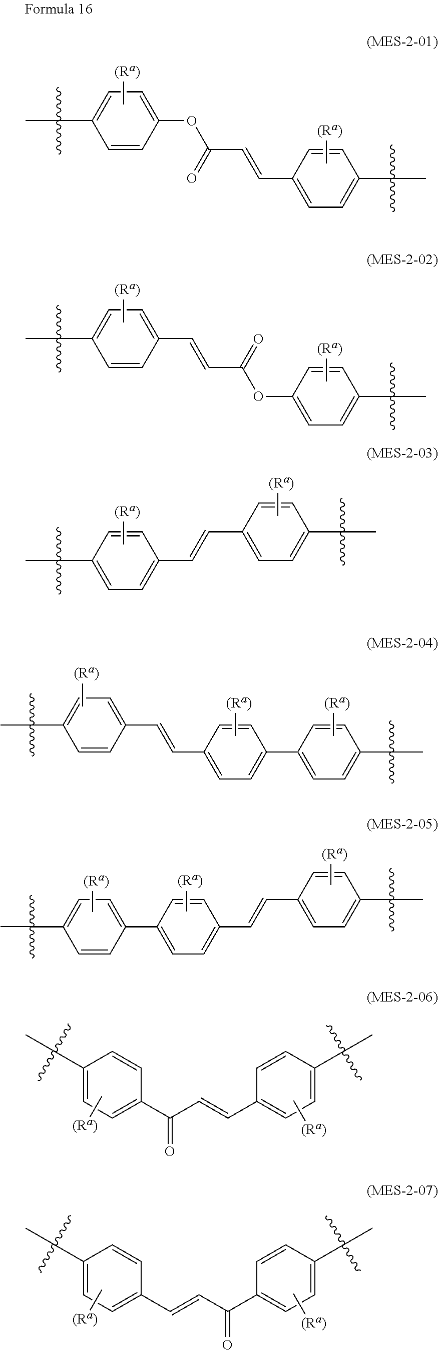

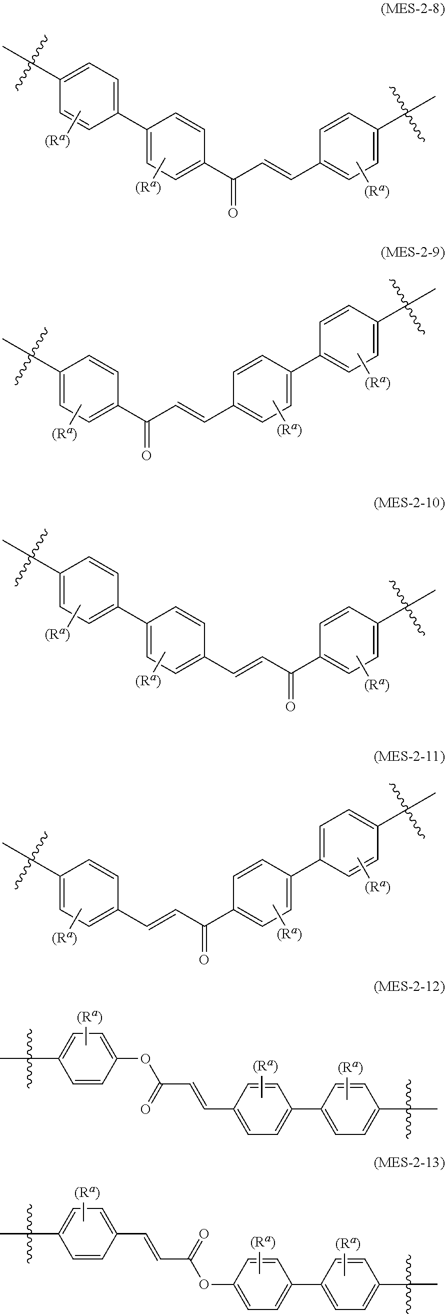

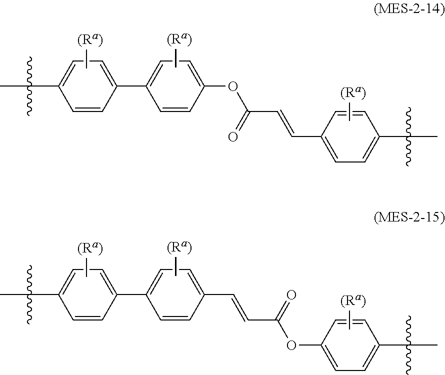

wherein

R^{a} is independently fluorine, chlorine, methyl or ethyl; and

a representation formed by connecting 1,4-phenylene with (R^{a}) by a straight line as shown below in the formulas indicates 1,4-phenylene in which one or two hydrogens may be replaced by R^{a}:

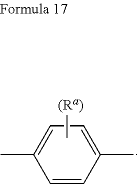

Item 9. A liquid crystal composition, containing at least one compound according to any one of items 1 to 8.

Item 10. The liquid crystal composition according to item 9, further containing at least one compound selected from the group of compounds represented by formula (2) to formula (4):

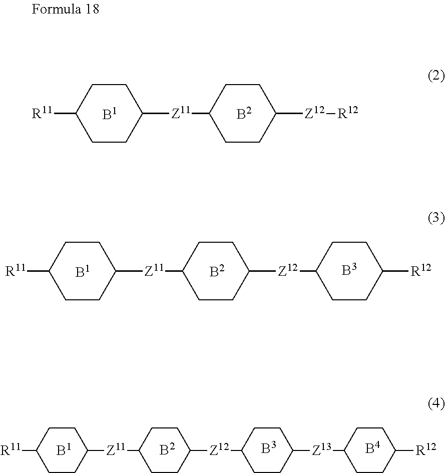

wherein, in formula (2) to formula (4),

R¹¹ and R¹² are independently alkyl having 1 to 10 carbons or alkenyl having 2 to 10 carbons, and in the alkyl and the alkenyl, at least one piece of —CH₂— may be replaced by —O—, and at least one hydrogen may be replaced by fluorine;

ring B¹, ring B², ring B³ and ring B⁴ are independently 1,4-cyclohexylene, 1,4-phenylene, 2-fluoro-1,4-phenylene, 2,5-difluoro-1,4-phenylene or pyrimidine-2,5-diyl; and

Z¹¹, Z¹² and Z¹³ are independently a single bond, —CH₂CH₂—, —CH═CH—, —C≡C— or —COO—.

Item 11. The liquid crystal composition according to item 9 or 10, further containing at least one compound selected from the group of compounds represented by formula (5) to formula (7):

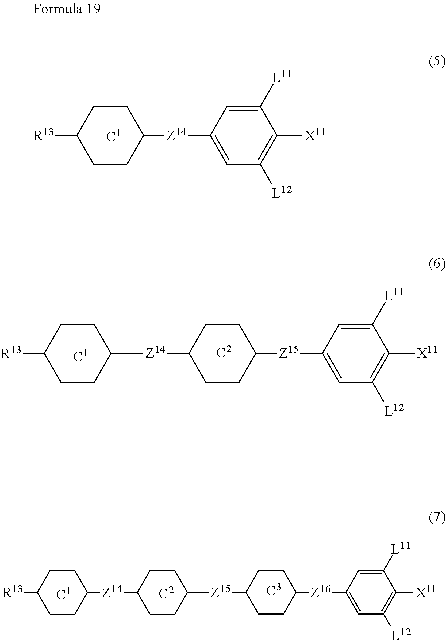

wherein, in formula (5) to formula (7),

R¹³ is alkyl having 1 to 10 carbons or alkenyl having 2 to 10 carbons, and in the alkyl and the alkenyl, at least one piece of —CH₂— may be replaced by —O—, and at least one hydrogen may be replaced by fluorine;

X¹¹ is fluorine, chlorine, —OCF₃, —OCHF₂, —CF₃, —CHF₂, —CH₂F, —OCF₂CHF₂ or —OCF₂CHFCF₃;

ring C¹, ring C² and ring C³ are independently 1,4-cyclohexylene, 1,4-phenylene in which at least one hydrogen may be replaced by fluorine, tetrahydropyran-2,5-diyl, 1,3-dioxane-2,5-diyl or pyrimidine-2,5-diyl;

Z¹⁴, Z¹⁵ and Z¹⁶ are independently a single bond, —CH₂CH₂—, —CH═CH—, —C≡C—, —COO—, —CF₂O—, —OCF₂—, —CH₂O—, —CF═CF—, —CH═CF— or —(CH₂)₄—; and

L¹¹ and L¹² are independently hydrogen or fluorine.

Item 12. The liquid crystal composition according to any one of items 9 to 11, further containing at least one compound of compounds represented by formula (8):

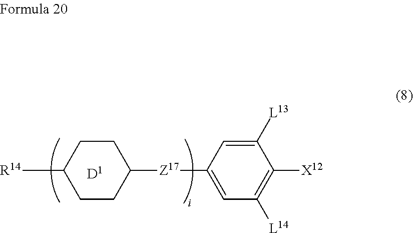

wherein, in formula (8),

R¹⁴ is alkyl having 1 to 10 carbons or alkenyl having 2 to 10 carbons, and in the alkyl and the alkenyl, at least one piece of —CH₂— may be replaced by —O—, and at least one hydrogen may be replaced by fluorine;

X¹² is —C≡N or —C≡C—C≡N;

ring D¹ is 1,4-cyclohexylene, 1,4-phenylene in which at least one hydrogen may be replaced by fluorine, tetrahydropyran-2,5-diyl, 1,3-dioxane-2,5-diyl or pyrimidine-2,5-diyl;

Z¹⁷ is a single bond, —CH₂CH₂—, —C≡C—, —COO—, —CF₂O—, —OCF₂— or —CH₂O—;

L¹³ and L¹⁴ are independently hydrogen or fluorine; and

i is 1, 2, 3 or 4.

Item 13. The liquid crystal composition according to any one of items 9 to 12, further containing at least one compound selected from the group of compounds represented by formula (9) to formula (15):

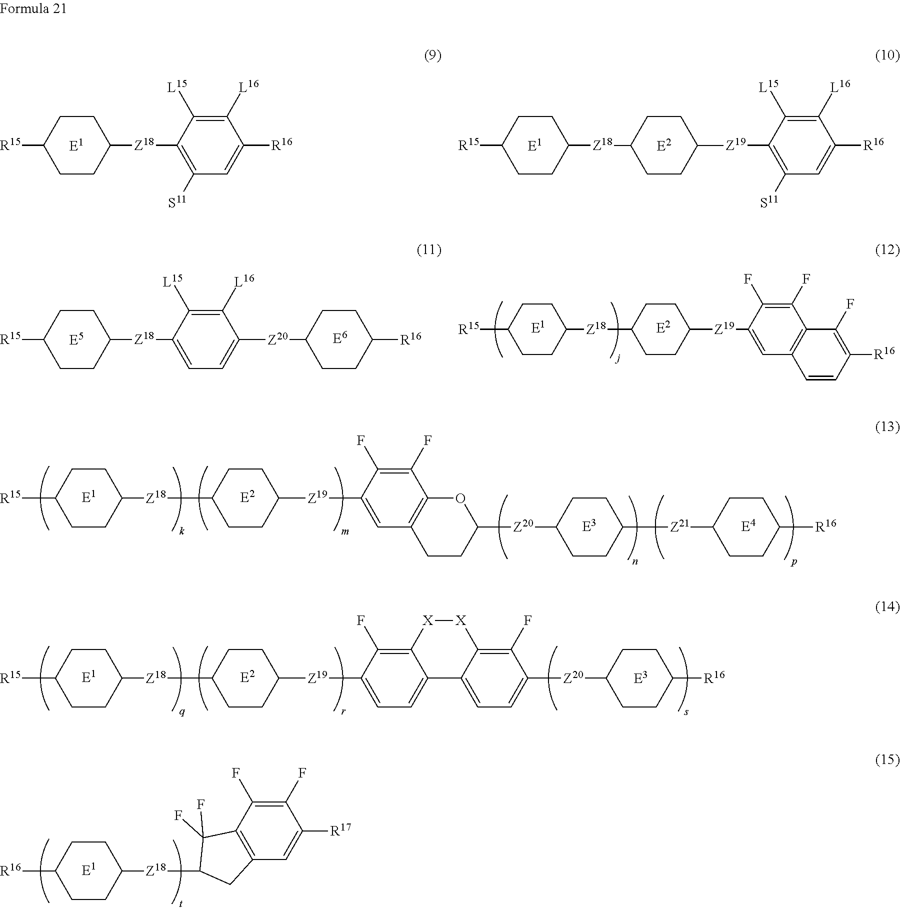

wherein, in formula (9) to formula (15),

R¹⁵ and R¹⁶ are independently alkyl having 1 to 10 carbons or alkenyl having 2 to 10 carbons, and in the alkyl and the alkenyl, at least one piece of —CH₂— may be replaced by —O—, and at least one hydrogen may be replaced by fluorine;

R¹⁷ is hydrogen, fluorine, alkyl having 1 to 10 carbons or alkenyl having 2 to 10 carbons, and in the alkyl and the alkenyl, at least one piece or —CH₂— may be replaced by —O—, and at least one hydrogen may be replaced by fluorine;

ring E¹, ring E², ring E³ and ring E⁴ are independently 1,4-cyclohexylene, 1,4-cyclohexenylene, 1,4-phenylene in which at least one hydrogen may be replaced by fluorine, tetrahydropyran-2,5-diyl or decahydronaphthalene-2, 6-diyl;

ring E⁵ and ring E⁶ are independently 1,4-cyclohexylene, 1,4-cyclohexenylene, 1,4-phenylene, tetrahydropyran-2,5-diyl or decahydronaphthalene-2,6-diyl;

Z¹⁸, Z¹⁹, Z²⁰ and Z²¹ are independently a single bond, —CH₂CH₂—, —COO—, —CH₂O—, —OCF₂— or —OCF₂CH₂CH₂—;

L¹⁵ and L¹⁶ are independently fluorine or chlorine;

S¹¹ is hydrogen or methyl;

X is —CHF— or —CF₂—; and

j, k, m, n, p, q, r and s are independently 0 or 1, a sum of k, m, n and p is 1 or 2, a sum of q, r and s is 0, 1, 2 or 3, and t is 1, 2 or 3.

Item 14. The liquid crystal composition according to any one of items 9 to 13, containing at least one polymerizable compound of compounds represented by formula (16):

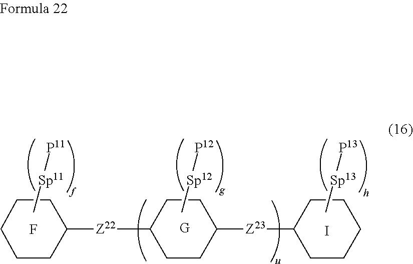

wherein, in formula (16),

ring F and ring I are independently cyclohexyl, cyclohexenyl, phenyl, 1-naphthyl, 2-naphthyl, tetrahydropyran-2-yl, 1,3-dioxane-2-yl, pyrimidine-2-yl or pyridine-2-yl, and in the rings, at least one hydrogen may be replaced by halogen, alkyl having 1 to 12 carbons, or alkyl having 1 to 12 carbons in which at least one hydrogen is replaced by halogen;

ring G is 1,4-cyclohexylene, 1,4-cyclohexenylene, 1,4-phenylene, naphthalene-1,2-diyl, naphthalene-1,3-diyl, naphthalene-1,4-diyl, naphthalene-1,5-diyl, naphthalene-1,6-diyl, naphthalene-1,7-diyl, naphthalene-1,8-diyl, naphthalene-2,3-diyl, naphthalene-2,6-diyl, naphthalene-2,7-diyl, tetrahydropyran-2,5-diyl, 1,3-dioxane-2,5-diyl, pyrimidine-2,5-diyl or pyridine-2,5-diyl, and in the rings, at least one hydrogen may be replaced by halogen, alkyl having 1 to 12 carbons, alkoxy having 1 to 12 carbons, or alkyl having 1 to 12 carbons in which at least one hydrogen is replaced by halogen;

Z²² and Z²³ are independently a single bond or alkylene having 1 to 10 carbons, and in the alkylene, at least one piece of —CH₂— may be replaced by —O—, —CO—, —COO— or —OCO—, and at least one piece of —CH₂CH₂— may be replaced by —CH═CH—, —C(CH₃)═CH—, —CH═C(CH₃)— or —C(CH₃)═C(CH₃)—, and in the groups, at least one hydrogen may be replaced by fluorine or chlorine; and

P¹¹, P¹² and P¹³ are independently a polymerizable group selected from the group of groups represented by formula (P-1) to formula (P-5);

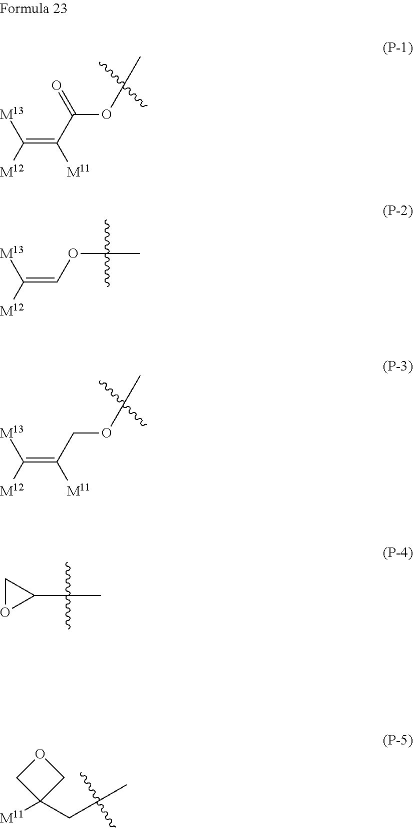

wherein

M¹¹, M¹² and M¹³ are independently hydrogen, fluorine, alkyl having 1 to 5 carbons, or alkyl having 1 to 5 carbons in which at least one hydrogen is replaced by fluorine or chlorine;

Sp¹¹, SP¹² and Sp¹³ are independently a single bond or alkylene having 1 to 10 carbons, and in the alkylene, at least one piece of —CH₂— may be replaced by —O—, —COO—, —OCO— or —OCOO—, and at least one piece of —CH₂CH₂— may be replaced by —CH═CH— or —C≡C—, and in the groups, at least one hydrogen may be replaced by fluorine or chlorine;

u is 0, 1 or 2; and

f, g and h are independently 0, 1, 2, 3 or 4, and a sum of f, g and h is 2 or more.

Item 15. The liquid crystal composition according to any one of items 9 to 14, containing at least one polymerizable compound selected from the group of compounds represented by formula (16-1) to formula (16-27):

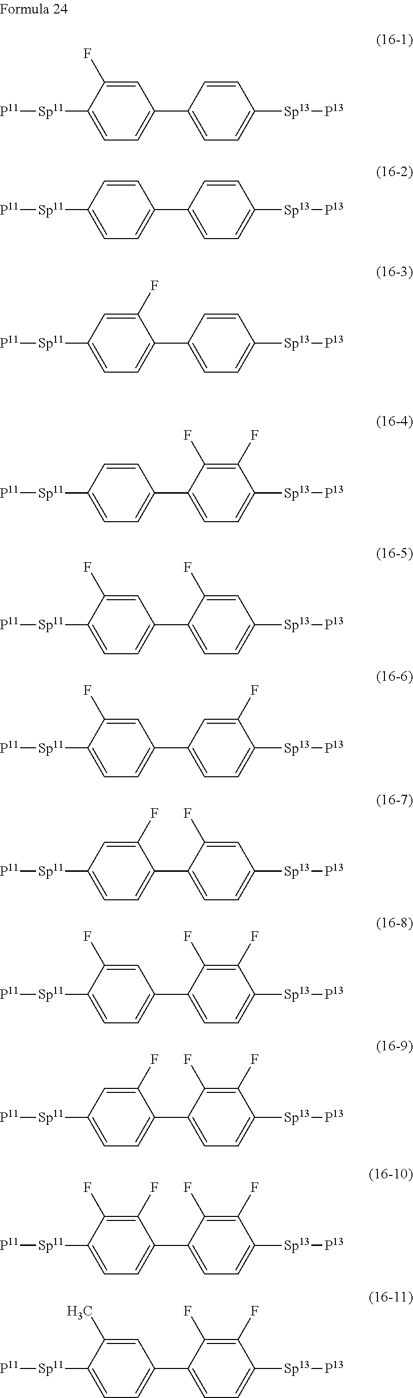

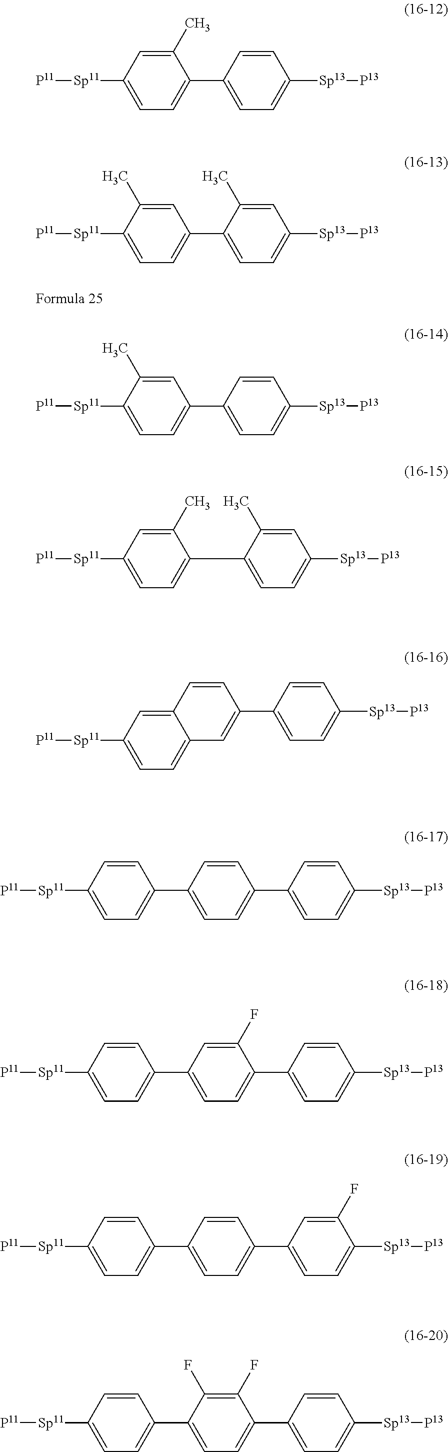

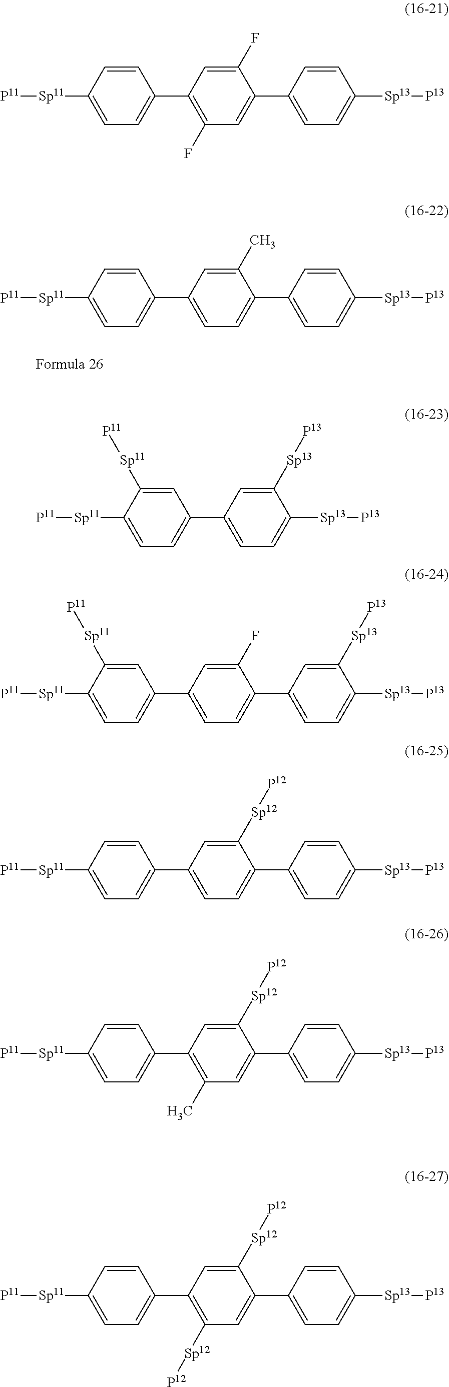

wherein, in formula (16-1) to formula (16-27),

P¹¹, P¹² and P¹³ are independently a polymerizable group selected from the group of groups represented by formula (P-1) to formula (P-3), in which M¹¹, M¹² and M¹³ are independently hydrogen, fluorine, alkyl having 1 to 5 carbons, or alkyl saving 1 to 5 carbons in which at least one hydrogen is replaced by halogen;

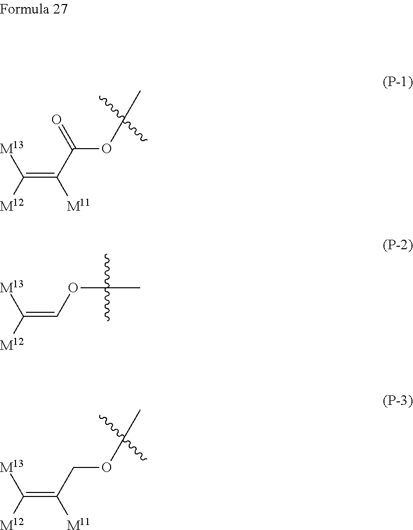

wherein

Sp¹¹, Sp¹² and Sp¹³ are independently a single bond or alkylene having 1 to 10 carbons, and in the alkylene, at least one piece of —CH₂— may be replaced by —O—, —COO—, —OCO—, or —OCOO—, and at least one piece of —CH₂CH₂— may be replaced by —CH═CH— or —C≡C—, and in the groups, at least one hydrogen may be replaced by fluorine or chlorine.

Item 16. The liquid crystal composition according to any one of items 9 to 15, further containing at least one of a polymerizable compound other than formula (1) and formula (16), a polymerization initiator, a polymerization inhibitor, an optically active compound, an antioxidant, an ultraviolet light absorber, a light stabilizer, a heat stabilizer and an antifoaming agent.

Item 17. A liquid crystal display device, including the liquid crystal composition according to any one of items 9 to 16.

The invention further includes the following items: (a) the liquid crystal composition, further containing at least two of additives such as a polymerizable compound, a polymerization initiator, a polymerization inhibitor, an optically active compound, an antioxidant, an ultraviolet light absorber, a light stabilizer, a heat stabilizer and an antifoaming agent; (b) a polymerizable composition, prepared by adding a polymerizable compound different from compound (1) or compound (16) to the liquid crystal composition; (c) a polymerizable composition, prepared by adding compound (1) and compound (16) to the liquid crystal composition; (d) a liquid crystal composite, prepared by polymerizing a polymerizable composition; (e) a polymer sustained alignment mode device, including the liquid crystal composite; and (f) a polymer sustained alignment mode device, prepared by using a polymerizable composition prepared by adding compound (1), compound (16) and a polymerizable compound different from compound (1) or compound (16) to the liquid crystal composition.

An aspect of compound (1), synthesis of compound (1), the liquid crystal composition and the liquid crystal display device will be described in the order.

### 1. Aspect of Compound (1)

Compound (1) according to an embodiment of the invention has features of being a polar compound having a mesogen moiety formed of at least one ring, and a plurality of kinds of polymerizable groups. Compound (1) has the plurality of kinds of polymerizable groups to further facilitate to adjust characteristics, as compared to a compound having one kind of polymerizable group. One of applications is an additive for the liquid crystal composition used in the liquid crystal display device. Compound (1) is added for the purpose of horizontally controlling alignment of liquid crystal molecules. Such an additive preferably has chemically high stability under conditions in which the additive is sealed in the device, high solubility in the liquid crystal composition, and a large voltage holding ratio when the additive is used in the liquid crystal display device. Compound (1) satisfies such characteristics to a significant extent.

Preferred examples of compound (1) will be described. Preferred examples of R¹, Z¹ to Z⁵, A¹ to A⁴, Sp¹, Sp², P¹, P², a and b in compound (1) apply also to a subordinate formula of formula (1) for compound (1). In compound (1), the characteristics can be arbitrarily adjusted by suitably combining kinds of the groups. Compound (1) may contain a larger amount of isotope such as ²H (deuterium) and ¹³C than an amount of natural abundance because no significant difference is caused in the characteristics of the compound.

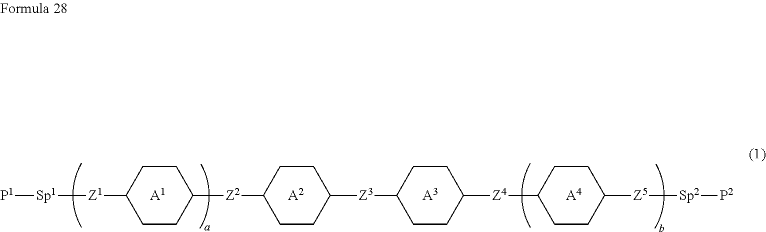

Ring A¹, A², A³ and A⁴ are independently 1,4-cyclohexylene, 1,4-cyclohexenylene, 1,4-phenylene, naphthalene-2,6-diyl, decahydronaphthalene-2,6-diyl, 1,2,3,4-tetrahydronaphthalene-2,6-diyl, tetrahydropyran-2,5-diyl, 1,3-dioxane-2,5-diyl, pyrimidine-2,5-diyl, pyridine-2,5-diyl, fluorene-2,7-diyl, phenanthrene-2,7-diyl, anthracene-2,6-diyl, perhydrocyclopenta[a]phenanthrene-3,17-diyl or 2,3,4,7,8,9,10,11,12,13,14,15,16,17-tetradecahydrocyclopenta[a]phenanthrene-3,17-diyl, and in the rings, at least one hydrogen may be replaced by fluorine, chlorine, alkyl having 1 to 12 carbons, alkenyl having 2 to 12 carbons, alkoxy having 1 to 11 carbons, alkenyloxy having 2 to 11 carbons, -Sp¹-P¹ or -Sp²-P², and in the groups, at least one hydrogen may be replaced by fluorine or chlorine, when a is 2, two of ring A¹ may be different, and when b is 2, two of ring A⁴ may be different.

Preferred ring A¹, A², A³ and A⁴ are independently 1,4-cyclohexylene, 1,4-cyclohexenylene, 1,4-phenylene, naphthalene-2,6-diyl, decahydronaphthalene-2,6-diyl, 1,2,3,4-tetrahydronaphthalene-2,6-diyl, tetrahydropyran-2,5-diyl, 1,3-dioxane-2,5-diyl, pyrimidine-2,5-diyl, pyridine-2,5-diyl, perhydrocyclopenta[a]phenanthrene-3,17-diyl or 2,3,4,7,8,9,10,11,12,13,14,15,16,17-tetradecahydrocyclopenta[a]phenanthrene-3,17-diyl, and in the rings, at least one hydrogen may be replaced by fluorine, chlorine, alkyl having 1 to 12 carbons, alkenyl having 2 to 12 carbons, alkoxy having 1 to 11 carbons or alkenyloxy having 2 to 11 carbons, and in the groups, at least one hydrogen may be replaced by fluorine or chlorine. Further preferred ring A¹, A², A³ and A⁴ are 1,4-cyclohexylene, 1,4-phenylene, perhydrocyclopenta[a]phenanthrene-3,17-diyl or 2,3,4,7,8,9,10,11,12,13,14,15,16,17-tetradecahydrocyclopenta[a]phenanthrene-3,17-diyl, and in the rings, at least one hydrogen may be replaced by fluorine or alkyl having 1 to 5 carbons. Particularly preferred ring A¹, A², A³ and A⁴ are 1,4-cyclohexylene, 1,4-phenylene or perhydrocyclopenta[a]phenanthrene-3,17-diyl, and in the rings, at least one hydrogen may be replaced by fluorine, methyl or ethyl.

Z¹, Z², Z³, Z⁴ and Z⁵ are independently a single bond or alkylene having 1 to 10 carbons, and in the alkylene, at least one piece of —CH₂— may be replaced by —O—, —CO—, —COO—, —OCO— or —OCOO—, and at least one piece of —(CH₂)₂— may be replaced by —CH═CH— or —C≡C— and in the groups, at least one hydrogen may be replaced by halogen, in which at least one in Z², Z³ or Z⁴ is any one of —COO—, —OCO—, —CH═CHCOO—, —OCOCH═CH—, —CH═CH—, —CH═CHCO— and —COCH═CH—, and when a is 2, two of Z¹ may be different, and two of Z⁵ may be different.

Preferred Z¹, Z², Z³, Z⁴ and Z⁵ are independently a single bond, —(CH₂)₂—, —CH═CH—, —C≡C—, —COO—, —OCO—, —CF₂O—, —OCF₂—, —CH₂O—, —OCH₂— or —CF═CF—. Further preferred Z¹, Z², Z³, Z⁴ and Z⁵ are a single bond, —(CH₂)₂— or —CH═CH—. Particularly preferred Z¹, Z², Z³, Z⁴ and Z³ are a single bond.

Sp¹ and Sp² are independently a single bond or alkylene having 1 to 10 carbons, and in the alkylene, at least one piece of —CH₂— may be replaced by —O—, —CO—, —COO—, —OCO— or —OCOO—, and at least one piece of —(CH₂)₂— may be replaced by —CH═CH— or —C≡C—, and in the groups, at least one hydrogen may be replaced by halogen.

Preferred Sp¹ and Sp² are independently a single bond, alkylene having 1 to 6 carbons, alkylene having 1 to 6 carbons in which one piece of —CH₂— is replaced by —O—, or —OCOO—. Further preferred Sp¹ and Sp² are alkylene having 1 to 6 carbons or —OCOO—.

P¹ and P² are independently a group represented by any one of formula (1b) to formula (1h).

Preferred P¹ and P² are independently (1b), (1c), (1d) and (1e).

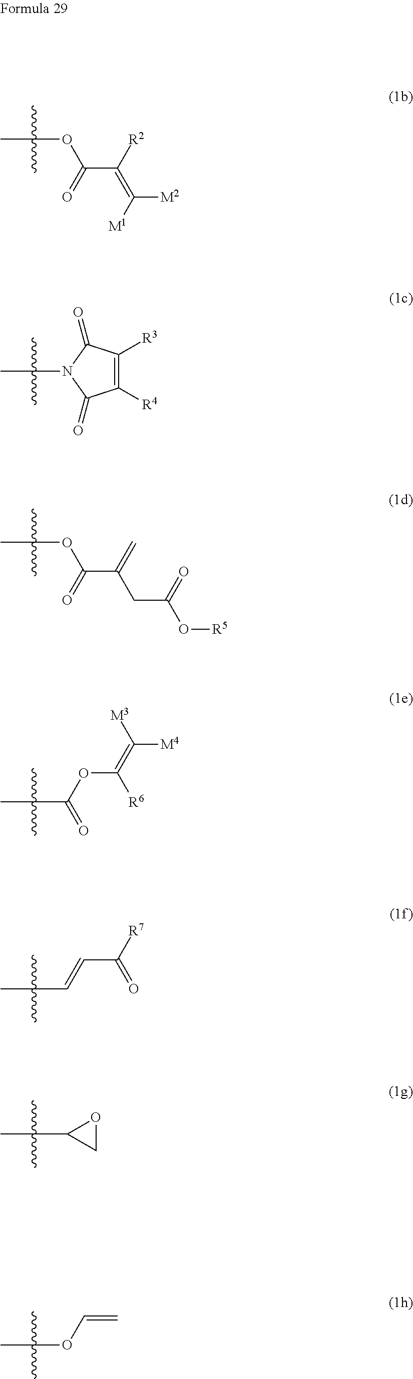

A further preferred group is a group represented by formula (1b-1), (1b-2), (1b-3), (1b-4), (1b-5), (1c-1), (1d-1), (1d-2) or (1e-1).

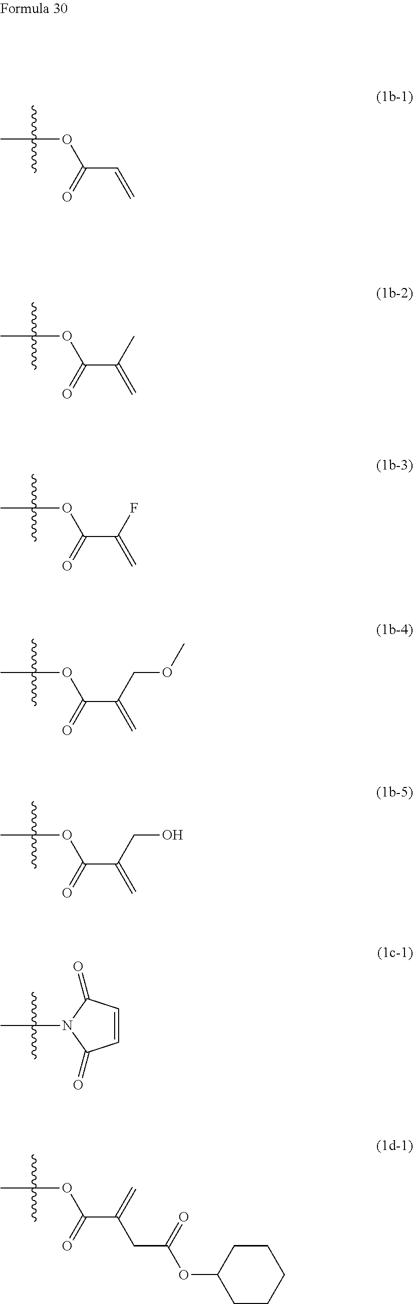

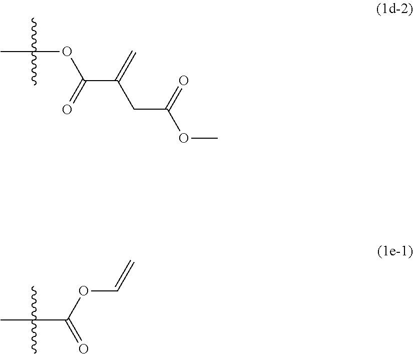

In formula (1b) to formula (1h), M¹ and M² are independently hydrogen, halogen, alkyl having 1 to 5 carbons, or alkyl having 1 to 5 carbons in which at least one hydrogen is replaced by halogen.

Preferred M¹, M², M³ and M⁴ are independently hydrogen, fluorine, methyl, ethyl or trifluoromethyl. Further preferred M¹, M², M³ and M⁴ are hydrogen.

R² is hydrogen, halogen or alkyl having 1 to 5 carbons, and in the alkyl, at least one hydrogen may be replaced by halogen, and at least one piece of —CH₂— may be replaced by —O—.

Preferred R² is hydrogen, fluorine, methyl, ethyl, methoxymethyl or trifluoromethyl. Further preferred R² is hydrogen.

R³, R⁴, R⁵, R⁶ and R⁷ are independently hydrogen, or straight-chain alkyl, branched-chain alkyl or cyclic alkyl having 1 to 15 carbons, and in the alkyl, at least one piece of —CH₂— may be replaced by —O— or —S—, and at least one piece of —(CH₂)₂— may be replaced by —CH═CH— or —C≡C—, and in the groups, at least one hydrogen may be replaced by halogen.

Preferred R³, R⁴, R⁵, R⁶ and R⁷ are independently hydrogen, straight-chain alkyl having 1 to 10 carbons, straight-chain alkenyl having 2 to 10 carbons, straight-chain alkoxy having 1 to 10 carbons, or cyclic alkyl having 3 to 6 carbons. Further Preferred R³, R⁴, R⁵, R⁶ and R⁷ are hydrogen, straight-chain alkyl having 2 to 6 carbons, straight-chain alkenyl having 2 to 6 carbons, straight-chain alkoxy having 1 to 5 carbons, or cyclic alkyl having 4 to 6 carbons.

Expressions: 0≤a+b≤2 preferably hold.

Preferred examples of compound (1) include formulas (1-1) to (1-3).

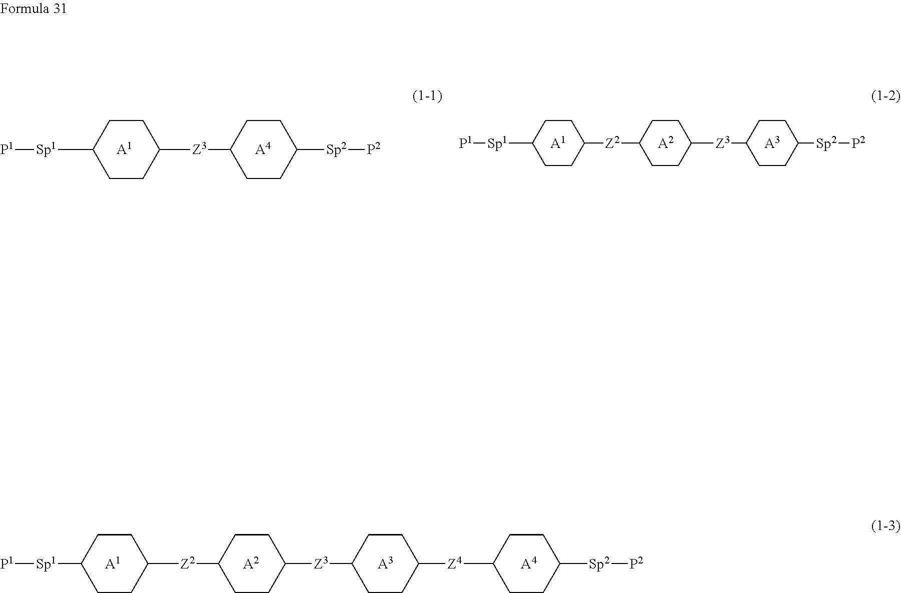

In formula (1-1) to formula (1-3),

ring A¹, ring A², ring A³ and ring A⁴ are independently 1,4-cyclohexylene, 1,4-phenylene, naphthalene-2,6-diyl, pyrimidine-2,5-diyl, pyridine-2,5-diyl, fluorene-2,7-diyl, phenanthrene-2,7-diyl or anthracene-2,6-diyl, and in the rings, at least one hydrogen may be replaced by fluorine, chlorine, alkyl having 1 to 12 carbons, alkenyl having 2 to 12 carbons, alkoxy having 1 to 11 carbons, alkenyloxy having 2 to 11 carbons, -Sp¹-P¹ or -Sp²-P², and in the groups, at least one hydrogen may be replaced by fluorine or chlorine;

Z², Z³ and Z⁴ are independently a single bond, —(CH₂)₂—, —CH═CH—, —C≡C—, —COO—, —OCO—, —CF₂O—, —OCF₂—, —CH₂O—, —OCH₂—, —CF═CF—, —CH═CHCOO—, —OCOCH═CH—, —CH═CHCO— or —COCH═CH—, in which at least one in Z², Z³ and Z⁴ is —COO—, —OCO—, —CH═CHCOO—, —OCOCH═CH—, —CH═CH—, —CH═CHCO— or —COCH═CH—;

Sp¹ and Sp² are independently a single bond or alkylene having 1 to 10 carbons, and in the alkylene, at least one piece of —CH₂— may be replaced by —O—, —COO—, —OCOO— or —OCO—, and at least one piece of —(CH₂)₂— may be replaced by —CH═CH—, and in the groups, at least one hydrogen may be replaced by fluorine or chlorine, and when a plurality of Sp¹ or Sp² are present in a structure, the plurality each may be different; and

P¹ and P² are independently a group represented by any one of formula (1b) to formula (1h), and when a plurality of P¹ or P² are present in a structure, the plurality each may be different, in which a case where all of P¹ and P² have an identical structure is excluded.

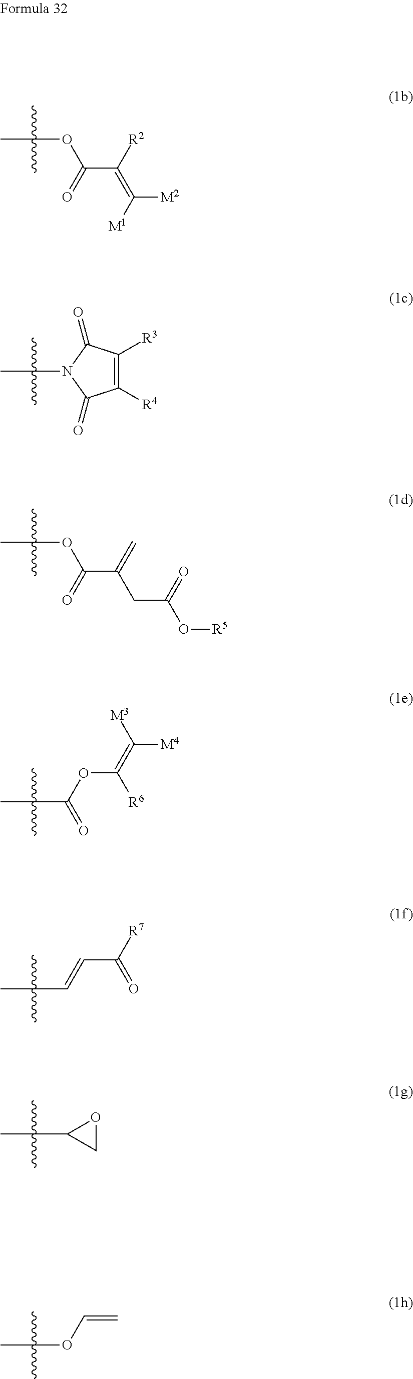

M¹, M², M³ and M⁴ are independently hydrogen, halogen, alkyl having 1 to 5 carbons, or alkyl having 1 to 5 carbons in which at least one hydrogen is replaced by halogen;

R² is hydrogen, halogen or alkyl having 1 to 5 carbons, and in the alkyl, at least one hydrogen may be replaced by halogen, and at least one piece of —CH₂— may be replaced by —O—; and

R³, R⁴, R⁵, R⁶ and R⁷ are independently hydrogen or alkyl having 1 to 15 carbons, and in the alkyl, at least one piece of —CH₂— may be replaced by —O— or —S—, and at least one piece of —(CH₂)₂— may be replaced by —CH═CH— or —C≡C—, and in the groups, at least one hydrogen may be replaced by halogen.

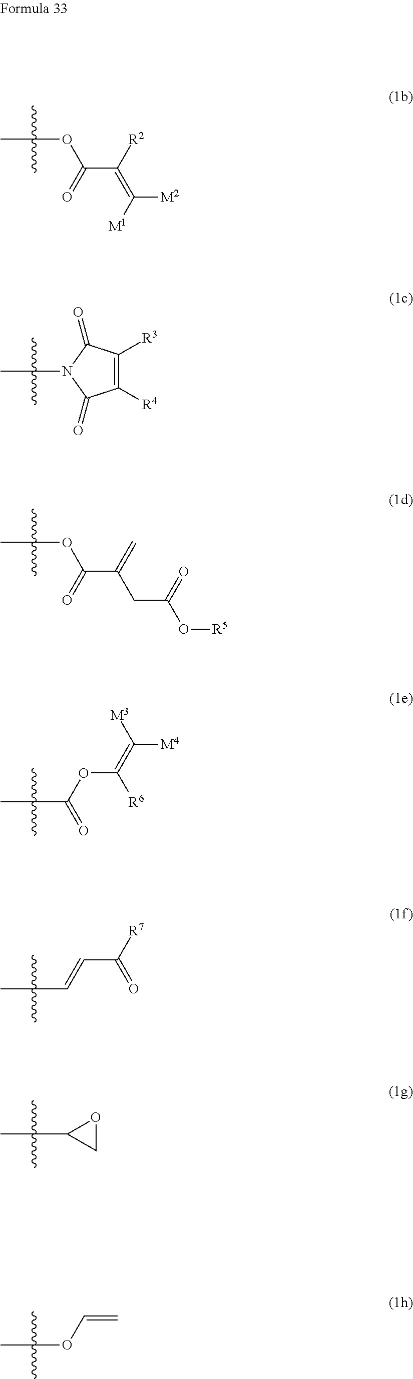

In formula (1b) to formula (1h),

M¹, M², M³ and M⁴ are independently hydrogen, halogen, alkyl having 1 to 5 carbons, or alkyl having 1 to 5 carbons in which at least one hydrogen is replaced by halogen;

R² is hydrogen, halogen or alkyl having 1 to 5 carbons, and in the alkyl, at least one hydrogen may be replaced by halogen, and at least one piece of —CH₂— may be replaced by —O—; and

R³, R⁴, R⁵, R⁶ and R⁷ are independently hydrogen or alkyl having 1 to 15 carbons, and in the alkyl, at least one piece of —CH₂— may be replaced by —O— or —S—, and at least one piece of —(CH₂)₂— may be replaced by —CH═CH— or —C≡C—, and in the groups, at least one hydrogen may be replaced by halogen.

In the compound represented by formula (1-1), formula (1-2) or formula (1-3) any one of Z², Z³ or Z⁴ is preferably —COO— or —OCO—.

Moreover, in the compound represented by formula (1-1), formula (1-2) or formula (1-3), any one of Z², Z³ or Z⁴ is preferably —CH═CHCOO—, —OCOCH═CH—, —CH═CH—, —CH═CHCO— or —COCH═CH—.

Compound (1) is preferably a compound represented by formula (1-A).

Formula 34

P¹-Sp¹-Y-Sp²-P² (1-A)

In formula (1-A),

P¹ and P² are independently a group represented by formula (1b-1), (1b-2), (1b-3), (1b-4), (1b-5), (1c-1), (1d-1), (1d-2) or (1e-1), in which a case where all of P¹ and P² have an identical structure is excluded, and a case where P¹ and P² are in a combination of only formulas (1b-1) and (1b-2) is excluded;

Sp¹ and Sp² are independently a single bond or alkylene having 1 to 10 carbons, and in the alkylene, at least one piece of —CH₂— may be replaced by —O—, —COO—, —OCOO— or —OCO—, and at least one piece of —(CH₂)₂— may be replaced by —CH═CH—.

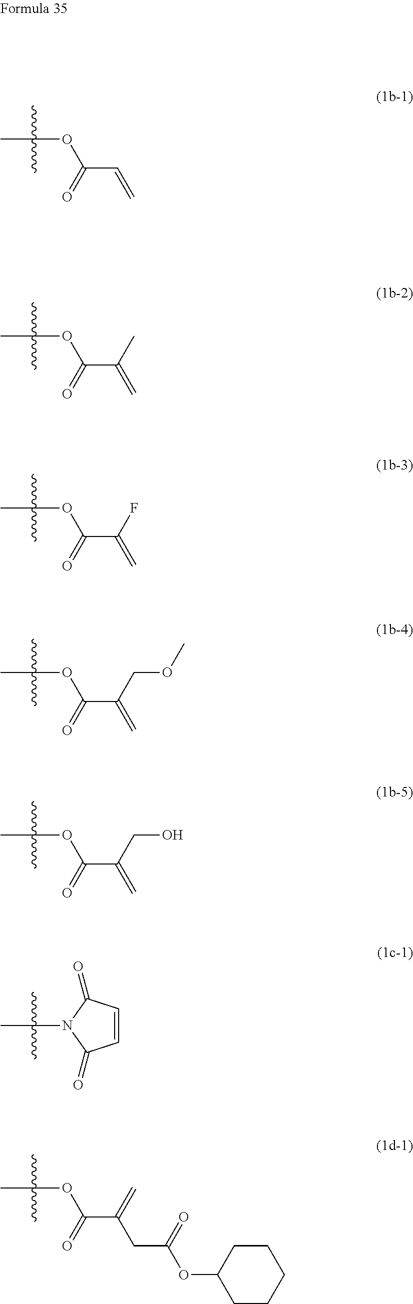

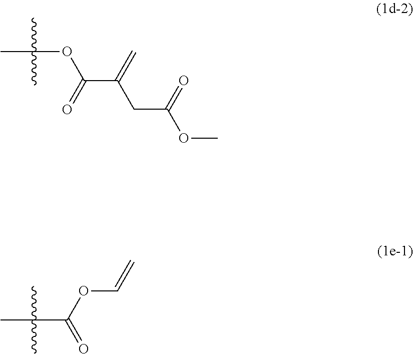

Y is a group represented by any one of formulas (MES-1-01) to (MES-1-10).

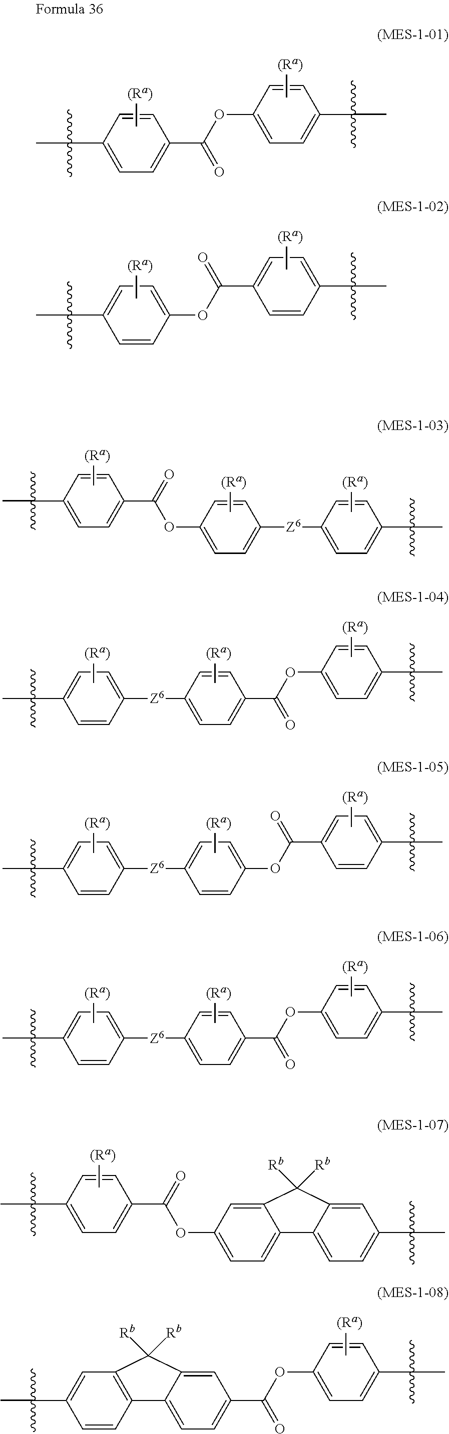

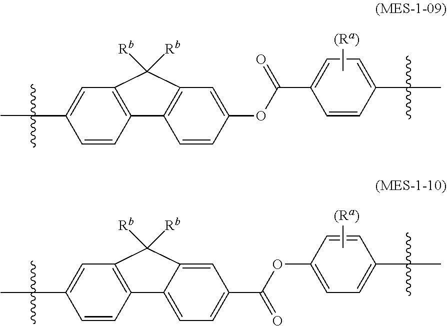

R^{a} is independently fluorine, chlorine, methyl or ethyl;

R^{b} is independently hydrogen, fluorine, methyl or ethyl;

Z⁶ is independently a single bond or —C≡C—; and

a representation formed by connecting 1,4-phenylene with (R^{a}) by a straight line as shown below in the formulas indicates 1,4-phenylene in which one or two hydrogens may be replaced by R^{a}.

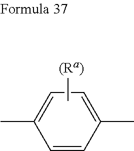

In another aspect of formula (1-A), P¹ and P² are independently a group represented by formula (1b-1), (1b-2), (1b-3), (1b-4), (1b-5), (1c-1), (1d-1), (1d-2) or (1e-1), in which a case where P¹ and P² have an identical structure is excluded, and a case where P¹ and P² are in a combination of only formulas (1b-1) and (1b-2) is excluded.

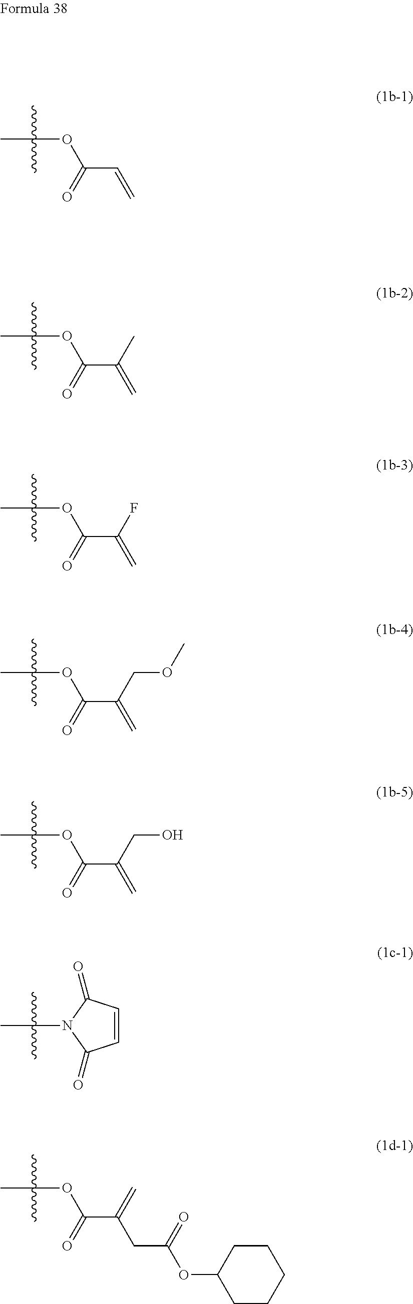

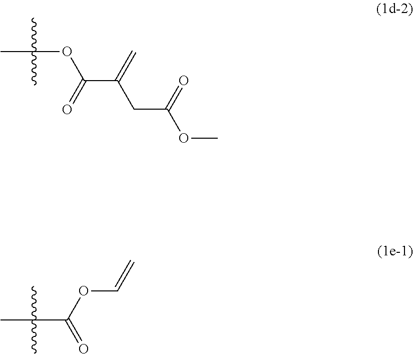

Sp¹ and Sp² are independently a single bond or alkylene having 1 to 10 carbons, and in the alkylene, at least one piece of —CH₂— may be replaced by —O—, —COO—, —OCOO— or —OCO—, and at least one piece of —(CH₂)₂— may be replaced by —CH═CH—; and

Y is a group represented by any one of (MES-2-01) to (MES-2-15).

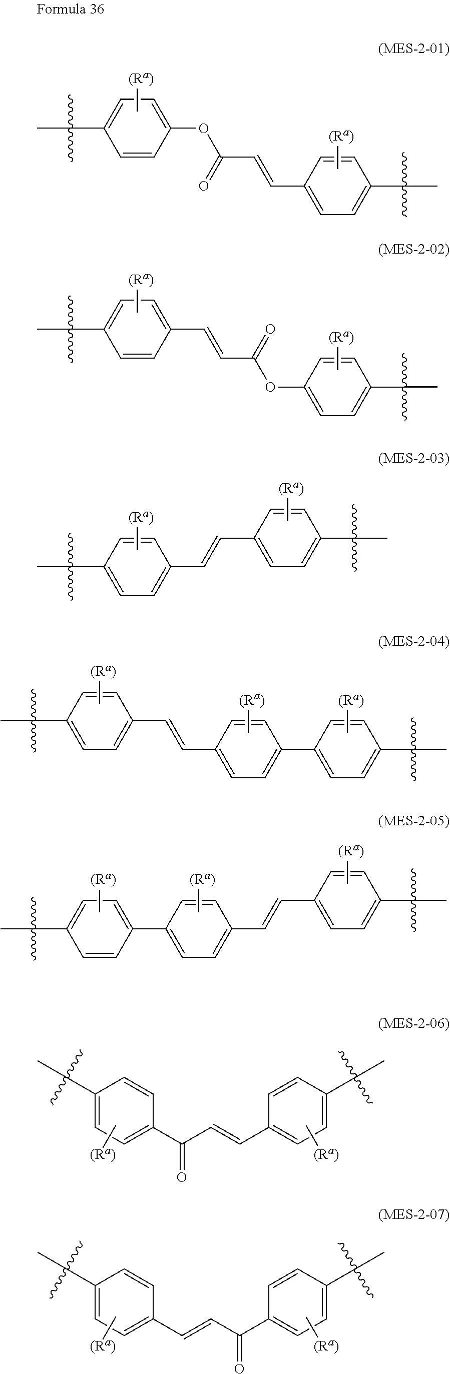

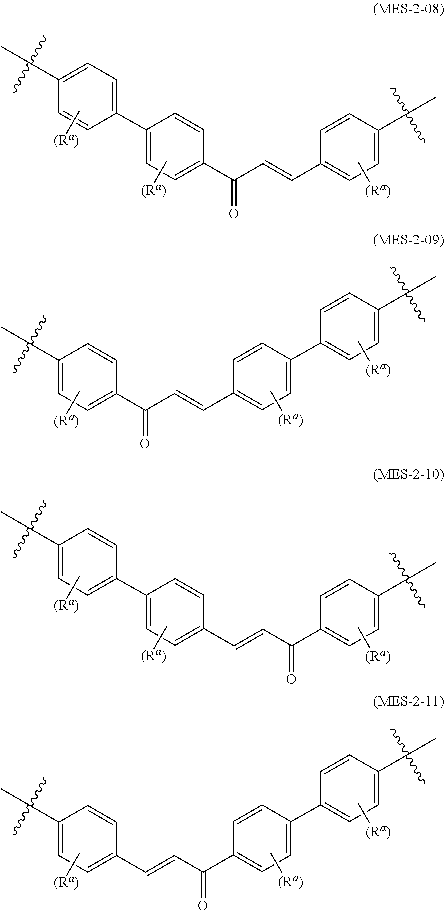

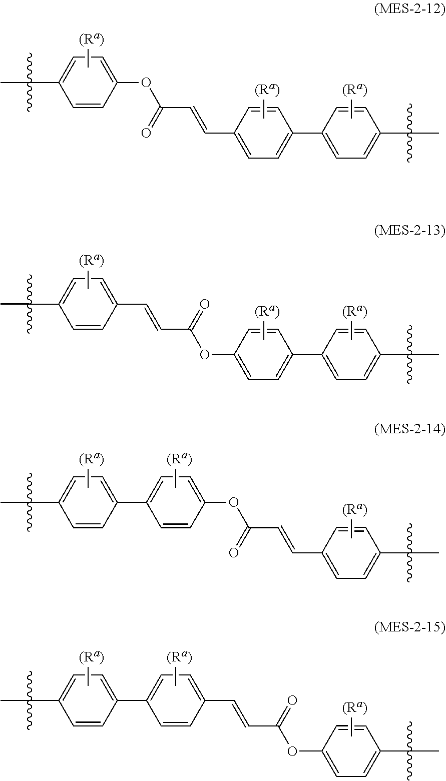

R^{a} is independently fluorine, chlorine, methyl or ethyl; and

a representation formed by connecting 1,4-phenylene with (R^{a}) by a straight line as shown below in the formulas indicates 1,4-phenylene in which one or two hydrogens may be replaced by R^{a}.

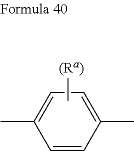

In addition, specific examples of compound (1) will be described in Examples described below.

Formulas (2) to (15) show a component compound of the liquid crystal composition. Compounds (2) to (4) have small dielectric anisotropy. Compounds (5) to (7) have large positive dielectric anisotropy. Compound (8) has a cyano group, and therefore has larger positive dielectric anisotropy. Compounds (9) to (16) have large negative dielectric anisotropy. Specific examples of the compounds will be described later.

In compound (16), P¹¹, P¹² and P¹³ are independently a polymerizable group.

Preferred P¹¹, P¹² and P¹³ are a polymerizable group selected from the group of groups represented by formula (P-1) to formula (P-5). Further preferred P¹¹, P¹² and P¹³ are group (P-1), group (P-2) or group (P-3). Particularly preferred group (P-1) is —OCO—CH═CH₂ or —OCO—C(CH₃)═CH₂. A wavy line in group (P-1) to group (P-5) represents a site to form a bonding.

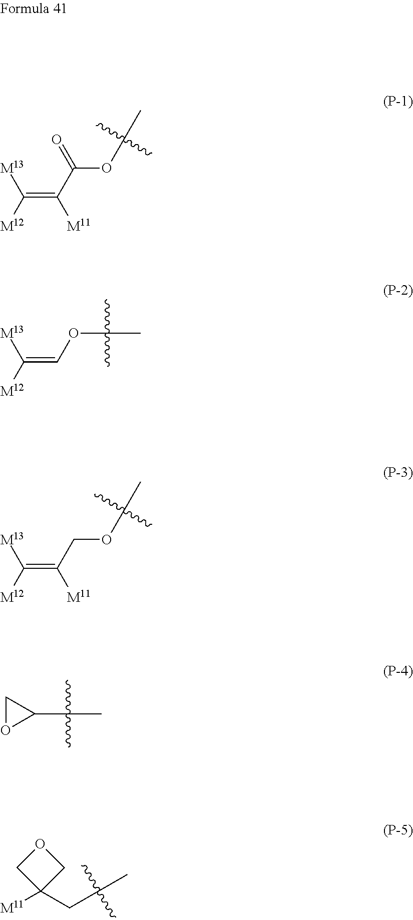

In group (P-1) to group (P-5), M¹¹, M¹² and M¹³ are independently hydrogen, fluorine, alkyl having 1 to 5 carbons, or alkyl having 1 to 5 carbons in which at least one hydrogen is replaced by halogen.

Preferred M¹¹, M¹² and M¹³ are hydrogen or methyl for increasing reactivity. Further preferred M¹¹ is methyl, and further preferred M¹² and M¹³ are hydrogen.

Sp¹¹, Sp¹² and Sp¹³ are independently a single bond or alkylene having 1 to 10 carbons, and in the alkylene, at least one piece of —CH₂— may be replaced by —O—, —COO—, —OCO— or —OCOO—, and at least one piece of —CH₂CH₂— may be replaced by —CH═CH— or —C≡C—, and in the groups, at least one hydrogen may be replaced by fluorine or chlorine.

Preferred Sp¹¹, Sp¹² and Sp¹³ are a single bond.

Ring F and ring I are independently cyclohexyl, cyclohexenyl, phenyl, 1-naphthyl, 2-naphthyl, tetrahydropyran-2-yl, 1,3-dioxane-2-yl, pyrimidine-2-yl or pyridine-2-yl, and in the rings, at least one hydrogen may be replaced by halogen, alkyl having 1 to 12 carbons, alkoxy having 1 to 12 carbons, or alkyl having 1 to 12 carbons in which at least one hydrogen is replaced by halogen.

Preferred ring F and ring I are phenyl. Ring G is 1,4-cyclohexylene, 1,4-cyclohexenylene, 1,4-phenylene, naphthalene-1,2-diyl, naphthalene-1,3-diyl, naphthalene-1,4-diyl, naphthalene-1,5-diyl, naphthalene-1,6-diyl, naphthalene-1,7-diyl, naphthalene-1,8-diyl, naphthalene-2,3-diyl, naphthalene-2,6-diyl, naphthalene-2,7-diyl, tetrahydropyran-2,5-diyl, 1,3-dioxane-2,5-diyl, pyrimidine-2,5-diyl or pyridine-2,5-diyl, and in the rings, at least one hydrogen may be replaced by halogen, alkyl having 1 to 12 carbons, alkoxy having 1 to 12 carbons, or alkyl having 1 to 12 carbons in which at least one hydrogen is replaced by halogen. Particularly preferred ring G is 1,4-phenylene or 2-fluoro-1,4-phenylene.

Z²² and Z²³ are independently a single bond or alkylene having 1 to 10 carbons, and in the alkylene, at least one piece of —CH₂— may be replaced by —O—, —CO—, —COO— or —OCO—, and at least one piece of —CH₂CH₂— may be replaced by —CH═CH—, —C(CH₃)═CH—, —CH═C(CH₃)— or —C(CH₃)═C(CH₃)—, and in the groups, at least one hydrogen may be replaced by fluorine or chlorine.

Preferred Z²² and Z²³ are a single bond, —CH₂CH₂—, —CH₂O—, —OCH₂—, —COO— or —OCO—. Further preferred Z²² and Z²³ are a single bond.

Then, u is 0, 1 or 2.

Preferred u is 0 or 1. Then, f, g and h are independently 0, 1, 2, 3 or 4, and a sum of f, g and h is 1 or more. Preferred f, g or h is 1 or 2.

### 2. Synthesis of Compound (1)

A synthesis method of compound (1) will be described. Compound (1) can be prepared by suitably combining methods in synthetic organic chemistry. Any compounds whose synthesis methods are not described above are prepared according to methods described in books such as “Organic Syntheses” (John Wiley & Sons, Inc.), “Organic Reactions” (John Wiley & Sons, Inc.), “Comprehensive Organic Synthesis” (Pergamon Press) and “New Experimental Chemistry Course (Shin Jikken Kagaku Koza in Japanese)” (Maruzen Co., Ltd.).

2-1. Formation of Bonding Groups Z¹, Z², Z³, Z⁴ and Z⁵

An example of a method for forming a bonding group in compound (1) is as described in a scheme below. In the scheme, MSG¹ (or MSG²) is a monovalent organic group having at least one ring. Monovalent organic groups represented by a plurality of MSG¹ (or MSG²) may be identical or different. Compounds (1A) to (1J) correspond to compound (1) or an intermediate of compound (1).

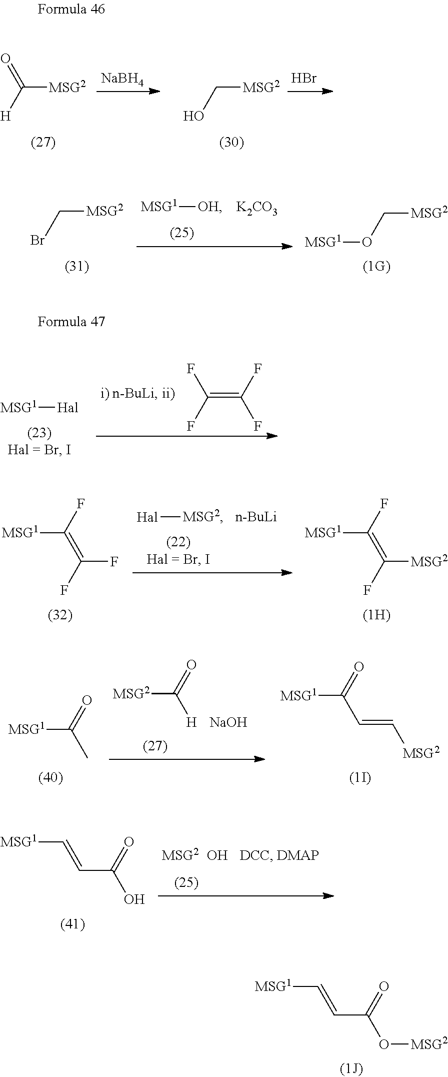

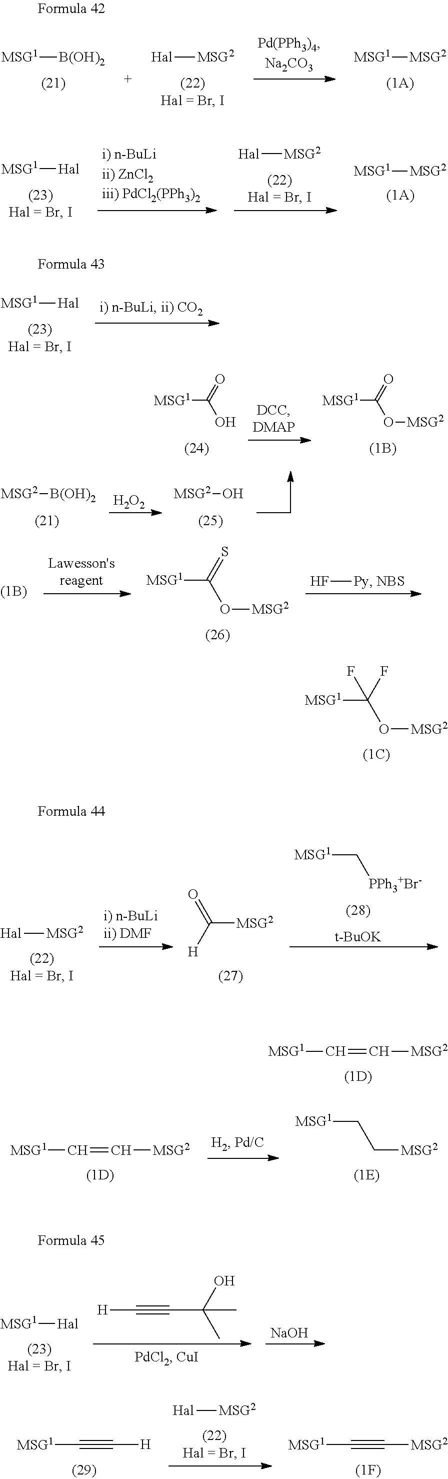

### (I) Formation of a Single Bond

Compound (1A) is prepared by allowing aryl boronic acid (21) to react with compound (22) in the presence of a carbonate and a tetrakis(triphenylphosphine)palladium catalyst. Compound (1A) is also prepared by allowing compound (23) to react with n-butyllithium and subsequently with zinc chloride, and further with compound (22) in the presence of a dichlorobis(triphenylphosphine)palladium catalyst.

### (II) Formation of —COO— and —OCO—

Carboxylic acid (24) is obtained by allowing compound (23) to react with n-butyllithium and subsequently with carbon dioxide. Compound (1B) having —COO— is prepared by dehydration of carboxylic acid (24) and phenol (25) derived from compound (21) in the presence of 1,3-dicyclohexylcarbodiimide (DCC) and 4-dimethylaminopyridine (DMAP). A compound having —OCO— is also prepared according to the method.

(III) Formation of —CF₂O— and —OCF₂—

Compound (26) is obtained by sulfurizing compound (1B) with a Lawesson's reagent. Compound (IC) having —CF₂O— is prepared by fluorinating compound (26) with a hydrogen fluoride-pyridine complex and N-bromosuccinimide (NBS). Refer to M. Kuroboshi et. al., Chem. Lett., 1992, 827. Compound (1C) is also prepared by fluorinating compound (26) with (diethylamino)sulfur trifluoride (DAST). Refer to W. H. Bunnelle et al., J. Org. Chem. 1990, 55, 768. A compound having —OCF₂— is also prepared according to the method.

### (IV) Formation of —CH═CH—

Aldehyde (27) is obtained by allowing compound (22) to react with n-butyllithium and subsequently with N,N-dimethylformamide (DMF). Compound (1D) is prepared by allowing phosphorus ylide generated by allowing phosphonium salt (28) to react with potassium t-butoxide to react with aldehyde (27). A cis isomer may be formed depending on reaction conditions, and therefore the cis isomer is isomerized into a trans isomer according to a publicly-known method when necessary.

(V) Formation of —CH₂CH₂—

Compound (1E) is prepared by hydrogenating compound (1D) in the presence of a palladium on carbon catalyst.

### (VI) Formation of —C≡C—

Compound (29) is obtained by allowing compound (23) to react with 2-methyl-3-butyn-2-ol in the presence of a catalyst of dichloropalladium and copper iodide and then performing deprotection of the resulting compound under basic conditions. Compound (1F) is prepared by allowing compound (29) to react with compound (22) in the presence of a catalyst of dichlorobis (triphenylphosphine)palladium and copper halide.

(VII) Formation of —CH₂O— and —OCH₂—

Compound (30) is obtained by reducing compound (27) with sodium borohydride. Compound (31) is obtained by brominating the obtained compound with hydrobromic acid. Compound (1G) is prepared by allowing compound (25) to react with compound (31) in the presence of potassium carbonate. A compound having —OCH₂— is also prepared according to the method.

### (VIII) Formation of —CF═CF—

Compound (32) is obtained by treating compound (23) with n-butyllithium and then allowing the treated compound to react with tetrafluoroethylene. Compound (1H) is prepared by treating compound (22) with n-butyllithium and then allowing the treated compound to react with compound (32).

### (VIV) Formation of —CH═CHCO— and —COCH═CH—

Compound (1I) is prepared by performing an aldol condensation reaction of compound (40) and compound (27) in the presence of NaOH.

### (X) Formation of —CH═CHCOO— and —OCOCH═CH—

Compound (1J) is prepared by dehydrating cinnamic acid (41) and compound (25) in the presence of 1,3-dicyclohexylcarbodiimide (DCC) and 4-dimethylaminopyridine (DMAP).

2-2. Formation of Rings A¹, A², A³ and A⁴

A starting material is commercially available or a synthesis method is well known with regard to a ring such as 1,4-cyclohexylene, 1,4-cyclohexenylene, 1,4-phenylene, 2-fluoro-1,4-phenylene, 2-methyl-1,4-phenylene, 2-ethyl-1,4-phenylene, naphthalene-2,6-diyl, decahydronaphthalene-2,6-diyl, 1,2,3,4-tetrahydronaphthalene-2,6-diyl, tetrahydropyran-2,5-diyl, 1,3-dioxane-2,5-diyl, pyrimidine-2,5-diyl, pyridine-2,5-diyl, perhydrocyclopenta[a]phenanthrene-3,17-diyl and 2,3,4,7,8,9,10,11,12,13,14,15,16,17-tetradecahydrocyclopenta[a]phenanthrene-3,17-diyl.

2-3. Formation of Linking Group Sp¹ or Sp² and Polymerizable Group P¹ or P²

Preferred examples of polymerizable group P¹ or P² include acryloyloxy (1b), maleimide (1c), an itaconic acid ester (1d), vinyl ester (1e), oxiranyl (1g) or vinyloxy (1h).

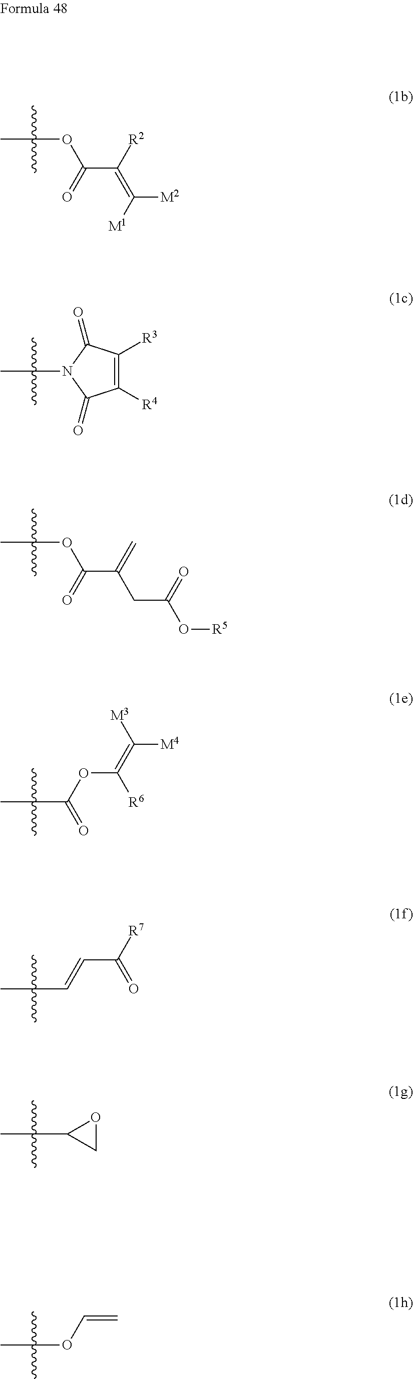

An example of a method for preparing a compound in which the polymerizable group is bonded to a ring through linking group Sp¹ or Sp² is as described below. First, an example in which linking group Sp¹ or Sp² is a single bond will be described.

(1) Synthesis of a Compound in Which Sp¹ or Sp² is a Single Bond

A method of preparing a compound in which Sp¹ or Sp² is a single bond is as described in a scheme below. In the scheme, MSG¹ is a monovalent organic group having at least one ring. Compounds (1S) to (1Z) correspond to compound (1). When the polymerizable group is an acrylate derivative, the acrylate derivative is prepared by performing esterification between the corresponding acrylic acid and HO-MSG¹. Vinyloxy is prepared by performing etherification between HO-MSG¹ and vinyl bromide. Oxiranyl is prepared by oxidation of a terminal double bond. A maleimide group is prepared by a reaction between an amino group and maleic anhydride. An itaconic acid ester is prepared by performing esterification between the corresponding itaconic acid and HO-MSG¹. Vinyl ester is prepared by a transesterification reaction between vinyl acetate and HOOC-MSG¹.

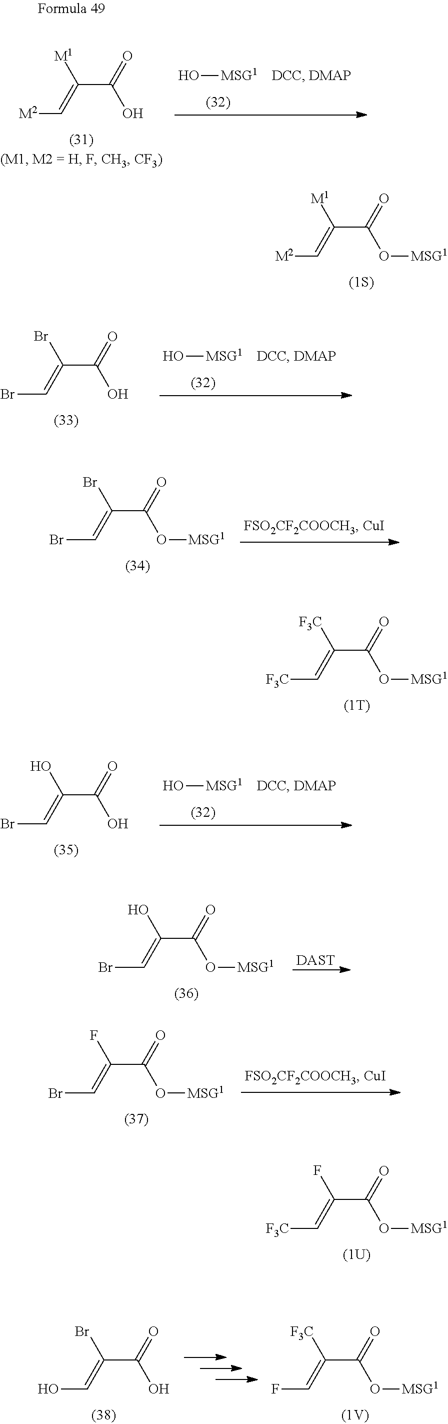

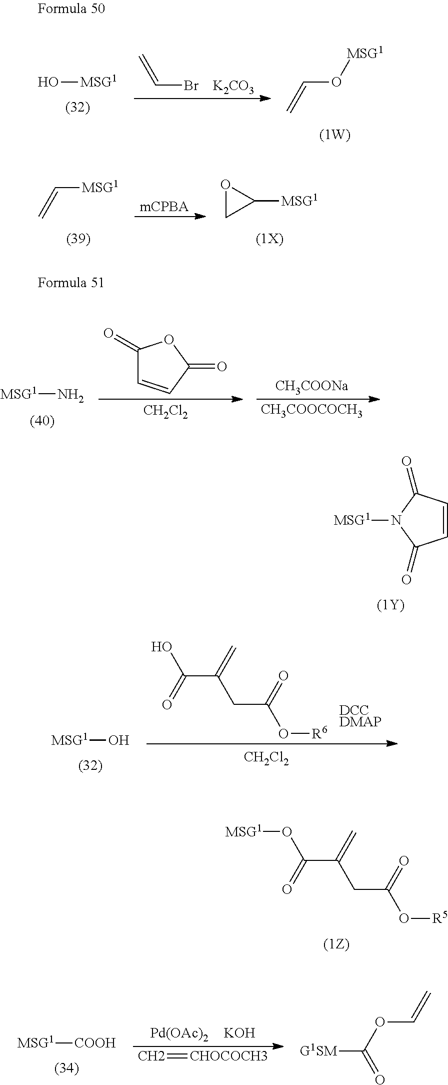

A synthesis method of the compound in which linking group Sp¹ or Sp² is the single bond is described above. As for a method of producing other linking groups, other linking groups can be prepared according to synthesis methods of bonding groups Z¹, Z², Z³, Z⁴ and Z⁵.

### 2-4. Synthesis Example

An example of a method for preparing compound (1) is as described below. In the compounds, MES is a mesogen group having at least one ring. Definitions of P¹, M¹, M², Sp¹ and Sp² are identical to the definitions described above.

Compound (51A) or compound (51B) is commercially available, or can be prepared according to a common organic synthesis method by using a mesogen (MES) having a suitable ring structure as a starting material. When a compound in which MES and Sp¹ are linked through an ether bond is prepared, compound (53) can be obtained by applying compound (51A) as a starting material, and performing etherification by using compound (52) and a base such as potassium hydroxide. Moreover, when a compound in which MES and Sp¹ are linked with a single bond is prepared, compound (53) can be obtained by applying compound (51B) as a starting material and performing a cross-coupling reaction by using compound (52), a metal catalyst such as palladium and a base. Compound (54) in which a protective group such as TMS and THP is allowed to act thereon may be derived from compound (53), when necessary.

Then, compound (56) can be obtained from compound (53) or compound (54) by performing etherification again in the presence of compound (55) and a base such as potassium hydroxide. On this occasion, when the protective group is allowed to act in a previous stage, the protective group is eliminated by a deprotection reaction.

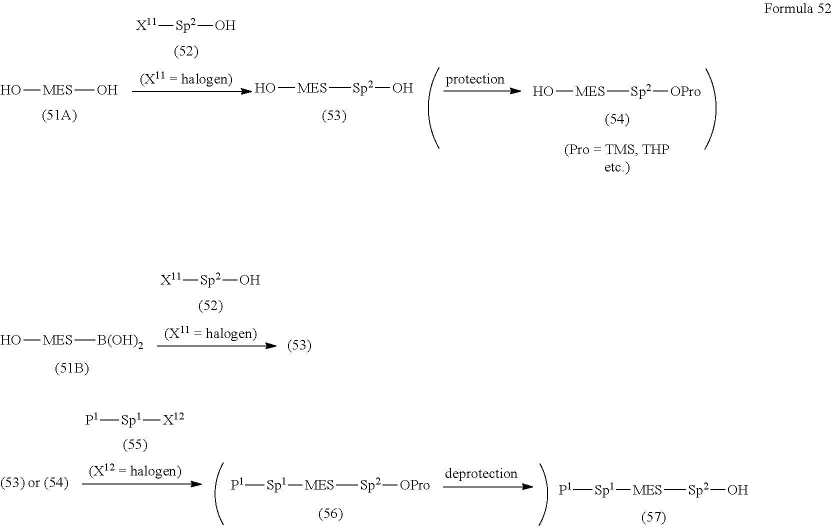

Compound (1A) in which P² is a group represented by formula (1b-3) can be prepared from compound (57) according to a method described below. Compound (1A) can be derived from compound (57) by performing an esterification reaction in the presence of compound (58), DCC and DMAP.

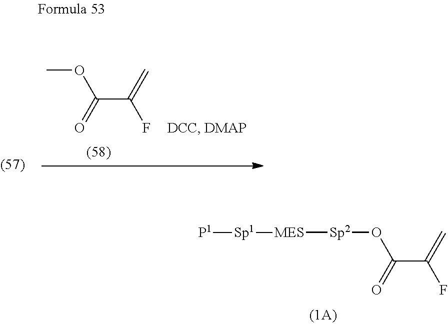

### 3. Liquid Crystal Composition

A liquid crystal composition according to an embodiment of the invention contains compound (1) as component A. Compound (1) can contribute to control of alignment of liquid crystal molecules by noncovalent interaction with a substrate of the device. The composition contains compound (1) as component A, and preferably further contains a liquid crystal compound selected from components B, C, D and E described below. Component B includes compounds (2) to (4). Component C includes compounds (5) to (7). Component B includes compound (8). Component E includes compounds (9) to (16). The composition may contain any other liquid crystal compound different from compounds (2) to (16). When the composition is prepared, components B, C, D and E are preferably selected by taking into account magnitude of positive or negative dielectric anisotropy, or the like. The composition in which the components are suitably selected has high maximum temperature, low minimum temperature, small viscosity, suitable optical anisotropy (more specifically, large optical anisotropy or small optical anisotropy), large positive or negative dielectric anisotropy, large specific resistance, high stability to heat or ultraviolet light and a suitable elastic constant (more specifically, a large elastic constant or a small elastic constant).

A preferred proportion of compound (1) is ordinarily about 0.01% by weight or more based on the weight of the liquid crystal composition for maintaining high stability to ultraviolet light, and ordinarily about 10% by weight or less for dissolution in the liquid crystal composition. A further preferred proportion is in the range of about 0.1% by weight to about 5% by weight based on the weight of the liquid crystal composition. A most preferred proportion is in the range of about 0.5% by weight to about 3% by weight based on the weight of the liquid crystal composition.

Component B is a compound in which two terminal groups are alkyl or the like. Preferred examples of component B include compounds (2-1) to (2-11), compounds (3-1) to (3-19) and compounds (4-1) to (4-7). In a compound of component B, R¹¹ and R¹² are independently alkyl having 1 to 10 carbons or alkenyl having 2 to 10 carbons, and in the alkyl or the alkenyl, at least one piece of —CH₂— may be replaced by —O—, and at least one hydrogen may be replaced by fluorine.

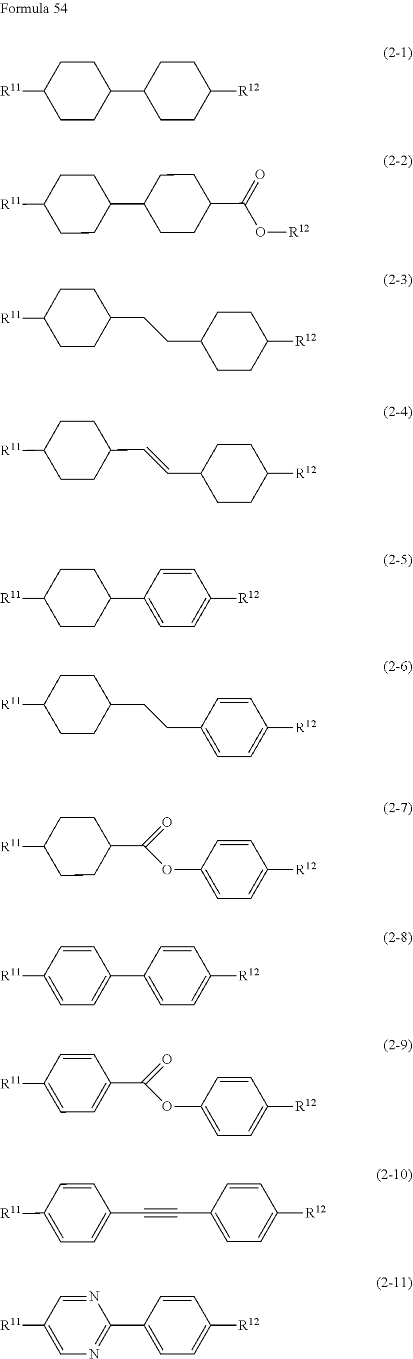

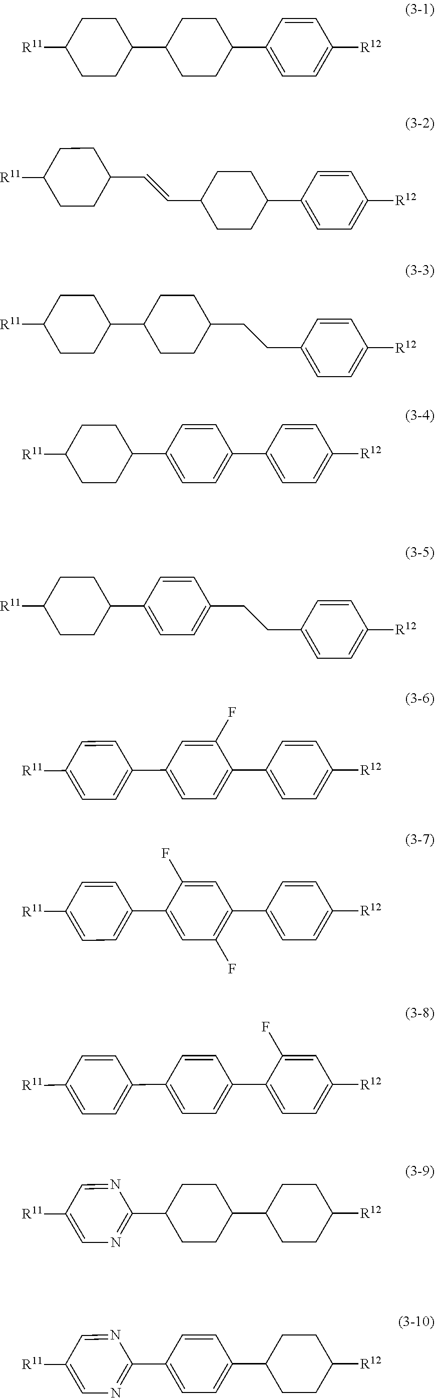

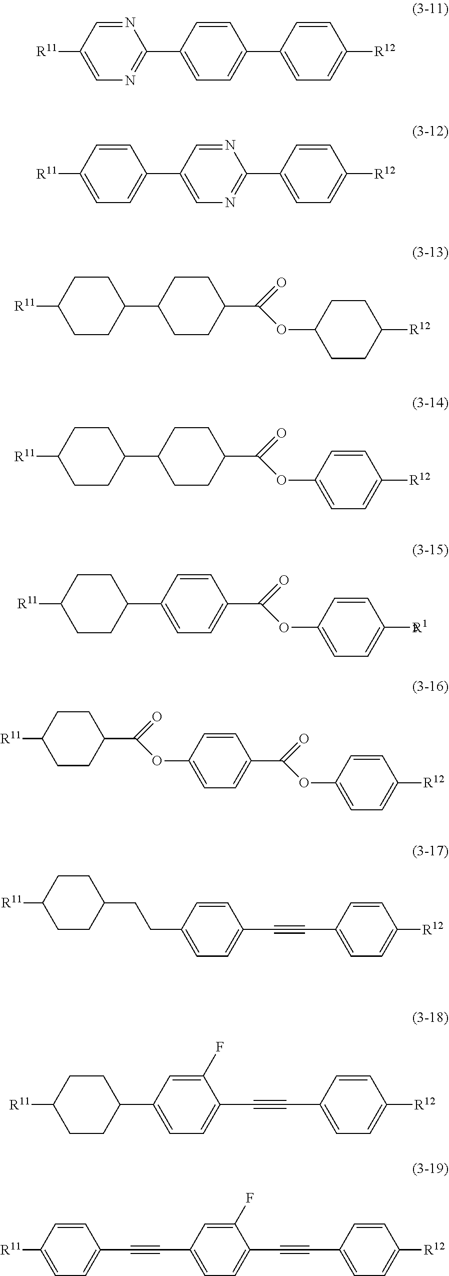

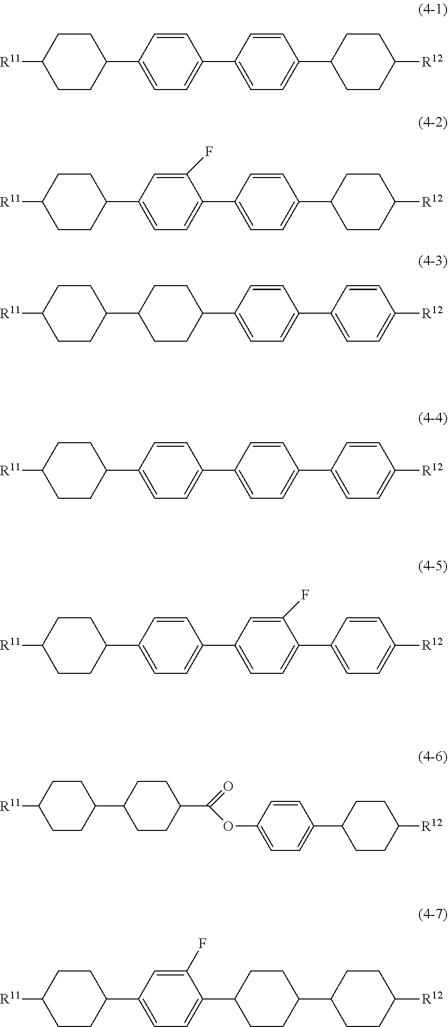

Component B has a small absolute value of dielectric anisotropy, and therefore is a compound close to neutrality. Compound (2) is mainly effective in decreasing the viscosity or adjusting the optical anisotropy. Compounds (3) and (4) are effective in extending a temperature range of a nematic phase by increasing the maximum temperature, or in adjusting the optical anisotropy.

As a content of component B increases, the dielectric anisotropy of the composition decreases, but the viscosity decreases. Thus, as long as a desired value of threshold voltage of a device is met, the content is preferably as large as possible. When a composition for an IPS mode, a VA mode or the like is prepared, the content of component B is preferably 30% by weight or more, and further preferably 40% by weight or more, based on the weight of the liquid crystal composition.

Component C is a compound having a halogen-containing group or a fluorine-containing group at a right terminal. Preferred examples of component C include compounds (5-1) to (5-16), compounds (6-1) to (6-120) and compounds (7-1) to (7-62). In the compounds of component C, R¹³ is alkyl having 1 to 10 carbons or alkenyl having 2 to 10 carbons, and in the alkyl and the alkenyl, at least one piece of —CH₂— may be replaced by —O—, and at least one hydrogen may be replaced by fluorine; and X¹¹ is fluorine, chlorine, —OCF₃, —OCHF₂, —CF₃, —CHF₂, —CH₂F, —OCF₂CHF₂ or —OCF₂CHFCF₃.

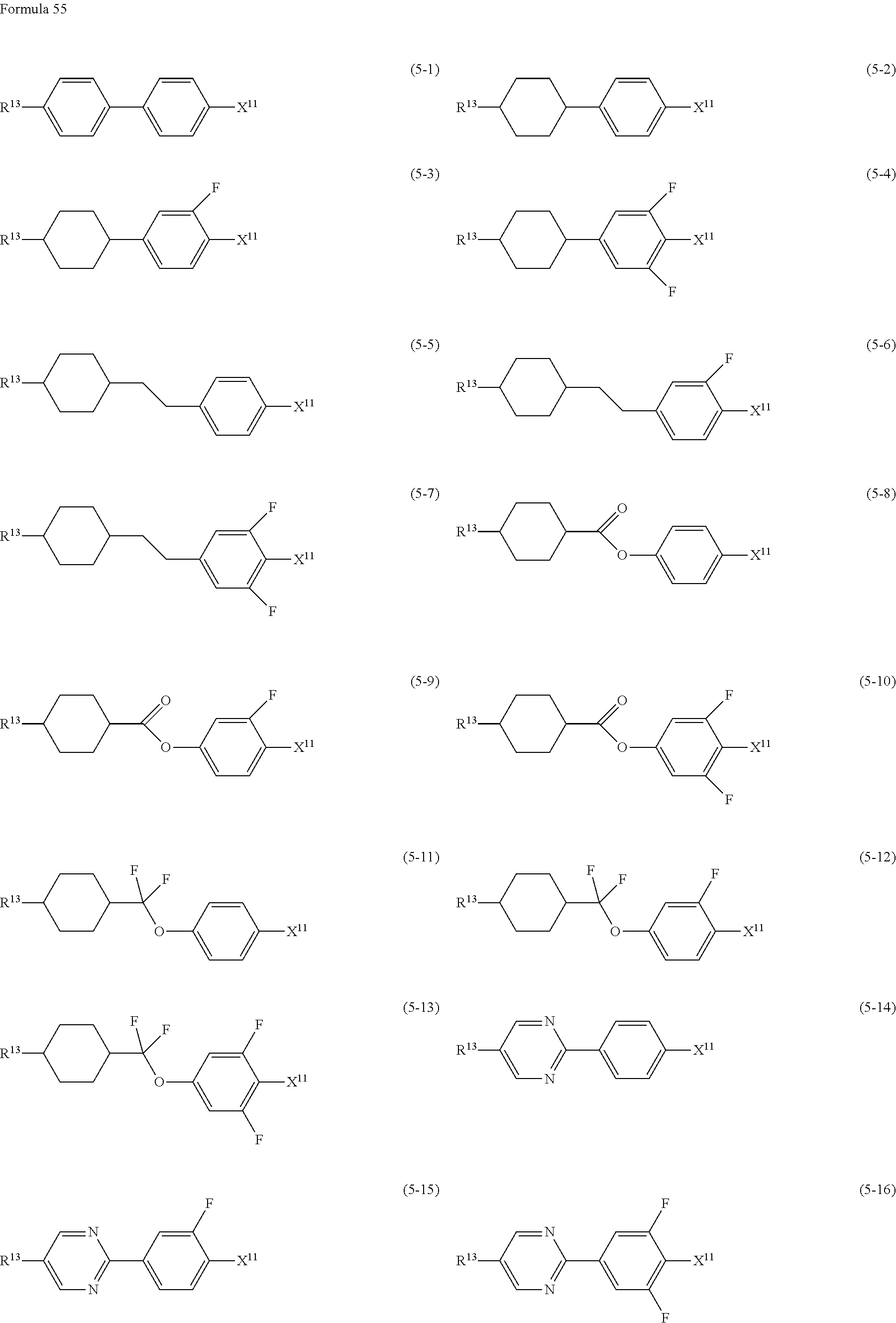

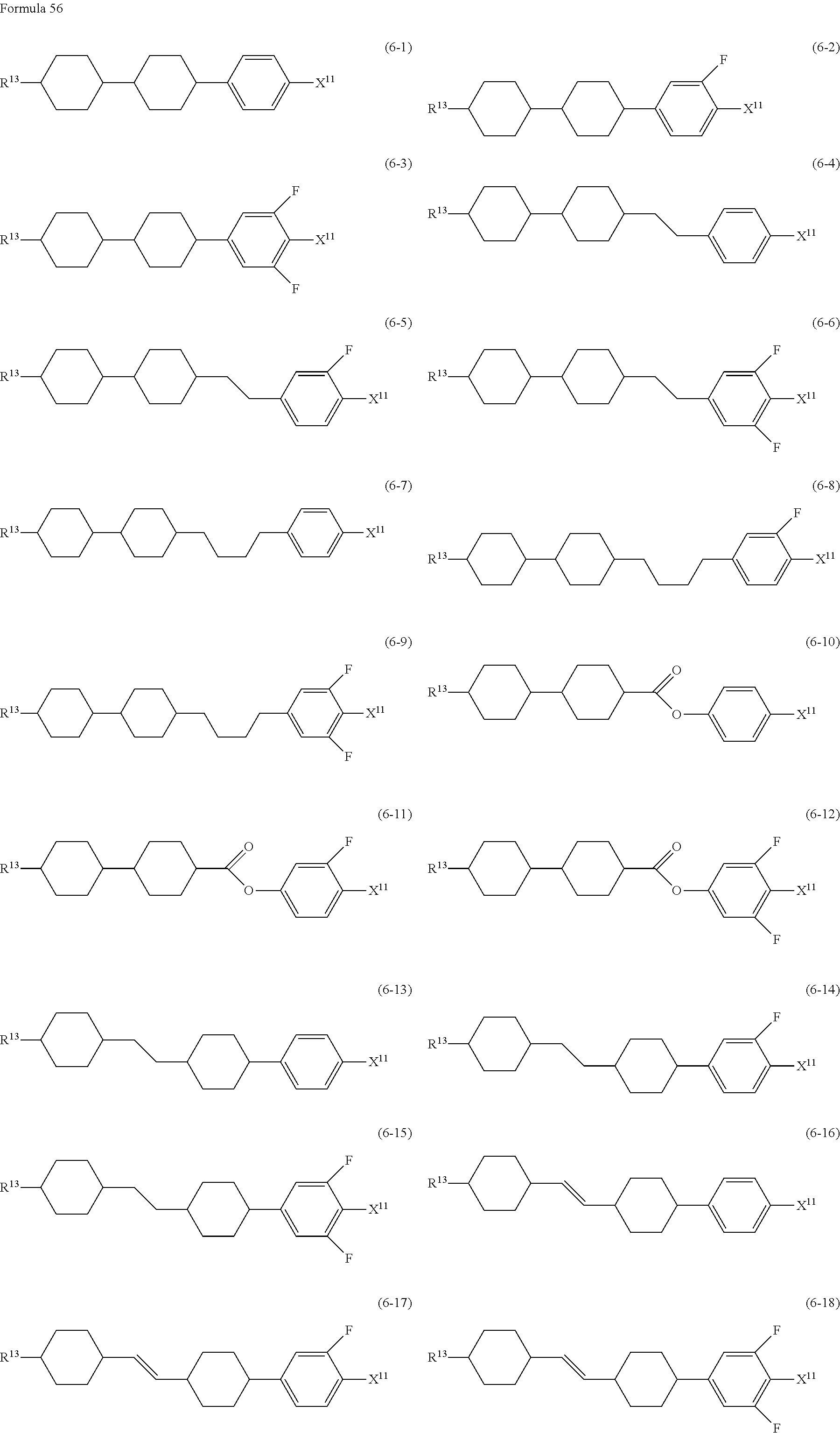

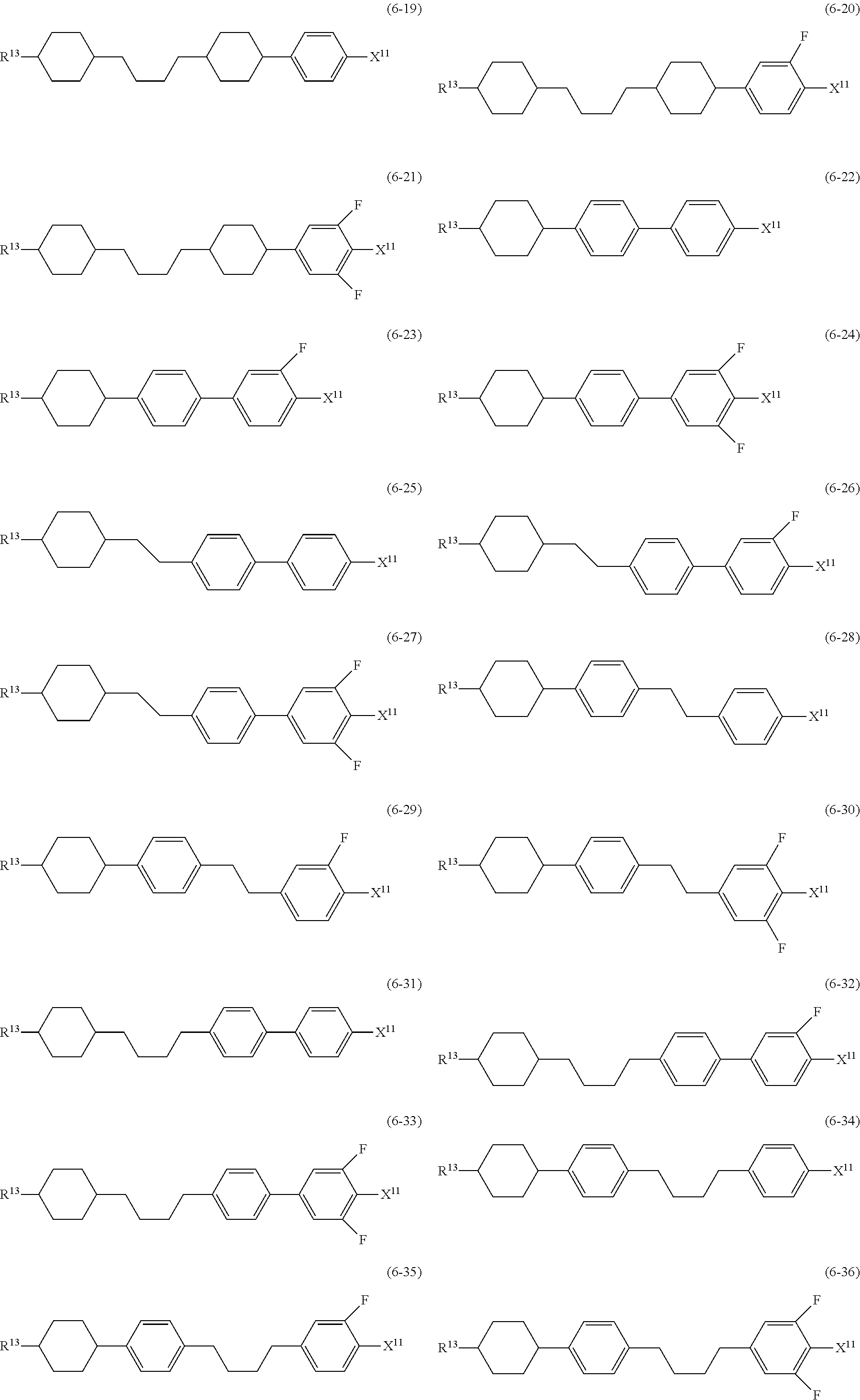

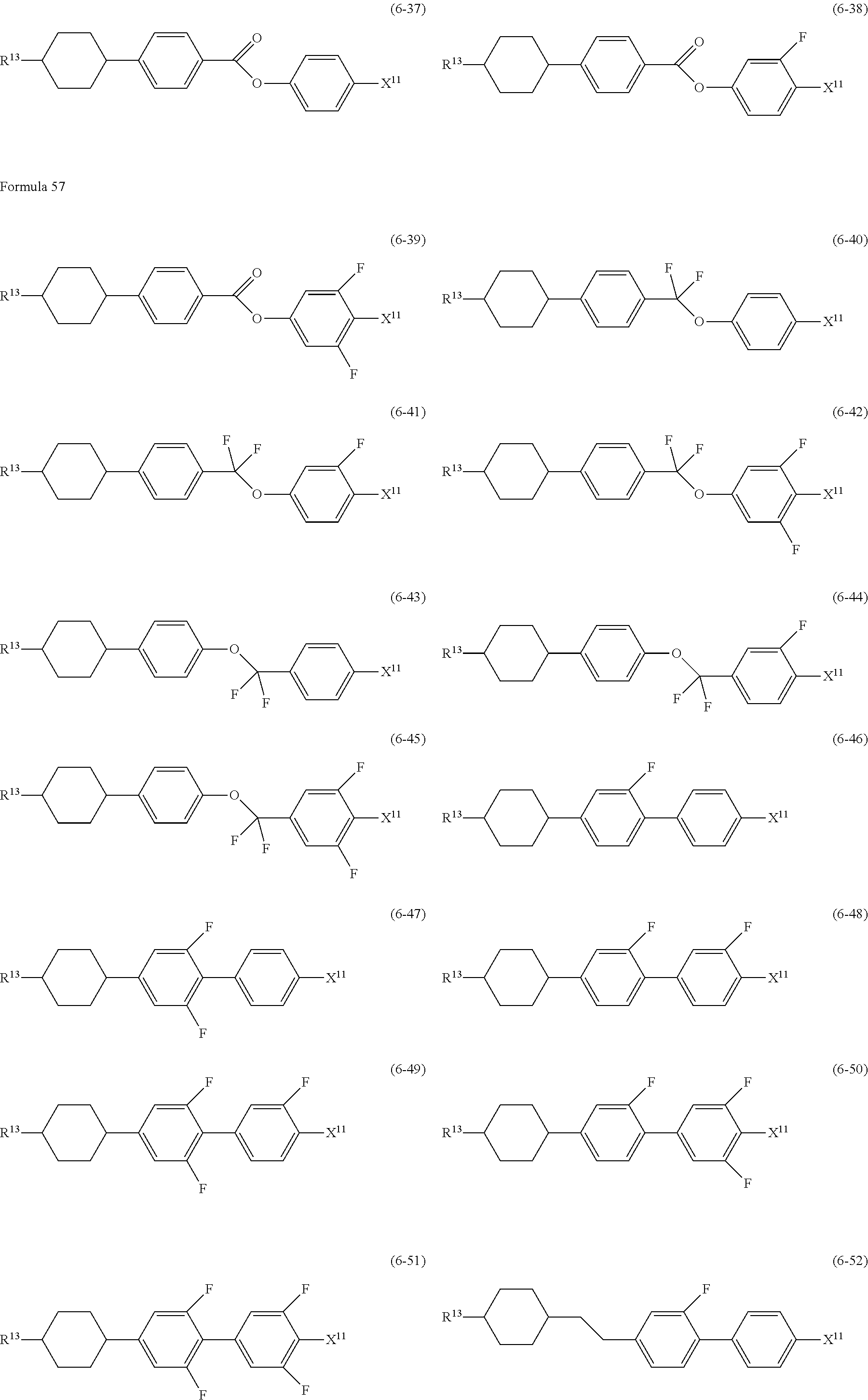

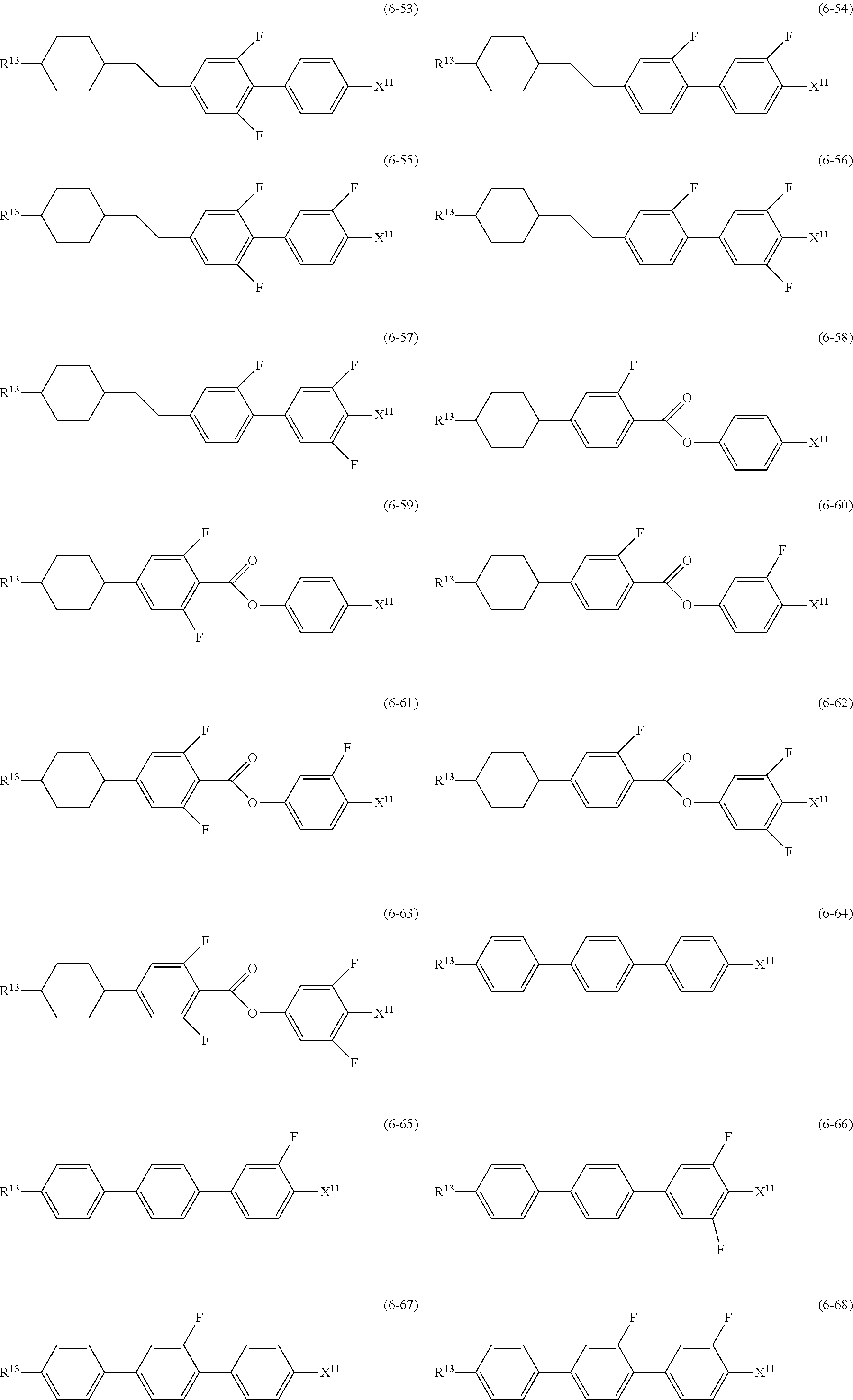

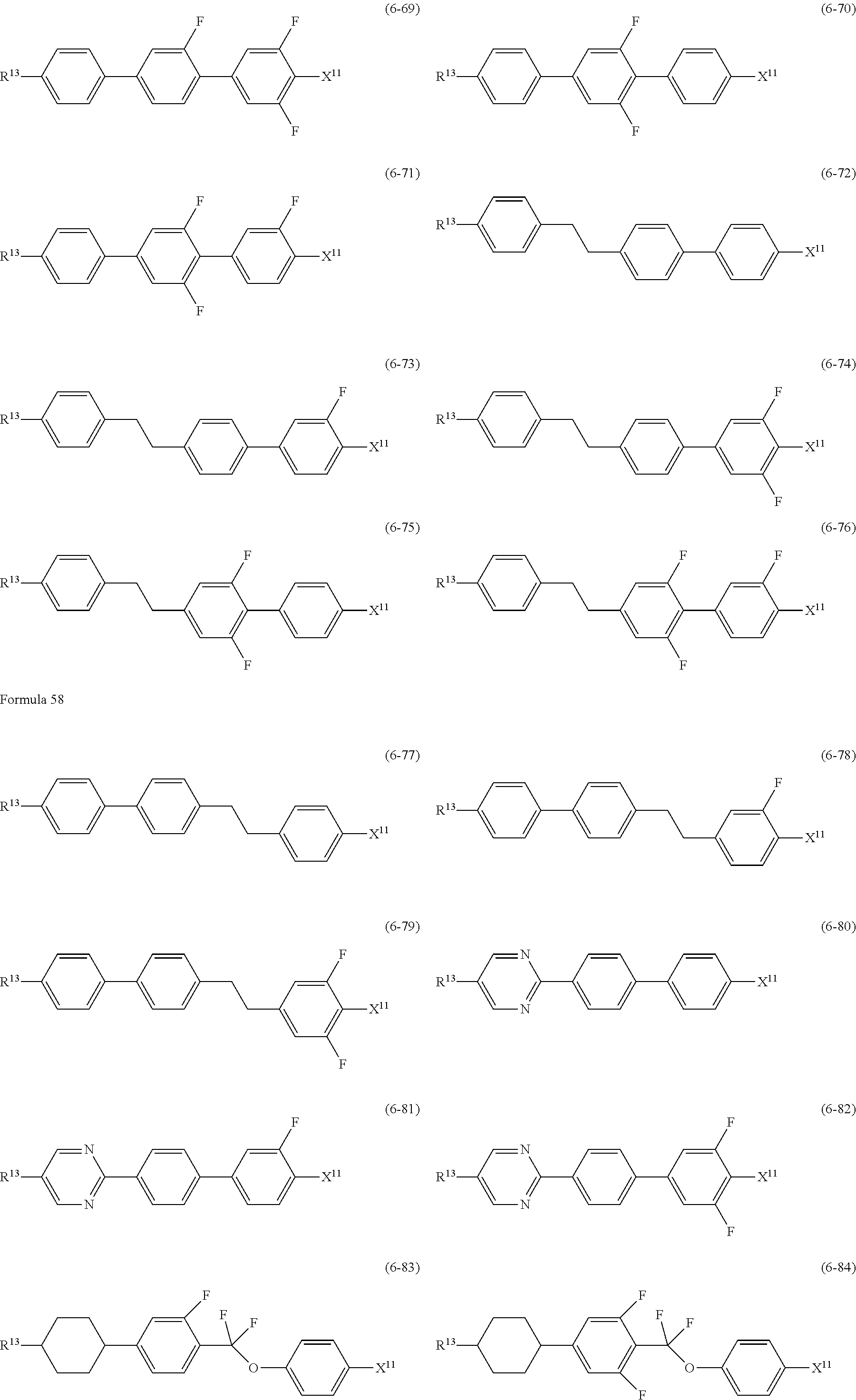

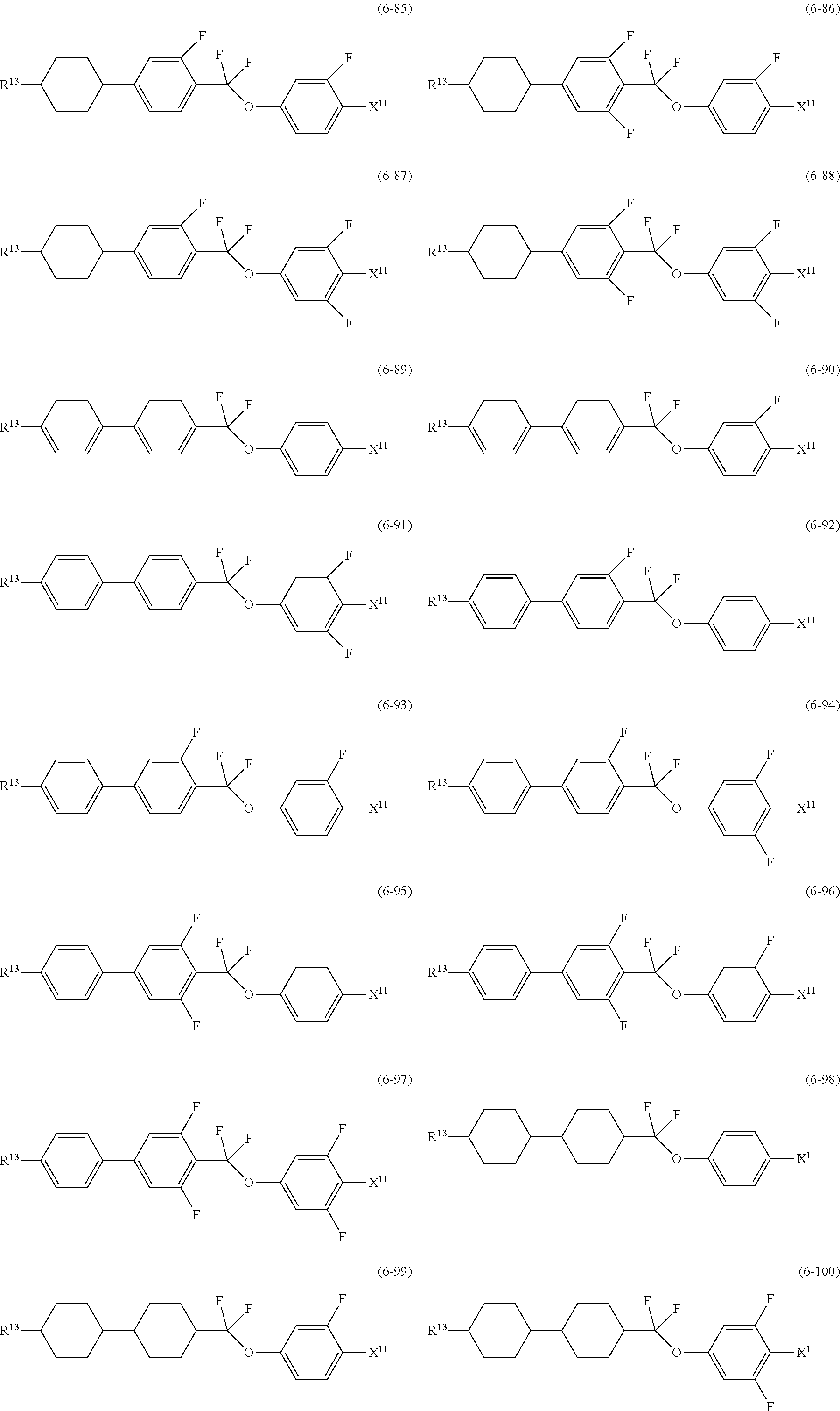

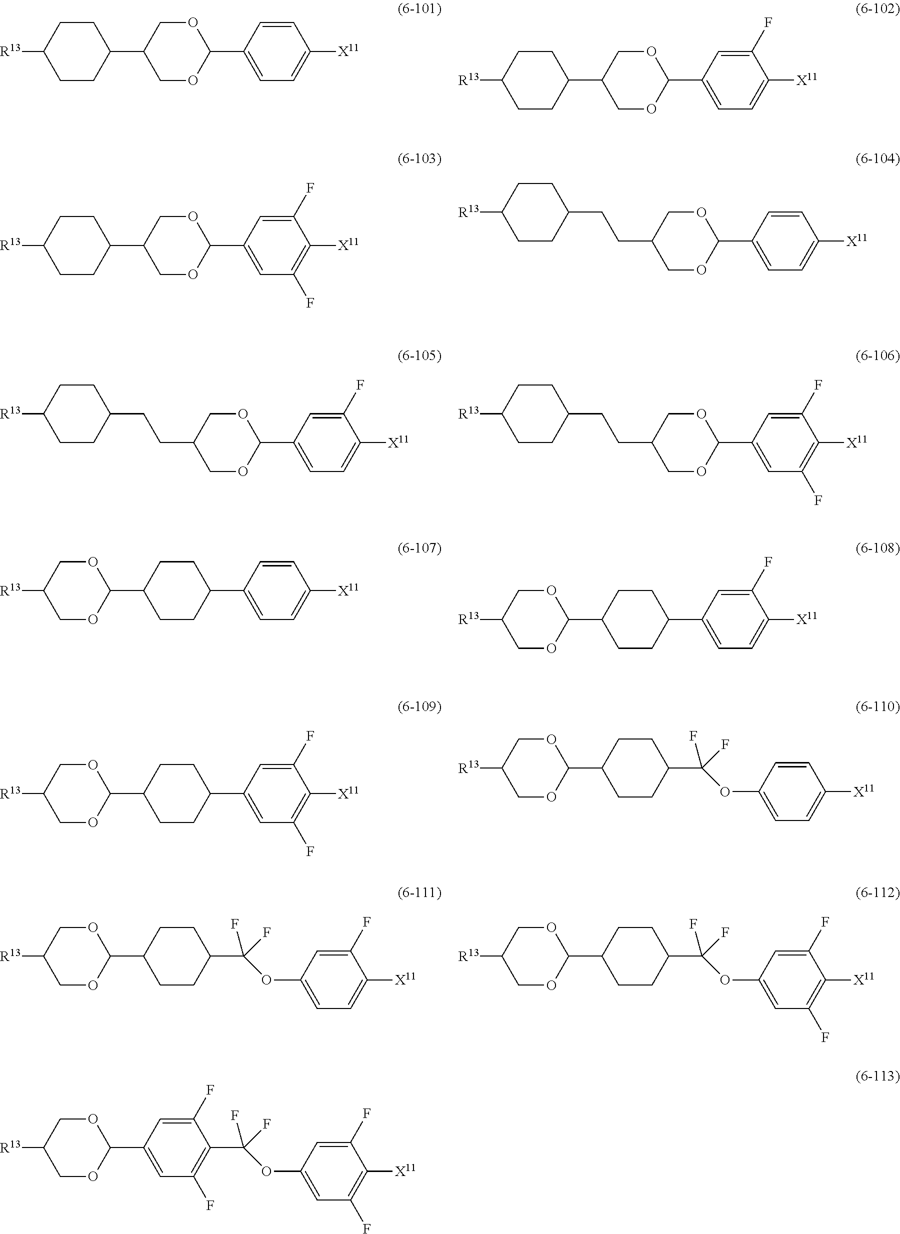

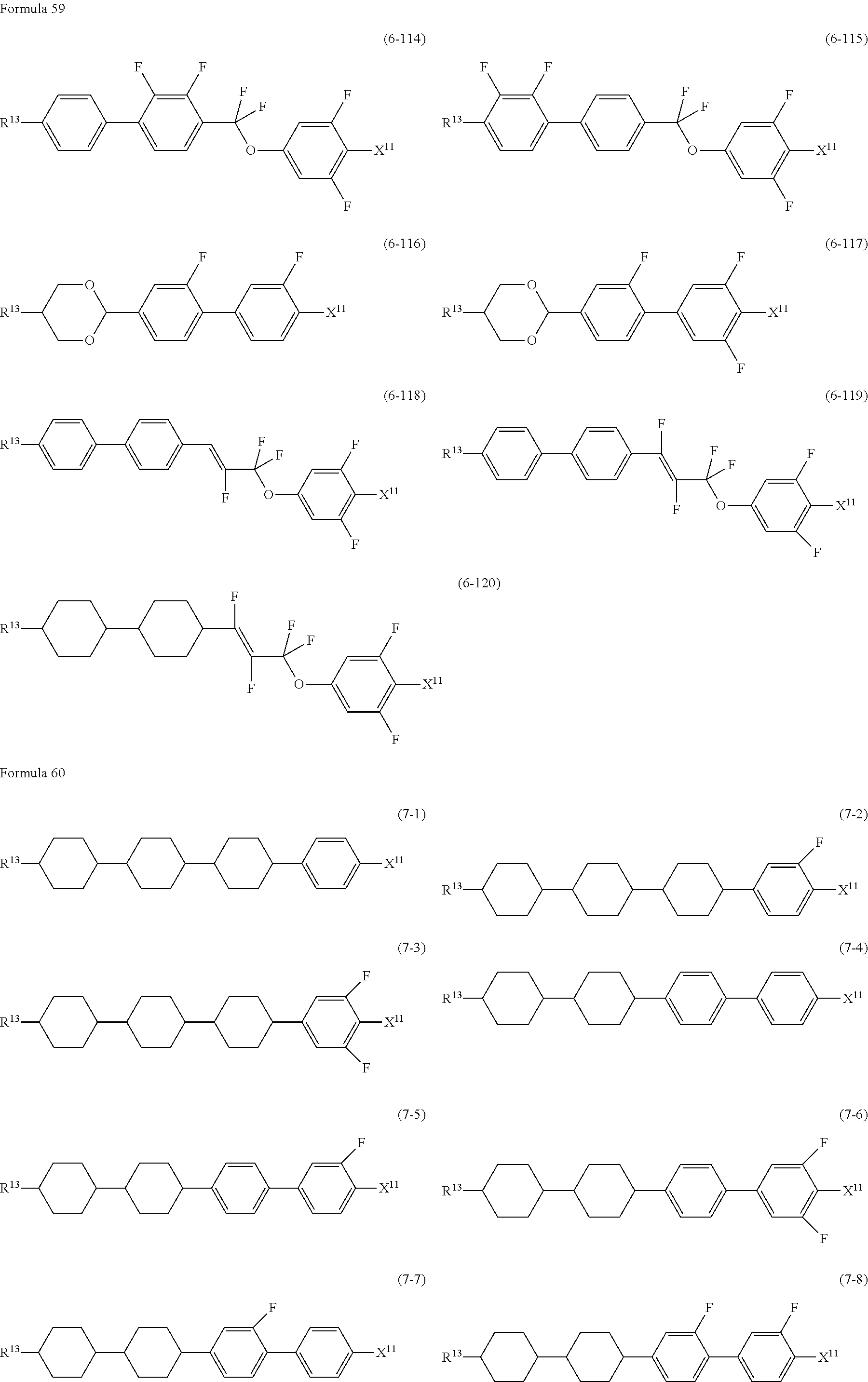

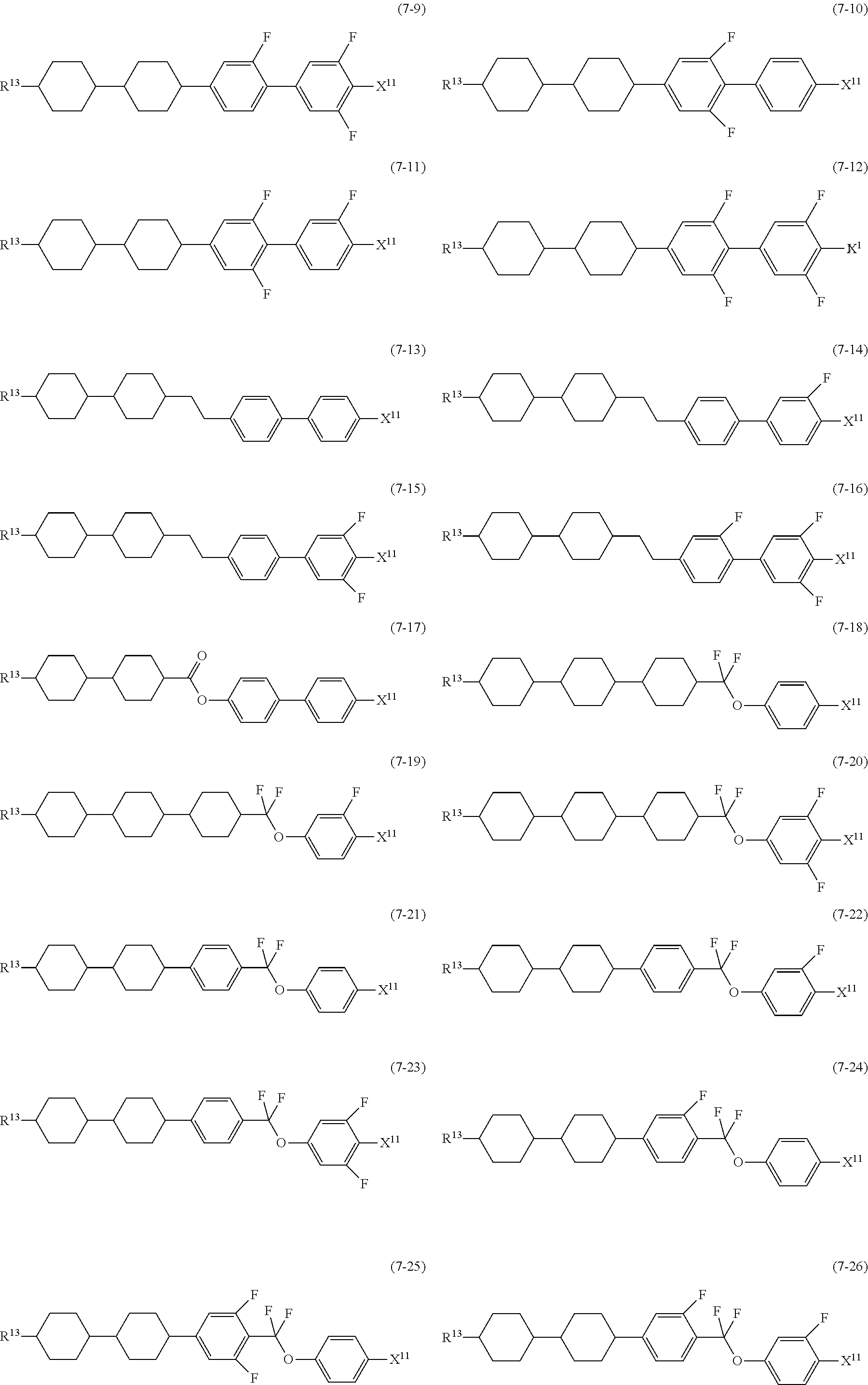

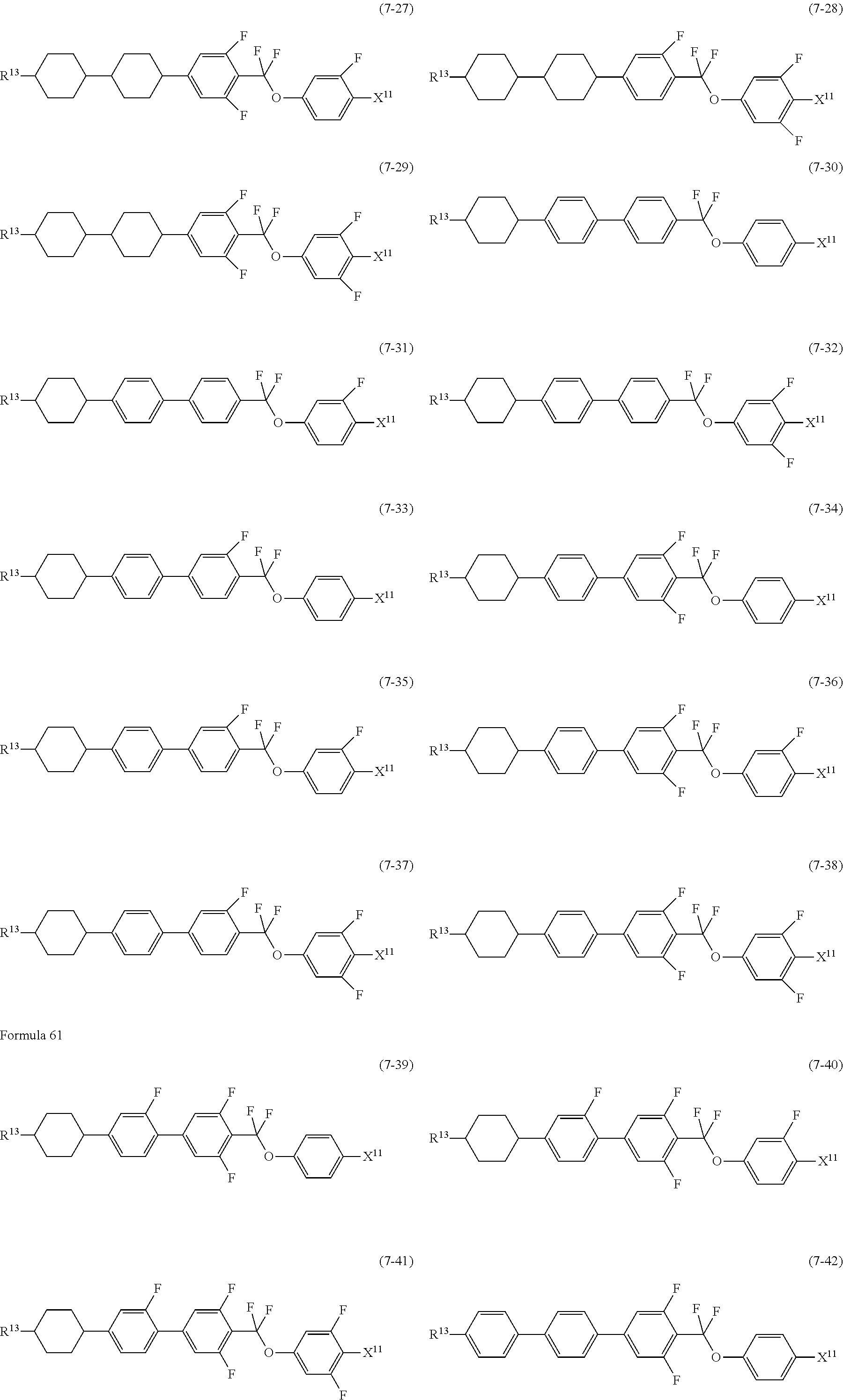

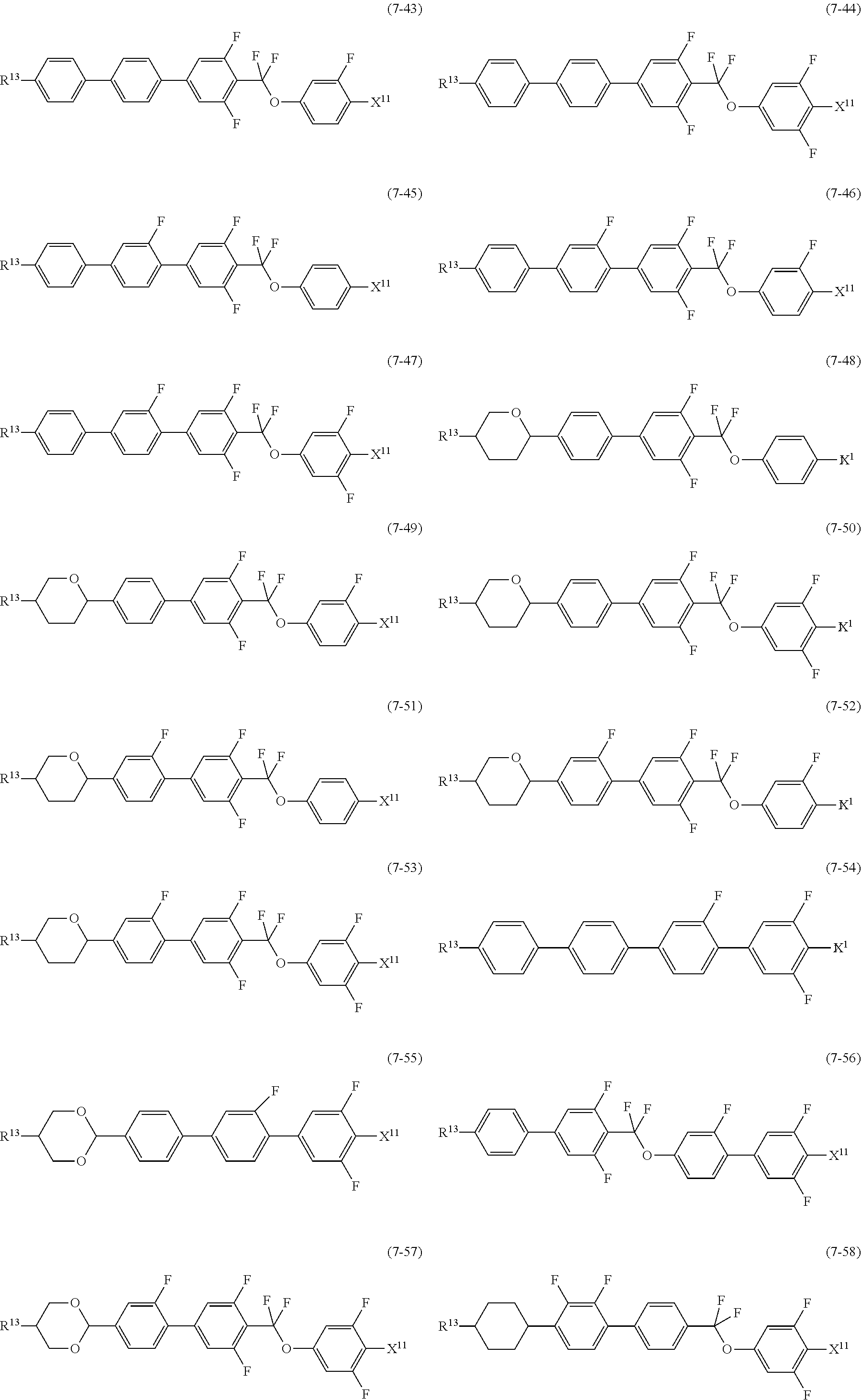

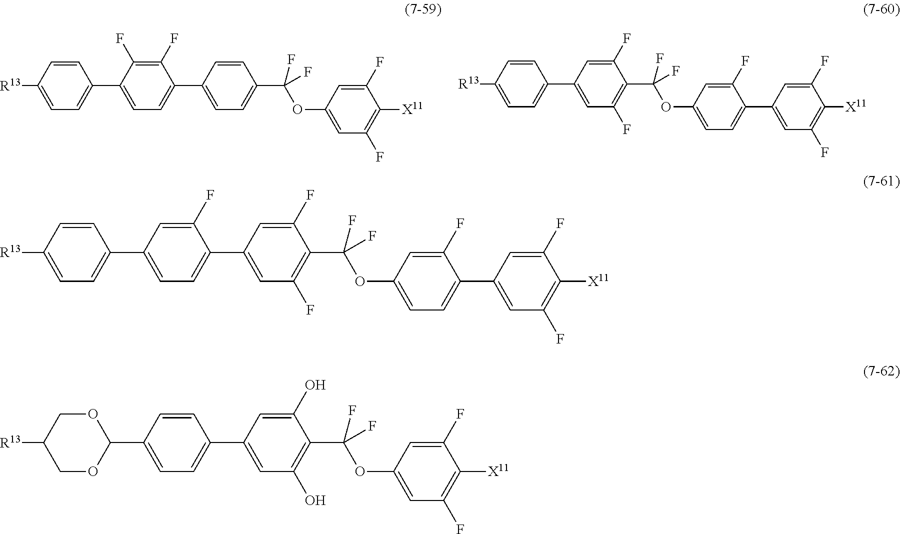

Component C has positive dielectric anisotropy, and superb stability to heat, light and so forth, and therefore is used when a composition for an IPS mode, an FFS mode, an OCB mode or the like is prepared. A content of component C is suitably in the range of 1% by weight to 99% by weight, preferably in the range of 10% by weight to 97% by weight, and further preferably in the range of 40% by weight to 95% by weight, based on the weight of the liquid crystal composition. When component C is added to a composition having negative dielectric anisotropy, the content of component C preferably 30% by weight or less based on the weight of the liquid crystal composition. Addition of component C allows adjustment of the elastic constant of the composition and adjustment of a voltage-transmittance curve of the device.

Component D is compound (8) in which a right-terminal group is —C≡N or —C≡C—C≡N. Preferred examples of component D include compounds (8-1) to (8-64). In the compounds of component D, R¹⁴ is alkyl having 1 to 10 carbons or alkenyl having 2 to 10 carbons, and in the alkyl and the alkenyl, at least one piece of —CH₂— may be replaced by —O—, and at least one hydrogen may be replaced by fluorine; and —X¹² is —C≡N or —C≡C—C≡N.

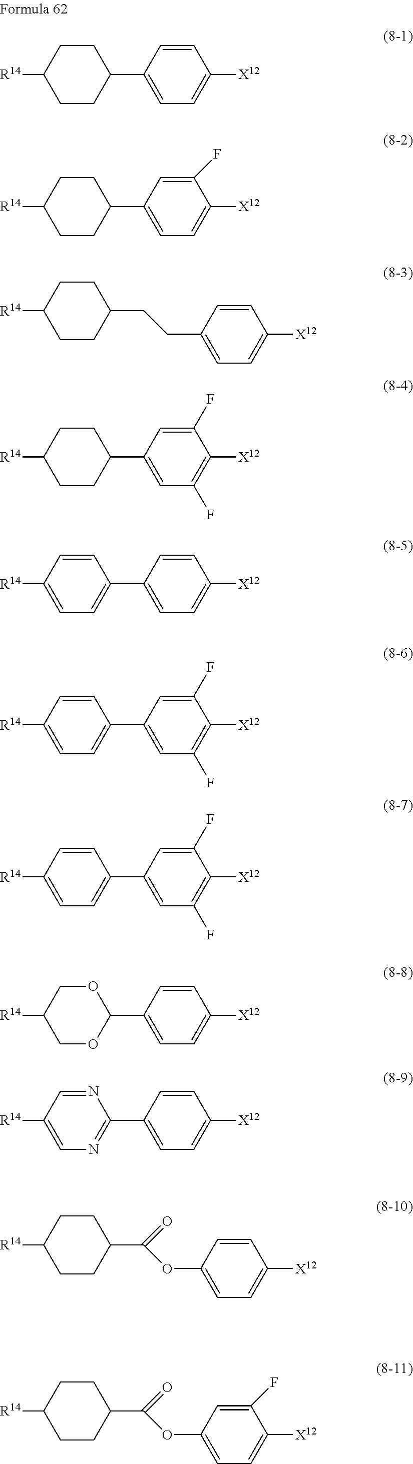

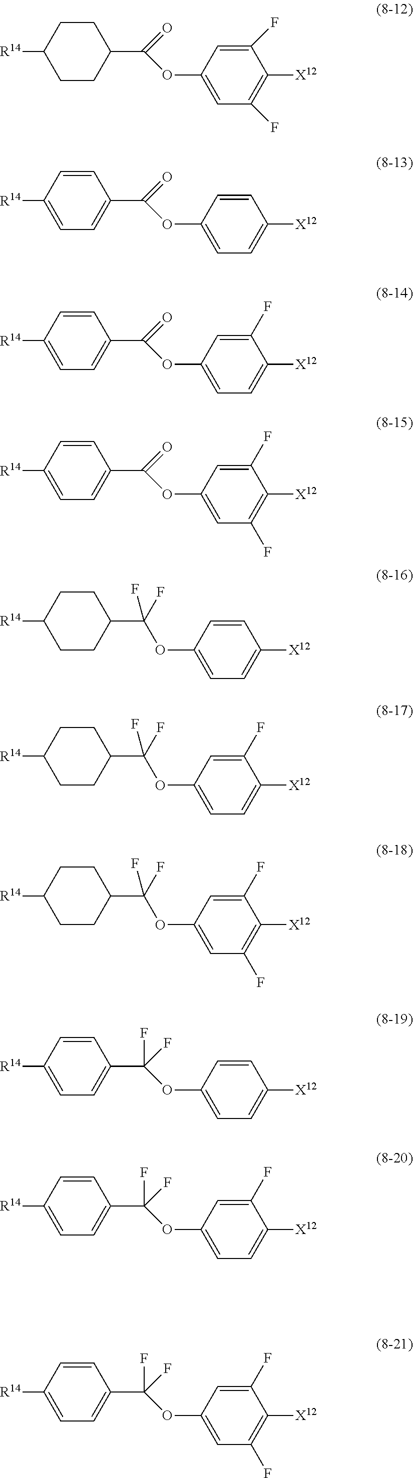

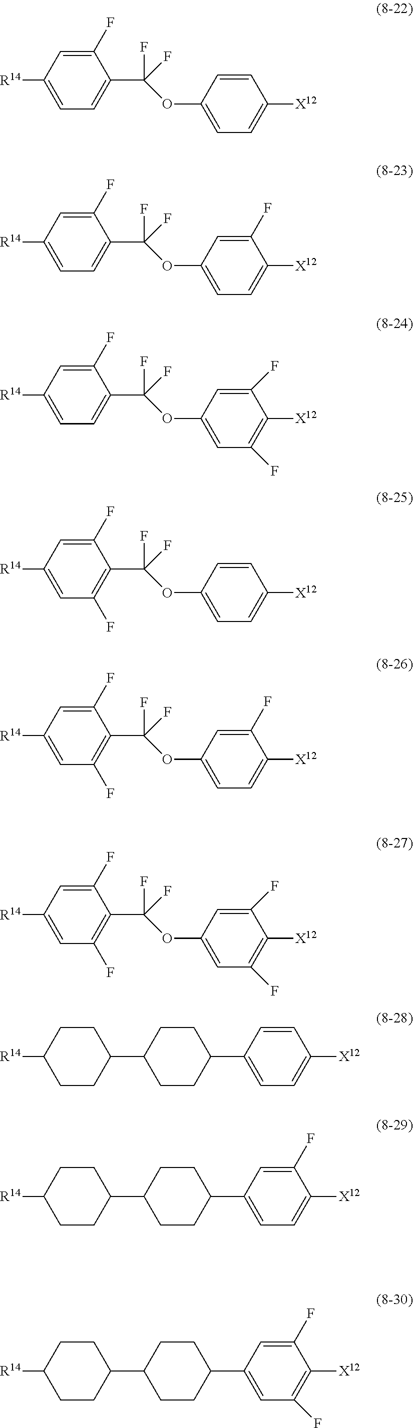

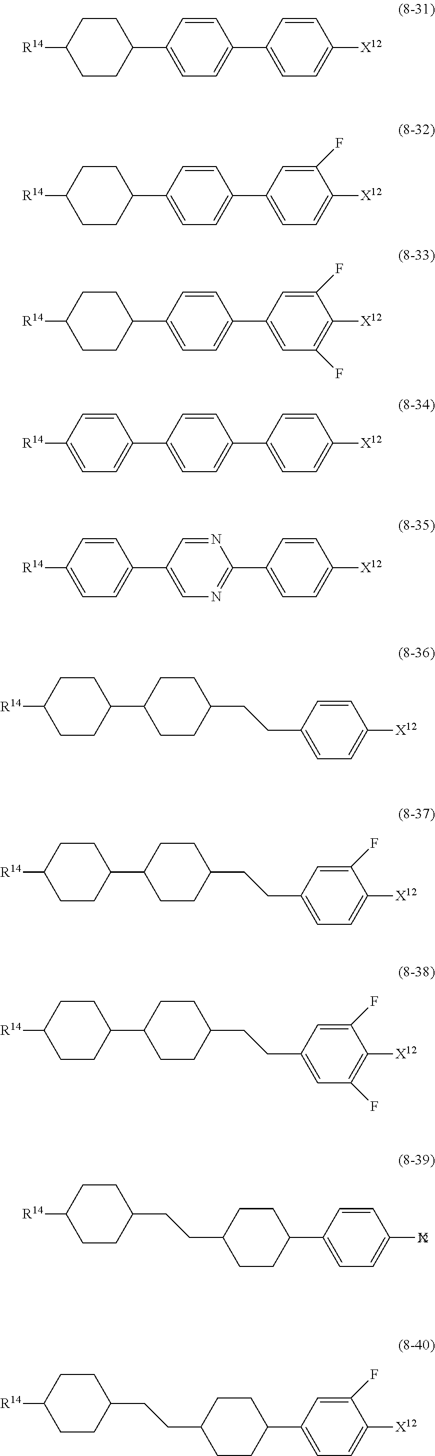

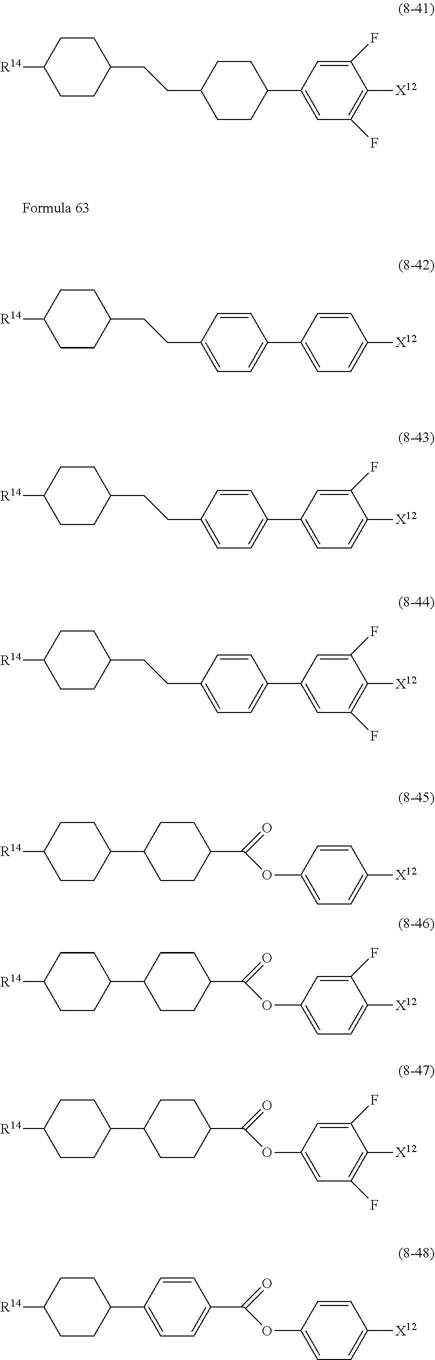

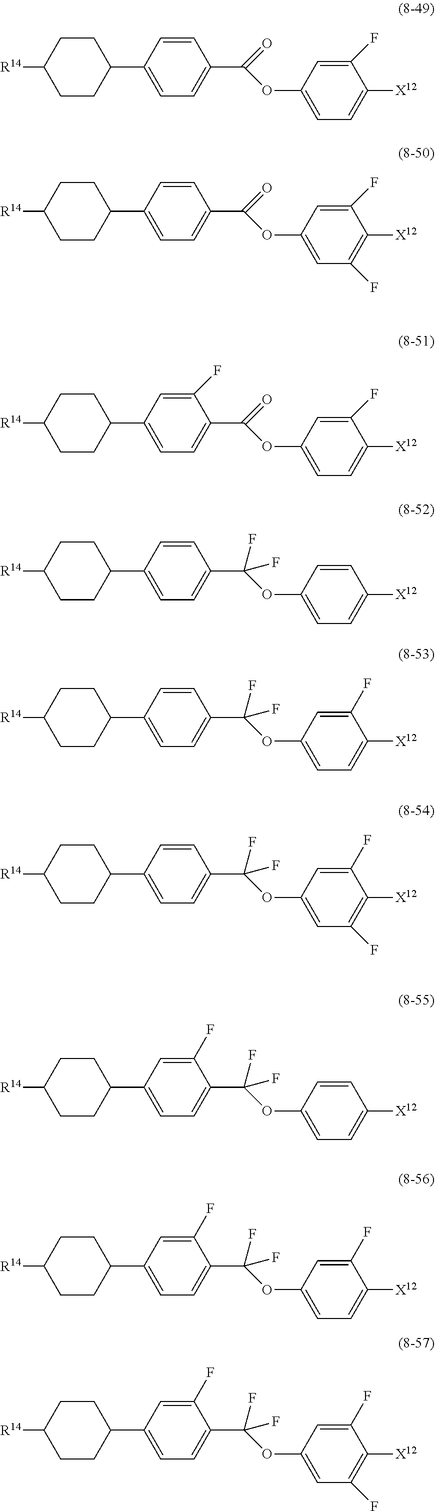

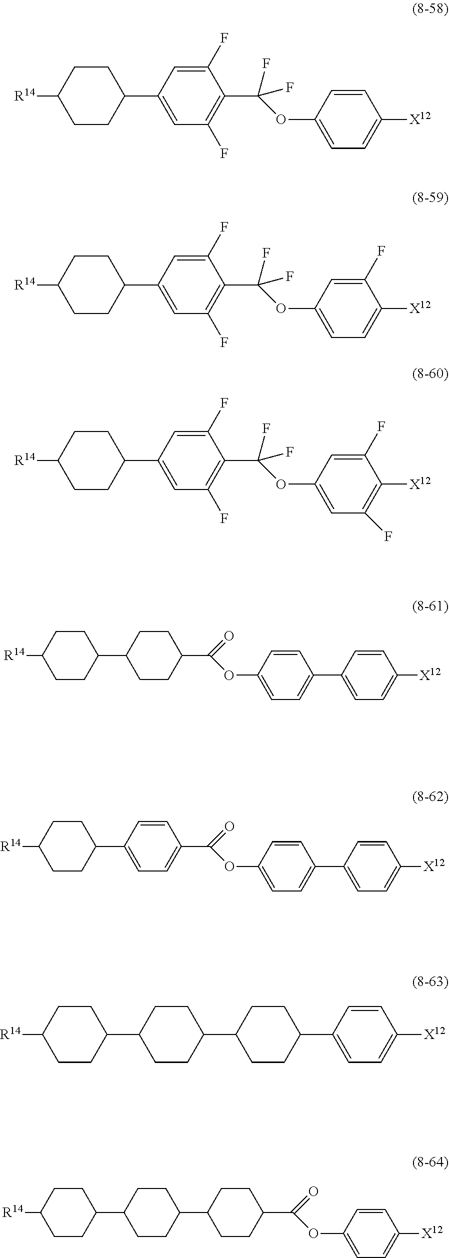

Component D has positive dielectric anisotropy and a large value thereof, and therefore is mainly used when a composition for a TN mode or the like is prepared. Addition of component D can increase the dielectric anisotropy of the composition. Component D produces an effect of extending a temperature range of a liquid crystal phase, adjusting the viscosity or adjusting the optical anisotropy. Component D is also useful for adjustment of the voltage-transmittance curve of the device.

When the composition for the TN mode or the like is prepared, a content of component D is suitably in the range of 1% by weight to 99% by weight, preferably in the range of 10% by weight to 97% by weight, and further preferably in the range of 40% by weight to 95% by weight, based on the weight of the liquid crystal composition. When component D is added to a composition having negative dielectric anisotropy, the content of component D is preferably 30% by weight or less based on the weight of the liquid crystal composition. Addition of component D allows adjustment of the elastic constant of the composition and adjustment of the voltage-transmittance curve of the device.

Component E includes compounds (9) to (16). The compounds have phenylene in which atoms in lateral positions are replaced by two halogens, such as 2,3-difluoro-1,4-phenylene.

Preferred examples of component E include compounds (9-1) to (9-8), compounds (10-1) to (10-17), compound (11-1), compounds (12-1) to (12-3), compounds 13-1) to (13-11), compounds (14-1) to (14-3), compounds (15-1) to (15-3) and compounds (16-1) to (16-3). In the compounds of component E, R¹⁵ and R¹⁶ are independently alkyl having 1 to 10 carbons or alkenyl having 2 to 10 carbons, and in the alkyl and the alkenyl, at least one piece of —CH₂— may be replaced by —O—, and at least one hydrogen may be replaced by fluorine; and R¹⁷ is hydrogen, fluorine, alkyl having 1 to 10 carbons or alkenyl having 2 to 10 carbons, and in the alkyl and the alkenyl, at least one piece of —CH₂— may be replaced by —O—, and at least one hydrogen may be replaced by fluorine.

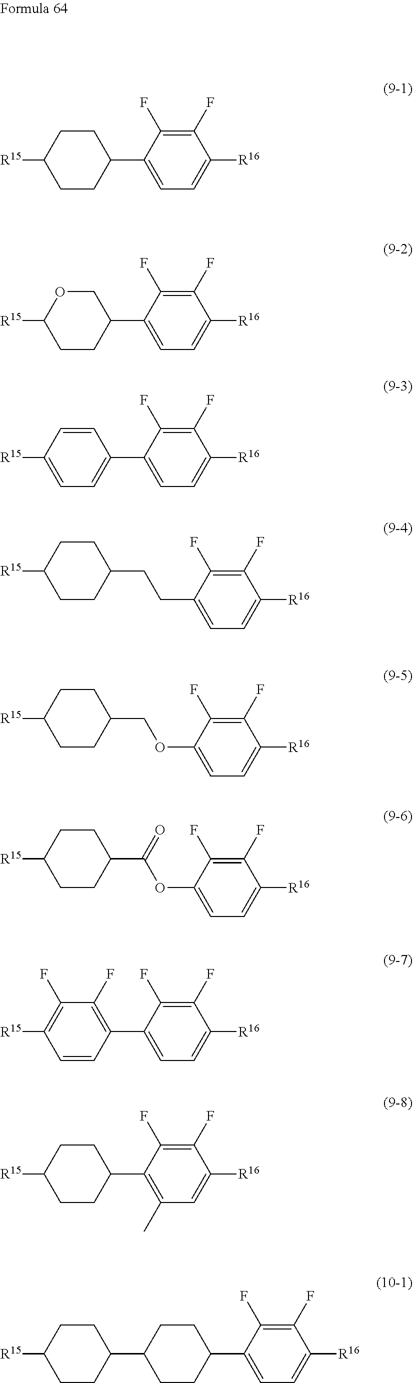

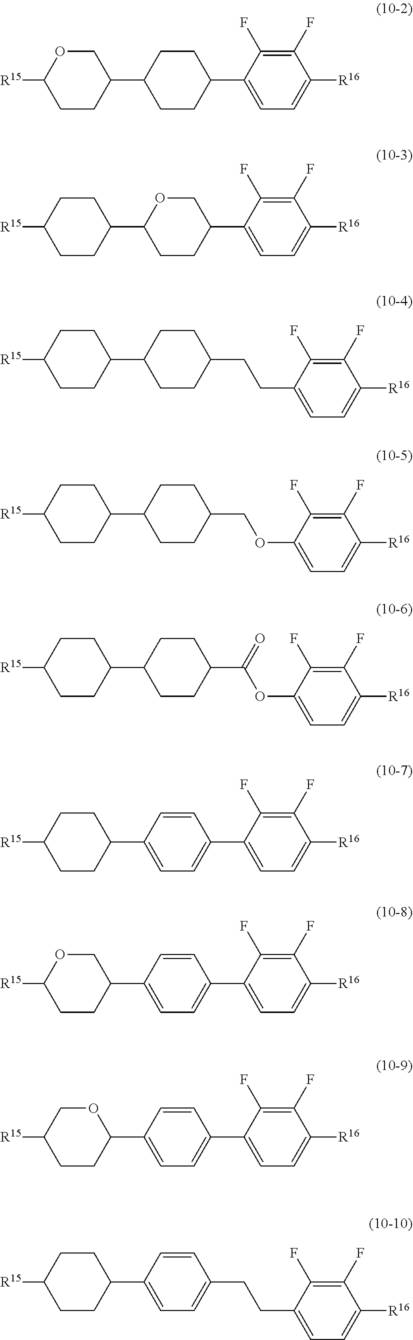

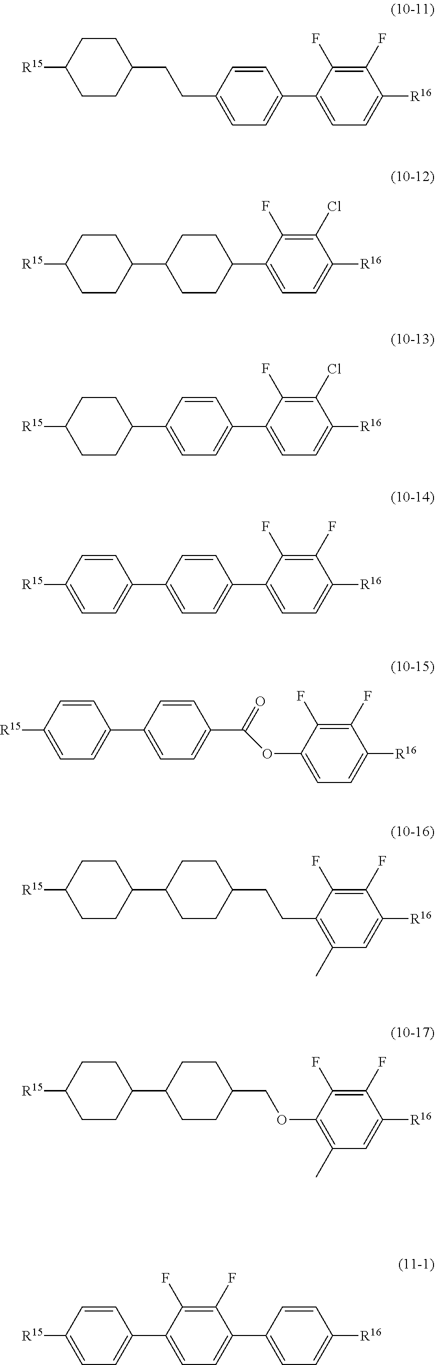

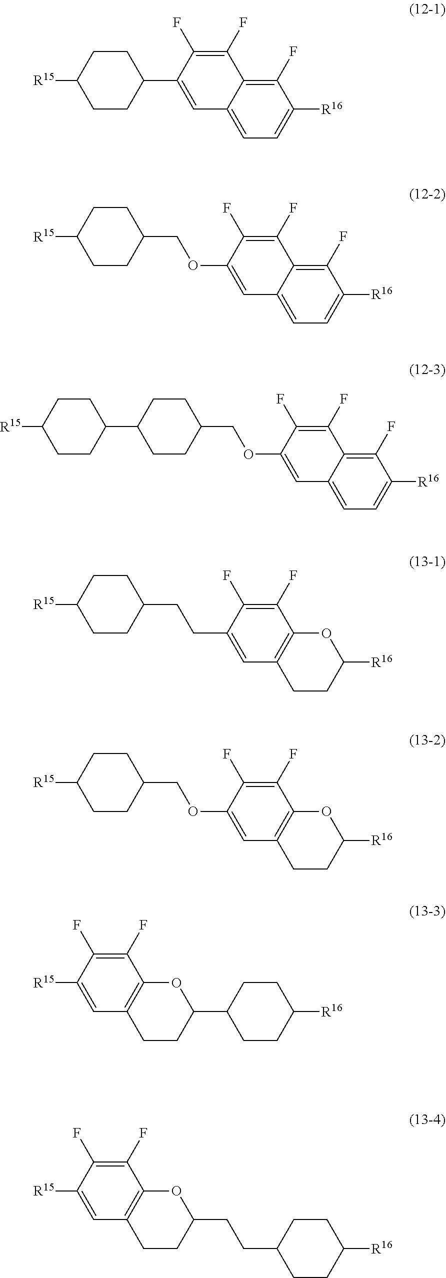

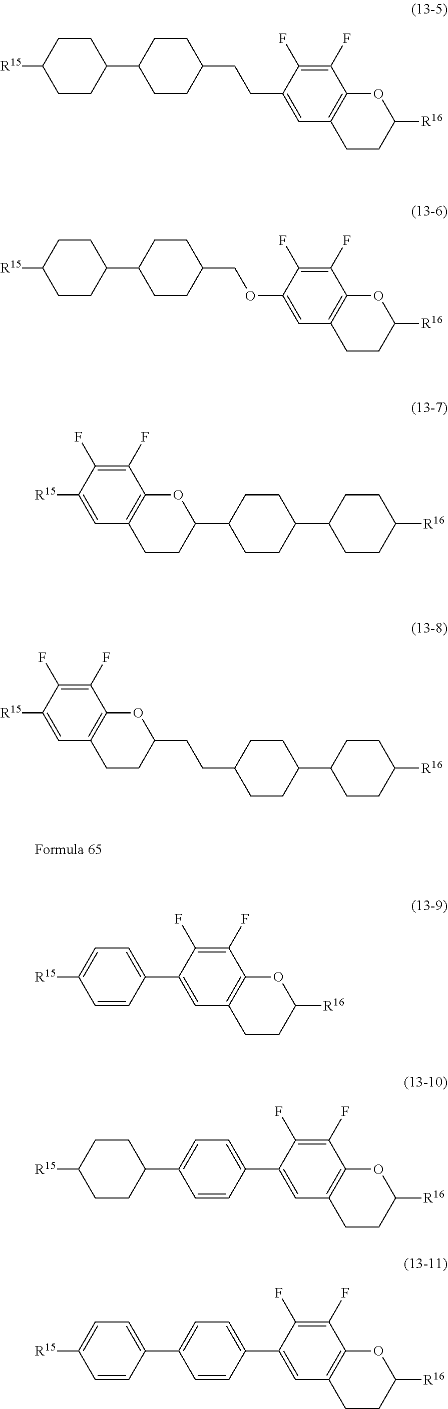

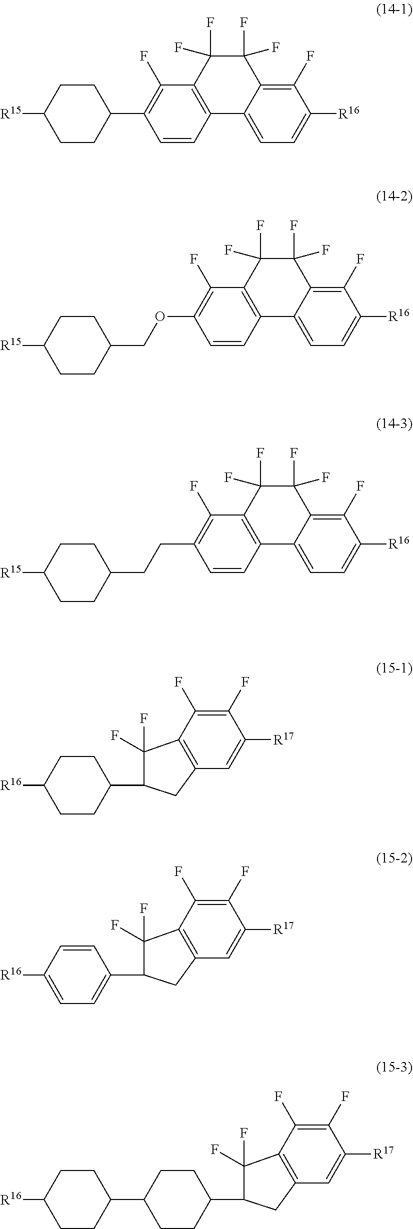

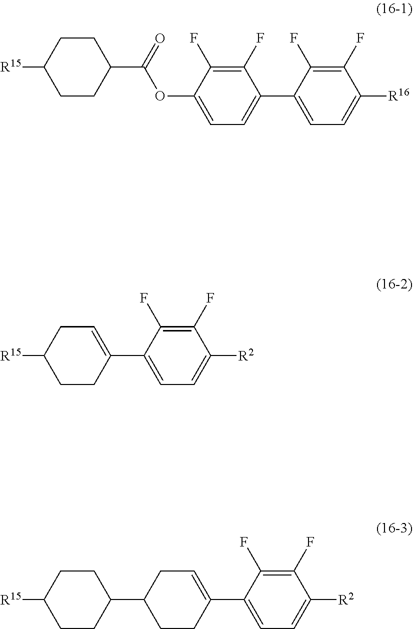

Component E has large negative dielectric anisotropy. Component E is used when a composition for the IPS mode, the VA mode, the PSA mode or the like is prepared. As a content of component E increases, the dielectric anisotropy of the composition negatively increases, but the viscosity increases. Thus, as long as a desired value of threshold voltage of the device is met, the content is preferably as small as possible. When the dielectric anisotropy at a degree of −5 is taken into account, the content of component E is preferably 40% by weight or more based on the weight of the liquid crystal composition in order to allow sufficient voltage driving.

Among types of component E, compound (9) is a bicyclic compound, and therefore is mainly effective in decreasing the viscosity, adjusting the optical anisotropy or increasing the dielectric anisotropy. Compounds (10) and (11) are a tricyclic compound, and therefore are effective in increasing the maximum temperature, the optical anisotropy or the dielectric anisotropy. Compounds (12) to (16) are effective in increasing the dielectric anisotropy.

When a composition for the IPS mode, the VA mode, the PSA mode or the like is prepared, the content of component E is preferably 40% by weight or more, and further preferably in the range of 50% by weight to 95% by weight, based on the weight of the liquid crystal composition. When component E is added to a composition having positive dielectric anisotropy, the content of component E is preferably 30% by weight or less based on the weight of the liquid crystal composition. Addition of component E allows adjustment of the elastic constant of the composition and adjustment of the voltage-transmittance curve of the device.

A liquid crystal composition satisfying at least one of characteristics such as high maximum temperature, low minimum temperature, small viscosity, suitable optical anisotropy, large positive or negative dielectric anisotropy, large specific resistance, high stability to ultraviolet light, high stability to heat and a large elastic constant can be prepared by suitably combining components B, C, D and E described above. A liquid crystal compound different from components B, C, D and E may be added thereto when necessary.

The liquid crystal composition is prepared according to a publicly-known method. For example, component compounds are mixed and dissolved in each other by heating. According to an application, an additive may be added to the composition. Specific examples of the additive include a polymerizable compound other than formula (1) and formula (16), a polymerization initiator, a polymerization inhibitor, an optically active compound, an antioxidant, an ultraviolet light absorber, a light stabilizer, a heat stabilizer and an antifoaming agent. Such an additive is well known to those skilled in the art, and described in literature.

The polymerizable compound is added for the purpose of forming the polymer in the liquid crystal composition. The polymerizable compound and compound (1) are copolymerized by irradiation with ultraviolet light in a state in which voltage is applied between electrodes to form the polymer in the liquid crystal composition. On the occasion, compound (1) is fixed in a state in which the polar group noncovalently interacts with a substrate surface of glass (or metal oxide). Thus, ability to control alignment of liquid crystal molecules is further improved, and simultaneously compound (1) no longer leaks out into the liquid crystal composition. Moreover, suitable pretilt can be obtained also on the substrate surface of glass (or metal oxide), and therefore the liquid crystal display device in which a response time is shortened and the voltage holding ratio is large can be obtained.

Preferred examples of the polymerizable compound include acrylate, methacrylate, a vinyl compound, a vinyloxy compound, propenyl ether, an epoxy compound (oxirane, oxetane) and vinyl ketone. Further preferred examples include a compound having at least one acryloyloxy, and a compound having at least one methacryloyloxy. Still further preferred examples also include a compound having both acryloyloxy and methacryloyloxy.

Still further preferred examples of the polymerizable compound include compounds (M-1) to (M-17). In compounds (M-1) to (M-17), R²⁵ to R³¹ are independently hydrogen or methyl; s, v and x are independently 0 or 1; t and u are independently an integer from 1 to 10; and L²¹ to L²⁶ are independently hydrogen or fluorine, and L²⁷ and L²⁸ are independently hydrogen, fluorine or methyl.

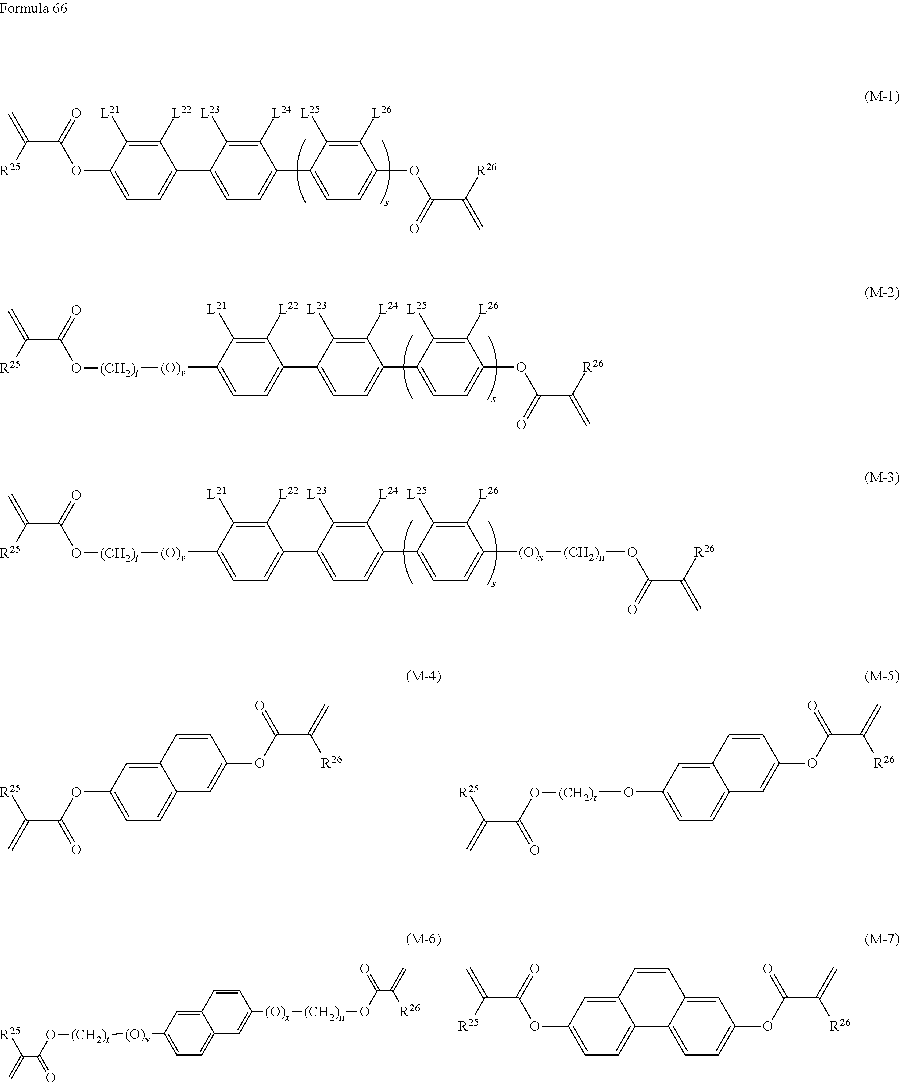

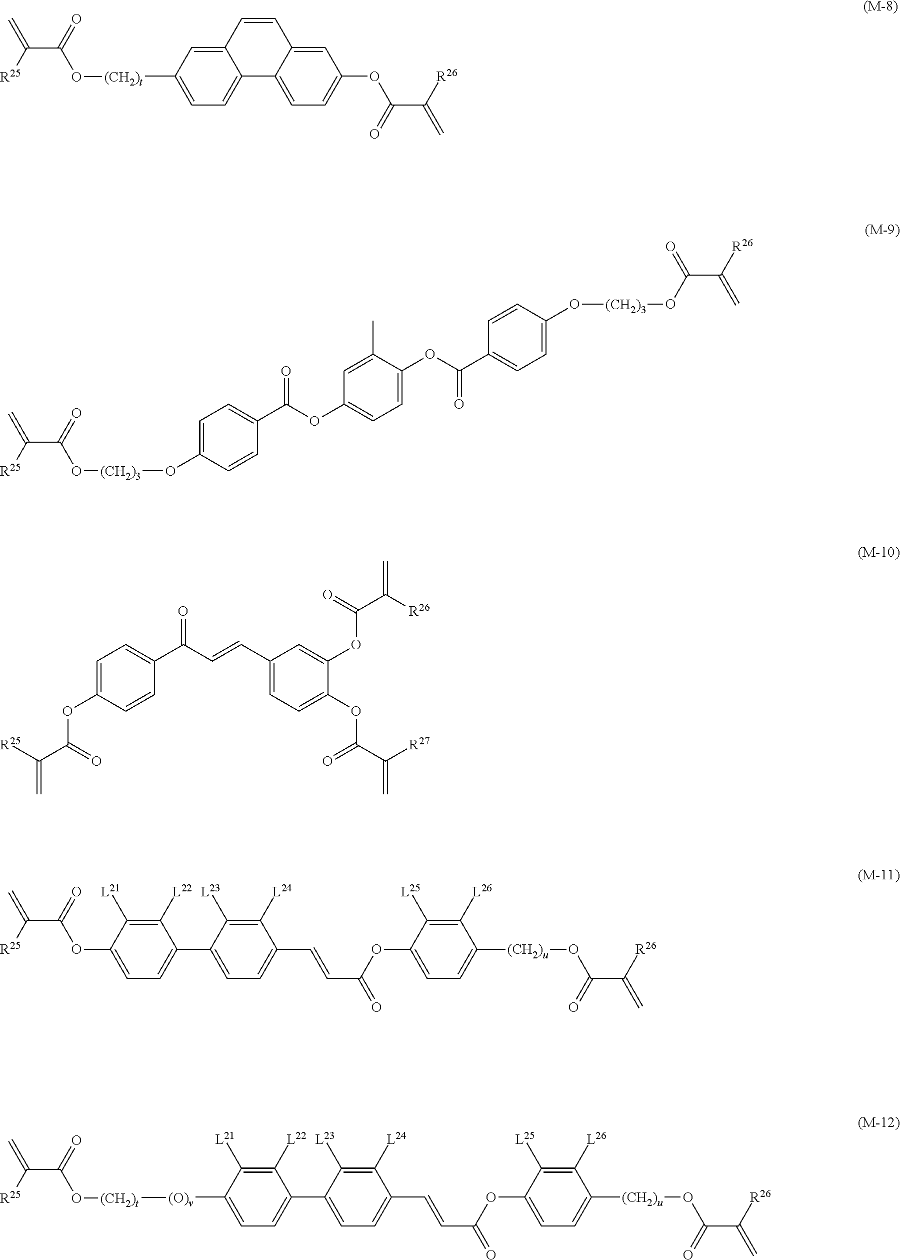

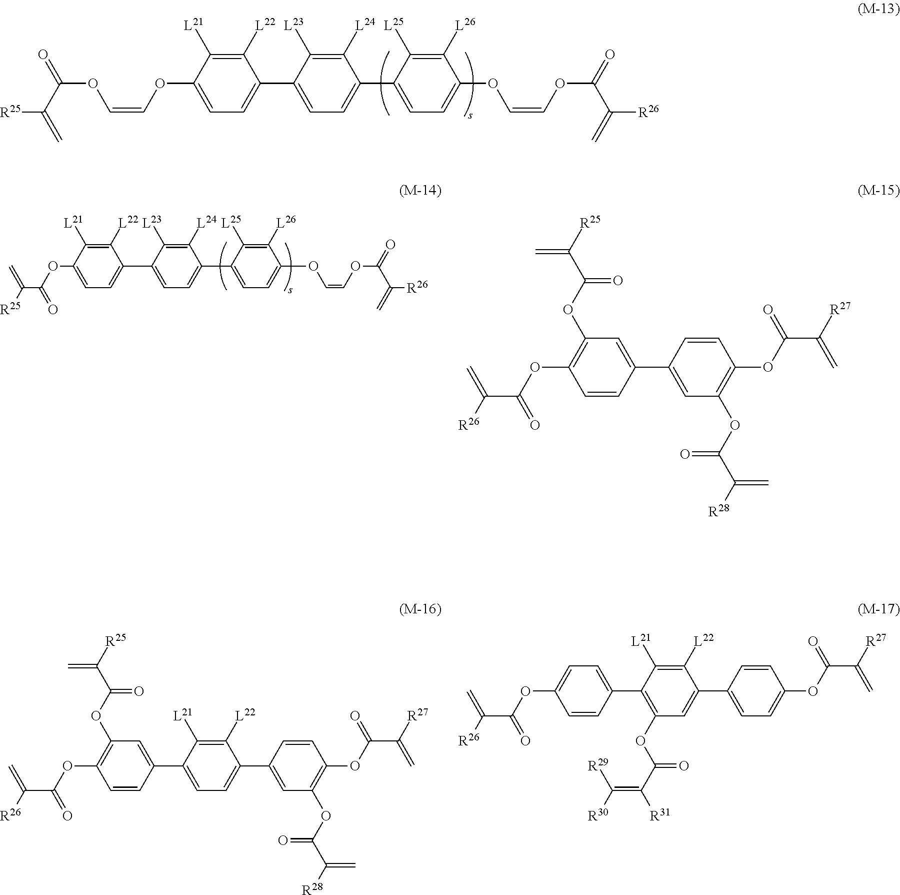

The polymerizable compound can be rapidly polymerized by adding the polymerization initiator. An amount of a remaining polymerizable compound can be decreased by optimizing a reaction temperature. Specific examples of a photoradical polymerization initiator include TPO, 1173 and 4265 from Darocur series of BASF SE, and 184, 369, 500, 651, 784, 819, 907, 1300, 1700, 1800, 1850 and 2959 from Irgacure series thereof.

Additional examples of the photoradical polymerization initiator include 4-methoxyphenyl-2,4-bis(trichloromethyl)triazine, 2-(4-butoxystyryl)-5-trichloromethyl-1,3,4-oxadiazole, 9-phenylacridine, 9,10-benzphenazine, a benzophenone-Michler's ketone mixture, a hexaarylbiimidazole-mercaptobenzimidazole mixture, 1-(4-isopropylphenyl)-2-hydroxy-2-methylpropane-1-one, benzyl dimethyl ketal, 2-methyl-1-[4-(methylthio) phenyl]-2-morpholinopropane-1-one, a mixture of 2,4-diethylxanthone and methyl p-dimethylaminobenzoate and a mixture of benzophenone and methyltriethanolamine.

After the photoradical polymerization initiator is added to the liquid crystal composition, polymerization can be performed by irradiation with ultraviolet light in a state in which an electric field is applied thereto. However, an unreacted polymerization initiator or a decomposition product of the polymerization initiator might cause a poor display such as image persistence in the device. In order to prevent such a phenomenon, photopolymerization may be performed with no addition of the polymerization initiator. A preferred wavelength of irradiation light is in the range of 150 nanometers to 500 nanometers. A further preferred wavelength is in the range of 250 nanometers to 450 nanometers, and a most preferred wavelength is in the range of 300 nanometers to 400 nanometers.

Upon mixing compound (1) having an ester bonding group, a cinnamic acid ester bond, a chalcone skeleton or a stilbene skeleton in the composition, a main effect of compound (1) that is component A on the characteristics of the composition is as described below. In the compound (1), when Fries rearrangement, photo-dimerization or cis-trans isomerization of a double bond occurs by polarized light, arrangement in a fixed direction is caused at a molecular level. Accordingly, a thin film prepared from the polar compound aligns the liquid crystal molecules in the same manner as an alignment film of polyimide or the like.

In the case of compound (1) having an aromatic ester and the polymerizable group, an aromatic ester moiety compound (1) is irradiated with ultraviolet light to cause photolysis. Thus, a radical is formed to cause photo-Fries rearrangement.

In the photo-Fries rearrangement, the photolysis of the aromatic ester moiety occurs when a polarization direction of polarized ultraviolet light and a major axis direction of the aromatic ester moiety are in the same direction. After the photolysis, recombination occurs, and a hydroxyl group is formed in the molecule by tautomerization. Interaction in a substrate interface is considered to be caused by the hydroxyl group to facilitate adsorption of the polar compound on a side of the substrate interface with anisotropy. Moreover, compound (1) has the polymerizable group, and therefore compound (1) reacting along a direction of polarized light by polymerization is immobilized without losing orientation thereof. The thin film capable of aligning the liquid crystal molecule can be prepared by utilizing the property. Linearly polarized light is suitable as ultraviolet light used for irradiation in order to prepare the thin film. First, compound (1) that is the polar compound is added to the liquid crystal composition in the range of 0.1% by weight to 10% by weight, and the resulting composition is warmed in order to dissolve the polar compound therein. The resulting composition is injected into a device having no alignment film. Next, the device is irradiated with the linearly polarized light while warming the device to cause the photo-Fries rearrangement of the polar compound to polymerize the compound.

In the photo-Fries rearranged polar compounds, arrangement in a fixed direction is caused, and the thin film formed after polymerization has a function as a liquid crystal alignment film.

Upon storing the polymerizable compound, the polymerization inhibitor may be added thereto for preventing polymerization. The polymerizable compound is ordinarily added to the composition without removing the polymerization inhibitor. Specific examples of the polymerization inhibitor include hydroquinone, a hydroquinone derivative such as methylhydroquinone, 4-t-butylcatechol, 4-methoxyphenol and phenothiazine.

The optically active compound produces an effect of inducing helical structure in the liquid crystal molecules to give a required twist angle, thereby preventing a reverse twist. A helical pitch can be adjusted by adding the optically active compound thereto. Two or more optically active compounds may be added for the purpose of adjusting temperature dependence of the helical pitch. Preferred examples of the optically active compound include compounds (Op-1) to (Op-18) described below. In compound (Op-18), ring J is 1,4-cyclohexylene or 1,4-phenylene, and R²⁸ is alkyl having 1 to 10 carbons.

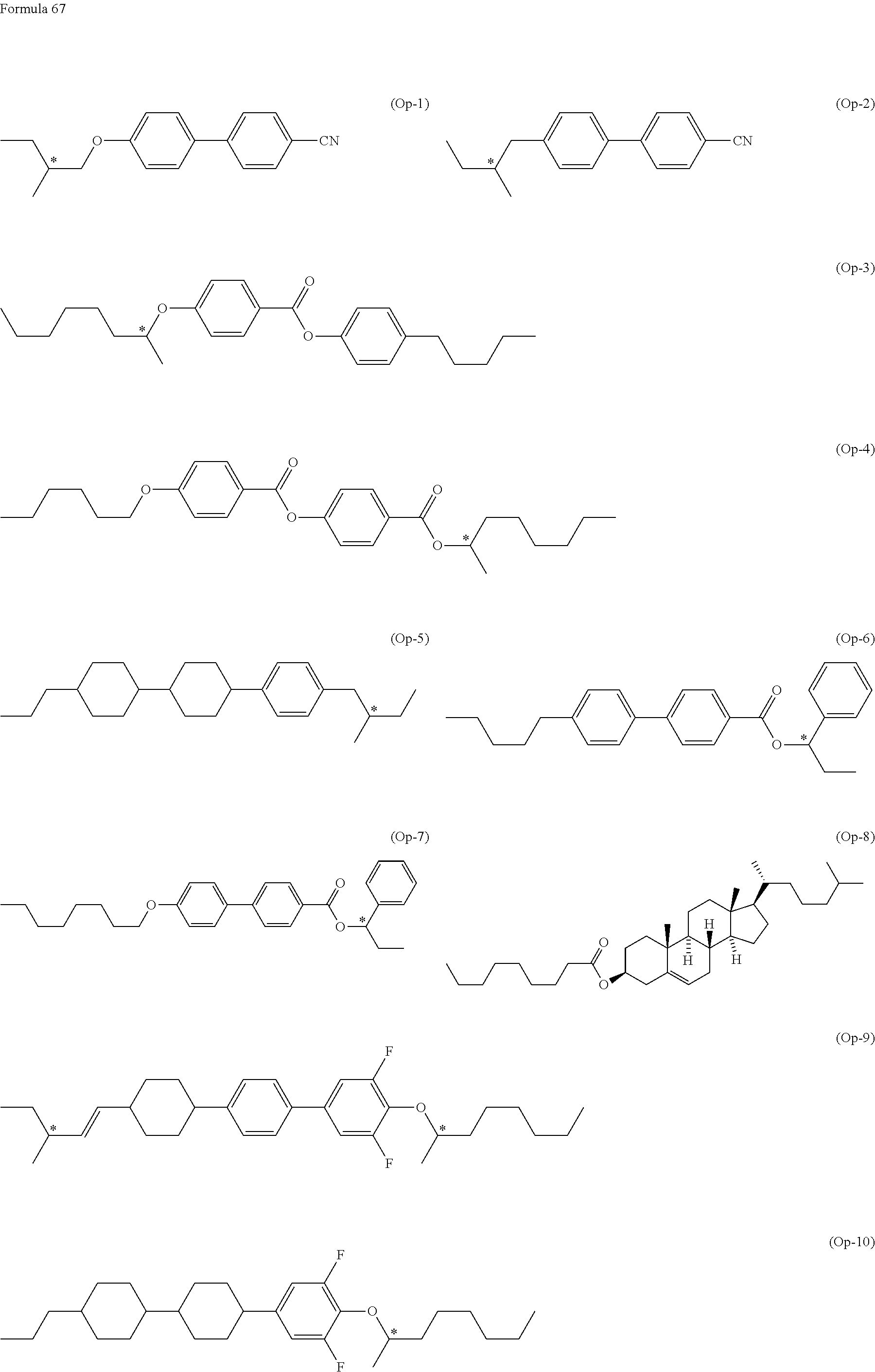

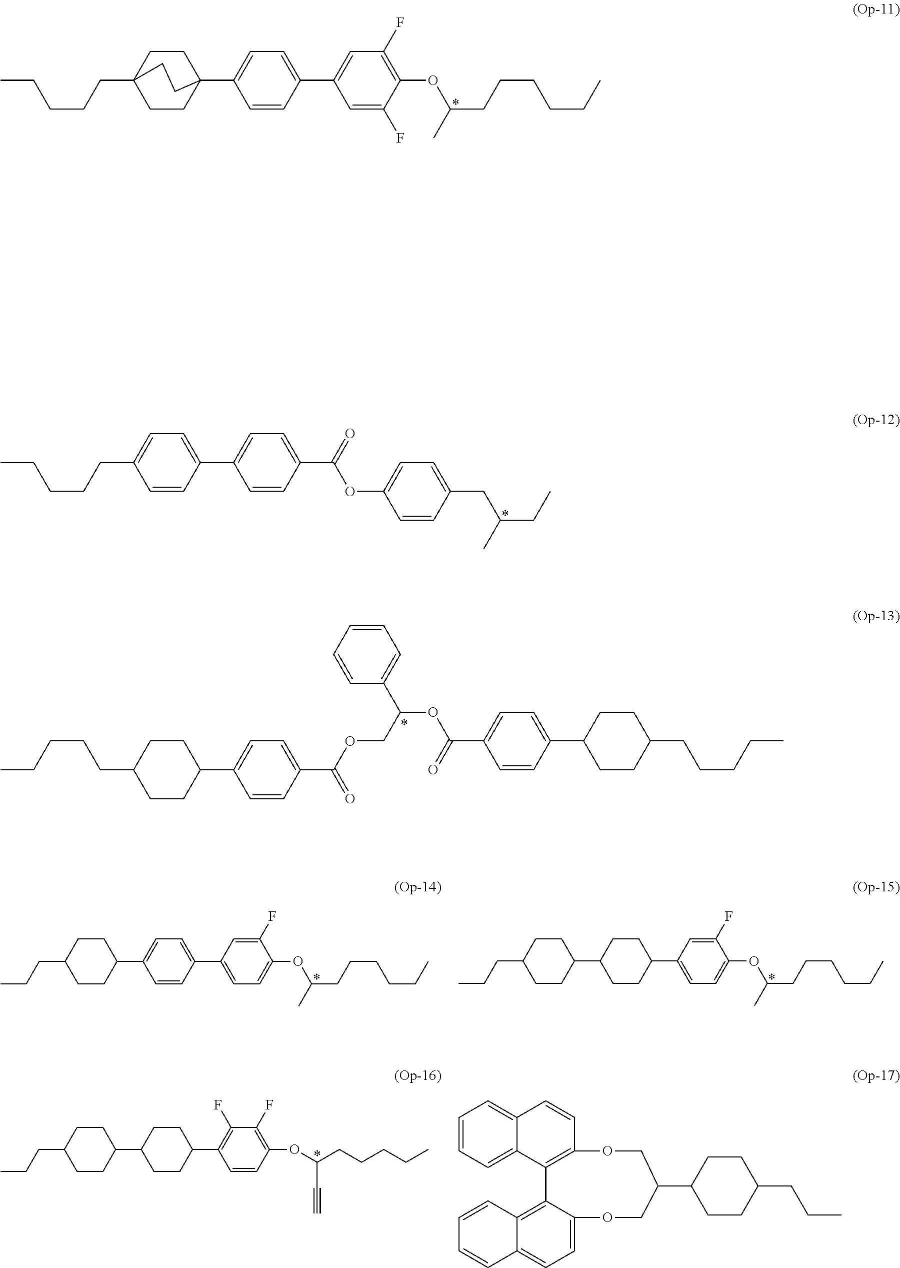

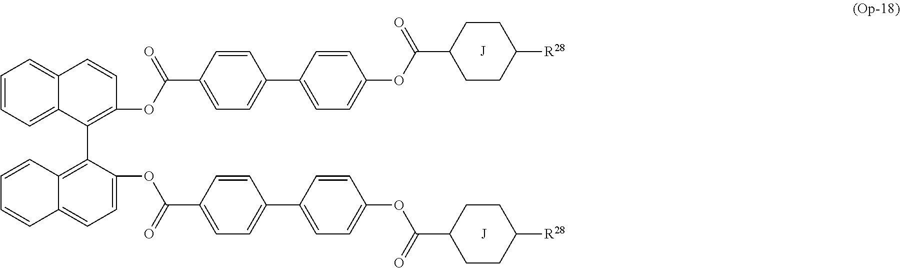

The antioxidant is effective for maintaining the large voltage holding ratio. Preferred examples of the antioxidant include compounds (AO-1) and (AO-2) described below; and IRGANOX 415, IRGANOX 565, IRGANOX 1010, IRGANOX 1035, IRGANOX 3114 and IRGANOX 1098 (trade names; BASF SE). The ultraviolet light absorber is effective for preventing a decrease of the maximum temperature. Preferred examples of the ultraviolet light absorber include a benzophenone derivative, a benzoate derivative and a triazole derivative. Specific examples thereof include compounds (AO-3) and (AO-4) described below; TINUVIN 329, TINUVIN P, TINUVIN 326, TINUVIN 234, TINUVIN 213, TINUVIN 400, TINUVIN 328 and TINUVIN 99-2 (trade names; BASF SE); and 1,4-diazabicyclo[2.2.2]octane (DABCO).

The light stabilizer such as an amine having steric hindrance is preferred for maintaining the large voltage holding ratio. Preferred examples of the light stabilizer include compounds (AO-5) and (AO-6) described below; and TINUVIN 144, TINUVIN 765 and TINUVIN 770DF (trade names: BASF SE). The heat stabilizer is also effective for maintaining the large voltage holding ratio, and preferred examples include IRGAFOS 168 (trade name: BASF SE). The antifoaming agent is effective for preventing foam formation. Preferred examples of the antifoaming agent include dimethyl silicone oil and methylphenyl silicone oil.

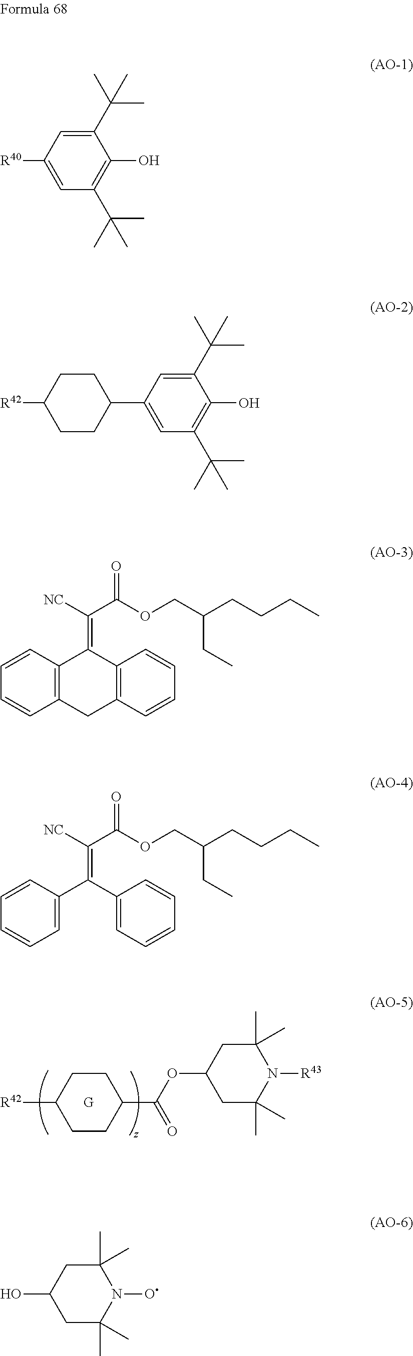

In compound (AO-1), R⁴⁰ is alkyl having 1 to 20 carbons, alkoxy having 1 to 20 carbons, —COOR⁴¹ or —CH₂CH₂COOR⁴¹, in which R⁴¹ is alkyl having 1 to 20 carbons. In compounds (AO-2) and (AO-5), R⁴² is alkyl having 1 to 20 carbons. In compound (AO-5), R⁴³ is hydrogen, methyl or O⁻ (oxygen radical), ring G is 1,4-cyclohexylene or 1,4-phenylene, and z is 1, 2 or 3.

### 4. Liquid Crystal Display Device

The liquid crystal composition can be used in a liquid crystal display device having an operating mode such as the PC mode, the TN mode, the STN mode, the OCB mode and the PSA mode, and driven by an active matrix mode. The composition can also be used in a liquid crystal display device having an operating mode such as the PC mode, the TN mode, the STN mode, the OCB mode, the VA mode and the IPS mode, and driven by a passive matrix mode. The devices can be applied to any of a reflective type, a transmissive type and a transflective type.

The composition can also be used in a nematic curvilinear aligned phase (NCAP) device prepared by microencapsulating a nematic liquid crystal, and a polymer dispersed liquid crystal display device (PDLCD) and a polymer network liquid crystal display device (PNLCD) prepared by forming a three-dimensional network-polymer in the liquid crystal. When an amount of adding the polymerizable compound is about 10% by weight or less based on the weight of the liquid crystal composition, the liquid crystal display device having the PSA mode can be prepared. A preferred proportion of the polymerizable compound is in the range of about 0.1% by weight to about 2% by weight based on the weight of the liquid crystal composition. A further preferred proportion is in the range of about 0.2% by weight to about 1.0% by weight based on the weight of the liquid crystal composition. The device having the PSA mode can be driven by a driving mode such as the active matrix mode and the passive matrix mode. Such a device can also be applied to any of a reflective type, a transmissive type and a transflective type. A device having a polymer dispersed mode can also be prepared by increasing the amount of adding the polymerizable compound.

In the polymer sustained alignment mode device, the polymer contained in the composition aligns the liquid crystal molecules. Compound (1) that is the polar compound supports arrangement of liquid crystal molecules. More specifically, compound (1) can be used in place of the alignment film. One example of a method for producing such a device is as described below.

A device having two substrates called an array substrate and a color filter substrate is arranged. The substrates have no alignment film. At least one of the substrates has an electrode layer. A liquid crystal composition is prepared by mixing liquid crystal compounds. A polymerizable compound and compound (1) that is a polar compound are added to the composition. An additive may be further added thereto when necessary. The composition is injected into a device. The device is irradiated with light. Ultraviolet light is preferred. The polymerizable compound is polymerized by irradiation with light. The composition containing the polymer is formed by polymerization, and a device having a PSA mode is prepared.

A method for producing the device will be described. First, the method includes a step of adding compound (1) that is a polar compound to a liquid crystal composition, and then warming the resulting composition at a temperature higher than the maximum temperature thereof to dissolve the composition. Second, the method includes a step of injecting the composition into a liquid crystal display device. Third, the method includes a step of irradiating the composition with polarized ultraviolet light while warming the liquid crystal composition at a temperature higher than the maximum temperature thereof. Compound (1) that is the polar compound causes any one of the photo-Fries rearrangement, photo-dimerization or cis-trans isomerization of a double bond by linearly polarized light, and simultaneously polymerization thereof also progresses. A polymer of compound (1) is formed as the thin film on the substrate, and immobilized thereon. In the polymer, arrangement in a fixed direction is caused at a molecular level, and therefore the thin film has the function as the liquid crystal alignment film. A liquid crystal display device having no alignment film of polyimide or the like can be produced by the method described above.

In the procedure, compound (1) that is the polar compound is eccentrically located on a substrate because the polar group interacts with the substrate surface. Compound (1) aligns the liquid crystal molecules by irradiation with polarized ultraviolet light, and simultaneously the polymerizable compound is polymerized by ultraviolet light, and therefore a polymer maintaining the alignment is formed. The alignment of the liquid crystal molecules is additionally stabilized by an effect of the polymer, and therefore the response time in the device is shortened. The image persistence is caused due to poor operation of the liquid crystal molecules, and therefore the persistence is also simultaneously improved by the effect of the polymer. In particular, compound (1) according to an embodiment of the invention is a polymerizable polar compound, and therefore aligns the liquid crystal molecules, and also is copolymerized with any other polymerizable compound. Thus, the polar compound no longer leaks out into the liquid crystal composition, and therefore the liquid crystal display device having the large voltage holding ratio can be obtained.

### EXAMPLES

Hereinafter, the invention will be described in greater detail by way of Examples (including Synthesis Examples and Use Examples of devices). However, the invention is not limited by the Examples. The invention includes a mixture of a composition in Use Example 1 and a composition in Use Example 2. The invention also includes a mixture prepared by mixing at least two compositions in each Use Example.

### 1. Example of Compound (1)

Compound (1) was prepared according to procedures shown in Example 1 or the like. Unless otherwise specified, a reaction was performed under a nitrogen atmosphere. The thus prepared compound was identified by methods such as an NMR analysis. Characteristics of compound (1), a liquid crystal compound, a composition and a device were measured by methods described below.

NMR analysis: For measurement, DRX-500 made by Bruker BioSpin Corporation was used. In ¹H-NMR measurement, a sample was dissolved in a deuterated solvent such as CDCl₃, and measurement was carried out under conditions of room temperature, 500 MHz and 16 times of accumulation. Tetramethylsilane was used as an internal standard. In ¹⁹F-NMR measurement, CFCl₃ was used as an internal standard, and measurement was carried out under conditions of 24 times of accumulation. In explaining nuclear magnetic resonance spectra obtained, s, d, t, q, quip, sex and m stand for a singlet, a doublet, a triplet, a quartet, a quintet, a sextet and a multiplet, and br being broad, respectively.

Gas chromatographic analysis: For measurement, GC-2010 Gas Chromatograph made by Shimadzu Corporation was used. As a column, a capillary column DB-1 (length 60 m, bore 0.25 mm, film thickness 0.25 μm) made by Agilent Technologies, Inc. was used. As a carrier gas, helium (1 mL/minute) was used. A temperature of a sample vaporizing chamber and a temperature of a detector (FID) part were set to 300° C. and 300° C., respectively. A sample was dissolved in acetone and prepared to be a 1 weight % solution, and then 1 microliter of the solution obtained was injected into the sample vaporizing chamber. As a recorder, GC Solution System made by Shimadzu Corporation or the like was used.

HPLC Analysis: For measurement, Prominence (LC-20AD; SPD-20A) made by Shimadzu Corporation was used. As a column, YMC-Pack ODS-A (length 150 mm, bore 4.6 mm, particle diameter 5 μm) made by YMC Co., Ltd. was used. As an eluate, acetonitrile and water were appropriately mixed and used. As a detector, a UV detector, an RI detector, a CORONA detector or the like was appropriately used. When the UV detector was used, a detection wavelength was set at 254 nanometers. A sample was dissolved in acetonitrile and prepared to be a 0.1 weight % solution, and then 1 microliter of the solution was injected into a sample chamber. As a recorder, C-R7Aplus made by Shimadzu Corporation was used.

Ultraviolet-Visible Spectrophotometry: For measurement, PharmaSpec UV-1700 made by Shimadzu Corporation was used. A detection wavelength was adjusted in the range of 190 nanometers to 700 nanometers. A sample was dissolved in acetonitrile and prepared to be a 0.01 mmol/L solution, and measurement was carried out by putting the solution in a quartz cell (optical path length: 1 cm).

Sample for measurement: Upon measuring phase structure and a transition temperature (a clearing point, a melting point, a polymerization starting temperature or the like), a compound itself was used as a sample.

Measuring method: Characteristics were measured according to methods described below. Most of the measuring methods are applied as described in the Standard of Japan Electronics and Information Technology Industries Association (hereinafter, abbreviated as JEITA) (JEITA ED-2521B) discussed and established by JEITA, or modified thereon. No thin film transistor (TFT) was attached to a TN device used for measurement.

### (1) Phase Structure

A sample was placed on a hot plate in a melting point apparatus (FP-52 Hot Stage made by Mettler-Toledo International Inc.) equipped with a polarizing microscope. A state of phase and a change thereof were observed with the polarizing microscope while the sample was heated at a rate of 3° C. per minute, and a kind of the phase was specified.

### (2) Transition Temperature (° C.)

For measurement, a scanning calorimeter, Diamond DSC System, made by PerkinElmer, Inc., or a high sensitivity differential scanning calorimeter, X-DSC7000, made by SII Nano Technology Inc. was used. A sample was heated and then cooled at a rate of 3° C. per minute, and a starting point of an endothermic peak or an exothermic peak caused by a phase change of the sample was determined by extrapolation, and thus a transition temperature was determined. A melting point and a polymeration starting temperature of a compound were also measured using the apparatus. Temperature at which a compound undergoes transition from a solid to a liquid crystal phase such as the smectic phase and the nematic phase may be occasionally abbreviated as “minimum temperature of the quid crystal phase.” Temperature at which the compound undergoes transition from the liquid crystal phase to liquid may be occasionally abbreviated as “clearing point.”

A crystal was expressed as C. When kinds of the crystals were distinguishable, each of the crystals was expressed as C₁ or C₂. The smectic phase and the nematic phase were expressed as S and N, respectively. When smectic A phase, smectic B phase, smectic C phase or smectic F phase was distinguishable among the smectic phases, the phases were expressed as S_{A}, S_{B}, S_{C} or S_{F}, respectively. A liquid (isotropic) was expressed as I. A transition temperature was expressed as “C 50.0 N 100.0 I,” for example. The expression indicates that a transition temperature from the crystals to the nematic phase is 50.0° C., and a transition temperature from the nematic phase to the liquid is 100.0° C.

### (3) Maximum Temperature of Nematic Phase (T_{NI} or NI; ° C.)

A sample was placed on a hot plate in a melting point apparatus equipped with a polarizing microscope, and heated at a rate of 1° C. per minute. Temperature when part of the sample began to change from a nematic phase to an isotropic liquid was measured. A maximum temperature of the nematic phase may be occasionally abbreviated as “maximum temperature.” When the sample was a mixture of compound (1) and a base liquid crystal, the maximum temperature was expressed in terms of a symbol T_{NI}. When the sample was a mixture of compound (1) and a compound such as component B, C or D, the maximum temperature was expressed as a symbol NI.

### (4) Minimum Temperature of Nematic Phase (T_{C}; ° C.)

Samples each having a nematic phase were kept in freezers at temperatures of 0° C., −10° C., −20° C., −30° C. and −40° C. for 10 days, and then liquid crystal phases were observed. For example, when the sample maintained the nematic phase at −20° C. and changed to crystals or the smectic phase at −30° C., T_{C} was expressed as T_{C}≤−20° C. A minimum temperature of the nematic phase may be occasionally abbreviated as “minimum temperature.”

(5) Viscosity (Bulk Viscosity; η; Measured at 20° C.; mPa·s)

For measurement, a cone-plate (E type) rotational viscometer made by Tokyo Keiki Inc. was used.

### (6) Optical Anisotropy (Refractive Index Anisotropy; Measured at 25° C.; Δn)

Measurement was carried out by an Abbe refractometer with a polarizing plate mounted on an ocular, using light at a wavelength of 589 nanometers. A surface of a main prism was rubbed in one direction, and then a sample was added dropwise onto the main prism. A refractive index (n∥) was measured when a direction of polarized light was parallel to a direction of rubbing. A refractive index (n⊥) was measured when the direction of polarized light was perpendicular to the direction of rubbing. A value of optical anisotropy (Δn) was calculated from an equation: Δn=n∥−n⊥.

### (7) Specific Resistance (ρ; Measured at 25° C.; Ω cm)

Into a vessel equipped with electrodes, 1.0 milliliter of sample was injected. A direct current voltage (10 V) was applied to the vessel, and a direct current after 10 seconds was measured. Specific resistance was calculated from the following equation: (specific resistance)={(voltage)×(electric capacity of a vessel)}/{(direct current)×(dielectric constant of vacuum)}.

Measuring methods of characteristics may be occasionally different between a sample having positive dielectric anisotropy and a sample having negative dielectric anisotropy. Measuring methods of the sample having positive dielectric anisotropy were described in sections (8a) to (12a). Measuring methods of the sample having negative dielectric anisotropy were described in sections (8b) to (12b).

(8a) Viscosity (Rotational Viscosity; γ1; Measured at 25° C.; mPa·s)

Positive dielectric anisotropy: Measurement was carried out according to a method described in M. Imai et al., Molecular Crystals and Liquid Crystals, Vol. 259, 37 (1995). A sample was put in a TN device in which a twist angle was 0 degrees and a distance (cell gap) between two glass substrates was 5 micrometers. Voltage was applied stepwise to the device in the range of 16 V to 19.5 V at an increment of 0.5 V. After a period of 0.2 second with no voltage application, voltage was repeatedly applied under conditions of only one rectangular wave (rectangular pulse; 0.2 second) and no voltage application (2 seconds). A peak current and a peak time of transient current generated by the applied rectangular waves were measured. A value of rotational viscosity was obtained from the measured values and calculation equation (8) described on page 40 of the paper presented by M. Imai et al. A value of dielectric anisotropy required for the calculation was determined using the device by which the rotational viscosity was measured and by a method described below.

(8b) Viscosity (Rotational Viscosity; γ1; Measured at 25° C.; mPa·s)

Negative dielectric anisotropy: Measurement was carried out according to a method described in M. Imai et al., Molecular Crystals and Liquid Crystals, Vol. 259, 37 (1995). A sample was put in a VA device in which a distance (cell gap) between two glass substrates was 20 micrometers. Voltage was applied stepwise to the device in the range of 39 V to 50 V at an increment of 1 V. After a period of 0.2 second with no voltage application, voltage was repeatedly applied under conditions of only one rectangular wave (rectangular pulse; 0.2 second) and no voltage application (2 seconds). A peak current and a peak time of transient current generated by the applied rectangular waves were measured. A value of rotational viscosity was obtained from the measured values and calculation equation (8) described on page 40 of the paper presented by M. Imai et al. In dielectric anisotropy required for the calculation, a value measured in a section of the dielectric anisotropy as described below was used.

### (9a) Dielectric Anisotropy (Δε; Measured at 25° C.)

Positive dielectric anisotropy: A sample was put in a TN device in which a distance (cell gap) between two glass substrates was 9 micrometers and a twist angle was 80 degrees. Sine waves (10 V, 1 kHz) were applied to the device, and after 2 seconds, a dielectric constant (ε∥) of liquid crystal molecules in a major axis direction was measured. Sine waves (0.5 V, 1 kHz) were applied to the device, and after 2 seconds, a dielectric constant (ε⊥) of liquid crystal molecules in a minor axis direction was measured. A value of dielectric anisotropy was calculated from an equation: Δε=ε∥−ε⊥.

### (9b) Dielectric Anisotropy (Δε; Measured at 25° C.)

Negative dielectric anisotropy: A value of dielectric anisotropy was calculated from an equation: Δε=ε∥−ε⊥. A dielectric constant (ε∥ and ε⊥) was measured as described below.

(1) Measurement of dielectric constant (ε∥): An ethanol (20 mL) solution of octadecyltriethoxysilane (0.16 mL) was applied to a well-cleaned glass substrate. After rotating the glass substrate with a spinner, the glass substrate was heated at 150° C. for 1 hour. A sample was put in a VA device in which a distance (cell gap) between two glass substrates was 4 micrometers, and the device was sealed with an ultraviolet-curable adhesive. Sine waves (0.5 V, 1 kHz) were applied to the device, and after 2 seconds, a dielectric constant (ε∥) of liquid crystal molecules in a major axis direction was measured.

(2) Measurement of dielectric constant (ε⊥): A polyimide solution was applied to a well-cleaned glass substrate. After calcining the glass substrate, rubbing treatment was applied to the alignment film obtained. A sample was put in a TN device in which a distance (cell gap) between two glass substrates was 9 micrometers and a twist angle was 80 degrees. Sine waves (0.5 V, 1 kHz) were applied to the device, and after 2 seconds, a dielectric constant (ε⊥) of liquid crystal molecules in a minor axis direction was measured.

### (10a) Elastic Constant (K; Measured at 25° C.; pN)

Positive dielectric anisotropy: For measurement, HP4284A LCR Meter made by Yokogawa-Hewlett-Packard Co. was used. A sample was put in a horizontal alignment device in which a distance (cell gap) between two glass substrates was 20 micrometers. An electric charge of 0 V to 20 V was applied to the device, and electrostatic capacity and applied voltage were measured. The measured values of electrostatic capacity (C) and applied voltage (V) were fitted to equation (2.98) and equation (2.101) on page 75 of “Liquid Crystal Device Handbook” (Ekisho Debaisu Handobukku in Japanese; Nikkan Kogyo Shimbun, Ltd.), and values of K₁₁ and K₃₃ were obtained from equation (2.99). Next, K₂₂ was calculated using the previously determined values of K₁₁ and K₃₃ in equation (3.18) on page 171. Elastic constant K was expressed in terms of a mean value of the thus determined K₁₁, K₂₂ and K₃₃.

(10b) Elastic Constant (K₁₁ and K₃₃; Measured at 25° C.; pN)

Negative dielectric anisotropy: For measurement, Elastic Constant Measurement System Model EC-1 made by TOYO Corporation was used. A sample was put in a vertical alignment device in which a distance (cell gap) between two glass substrates was 20 micrometers. An electric charge of 20 V to 0 V was applied to the device, and electrostatic capacity and applied voltage were measured. The measured values of electrostatic capacity (C) and applied voltage (V) were fitted to equation (2.98) and equation (2.101) on page 75 of “Liquid Crystal Device Handbook” (Ekisho Debaisu Handobukku, in Japanese; Nikkan Kogyo Shimbun, Ltd.), and values of elastic constant were obtained from equation (2.100).

### (11a) Threshold Voltage (Vth; Measured at 25° C.; V)

Positive dielectric anisotropy: For measurement, an LCD-5100 luminance meter made by Otsuka Electronics Co., Ltd. was used. A light source was a halogen lamp. A sample was put in a normally white mode TN device in which a distance (cell gap) between two glass substrates was 0.45/Δn (μm) and a twist angle was 80 degrees. A voltage (32 Hz, rectangular waves) to be applied to the device was stepwise increased from 0 V to 10 V at an increment of 0.02 V. On the occasion, the device was irradiated with light from a direction perpendicular to the device, and an amount of light transmitted through the device was measured. A voltage-transmittance curve was prepared, in which the maximum amount of light corresponds to 100% transmittance and the minimum amount of light corresponds to 0% transmittance. Threshold voltage was expressed in terms of voltage at 90% transmittance.

### (11b) Threshold Voltage (Vth; Measured at 25° C.; V)

Negative dielectric anisotropy: For measurement, an LCD-5100 luminance meter made by Otsuka Electronics Co., Ltd. was used. A light source was a halogen lamp. A sample was put in a normally black mode VA device in which a distance (cell gap) between two glass substrates was 4 micrometers and a rubbing direction was anti-parallel, and the device was sealed with an ultraviolet-curable adhesive. A voltage (60 Hz, rectangular waves) to be applied to the device was stepwise increased from 0 V to 20 V at an increment of 0.02 V. On the occasion, the device was irradiated with light from a direction perpendicular to the device, and an amount of light transmitted through the device was measured. A voltage-transmittance curve was prepared, in which the maximum amount of light corresponds to 100% transmittance and the minimum amount of light corresponds to 0% transmittance. Threshold voltage was expressed in terms of voltage at 10% transmittance.

### (12a) Response Time (τ; Measured at 25° C.; ms)

Positive dielectric anisotropy: For measurement, an LCD-5100 luminance meter made by Otsuka Electronics Co., Ltd. was used. A light source was a halogen lamp. A low-pass filter was set to 5 kHz. A sample was put in a normally white mode TN device in which a distance (cell gap) between two glass substrates was 5.0 micrometers and a twist angle was 80 degrees. A voltage (rectangular waves; 60 Hz, 5 V, 0.5 second) was applied to the device. On the occasion, the device was irradiated with light from a direction perpendicular to the device, and an amount of light transmitted through the device was measured. The maximum amount of light corresponds to 100% transmittance, and the minimum amount of light corresponds to 0% transmittance. A rise time (τr; millisecond) was expressed in terms of time required for a change from 90% transmittance to 10% transmittance. A fall time (τf; millisecond) was expressed in terms of time required for a change from 10% transmittance to 90% transmittance. A response time was expressed in terms of a sum of the rise time and the fall time thus obtained.

### (12b) Response Time (τ; Measured at 25° C.; ms)

Negative dielectric anisotropy: For measurement, an LCD-5100 luminance meter made by Otsuka Electronics Co., Ltd. was used. A light source was a halogen lamp. A low-pass filter was set to 5 kHz. A sample was put in a normally black mode PVA device in which a distance (cell gap) between two glass substrates was 3.2 micrometers and a rubbing direction was anti-parallel. The device was sealed with an ultraviolet-curable adhesive. Voltage at a degree of a little over threshold voltage was applied to the device for 1 minute, and then the device was irradiated with ultraviolet light of 23.5 mW/cm² for 8 minutes while voltage of 5.6 V was applied to the device. A voltage (rectangular waves; 60 Hz, 10 V, 0.5 second) was applied to the device. On the occasion, the device was irradiated with light from a direction perpendicular to the device, and an amount of light transmitted through the device was measured. The maximum amount of light corresponds to 100% transmittance, and the minimum amount of light corresponds to 0% transmittance. A response time was expressed in terms of time required fora change from 90% transmittance to 10% transmittance (fall time; millisecond).

### Raw Material

Solmix (registered trademark) A-11 is a mixture of ethanol (85.5%), methanol (13.4%) and isopropanol (1.1%), and was purchased from Japan Alcohol Trading Co., Ltd.

### Synthesis Example 1

### Synthesis of Compound (No. 156)

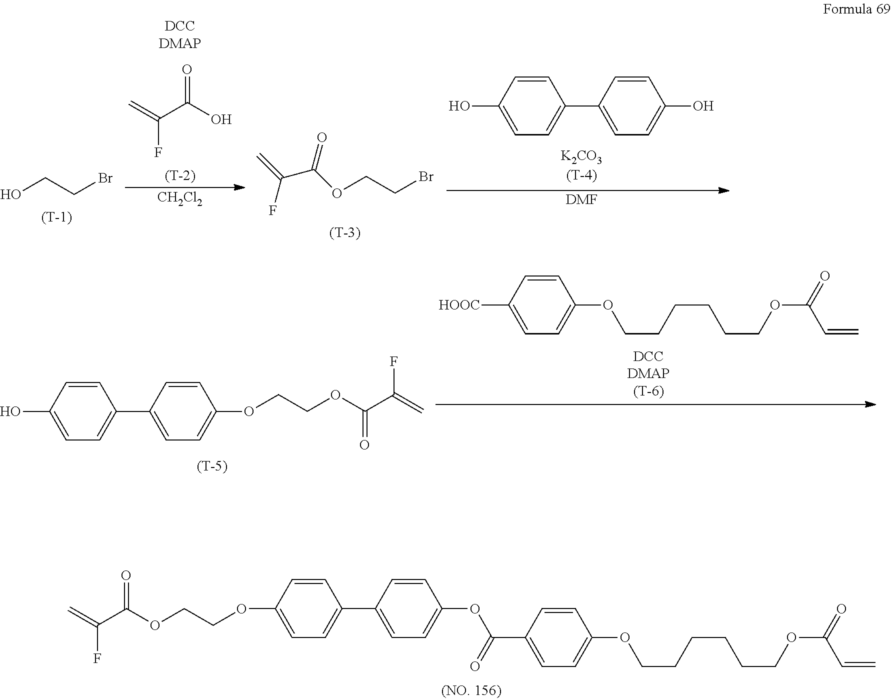

### First Step

Compound (T-1) (2.77 g), compound (T-2) (2.00 g) DMAP (0.27 g) and dichloromethane (100 mL) were put in a reaction vessel, and the resulting mixture was cooled down to 0° C. Thereto, DCC (4.81 g) was added, and the resulting mixture was stirred for 12 hours while returning to room temperature. An insoluble matter was filtered off, and then the resulting reaction mixture was poured into water, and an aqueous layer was subjected to extraction with dichloromethane. The resulting organic layer was washed with water, and dried over anhydrous magnesium sulfate. The solution was concentrated under reduced pressure, and the residue was purified by silica gel chromatography (dichloromethane) to obtain compound (T-3) (4.38 g; 100%).

### Second Step

Compound (T-3) (4.38 g), potassium carbonate (6.15 g), 4,4′-biphenyldiol (T-4) (16.5 g) and DMF (100 mL) were put in a reaction vessel, and the resulting mixture was stirred at 60° C. for 2 hours. The resulting reaction mixture was poured into water, and an aqueous layer was subjected to extraction with ethyl acetate. The resulting organic layer was washed with water, and dried over anhydrous magnesium sulfate. The solution was concentrated under reduced pressure, and the residue was purified by silica gel chromatography (ethyl acetate:toluene=1:4 in a volume ratio) to obtain compound (T-5) (3.00 g; 45%).

### Third Step

Compound (T-5) (1.20 g), compound (T-6) (1.27 g), DMAP (0.10 g) and dichloromethane (100 mL) were put in a reaction vessel, and the resulting mixture was cooled down to 0° C. Thereto, DCC (0.90 g) was added, and the resulting mixture was stirred for 12 hours while returning to room temperature. An insoluble matter was filtered off, and then the resulting reaction mixture was poured into water, and an aqueous layer was subjected to extraction with dichloromethane. The resulting organic layer was washed with water, and dried over anhydrous magnesium sulfate. The solution was concentrated under reduced pressure, and the residue was purified by silica gel chromatography (ethyl acetate:toluene=1:9 in a volume ratio) to obtain compound (No. 156) (2.00 g; 87%). In addition, compound (T-6) is a known substance, and those skilled in the art can easily obtain a synthesis method.

An NMR analysis value of the resulting compound (No. 156) was as described below.

¹H-NMR: chemical shift δ (ppm; CDCl₃): 8.16 (d, 2H), 7.57 (d, 2H) 7.53 (d, 2H), 7.25 (d, 2H), 7.00 (d, 2H), 6.98 (d, 2H), 6.41 (dd, 1H), 6.13 (dd, 1H), 5.82 (dd, 1H), 5.62 (dd, 1H), 5.37 (dd, 1H), 4.61 (t, 2H), 4.28 (t, 2H), 4.19 (t, 2H), 4.05 (t, 2H), 1.85 (quint, 2H), 1.73 (quint, 2H), 1.55 (quint, 2H), 1.46 (quint, 2H).

Physical properties of compound (No. 156) were as described below.

Transition temperature (° C.): C 91.8 I. Polymerization temperature (° C.): 132.1.

### Synthesis Example 2

### Synthesis of Compound (No. 157)

Compound (No. 157) was prepared by using compound (T-7) in place of compound (T-6) in Synthesis Example 1. In addition, compound (T-7) is a known substance, and those skilled in the art can easily obtain a synthesis method.

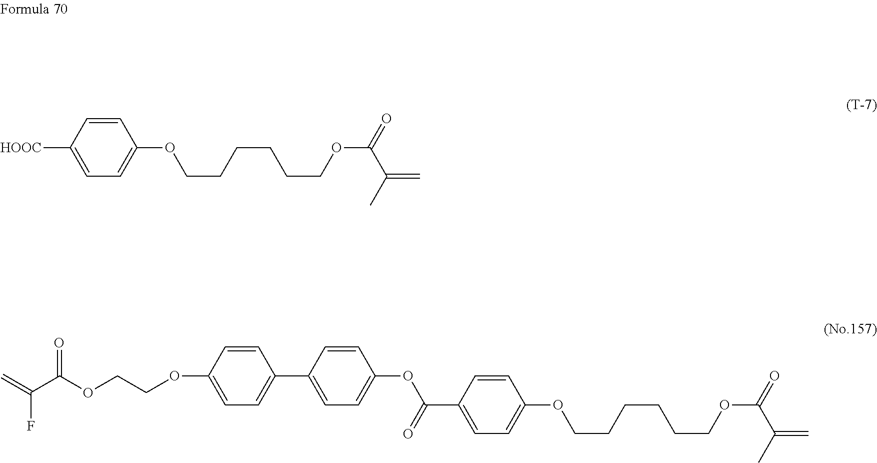 [SMILES: C=C(C)C(=O)OCCCCCCOc1ccc(C(=O)O)cc1 ; C=C(C)C(=O)OCCCCCCOc1ccc(C(=O)Oc2ccc(-c3ccc(OCCOC(=O)C(=C)F)cc3)cc2)cc1]

An NMR analysis value of the resulting compound (No. 157) was as described below.

¹H-NMR: chemical shift δ (ppm; CDCl₃): 8.16 (d, 2H), 7.58 (d, 2H), 7.52 (d, 2H), 7.25 (d, 2H), 7.00 (d, 2H), 6.97 (d, 2H), 6.10 (s, 1H), 5.72 (dd, 1H), 5.55 (t, 1H), 5.37 (dd, 1H), 4.61 (t, 2H), 4.29 (t, 2H), 4.17 (t, 2H), 4.05 (t, 2H), 1.94 (s, 3H), 1.83 (quint, 2H), 1.71 (quint, 2H), 1.55 (quint, 2H), 1.46 (quint, 2H).

Physical properties of compound (No. 157) were as described below.

Transition temperature (° C.): C 91.8 I. Polymerization temperature (° C.) 168.9.

### Synthesis Example 3

### Synthesis of Compound (No. 158)

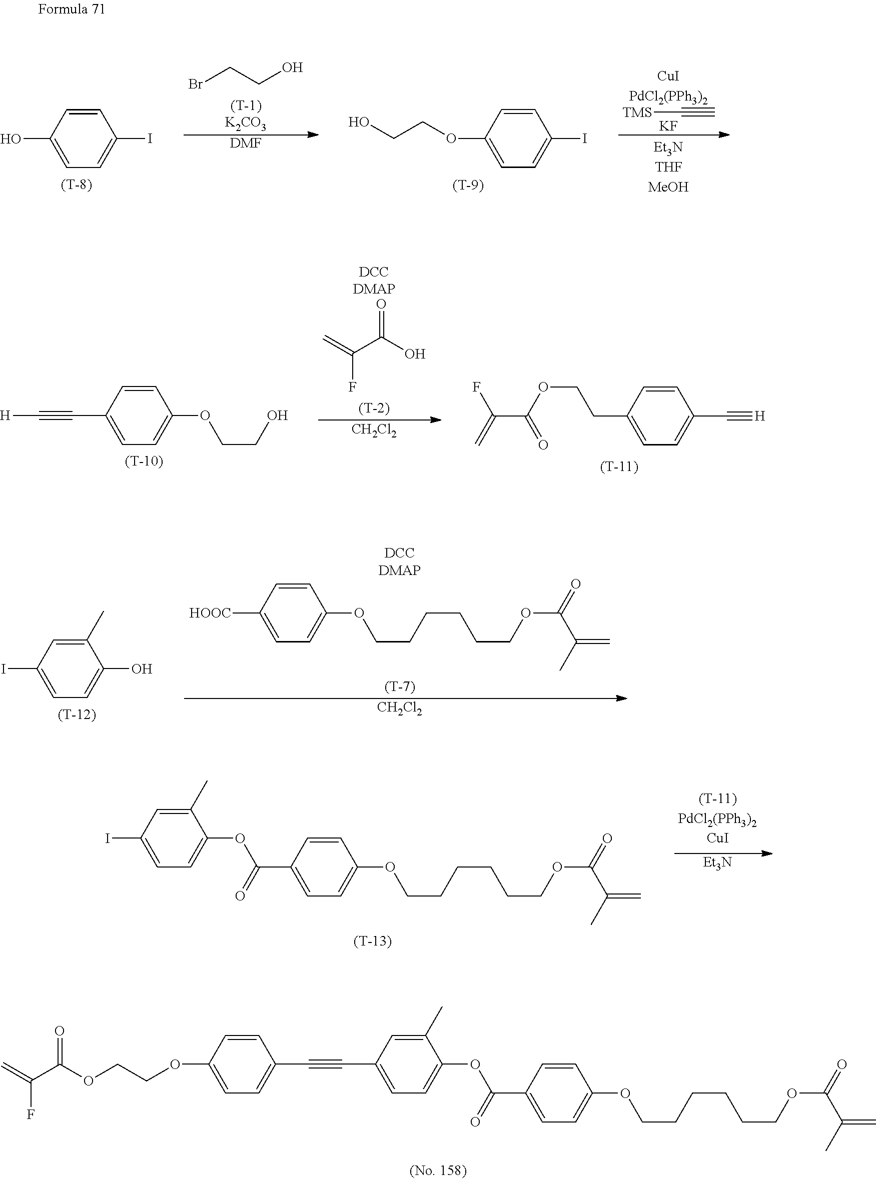

### First Step

Compound (T-8) (30.0 g), potassium carbonate (38.0 g), compound (T-1) (17.0 g) and DMF (300 mL) were put in a reaction vessel, and the resulting mixture was stirred at 100° C. for 10 hours. The resulting reaction mixture was poured into water, and an aqueous layer was subjected to extraction with ethyl acetate. The resulting organic layer was washed with water, and dried over anhydrous magnesium sulfate. The solution was concentrated under reduced pressure, and the residue was purified by silica gel chromatography (ethyl acetate:toluene=1:3 in a volume ratio) to obtain compound (T-9) (35.0 g; 97%).

### Second Step

Compound (T-9) (35.0 g) trimethylsilylacetylene (15.6 g), copper iodide (2.5 g), Pd(PPh₃)₂Cl₂ (4.67 g) and triethylamine (200 mL) were put in a vessel, and the resulting mixture was stirred overnight. The resulting reaction mixture was poured into water and subjected to extraction with toluene, and the resulting layer was washed with water, and then dried over anhydrous magnesium sulfate, and concentrated under reduced pressure to obtain a pale brown solid. The solid was dissolved into a solution, and purified by silica gel column chromatography (ethyl acetate:toluene=1:4 in a volume ratio), and the resulting material was dissolved in a mixed solution of methanol (100 mL) and THF (100 mL). Thereto, KF (7.7 g) was added, and the resulting mixture was stirred at room temperature overnight. The resulting material was concentrated, and purified by silica gel chromatography (ethyl acetate:toluene=1:4 in a volume ratio) to obtain compound (T-10) (17.9 g; 83%).

### Third Step

Compound (T-10) (3.25 g), compound (T-2) (2.00 g), DMAP (0.27 g) and dichloromethane (100 mL) were put in a reaction vessel, and the resulting mixture was cooled down to 0° C. Thereto, DCC (4.81 g) was added, and the resulting mixture was stirred for 12 hours while returning to room temperature. An insoluble matter was filtered off, and then the resulting reaction mixture was poured into water, and an aqueous layer was subjected to extraction with dichloromethane. The resulting organic layer was washed with water, and dried over anhydrous magnesium sulfate. The solution was concentrated under reduced pressure, and the residue was purified by silica gel chromatography (toluene) to obtain compound (T-11) (4.5 g; 93%).

### Fourth Step

Compound (T-12) (5.00 g), compound (T-7) (6.55 g), DMAP (0.52 g) and dichloromethane (100 mL) were put in a reaction vessel, and the resulting mixture was cooled down to 0° C. Thereto, DCC (4.62 g) was added, and the resulting mixture was stirred for 12 hours while returning to room temperature. An insoluble matter was filtered off, and then the resulting reaction mixture was poured into water, and an aqueous layer was subjected to extraction with dichloromethane. The resulting organic layer was washed with water, and dried over anhydrous magnesium sulfate. The solution was concentrated under reduced pressure, and the residue was purified by silica gel chromatography (ethyl acetate:toluene=1:9 in a volume ratio) to obtain compound (T-13) (7.1 g; 63%).

### Fifth Step

Compound (T-13) (4.9 g), compound (T-11) (2.2 g), copper iodide (0.17 g), Pd(PPh₃)₂Cl₂ (0.32 g) and triethylamine (1.00 mL) were put in a vessel, and the resulting mixture was stirred overnight. The resulting reaction mixture was poured into water and subjected to extraction with toluene, and the resulting layer was washed with water, and then dried over anhydrous magnesium sulfate, and concentrated under reduced pressure to obtain a pale brown solid. The solid was dissolved into a solution, and purified by silica gel column chromatography (ethyl acetate:toluene=1:9 in a volume ratio) to obtain compound (No. 158) (4.3 g; 73%).

An NMR analysis value of the resulting compound (No. 158) was as described below.

¹H-NMR: chemical shift δ (ppm; CDCl₃): 8.16 (d, 2H), 7.47 (d, 2H), 7.43 (s, 1H), 7.39 (dd, 1H), 7.11 (d, 1H), 6.97 (d, 2H), 6.90 (d, 2H), 6.10 (s, 1H), 5.72 (dd, 1H), 5.55 (t, 1H), 5.37 (dd, 1H), 4.60 (t, 2H), 4.26 (t, 2H), 4.17 (t, 2H), 4.06 (t, 2H), 2.22 (s, 3H), 1.95 (s, 3H), 1.85 (quint, 2H), 1.72 (quint, 2H), 1.55 (quint, 2H), 1.47 (quint, 2H).

Physical properties of compound (No. 158) were as described below.

Transition temperature (° C): C 75.71 I. Polymerization temperature (° C.) 261.67.

### Synthesis Example 4

### Synthesis of Compound (No. 81)

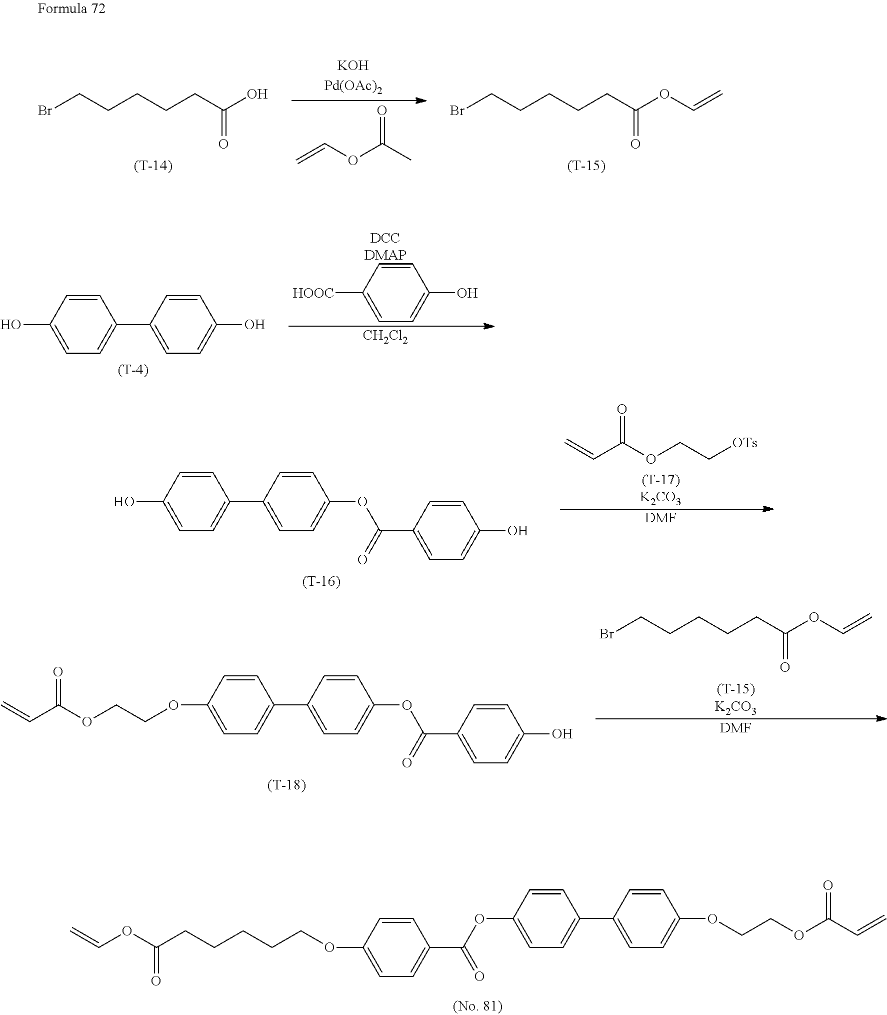

### First Step

Compound (T-14) (10.0 g), potassium hydroxide (0.33 g), palladium acetate (1.84 g) and vinyl acetate (100 mL) were put in a reaction vessel, and the resulting mixture was stirred at room temperature for 2 days. The resulting reaction mixture was concentrated under reduced pressure, and the residue was purified by silica gel chromatography (heptane:toluene=1:2 in a volume ratio) to obtain compound (T-15) (8.7 g; 77%).

### Second Step

Then, 4,4′-biphenyldiol (T-4) (10 g), 4-hydroxybenzoic acid (7.4 g) 4-dimethylaminopyridine (DMAP) (0.34 g) and dichloromethane (200 mL) were put in a reaction vessel, and the resulting mixture was cooled down to 0° C. Thereto, DCC (11 g) was added, and the resulting mixture was stirred for 12 hours while returning to room temperature. An insoluble matter was filtered off, and then the resulting reaction mixture was poured into water, and an aqueous layer was subjected to extraction with ethyl acetate. The resulting organic layer was washed with water, and dried over anhydrous magnesium sulfate. The solution was concentrated under reduced pressure, and the residue was purified by silica gel chromatography (ethyl acetate:toluene=1:1 in a volume ratio) to obtain, compound. (T-16) (3 g; 40%).

### Third Step

Compound (T-16) (3.26 g), potassium carbonate (2.3 g), compound (T-17) (2.48 g) and DMF (100 mL) were put in a reaction vessel, and the resulting mixture was stirred at 70° C. for 10 hours. The resulting reaction mixture was poured into water, and an aqueous layer was subjected to extraction with ethyl acetate. The resulting organic layer was washed with water, and dried over anhydrous magnesium sulfate. The solution was concentrated under reduced pressure, and the residue was purified by silica gel chromatography (ethyl acetate:toluene=1:4 in a volume ratio) to obtain compound (T-18) (1.62 g; 45%).

### Fourth Step

Compound (T-18) (1.62 g), potassium carbonate (1.1 g), compound (T-15) (0.97 g) and DMF (100 mL) were put in a reaction vessel, and the resulting mixture was stirred at 60° C. for 3 hours. The resulting reaction mixture was poured into water, and an aqueous layer was subjected to extraction with ethyl acetate. The resulting organic layer was washed with water, and dried over anhydrous magnesium sulfate. The solution was concentrated under reduced pressure, and the residue was purified by silica gel chromatography (ethyl acetate:toluene=1:9 in a volume ratio) to obtain compound (No. 81) (1.20 g; 55%).

An NMR analysis value of the resulting compound (No. 81) was as described below.

¹H-NMR: chemical shift δ (ppm; CDCl₃): 8.16 (d, 2H), 7.58 (d, 2H), 7.53 (d, 2H), 7.30 (dd, 1H), 7.25 (d, 2H), 7.00 (d, 2H), 6.97 (d, 2H), 6.46 (dd, 1H), 6.18 (dd, 1H), 5.87 (dd, 1H), 4.89 (dd, 1H), 4.58 (dd, 1H), 4.54 (t, 2H), 4.26 (t, 2H), 4.06 (t, 2H), 2.45 (t, 2H), 1.85 (quint, 2H), 1.76 (quint, 2H), 1.57 (quint, 2H).

Physical properties of compound (No. 81) were as described below.

Transition temperature (° C.): C 107.7 I. Polymerization temperature (° C.): 162.11.

### Synthesis Example 5

### Synthesis of Compound (No. 281)

In Synthesis Example 3, compound (No. 281) was prepared by using compound (T-6) in place of compound (T-7).

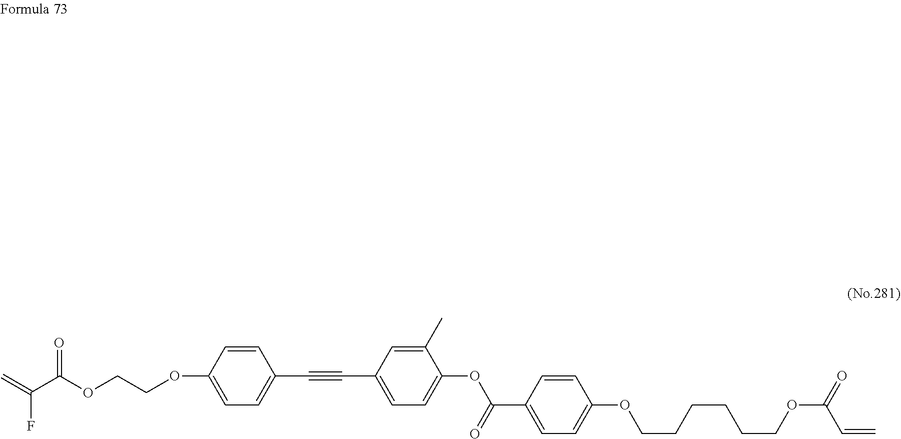 [SMILES: C=CC(=O)OCCCCCCOc1ccc(C(=O)Oc2ccc(C#Cc3ccc(OCCOC(=O)C(=C)F)cc3)cc2C)cc1]

An NMR analysis value of the resulting compound (No. 281) was as described below.

¹H-NMR: chemical shift δ (ppm; CDCl₃): 8.15 (d, 2H), 7.47 (d, 2H), 7.43 (s, 1H), 7.39 (dd, 1H), 7.11 (d, 1H), 6.97 (d, 2H), 6.89 (d, 2H), 6.31 (d, 1H), 6.11 (dd, 1H), 5.82 (d, 1H), 5.77 (dd, 1H), 5.37 (dd, 1H), 4.59 (t, 2H), 4.26 (t, 2H), 4.18 (t, 2H), 4.05 (t, 2H), 2.22 (s, 3H), 1.85 (quint, 2H), 1.73 (quint, 2H), 1.55 (quint, 2H), 1.47 (quint, 2H).

Physical properties of compound (No. 281) were as described below

Transition temperature (° C.): C 68.05 I. Polymerization temperature (° C.): 253.5.

### Synthesis Example 6

### Synthesis of Compound (No. 282)

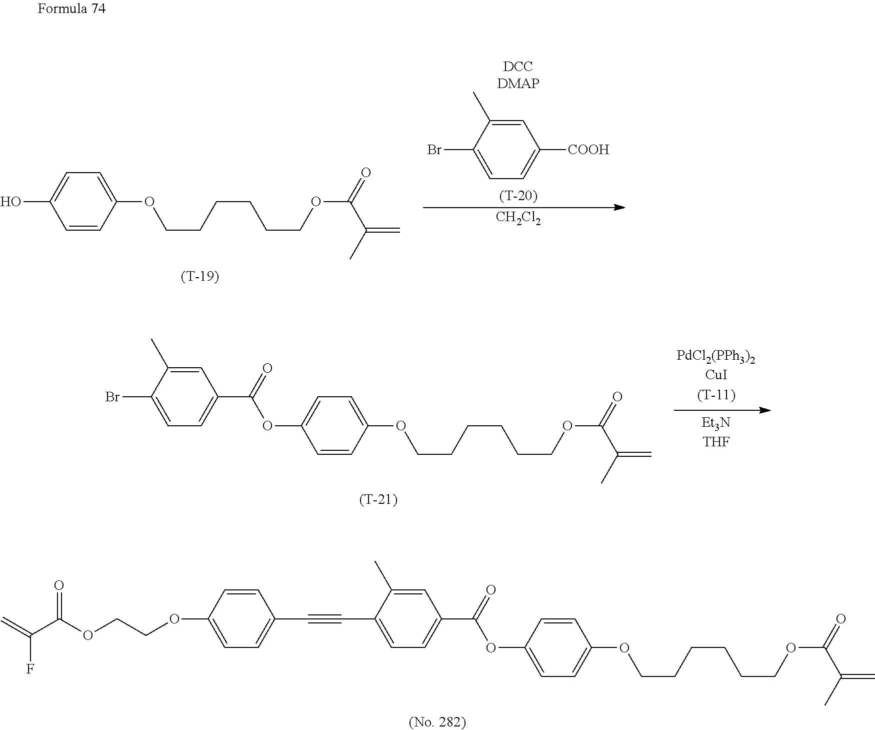

### First Step

Compound (T-19) (2.5 g), compound (T-20) (2.12 g), DMAP (0.11 g) and dichloromethane (100 mL) were put in a reaction vessel, and the resulting mixture was cooled down to 0° C. Thereto, DCC (2.04 g) was added, and the resulting mixture was stirred for 12 hours while returning to room temperature. An insoluble matter was filtered off, and then the resulting reaction mixture was poured into water, and an aqueous layer was subjected to extraction with dichloromethane. The resulting organic layer was washed with water, and dried over anhydrous magnesium sulfate. The solution was concentrated under reduced pressure, and the residue was purified by silica gel chromatography (toluene) to obtain compound (T-21) (4.15 g; 97%). In addition, compound (T-19) is a known substance, and those skilled in the art can easily obtain a synthesis method.

### Second Step

Compound (T-21) (4.15 g), compound (T-11) (2.04 g), copper iodide (0.17 g) Pd(PPh₃)₂Cl₂ (0.32 g) and triethylamine (100 mL) were put in a vessel, and the resulting mixture was stirred overnight. The resulting reaction mixture was poured into water and subjected to extraction with toluene, and the resulting layer was washed with water, and then dried over anhydrous magnesium sulfate, and concentrated under reduced pressure to obtain a pale brown solid. The solid was dissolved into a solution, and purified by silica gel column chromatography (ethyl acetate:toluene=1:9 in a volume ratio) to obtain compound (No. 282) (0.53 g; 9.7%).

An NMR analysis value of the resulting compound (No. 282) was as described below.

¹H-NMR: chemical shift δ (ppm; CDCl₃): 8.05 (s, 1H), 7.98 (d, 1H), 7.58 (d, 1H), 7.51 (d, 2H), 7.10 (d, 2H), 6.93 (d, 4H), 6.10 (s, 1H), 5.72 (dd, 1H), 5.55 (t, 1H), 5.37 (dd, 1H), 4.61 (t, 2H), 4.27 (t, 2H), 4.16 (t, 2H), 3.96 (t, 2H), 2.58 (s, 3H), 1.95 (s, 3H), 1.85 (quint, 2H), 1.72 (quint, 2H), 1.55 (quint, 2H), 1.47 (quint, 2H).

Physical properties of compound (No. 282) were as described below.

Transition temperature (° C.): C 118.6 I. Polymerization temperature (° C.): 140.24.

Compounds (No. 1) to (No. 392) described below can be prepared according to the synthesis methods described in Synthesis Examples.

No.

Formula 75

| | |
|---|---|
| 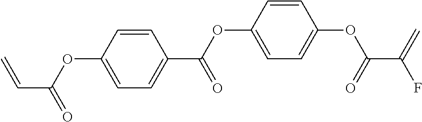 [SMILES: C=CC(=O)Oc1ccc(C(=O)Oc2ccc(OC(=O)C(=C)F)cc2)cc1] | 1 |
| 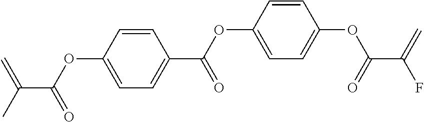 [SMILES: C=C(C)C(=O)Oc1ccc(C(=O)Oc2ccc(OC(=O)C(=C)F)cc2)cc1] | 2 |
| 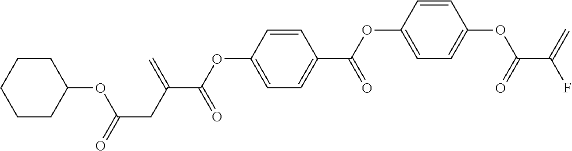 [SMILES: C=C(F)C(=O)Oc1ccc(OC(=O)c2ccc(OC(=O)C(=C)CC(=O)OC3CCCCC3)cc2)cc1] | 3 |
| 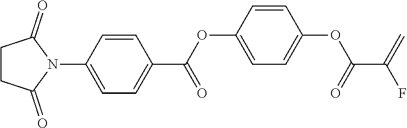 [SMILES: C=C(F)C(=O)Oc1ccc(OC(=O)c2ccc(N3C(=O)CCC3=O)cc2)cc1] | 4 |
| 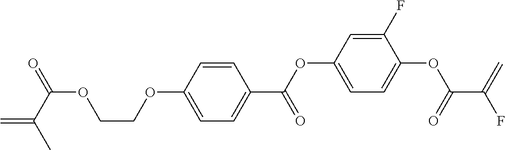 [SMILES: C=C(C)C(=O)OCCOc1ccc(C(=O)Oc2ccc(OC(=O)C(=C)F)c(F)c2)cc1] | 5 |
| 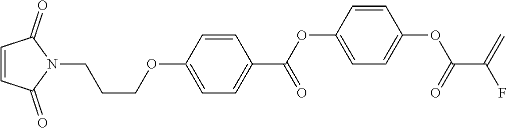 [SMILES: C=C(F)C(=O)Oc1ccc(OC(=O)c2ccc(OCCCN3C(=O)C=CC3=O)cc2)cc1] | 6 |
| 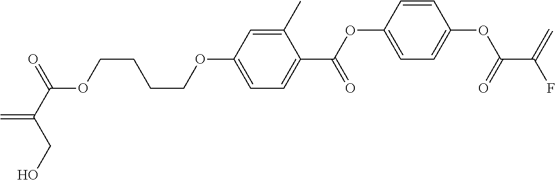 [SMILES: C=C(F)C(=O)Oc1ccc(OC(=O)c2ccc(OCCCCOC(=O)C(=C)CO)cc2C)cc1] | 7 |
| 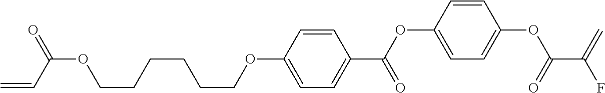 [SMILES: C=CC(=O)OCCCCCCOc1ccc(C(=O)Oc2ccc(OC(=O)C(=C)F)cc2)cc1] | 8 |
| 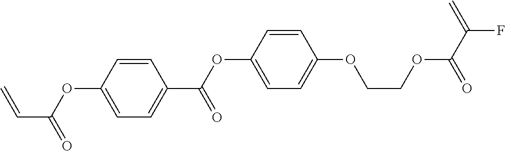 [SMILES: C=CC(=O)Oc1ccc(C(=O)Oc2ccc(OCCOC(=O)C(=C)F)cc2)cc1] | 9 |
| 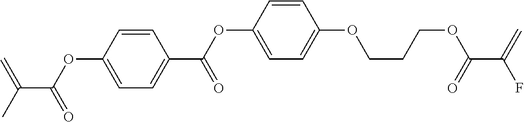 [SMILES: C=C(C)C(=O)Oc1ccc(C(=O)Oc2ccc(OCCCOC(=O)C(=C)F)cc2)cc1] | 10 |
| 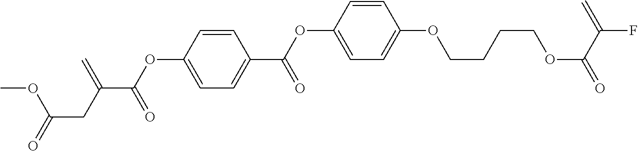 [SMILES: C=C(F)C(=O)OCCCCOc1ccc(OC(=O)c2ccc(OC(=O)C(=C)CC(=O)OC)cc2)cc1] | 11 |
| 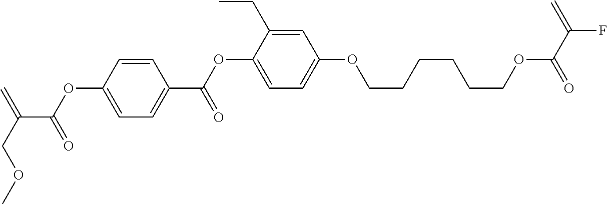 [SMILES: C=C(F)C(=O)OCCCCCCOc1ccc(OC(=O)c2ccc(OC(=O)C(=C)COC)cc2)c(CC)c1] | 12 |
| 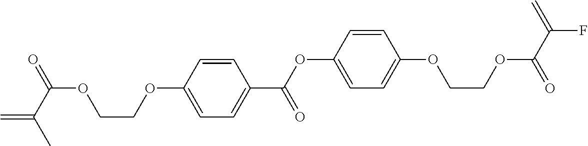 [SMILES: C=C(C)C(=O)OCCOc1ccc(C(=O)Oc2ccc(OCCOC(=O)C(=C)F)cc2)cc1] | 13 |
| 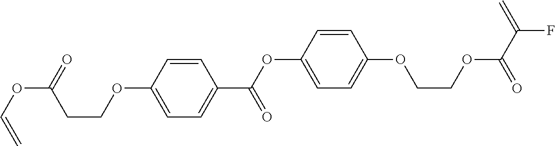 [SMILES: C=COC(=O)CCOc1ccc(C(=O)Oc2ccc(OCCOC(=O)C(=C)F)cc2)cc1] | 10 |
| 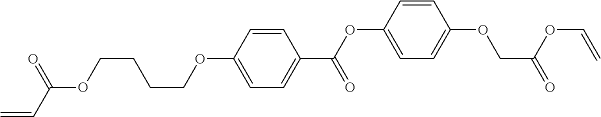 [SMILES: C=COC(=O)COc1ccc(OC(=O)c2ccc(OCCCCOC(=O)C=C)cc2)cc1] | 15 |
| 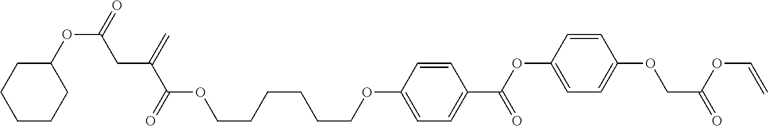 [SMILES: C=COC(=O)COc1ccc(OC(=O)c2ccc(OCCCCCCOC(=O)C(=C)CC(=O)OC3CCCCC3)cc2)cc1] | 16 |
| 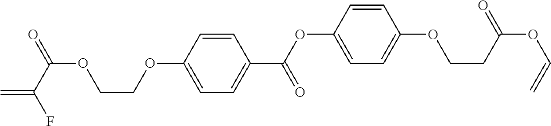 [SMILES: C=COC(=O)CCOc1ccc(OC(=O)c2ccc(OCCOC(=O)C(=C)F)cc2)cc1] | 17 |
| 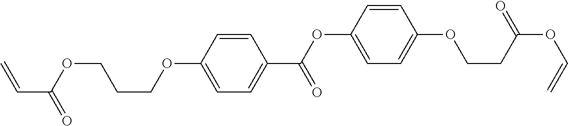 [SMILES: C=COC(=O)CCOc1ccc(OC(=O)c2ccc(OCCCOC(=O)C=C)cc2)cc1] | 18 |
| 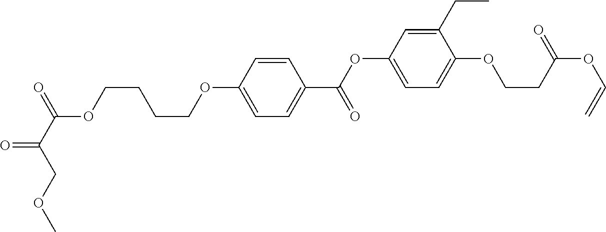 [SMILES: C=COC(=O)CCOc1ccc(OC(=O)c2ccc(OCCCCOC(=O)C(=O)COC)cc2)cc1CC] | 19 |
| 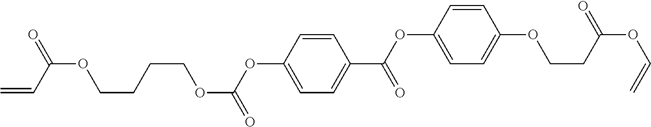 [SMILES: C=COC(=O)CCOc1ccc(OC(=O)c2ccc(OC(=O)OCCCCOC(=O)C=C)cc2)cc1] | 20 |
| 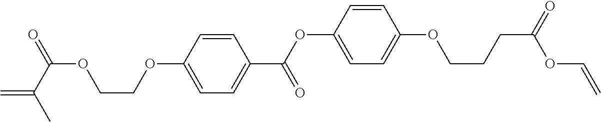 [SMILES: C=COC(=O)CCCOc1ccc(OC(=O)c2ccc(OCCOC(=O)C(=C)C)cc2)cc1] | 21 |
| 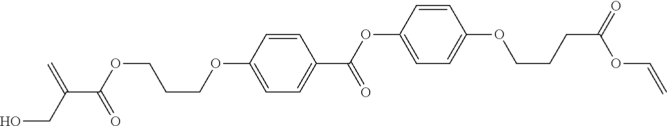 [SMILES: C=COC(=O)CCCOc1ccc(OC(=O)c2ccc(OCCCOC(=O)C(=C)CO)cc2)cc1] | 22 |
| 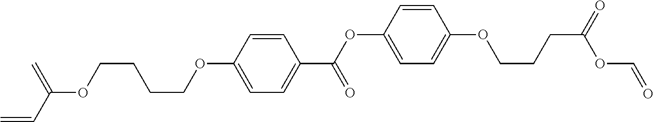 [SMILES: C=CC(=C)OCCCCOc1ccc(C(=O)Oc2ccc(OCCCC(=O)OC=O)cc2)cc1] | 23 |
| 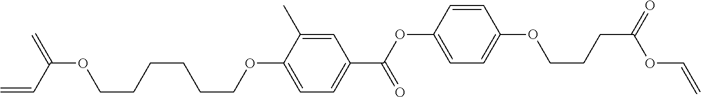 [SMILES: C=COC(=O)CCCOc1ccc(OC(=O)c2ccc(OCCCCCCOC(=C)C=C)c(C)c2)cc1] | 24 |
| 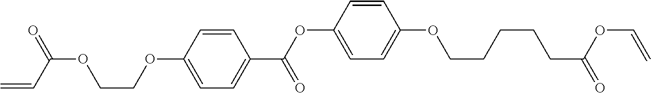 [SMILES: C=COC(=O)CCCCCOc1ccc(OC(=O)c2ccc(OCCOC(=O)C=C)cc2)cc1] | 25 |
| 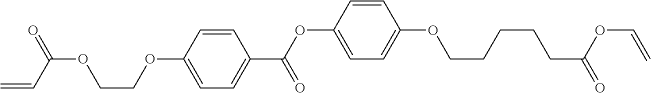 [SMILES: C=COC(=O)CCCCCOc1ccc(OC(=O)c2ccc(OCCOC(=O)C=C)cc2)cc1] | 26 |
| 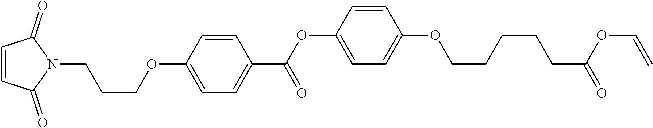 [SMILES: C=COC(=O)CCCCCOc1ccc(OC(=O)c2ccc(OCCCN3C(=O)C=CC3=O)cc2)cc1] | 27 |
| 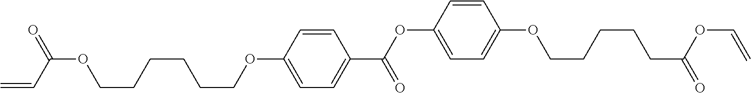 [SMILES: C=COC(=O)CCCCCOc1ccc(OC(=O)c2ccc(OCCCCCCOC(=O)C=C)cc2)cc1] | 28 |

Formula 76

| | |
|---|---|
| 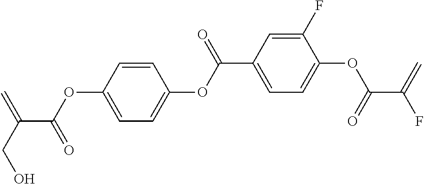 [SMILES: C=C(F)C(=O)Oc1ccc(C(=O)Oc2ccc(OC(=O)C(=C)CO)cc2)cc1F] | 29 |
| 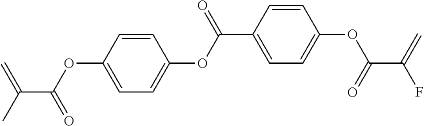 [SMILES: C=C(C)C(=O)Oc1ccc(OC(=O)c2ccc(OC(=O)C(=C)F)cc2)cc1] | 30 |
| 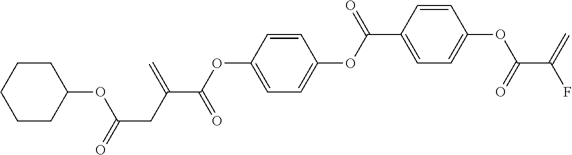 [SMILES: C=C(F)C(=O)Oc1ccc(C(=O)Oc2ccc(OC(=O)C(=C)CC(=O)OC3CCCCC3)cc2)cc1] | 31 |
| 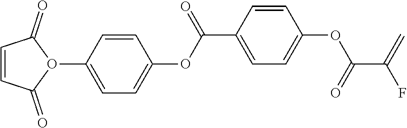 | 32 |
| 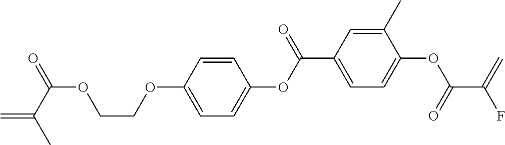 [SMILES: C=C(C)C(=O)OCCOc1ccc(OC(=O)c2ccc(OC(=O)C(=C)F)c(C)c2)cc1] | 33 |
| 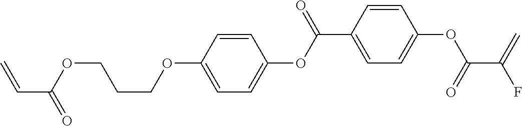 [SMILES: C=CC(=O)OCCCOc1ccc(OC(=O)c2ccc(OC(=O)C(=C)F)cc2)cc1] | 34 |
| 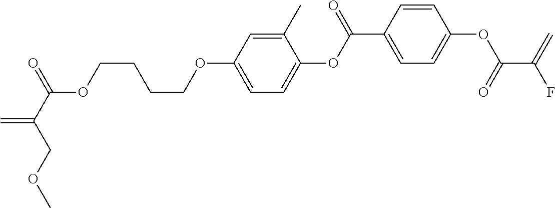 [SMILES: C=C(F)C(=O)Oc1ccc(C(=O)Oc2ccc(OCCCCOC(=O)C(=C)COC)cc2C)cc1] | 35 |
| 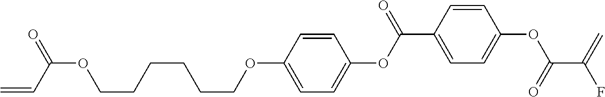 [SMILES: C=CC(=O)OCCCCCCOc1ccc(OC(=O)c2ccc(OC(=O)C(=C)F)cc2)cc1] | 36 |
| 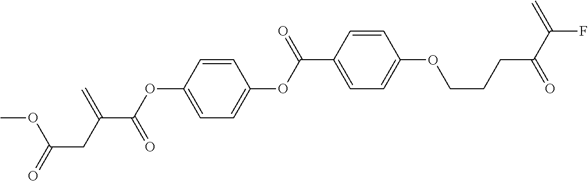 [SMILES: C=C(F)C(=O)CCCOc1ccc(C(=O)Oc2ccc(OC(=O)C(=C)CC(=O)OC)cc2)cc1] | 37 |
| 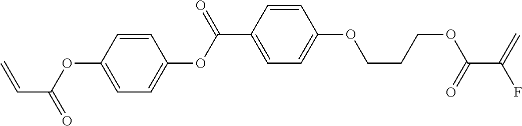 [SMILES: C=CC(=O)Oc1ccc(OC(=O)c2ccc(OCCCOC(=O)C(=C)F)cc2)cc1] | 38 |
| 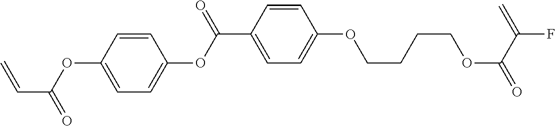 [SMILES: C=CC(=O)Oc1ccc(OC(=O)c2ccc(OCCCCOC(=O)C(=C)F)cc2)cc1] | 39 |
| 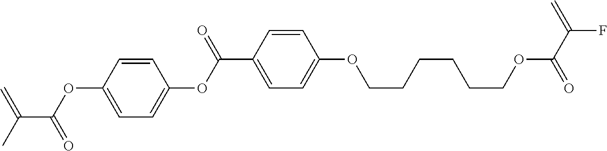 [SMILES: C=C(C)C(=O)Oc1ccc(OC(=O)c2ccc(OCCCCCCOC(=O)C(=C)F)cc2)cc1] | 40 |
| 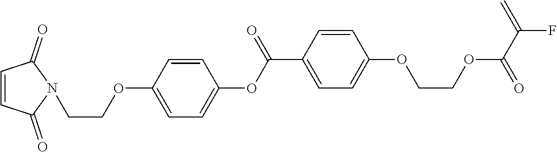 [SMILES: C=C(F)C(=O)OCCOc1ccc(C(=O)Oc2ccc(OCCN3C(=O)C=CC3=O)cc2)cc1] | 41 |
| 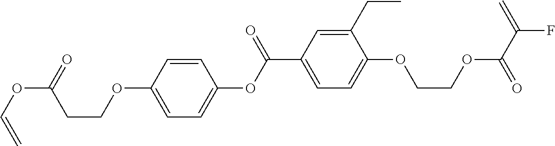 [SMILES: C=COC(=O)CCOc1ccc(OC(=O)c2ccc(OCCOC(=O)C(=C)F)c(CC)c2)cc1] | 42 |
| 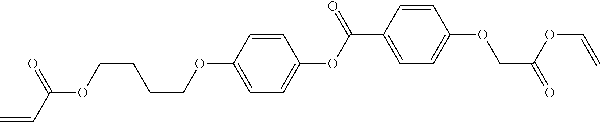 [SMILES: C=COC(=O)COc1ccc(C(=O)Oc2ccc(OCCCCOC(=O)C=C)cc2)cc1] | 43 |
| 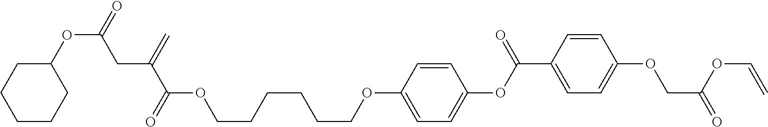 [SMILES: C=COC(=O)COc1ccc(C(=O)Oc2ccc(OCCCCCCOC(=O)C(=C)CC(=O)OC3CCCCC3)cc2)cc1] | 44 |
| 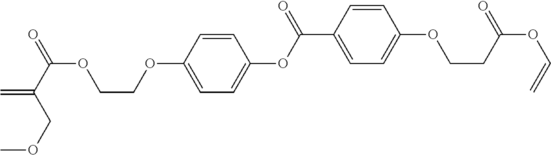 [SMILES: C=COC(=O)CCOc1ccc(C(=O)Oc2ccc(OCCOC(=O)C(=C)COC)cc2)cc1] | 45 |
| 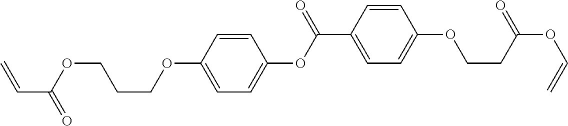 [SMILES: C=COC(=O)CCOc1ccc(C(=O)Oc2ccc(OCCCOC(=O)C=C)cc2)cc1] | 46 |
| 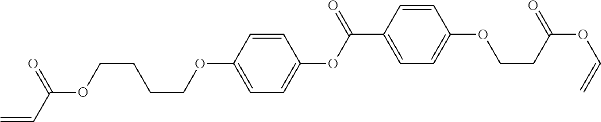 [SMILES: C=COC(=O)CCOc1ccc(C(=O)Oc2ccc(OCCCCOC(=O)C=C)cc2)cc1] | 47 |
| 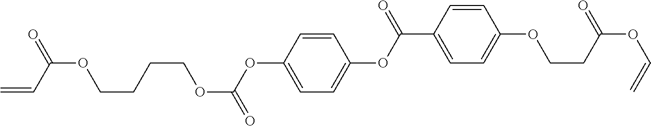 [SMILES: C=COC(=O)CCOc1ccc(C(=O)Oc2ccc(OC(=O)OCCCCOC(=O)C=C)cc2)cc1] | 48 |
| 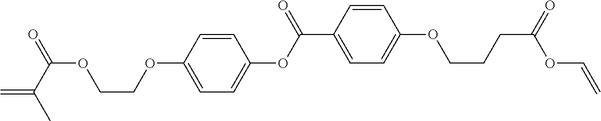 [SMILES: C=COC(=O)CCCOc1ccc(C(=O)Oc2ccc(OCCOC(=O)C(=C)C)cc2)cc1] | 49 |
| 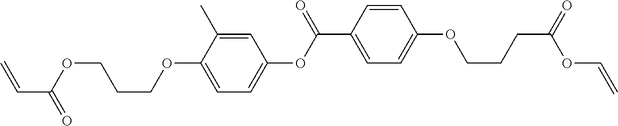 [SMILES: C=COC(=O)CCCOc1ccc(C(=O)Oc2ccc(OCCCOC(=O)C=C)c(C)c2)cc1] | 50 |
| 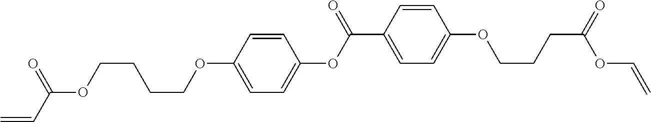 [SMILES: C=COC(=O)CCCOc1ccc(C(=O)Oc2ccc(OCCCCOC(=O)C=C)cc2)cc1] | 51 |
| 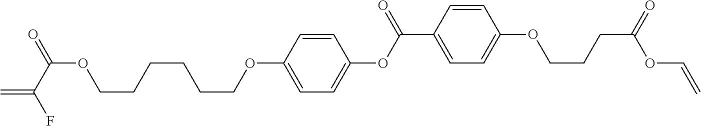 [SMILES: C=COC(=O)CCCOc1ccc(C(=O)Oc2ccc(OCCCCCCOC(=O)C(=C)F)cc2)cc1] | 52 |
| 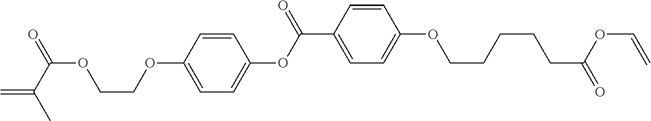 [SMILES: C=COC(=O)CCCCCOc1ccc(C(=O)Oc2ccc(OCCOC(=O)C(=C)C)cc2)cc1] | 53 |
| 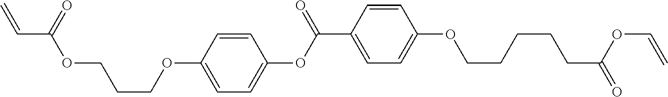 [SMILES: C=COC(=O)CCCCCOc1ccc(C(=O)Oc2ccc(OCCCOC(=O)C=C)cc2)cc1] | 54 |
| 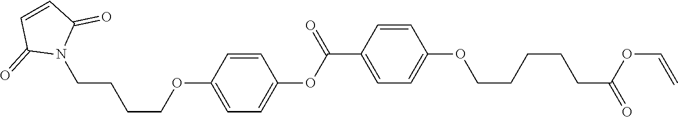 [SMILES: C=COC(=O)CCCCCOc1ccc(C(=O)Oc2ccc(OCCCCN3C(=O)C=CC3=O)cc2)cc1] | 55 |
| 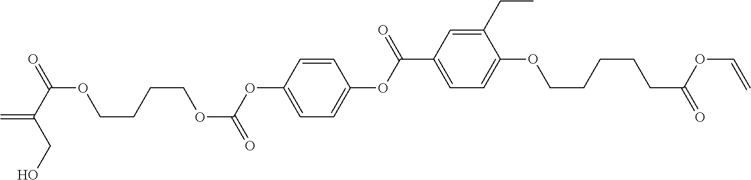 [SMILES: C=COC(=O)CCCCCOc1ccc(C(=O)Oc2ccc(OC(=O)OCCCCOC(=O)C(=C)CO)cc2)cc1CC] | 56 |

Formula 77

| | |
|---|---|
| 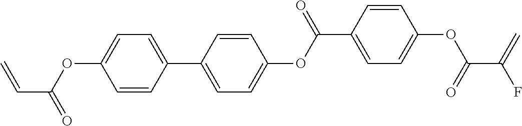 [SMILES: C=CC(=O)Oc1ccc(-c2ccc(OC(=O)c3ccc(OC(=O)C(=C)F)cc3)cc2)cc1] | 57 |
| 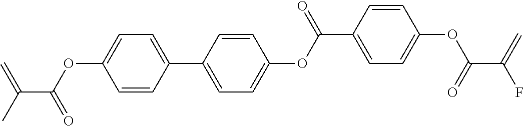 [SMILES: C=C(C)C(=O)Oc1ccc(-c2ccc(OC(=O)c3ccc(OC(=O)C(=C)F)cc3)cc2)cc1] | 58 |
| 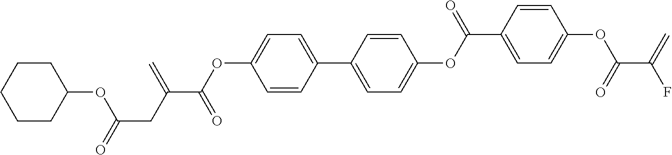 [SMILES: C=C(F)C(=O)Oc1ccc(C(=O)Oc2ccc(-c3ccc(OC(=O)C(=C)CC(=O)OC4CCCCC4)cc3)cc2)cc1] | 59 |
| 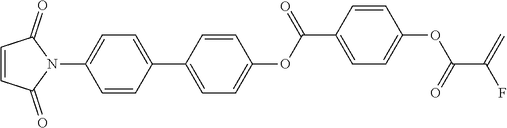 [SMILES: C=C(F)C(=O)Oc1ccc(C(=O)Oc2ccc(-c3ccc(N4C(=O)C=CC4=O)cc3)cc2)cc1] | 60 |
| 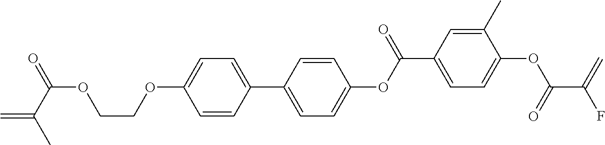 [SMILES: C=C(C)C(=O)OCCOc1ccc(-c2ccc(OC(=O)c3ccc(OC(=O)C(=C)F)c(C)c3)cc2)cc1] | 61 |
| 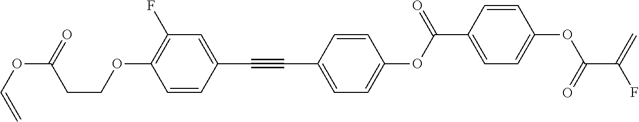 [SMILES: C=COC(=O)CCOc1ccc(C#Cc2ccc(OC(=O)c3ccc(OC(=O)C(=C)F)cc3)cc2)cc1F] | 62 |
| 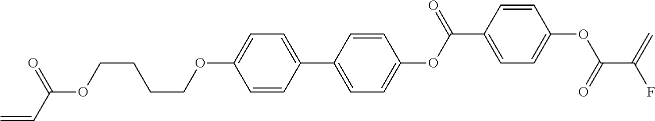 [SMILES: C=CC(=O)OCCCCOc1ccc(-c2ccc(OC(=O)c3ccc(OC(=O)C(=C)F)cc3)cc2)cc1] | 63 |
| 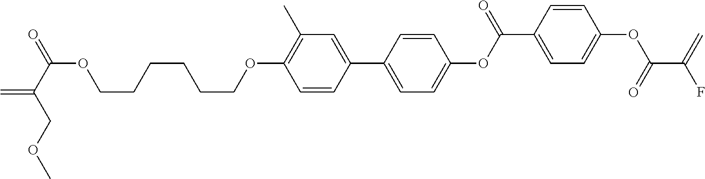 [SMILES: C=C(F)C(=O)Oc1ccc(C(=O)Oc2ccc(-c3ccc(OCCCCCCOC(=O)C(=C)COC)c(C)c3)cc2)cc1] | 64 |
| 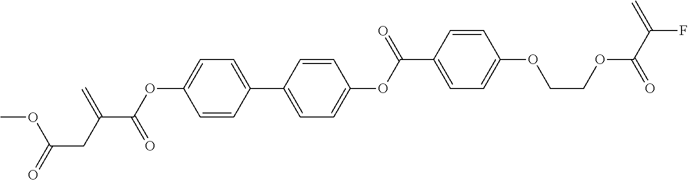 [SMILES: C=C(F)C(=O)OCCOc1ccc(C(=O)Oc2ccc(-c3ccc(OC(=O)C(=C)CC(=O)OC)cc3)cc2)cc1] | 65 |
| 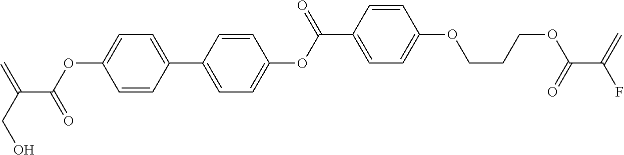 [SMILES: C=C(F)C(=O)OCCCOc1ccc(C(=O)Oc2ccc(-c3ccc(OC(=O)C(=C)CO)cc3)cc2)cc1] | 66 |
| 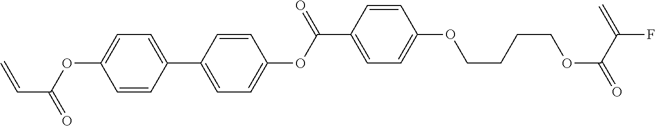 [SMILES: C=CC(=O)Oc1ccc(-c2ccc(OC(=O)c3ccc(OCCCCOC(=O)C(=C)F)cc3)cc2)cc1] | 67 |
| 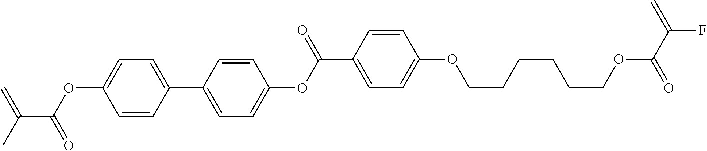 [SMILES: C=C(C)C(=O)Oc1ccc(-c2ccc(OC(=O)c3ccc(OCCCCCCOC(=O)C(=C)F)cc3)cc2)cc1] | 68 |
| 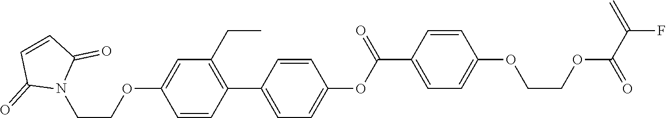 [SMILES: C=C(F)C(=O)OCCOc1ccc(C(=O)Oc2ccc(-c3ccc(OCCN4C(=O)C=CC4=O)cc3CC)cc2)cc1] | 69 |
| 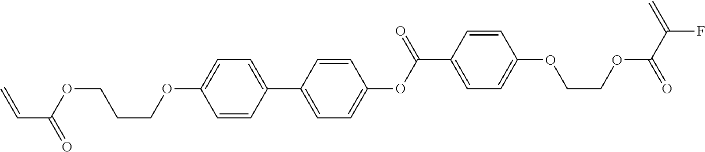 [SMILES: C=CC(=O)OCCCOc1ccc(-c2ccc(OC(=O)c3ccc(OCCOC(=O)C(=C)F)cc3)cc2)cc1] | 70 |
| 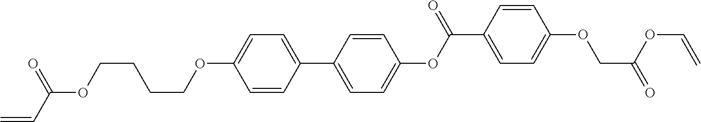 [SMILES: C=COC(=O)COc1ccc(C(=O)Oc2ccc(-c3ccc(OCCCCOC(=O)C=C)cc3)cc2)cc1] | 71 |
| 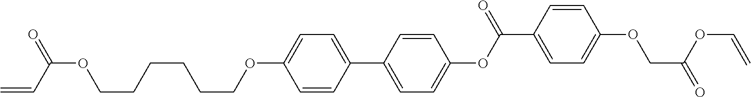 [SMILES: C=COC(=O)COc1ccc(C(=O)Oc2ccc(-c3ccc(OCCCCCCOC(=O)C=C)cc3)cc2)cc1] | 72 |
| 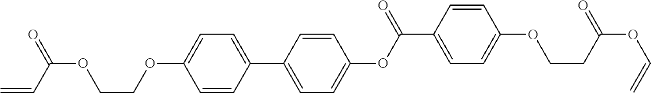 [SMILES: C=COC(=O)CCOc1ccc(C(=O)Oc2ccc(-c3ccc(OCCOC(=O)C=C)cc3)cc2)cc1] | 73 |
| 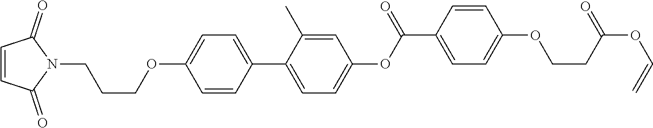 [SMILES: C=COC(=O)CCOc1ccc(C(=O)Oc2ccc(-c3ccc(OCCCN4C(=O)C=CC4=O)cc3)c(C)c2)cc1] | 74 |
| 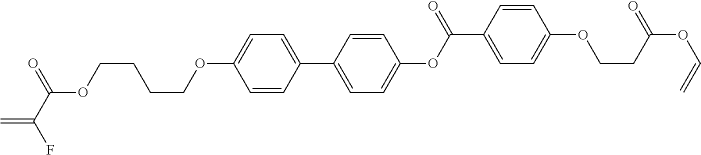 [SMILES: C=COC(=O)CCOc1ccc(C(=O)Oc2ccc(-c3ccc(OCCCCOC(=O)C(=C)F)cc3)cc2)cc1] | 75 |
| 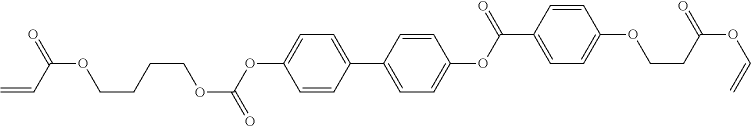 [SMILES: C=COC(=O)CCOc1ccc(C(=O)Oc2ccc(-c3ccc(OC(=O)OCCCCOC(=O)C=C)cc3)cc2)cc1] | 76 |
| 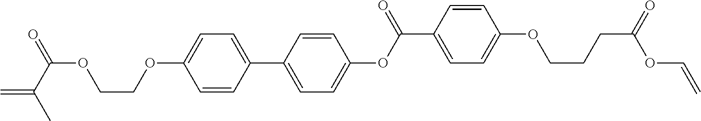 [SMILES: C=COC(=O)CCCOc1ccc(C(=O)Oc2ccc(-c3ccc(OCCOC(=O)C(=C)C)cc3)cc2)cc1] | 77 |
| 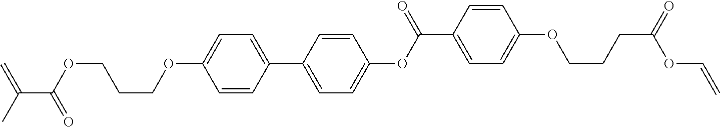 [SMILES: C=COC(=O)CCCOc1ccc(C(=O)Oc2ccc(-c3ccc(OCCCOC(=O)C(=C)C)cc3)cc2)cc1] | 78 |
| 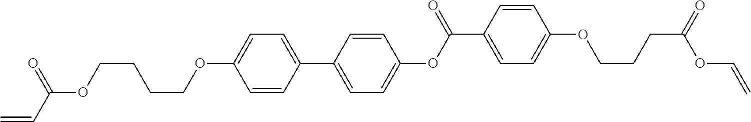 [SMILES: C=COC(=O)CCCOc1ccc(C(=O)Oc2ccc(-c3ccc(OCCCCOC(=O)C=C)cc3)cc2)cc1] | 79 |
| 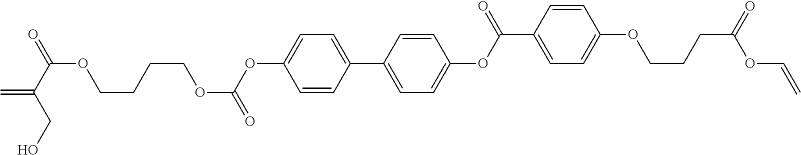 [SMILES: C=COC(=O)CCCOc1ccc(C(=O)Oc2ccc(-c3ccc(OC(=O)OCCCCOC(=O)C(=C)CO)cc3)cc2)cc1] | 80 |
| 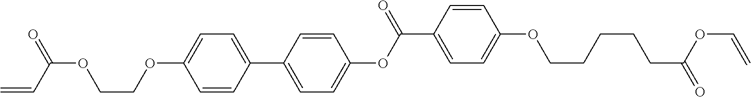 [SMILES: C=COC(=O)CCCCCOc1ccc(C(=O)Oc2ccc(-c3ccc(OCCOC(=O)C=C)cc3)cc2)cc1] | 81 |
| 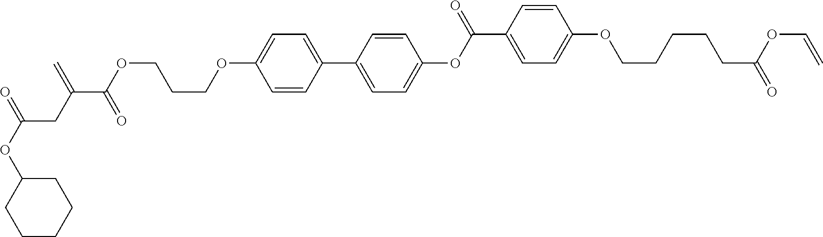 [SMILES: C=COC(=O)CCCCCOc1ccc(C(=O)Oc2ccc(-c3ccc(OCCCOC(=O)C(=C)CC(=O)OC4CCCCC4)cc3)cc2)cc1] | 82 |
| 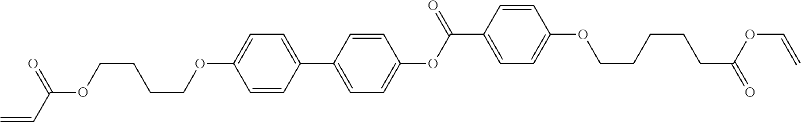 [SMILES: C=COC(=O)CCCCCOc1ccc(C(=O)Oc2ccc(-c3ccc(OCCCCOC(=O)C=C)cc3)cc2)cc1] | 83 |
| 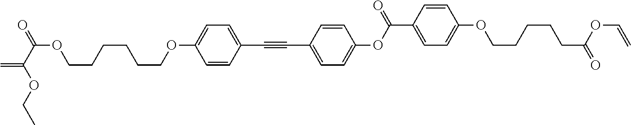 [SMILES: C=COC(=O)CCCCCOc1ccc(C(=O)Oc2ccc(C#Cc3ccc(OCCCCCCOC(=O)C(=C)OCC)cc3)cc2)cc1] | 84 |

Formula 78

| | |
|---|---|
| 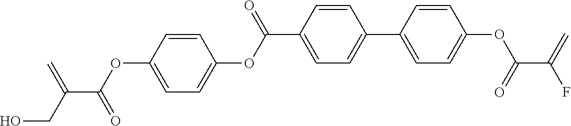 [SMILES: C=C(F)C(=O)Oc1ccc(-c2ccc(C(=O)Oc3ccc(OC(=O)C(=C)CO)cc3)cc2)cc1] | 85 |
| 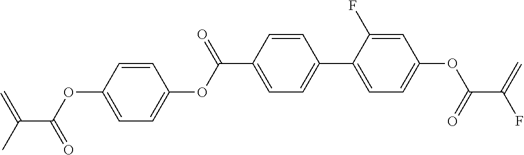 [SMILES: C=C(C)C(=O)Oc1ccc(OC(=O)c2ccc(-c3ccc(OC(=O)C(=C)F)cc3F)cc2)cc1] | 86 |
| 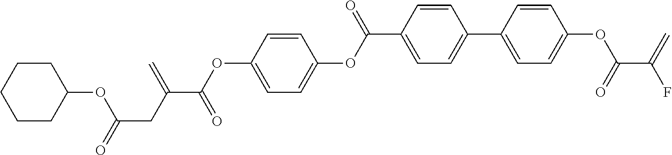 [SMILES: C=C(F)C(=O)Oc1ccc(-c2ccc(C(=O)Oc3ccc(OC(=O)C(=C)CC(=O)OC4CCCCC4)cc3)cc2)cc1] | 87 |
| 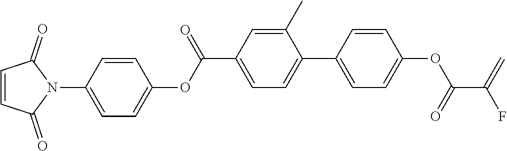 [SMILES: C=C(F)C(=O)Oc1ccc(-c2ccc(C(=O)Oc3ccc(N4C(=O)C=CC4=O)cc3)cc2C)cc1] | 88 |
| 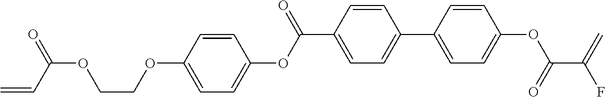 [SMILES: C=CC(=O)OCCOc1ccc(OC(=O)c2ccc(-c3ccc(OC(=O)C(=C)F)cc3)cc2)cc1] | 89 |
| 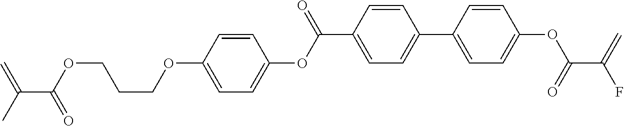 [SMILES: C=C(C)C(=O)OCCCOc1ccc(OC(=O)c2ccc(-c3ccc(OC(=O)C(=C)F)cc3)cc2)cc1] | 90 |
| 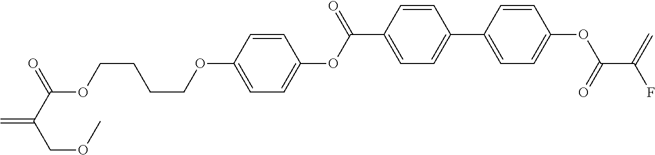 [SMILES: C=C(F)C(=O)Oc1ccc(-c2ccc(C(=O)Oc3ccc(OCCCCOC(=O)C(=C)COC)cc3)cc2)cc1] | 91 |
| 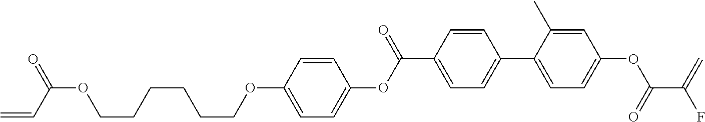 [SMILES: C=CC(=O)OCCCCCCOc1ccc(OC(=O)c2ccc(-c3ccc(OC(=O)C(=C)F)cc3C)cc2)cc1] | 92 |
| 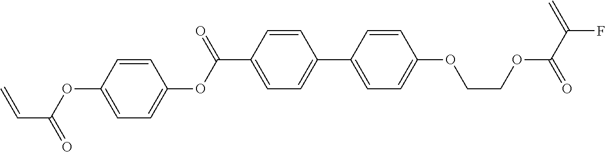 [SMILES: C=CC(=O)Oc1ccc(OC(=O)c2ccc(-c3ccc(OCCOC(=O)C(=C)F)cc3)cc2)cc1] | 93 |
| 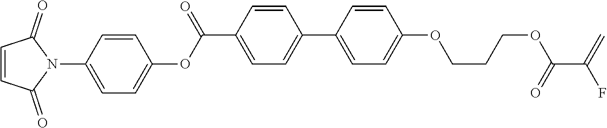 [SMILES: C=C(F)C(=O)OCCCOc1ccc(-c2ccc(C(=O)Oc3ccc(N4C(=O)C=CC4=O)cc3)cc2)cc1] | 94 |
| 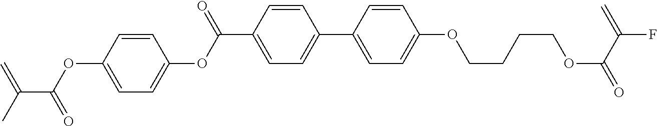 [SMILES: C=C(C)C(=O)Oc1ccc(OC(=O)c2ccc(-c3ccc(OCCCCOC(=O)C(=C)F)cc3)cc2)cc1] | 95 |
| 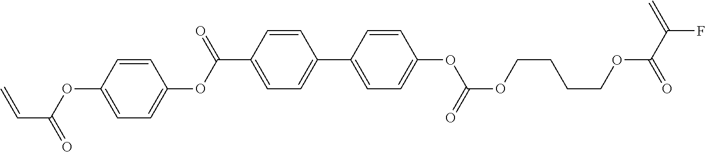 [SMILES: C=CC(=O)Oc1ccc(OC(=O)c2ccc(-c3ccc(OC(=O)OCCCCOC(=O)C(=C)F)cc3)cc2)cc1] | 96 |
| 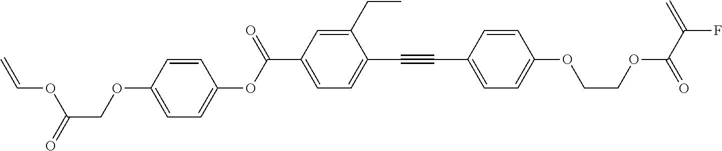 [SMILES: C=COC(=O)COc1ccc(OC(=O)c2ccc(C#Cc3ccc(OCCOC(=O)C(=C)F)cc3)c(CC)c2)cc1] | 97 |
| 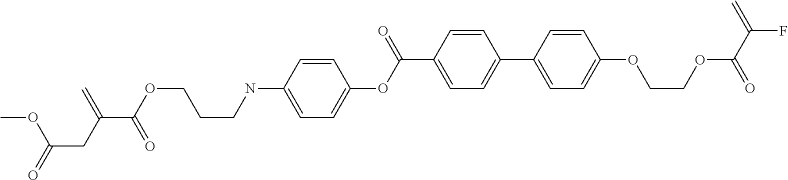 [SMILES: C=C(F)C(=O)OCCOc1ccc(-c2ccc(C(=O)Oc3ccc(NCCCOC(=O)C(=C)CC(=O)OC)cc3)cc2)cc1] | 98 |
| 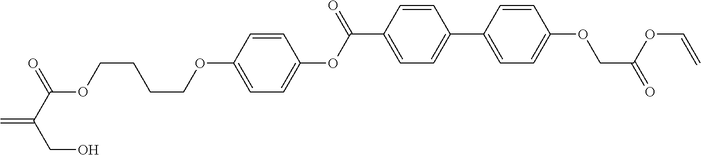 [SMILES: C=COC(=O)COc1ccc(-c2ccc(C(=O)Oc3ccc(OCCCCOC(=O)C(=C)CO)cc3)cc2)cc1] | 99 |
| 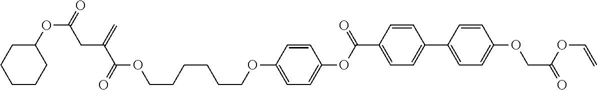 [SMILES: C=COC(=O)COc1ccc(-c2ccc(C(=O)Oc3ccc(OCCCCCCOC(=O)C(=C)CC(=O)OC4CCCCC4)cc3)cc2)cc1] | 100 |
| 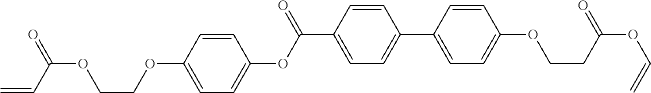 [SMILES: C=COC(=O)CCOc1ccc(-c2ccc(C(=O)Oc3ccc(OCCOC(=O)C=C)cc3)cc2)cc1] | 101 |
| 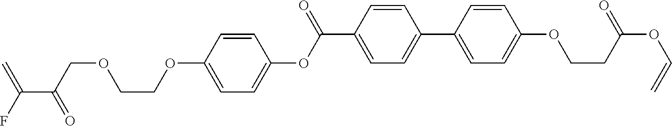 [SMILES: C=COC(=O)CCOc1ccc(-c2ccc(C(=O)Oc3ccc(OCCOCC(=O)C(=C)F)cc3)cc2)cc1] | 102 |
| 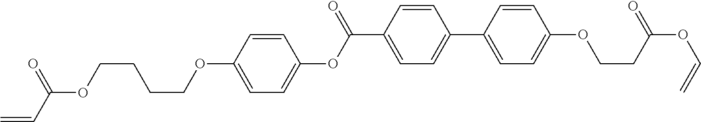 [SMILES: C=COC(=O)CCOc1ccc(-c2ccc(C(=O)Oc3ccc(OCCCCOC(=O)C=C)cc3)cc2)cc1] | 103 |
| 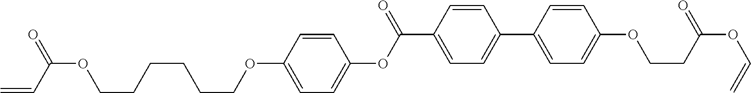 [SMILES: C=COC(=O)CCOc1ccc(-c2ccc(C(=O)Oc3ccc(OCCCCCCOC(=O)C=C)cc3)cc2)cc1] | 104 |
| 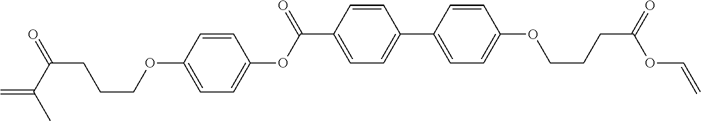 [SMILES: C=COC(=O)CCCOc1ccc(-c2ccc(C(=O)Oc3ccc(OCCCC(=O)C(=C)C)cc3)cc2)cc1] | 105 |
| 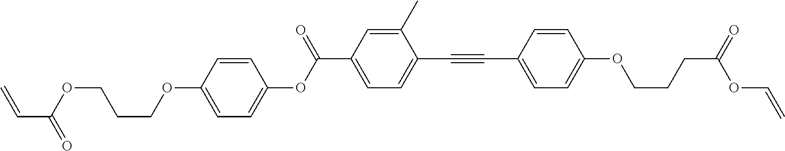 [SMILES: C=COC(=O)CCCOc1ccc(C#Cc2ccc(C(=O)Oc3ccc(OCCCOC(=O)C=C)cc3)cc2C)cc1] | 106 |
| 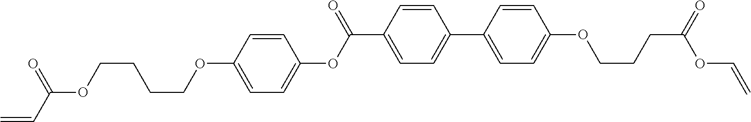 [SMILES: C=COC(=O)CCCOc1ccc(-c2ccc(C(=O)Oc3ccc(OCCCCOC(=O)C=C)cc3)cc2)cc1] | 107 |
| 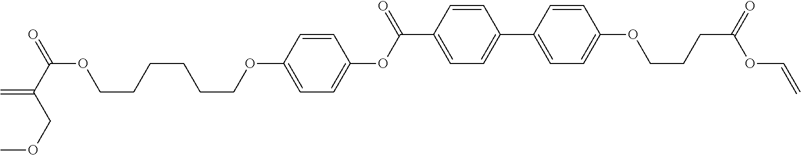 [SMILES: C=COC(=O)CCCOc1ccc(-c2ccc(C(=O)Oc3ccc(OCCCCCCOC(=O)C(=C)COC)cc3)cc2)cc1] | 108 |
| 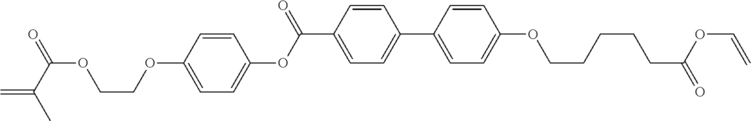 [SMILES: C=COC(=O)CCCCCOc1ccc(-c2ccc(C(=O)Oc3ccc(OCCOC(=O)C(=C)C)cc3)cc2)cc1] | 109 |
| 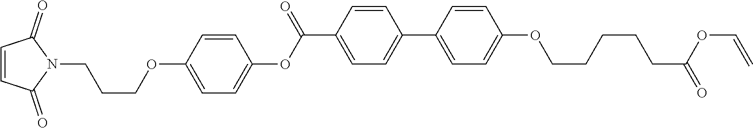 [SMILES: C=COC(=O)CCCCCOc1ccc(-c2ccc(C(=O)Oc3ccc(OCCCN4C(=O)C=CC4=O)cc3)cc2)cc1] | 110 |
| 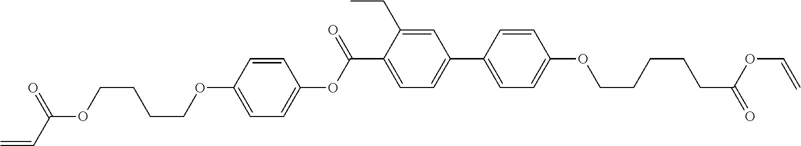 [SMILES: C=COC(=O)CCCCCOc1ccc(-c2ccc(C(=O)Oc3ccc(OCCCCOC(=O)C=C)cc3)c(CC)c2)cc1] | 111 |
| 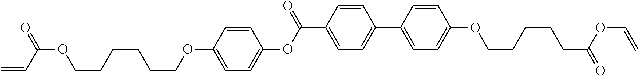 [SMILES: C=COC(=O)CCCCCOc1ccc(-c2ccc(C(=O)Oc3ccc(OCCCCCCOC(=O)C=C)cc3)cc2)cc1] | 112 |

Formula 79

| | |
|---|---|
| 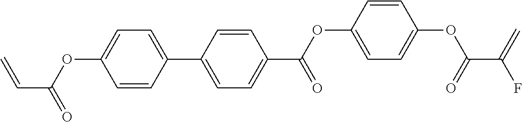 [SMILES: C=CC(=O)Oc1ccc(-c2ccc(C(=O)Oc3ccc(OC(=O)C(=C)F)cc3)cc2)cc1] | 113 |
| 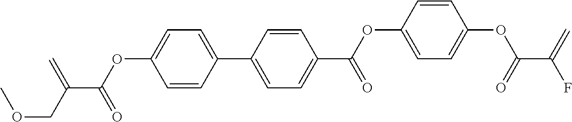 [SMILES: C=C(F)C(=O)Oc1ccc(OC(=O)c2ccc(-c3ccc(OC(=O)C(=C)COC)cc3)cc2)cc1] | 114 |
| 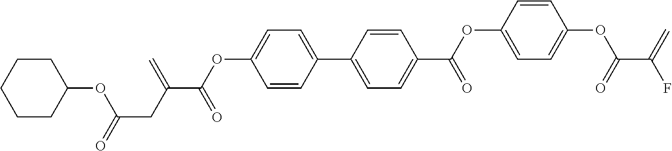 [SMILES: C=C(F)C(=O)Oc1ccc(OC(=O)c2ccc(-c3ccc(OC(=O)C(=C)CC(=O)OC4CCCCC4)cc3)cc2)cc1] | 115 |
| 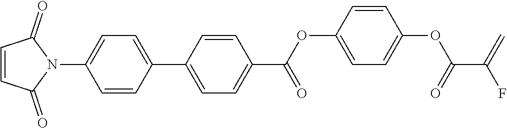 [SMILES: C=C(F)C(=O)Oc1ccc(OC(=O)c2ccc(-c3ccc(N4C(=O)C=CC4=O)cc3)cc2)cc1] | 116 |
| 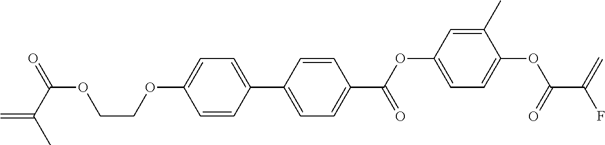 [SMILES: C=C(C)C(=O)OCCOc1ccc(-c2ccc(C(=O)Oc3ccc(OC(=O)C(=C)F)c(C)c3)cc2)cc1] | 117 |
| 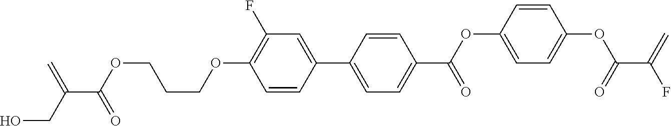 [SMILES: C=C(F)C(=O)Oc1ccc(OC(=O)c2ccc(-c3ccc(OCCCOC(=O)C(=C)CO)c(F)c3)cc2)cc1] | 118 |
| 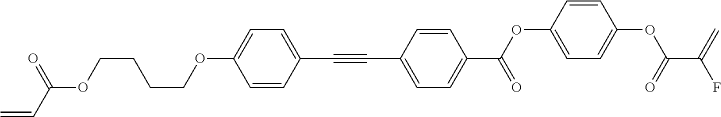 [SMILES: C=CC(=O)OCCCCOc1ccc(C#Cc2ccc(C(=O)Oc3ccc(OC(=O)C(=C)F)cc3)cc2)cc1] | 119 |
| 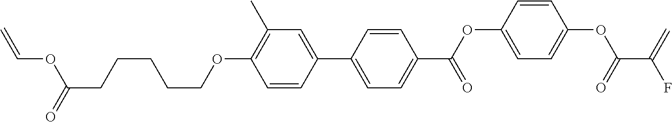 [SMILES: C=COC(=O)CCCCCOc1ccc(-c2ccc(C(=O)Oc3ccc(OC(=O)C(=C)F)cc3)cc2)cc1C] | 120 |
| 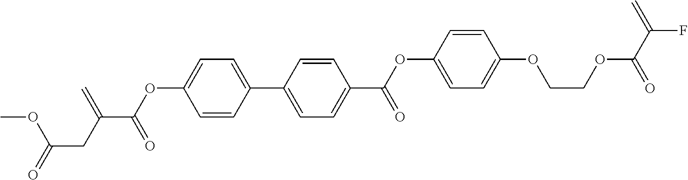 [SMILES: C=C(F)C(=O)OCCOc1ccc(OC(=O)c2ccc(-c3ccc(OC(=O)C(=C)CC(=O)OC)cc3)cc2)cc1] | 121 |
| 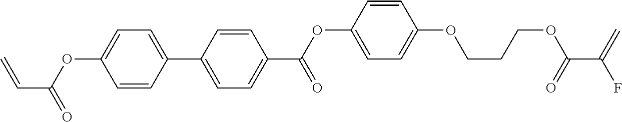 [SMILES: C=CC(=O)Oc1ccc(-c2ccc(C(=O)Oc3ccc(OCCCOC(=O)C(=C)F)cc3)cc2)cc1] | 122 |
| 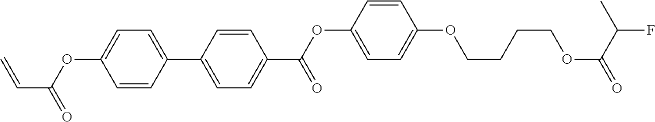 [SMILES: C=CC(=O)Oc1ccc(-c2ccc(C(=O)Oc3ccc(OCCCCOC(=O)C(C)F)cc3)cc2)cc1] | 123 |
| 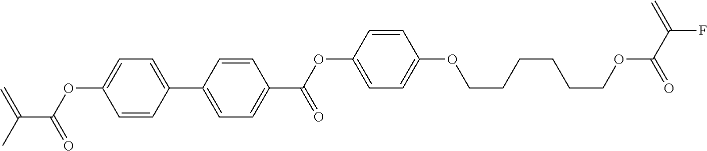 [SMILES: C=C(C)C(=O)Oc1ccc(-c2ccc(C(=O)Oc3ccc(OCCCCCCOC(=O)C(=C)F)cc3)cc2)cc1] | 124 |
| 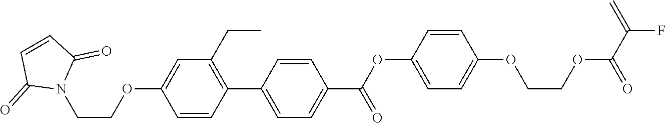 [SMILES: C=C(F)C(=O)OCCOc1ccc(OC(=O)c2ccc(-c3ccc(OCCN4C(=O)C=CC4=O)cc3CC)cc2)cc1] | 125 |
| 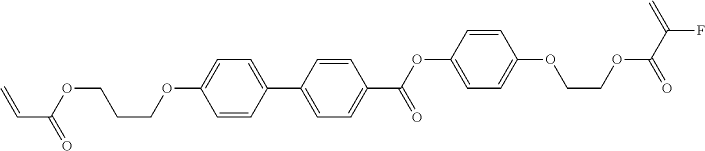 [SMILES: C=CC(=O)OCCCOc1ccc(-c2ccc(C(=O)Oc3ccc(OCCOC(=O)C(=C)F)cc3)cc2)cc1] | 126 |
| 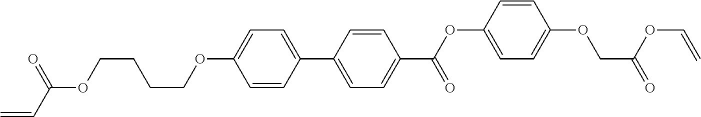 [SMILES: C=COC(=O)COc1ccc(OC(=O)c2ccc(-c3ccc(OCCCCOC(=O)C=C)cc3)cc2)cc1] | 127 |
| 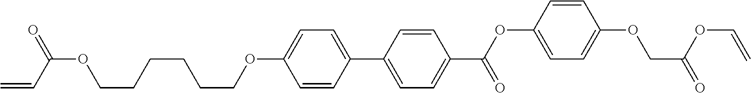 [SMILES: C=COC(=O)COc1ccc(OC(=O)c2ccc(-c3ccc(OCCCCCCOC(=O)C=C)cc3)cc2)cc1] | 128 |
| 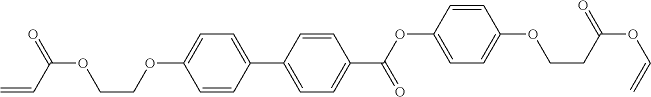 [SMILES: C=COC(=O)CCOc1ccc(OC(=O)c2ccc(-c3ccc(OCCOC(=O)C=C)cc3)cc2)cc1] | 129 |
| 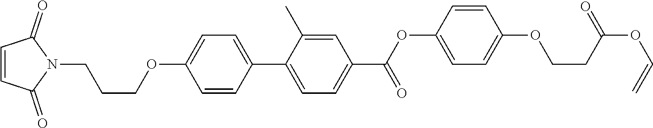 [SMILES: C=COC(=O)CCOc1ccc(OC(=O)c2ccc(-c3ccc(OCCCN4C(=O)C=CC4=O)cc3)c(C)c2)cc1] | 130 |
| 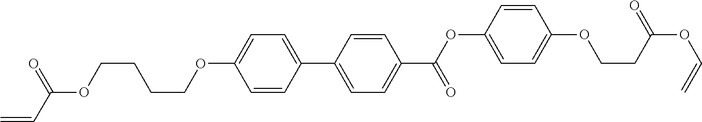 [SMILES: C=COC(=O)CCOc1ccc(OC(=O)c2ccc(-c3ccc(OCCCCOC(=O)C=C)cc3)cc2)cc1] | 131 |
| 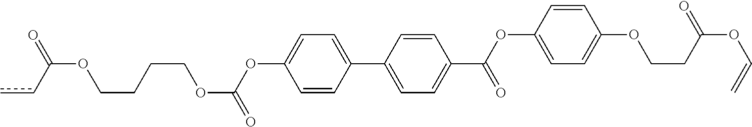 | 132 |
| 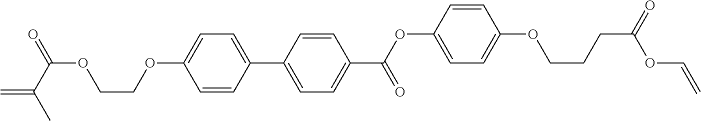 [SMILES: C=COC(=O)CCCOc1ccc(OC(=O)c2ccc(-c3ccc(OCCOC(=O)C(=C)C)cc3)cc2)cc1] | 133 |
| 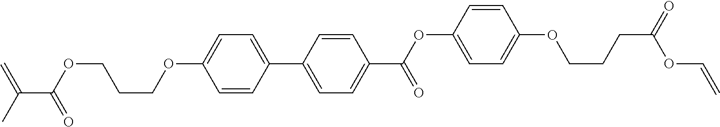 [SMILES: C=COC(=O)CCCOc1ccc(OC(=O)c2ccc(-c3ccc(OCCCOC(=O)C(=C)C)cc3)cc2)cc1] | 134 |
| 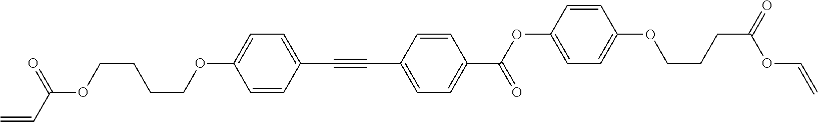 [SMILES: C=COC(=O)CCCOc1ccc(OC(=O)c2ccc(C#Cc3ccc(OCCCCOC(=O)C=C)cc3)cc2)cc1] | 135 |
| 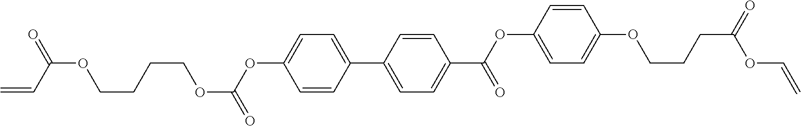 [SMILES: C=COC(=O)CCCOc1ccc(OC(=O)c2ccc(-c3ccc(OC(=O)OCCCCOC(=O)C=C)cc3)cc2)cc1] | 136 |
| 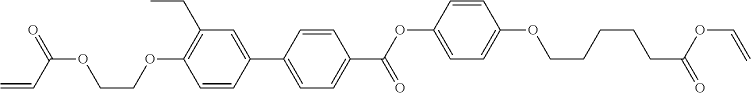 [SMILES: C=COC(=O)CCCCCOc1ccc(OC(=O)c2ccc(-c3ccc(OCCOC(=O)C=C)c(CC)c3)cc2)cc1] | 137 |
| 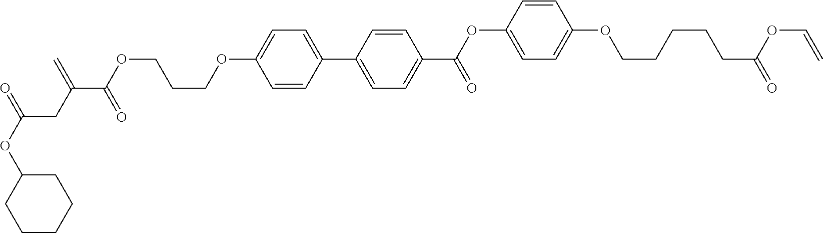 [SMILES: C=COC(=O)CCCCCOc1ccc(OC(=O)c2ccc(-c3ccc(OCCCOC(=O)C(=C)CC(=O)OC4CCCCC4)cc3)cc2)cc1] | 138 |
| 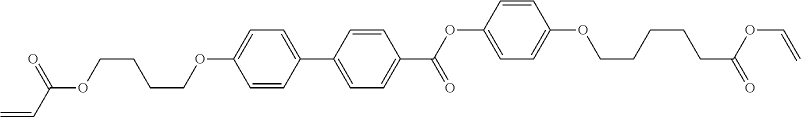 [SMILES: C=COC(=O)CCCCCOc1ccc(OC(=O)c2ccc(-c3ccc(OCCCCOC(=O)C=C)cc3)cc2)cc1] | 139 |
| 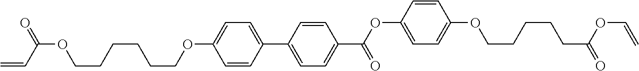 [SMILES: C=COC(=O)CCCCCOc1ccc(OC(=O)c2ccc(-c3ccc(OCCCCCCOC(=O)C=C)cc3)cc2)cc1] | 140 |

Formula 80

| | |
|---|---|
| 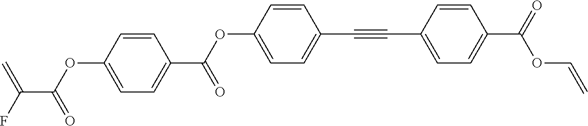 [SMILES: C=COC(=O)c1ccc(C#Cc2ccc(OC(=O)c3ccc(OC(=O)C(=C)F)cc3)cc2)cc1] | 141 |
| 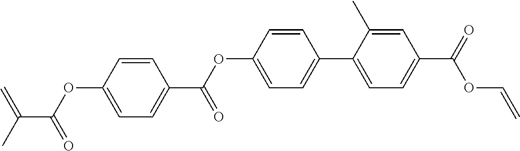 [SMILES: C=COC(=O)c1ccc(-c2ccc(OC(=O)c3ccc(OC(=O)C(=C)C)cc3)cc2)c(C)c1] | 142 |
| 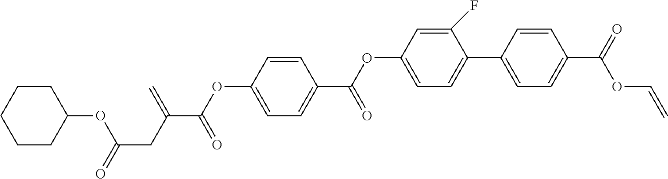 [SMILES: C=COC(=O)c1ccc(-c2ccc(OC(=O)c3ccc(OC(=O)C(=C)CC(=O)OC4CCCCC4)cc3)cc2F)cc1] | 143 |
| 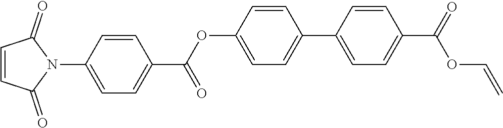 [SMILES: C=COC(=O)c1ccc(-c2ccc(OC(=O)c3ccc(N4C(=O)C=CC4=O)cc3)cc2)cc1] | 144 |
| 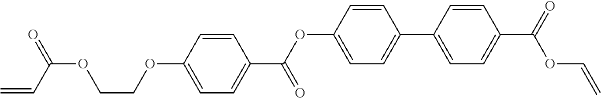 [SMILES: C=COC(=O)c1ccc(-c2ccc(OC(=O)c3ccc(OCCOC(=O)C=C)cc3)cc2)cc1] | 145 |
| 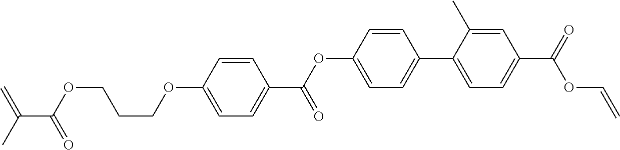 [SMILES: C=COC(=O)c1ccc(-c2ccc(OC(=O)c3ccc(OCCCOC(=O)C(=C)C)cc3)cc2)c(C)c1] | 146 |
| 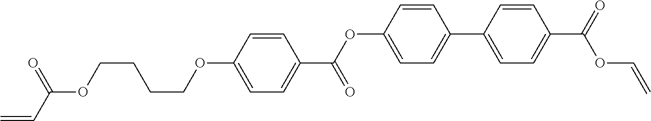 [SMILES: C=COC(=O)c1ccc(-c2ccc(OC(=O)c3ccc(OCCCCOC(=O)C=C)cc3)cc2)cc1] | 147 |
| 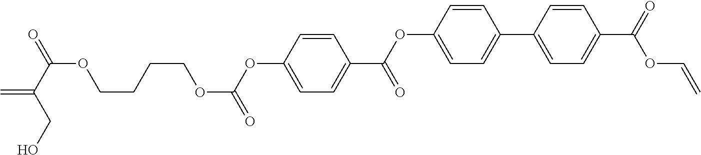 [SMILES: C=COC(=O)c1ccc(-c2ccc(OC(=O)c3ccc(OC(=O)OCCCCOC(=O)C(=C)CO)cc3)cc2)cc1] | 148 |
| 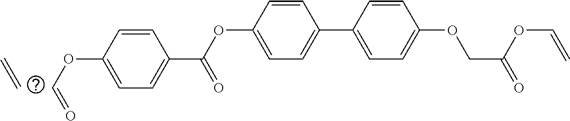 | 149 |
| 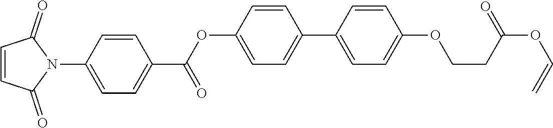 [SMILES: C=COC(=O)CCOc1ccc(-c2ccc(OC(=O)c3ccc(N4C(=O)C=CC4=O)cc3)cc2)cc1] | 150 |
| 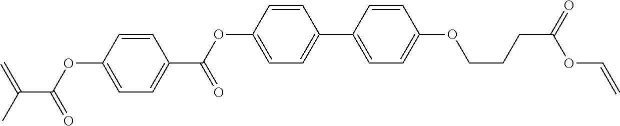 [SMILES: C=COC(=O)CCCOc1ccc(-c2ccc(OC(=O)c3ccc(OC(=O)C(=C)C)cc3)cc2)cc1] | 151 |
| 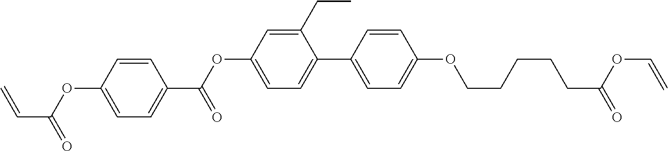 [SMILES: C=COC(=O)CCCCCOc1ccc(-c2ccc(OC(=O)c3ccc(OC(=O)C=C)cc3)cc2CC)cc1] | 152 |
| 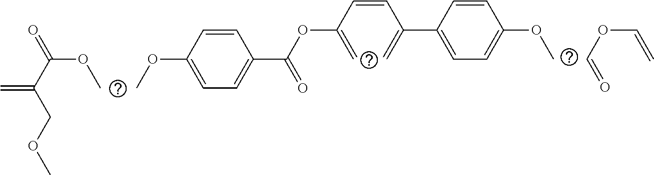 | 153 |
| 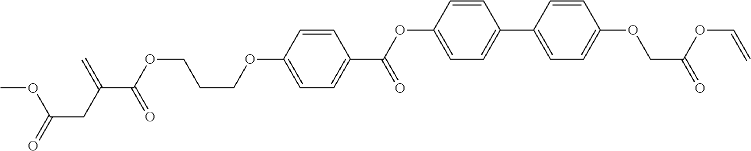 [SMILES: C=COC(=O)COc1ccc(-c2ccc(OC(=O)c3ccc(OCCCOC(=O)C(=C)CC(=O)OC)cc3)cc2)cc1] | 154 |
| 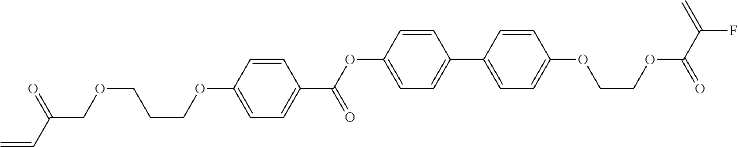 [SMILES: C=CC(=O)COCCCOc1ccc(C(=O)Oc2ccc(-c3ccc(OCCOC(=O)C(=C)F)cc3)cc2)cc1] | 155 |
| 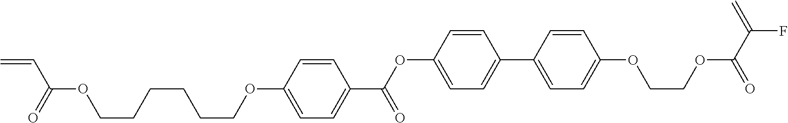 [SMILES: C=CC(=O)OCCCCCCOc1ccc(C(=O)Oc2ccc(-c3ccc(OCCOC(=O)C(=C)F)cc3)cc2)cc1] | 156 |
| 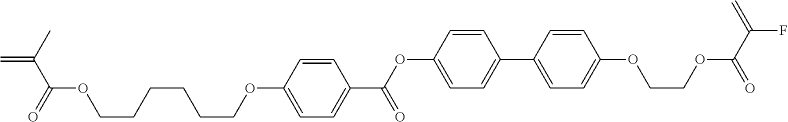 [SMILES: C=C(C)C(=O)OCCCCCCOc1ccc(C(=O)Oc2ccc(-c3ccc(OCCOC(=O)C(=C)F)cc3)cc2)cc1] | 157 |
| 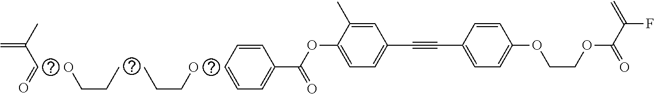 | 158 |
| 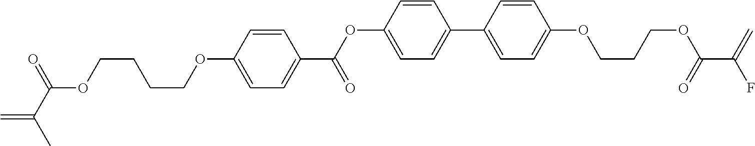 [SMILES: C=C(C)C(=O)OCCCCOc1ccc(C(=O)Oc2ccc(-c3ccc(OCCCOC(=O)C(=C)F)cc3)cc2)cc1] | 159 |
| 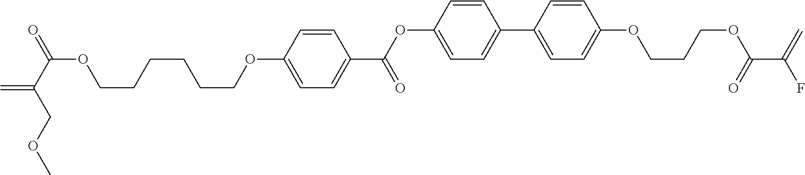 [SMILES: C=C(F)C(=O)OCCCOc1ccc(-c2ccc(OC(=O)c3ccc(OCCCCCCOC(=O)C(=C)COC)cc3)cc2)cc1] | 160 |
| 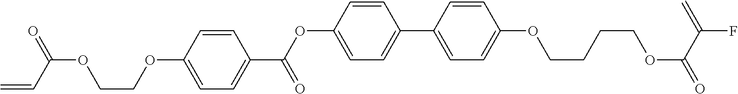 [SMILES: C=CC(=O)OCCOc1ccc(C(=O)Oc2ccc(-c3ccc(OCCCCOC(=O)C(=C)F)cc3)cc2)cc1] | 161 |
| 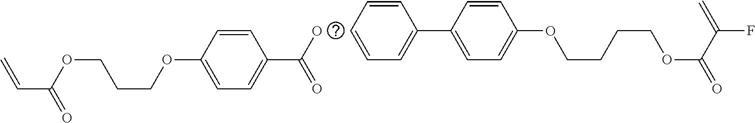 | 162 |
| 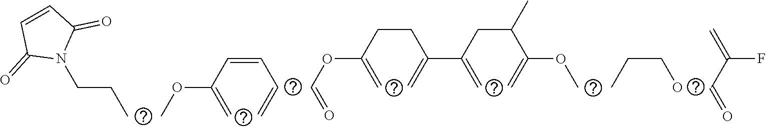 | 163 |
| 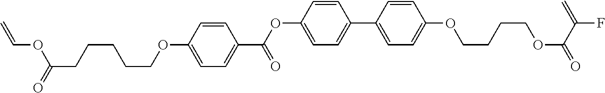 [SMILES: C=COC(=O)CCCCCOc1ccc(C(=O)Oc2ccc(-c3ccc(OCCCCOC(=O)C(=C)F)cc3)cc2)cc1] | 164 |
| 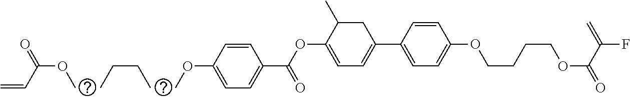 | 165 |
| 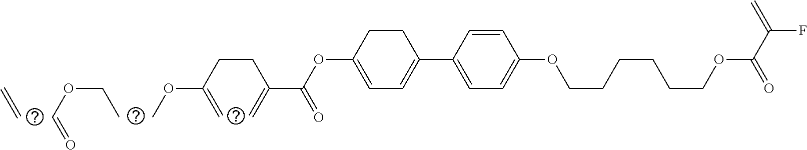 | 166 |
| 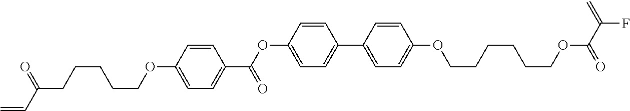 [SMILES: C=CC(=O)CCCCCOc1ccc(C(=O)Oc2ccc(-c3ccc(OCCCCCCOC(=O)C(=C)F)cc3)cc2)cc1] | 167 |
| 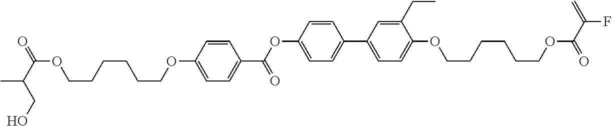 [SMILES: C=C(F)C(=O)OCCCCCCOc1ccc(-c2ccc(OC(=O)c3ccc(OCCCCCCOC(=O)C(C)CO)cc3)cc2)cc1CC] | 168 |

Formula 81

| | |
|---|---|
| 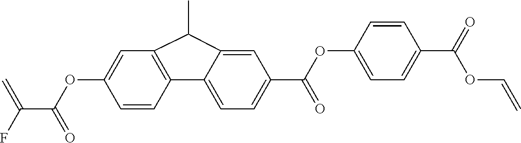 [SMILES: C=COC(=O)c1ccc(OC(=O)c2ccc3c(c2)C(C)c2cc(OC(=O)C(=C)F)ccc2-3)cc1] | 169 |
| 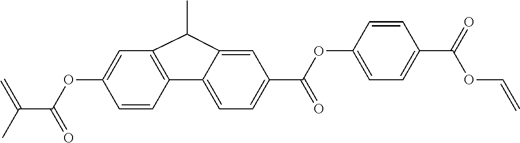 [SMILES: C=COC(=O)c1ccc(OC(=O)c2ccc3c(c2)C(C)c2cc(OC(=O)C(=C)C)ccc2-3)cc1] | 170 |
| 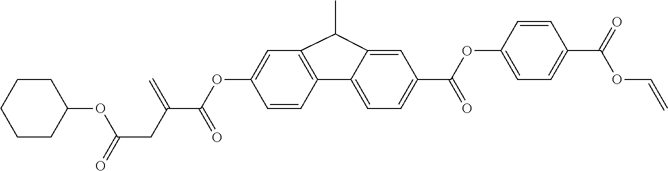 [SMILES: C=COC(=O)c1ccc(OC(=O)c2ccc3c(c2)C(C)c2cc(OC(=O)C(=C)CC(=O)OC4CCCCC4)ccc2-3)cc1] | 171 |
| 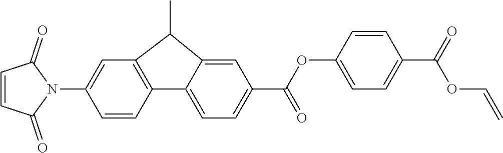 [SMILES: C=COC(=O)c1ccc(OC(=O)c2ccc3c(c2)C(C)c2cc(N4C(=O)C=CC4=O)ccc2-3)cc1] | 172 |
| 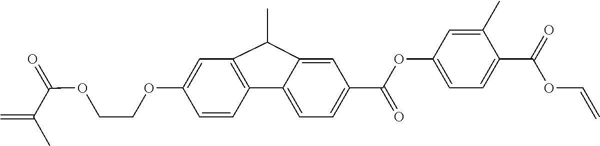 [SMILES: C=COC(=O)c1ccc(OC(=O)c2ccc3c(c2)C(C)c2cc(OCCOC(=O)C(=C)C)ccc2-3)cc1C] | 173 |
| 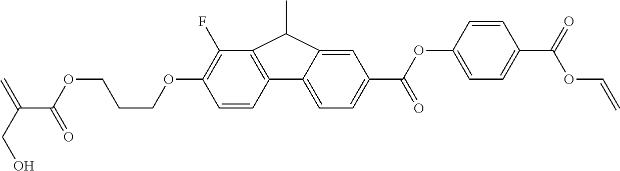 [SMILES: C=COC(=O)c1ccc(OC(=O)c2ccc3c(c2)C(C)c2c-3ccc(OCCCOC(=O)C(=C)CO)c2F)cc1] | 174 |
| 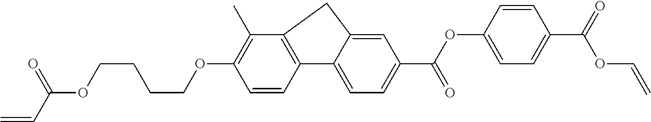 [SMILES: C=COC(=O)c1ccc(OC(=O)c2ccc3c(c2)Cc2c-3ccc(OCCCCOC(=O)C=C)c2C)cc1] | 175 |
| 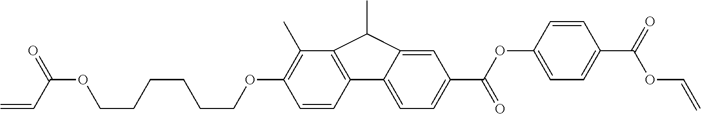 [SMILES: C=COC(=O)c1ccc(OC(=O)c2ccc3c(c2)C(C)c2c-3ccc(OCCCCCCOC(=O)C=C)c2C)cc1] | 176 |
| 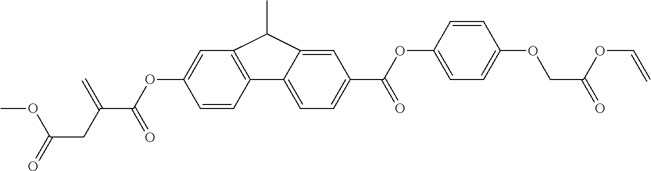 [SMILES: C=COC(=O)COc1ccc(OC(=O)c2ccc3c(c2)C(C)c2cc(OC(=O)C(=C)CC(=O)OC)ccc2-3)cc1] | 177 |
| 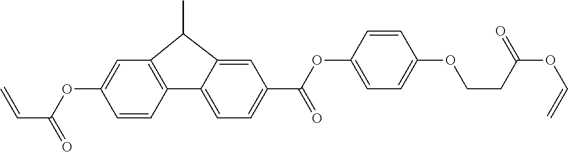 [SMILES: C=COC(=O)CCOc1ccc(OC(=O)c2ccc3c(c2)C(C)c2cc(OC(=O)C=C)ccc2-3)cc1] | 178 |
| 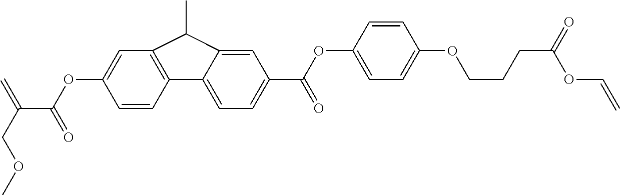 [SMILES: C=COC(=O)CCCOc1ccc(OC(=O)c2ccc3c(c2)C(C)c2cc(OC(=O)C(=C)COC)ccc2-3)cc1] | 179 |
| 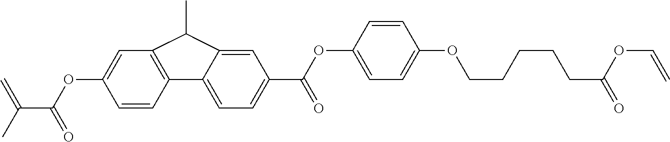 [SMILES: C=COC(=O)CCCCCOc1ccc(OC(=O)c2ccc3c(c2)C(C)c2cc(OC(=O)C(=C)C)ccc2-3)cc1] | 180 |
| 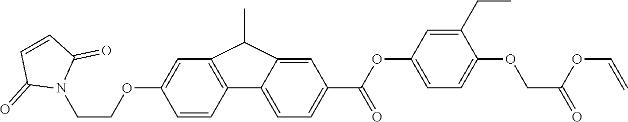 [SMILES: C=COC(=O)COc1ccc(OC(=O)c2ccc3c(c2)C(C)c2cc(OCCN4C(=O)C=CC4=O)ccc2-3)cc1CC] | 181 |
| 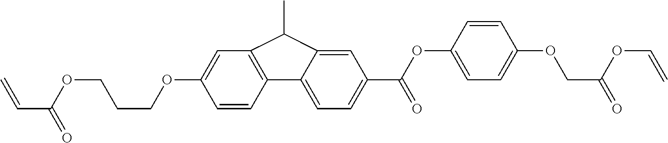 [SMILES: C=COC(=O)COc1ccc(OC(=O)c2ccc3c(c2)C(C)c2cc(OCCCOC(=O)C=C)ccc2-3)cc1] | 182 |
| 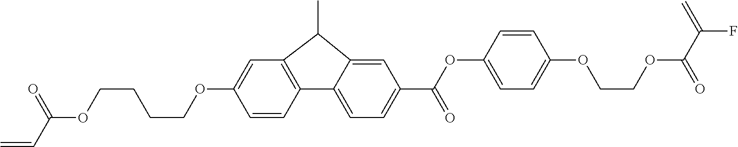 [SMILES: C=CC(=O)OCCCCOc1ccc2c(c1)C(C)c1cc(C(=O)Oc3ccc(OCCOC(=O)C(=C)F)cc3)ccc1-2] | 183 |
| 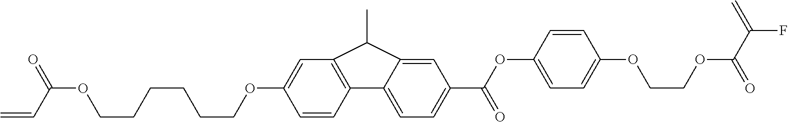 [SMILES: C=CC(=O)OCCCCCCOc1ccc2c(c1)C(C)c1cc(C(=O)Oc3ccc(OCCOC(=O)C(=C)F)cc3)ccc1-2] | 184 |
| 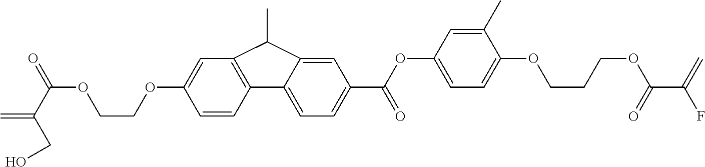 [SMILES: C=C(F)C(=O)OCCCOc1ccc(OC(=O)c2ccc3c(c2)C(C)c2cc(OCCOC(=O)C(=C)CO)ccc2-3)cc1C] | 185 |
| 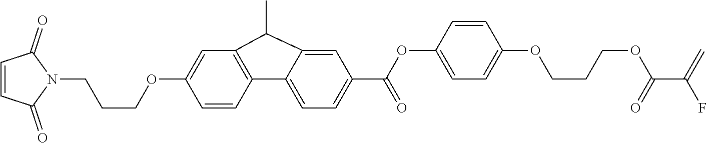 [SMILES: C=C(F)C(=O)OCCCOc1ccc(OC(=O)c2ccc3c(c2)C(C)c2cc(OCCCN4C(=O)C=CC4=O)ccc2-3)cc1] | 186 |
| 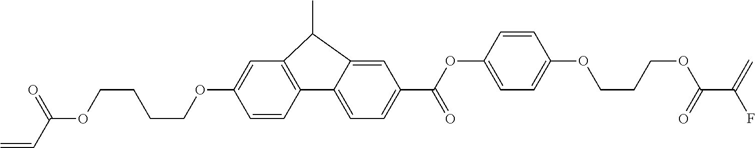 [SMILES: C=CC(=O)OCCCCOc1ccc2c(c1)C(C)c1cc(C(=O)Oc3ccc(OCCCOC(=O)C(=C)F)cc3)ccc1-2] | 187 |
| 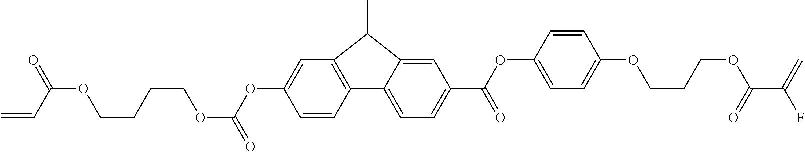 [SMILES: C=CC(=O)OCCCCOC(=O)Oc1ccc2c(c1)C(C)c1cc(C(=O)Oc3ccc(OCCCOC(=O)C(=C)F)cc3)ccc1-2] | 188 |
| 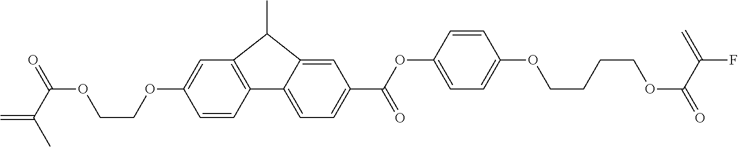 [SMILES: C=C(C)C(=O)OCCOc1ccc2c(c1)C(C)c1cc(C(=O)Oc3ccc(OCCCCOC(=O)C(=C)F)cc3)ccc1-2] | 189 |
| 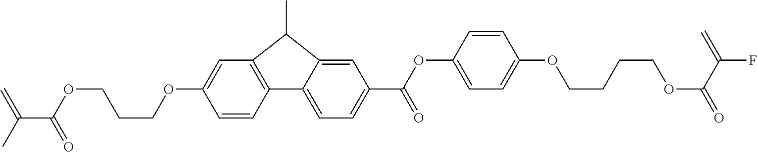 [SMILES: C=C(C)C(=O)OCCCOc1ccc2c(c1)C(C)c1cc(C(=O)Oc3ccc(OCCCCOC(=O)C(=C)F)cc3)ccc1-2] | 190 |
| 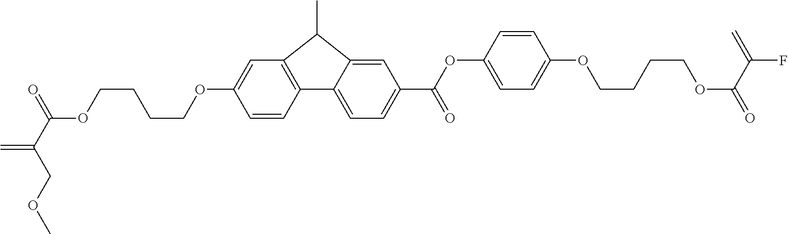 [SMILES: C=C(F)C(=O)OCCCCOc1ccc(OC(=O)c2ccc3c(c2)C(C)c2cc(OCCCCOC(=O)C(=C)COC)ccc2-3)cc1] | 191 |
| 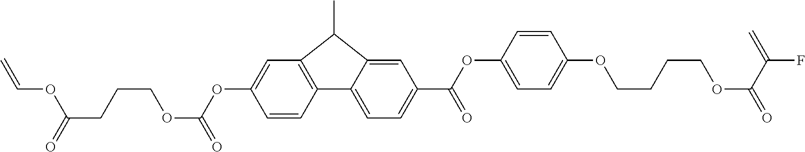 [SMILES: C=COC(=O)CCCOC(=O)Oc1ccc2c(c1)C(C)c1cc(C(=O)Oc3ccc(OCCCCOC(=O)C(=C)F)cc3)ccc1-2] | 192 |
| 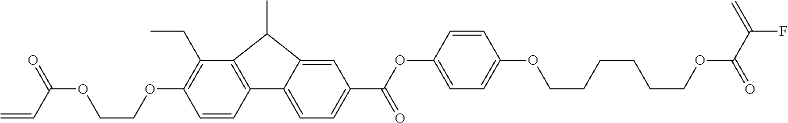 [SMILES: C=CC(=O)OCCOc1ccc2c(c1CC)C(C)c1cc(C(=O)Oc3ccc(OCCCCCCOC(=O)C(=C)F)cc3)ccc1-2] | 193 |
| 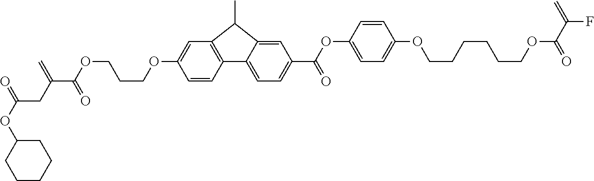 [SMILES: C=C(F)C(=O)OCCCCCCOc1ccc(OC(=O)c2ccc3c(c2)C(C)c2cc(OCCCOC(=O)C(=C)CC(=O)OC4CCCCC4)ccc2-3)cc1] | 194 |
| 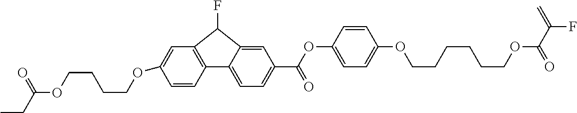 [SMILES: C=C(F)C(=O)OCCCCCCOc1ccc(OC(=O)c2ccc3c(c2)C(F)c2cc(OCCCCOC(=O)CC)ccc2-3)cc1] | 195 |
| 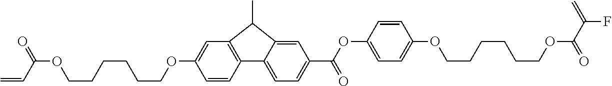 [SMILES: C=CC(=O)OCCCCCCOc1ccc2c(c1)C(C)c1cc(C(=O)Oc3ccc(OCCCCCCOC(=O)C(=C)F)cc3)ccc1-2] | 196 |

Formula 82

| | |
|---|---|
| 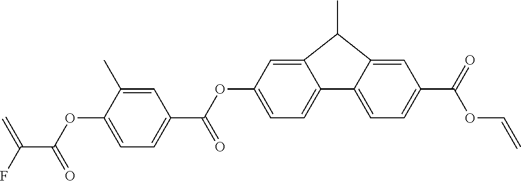 [SMILES: C=COC(=O)c1ccc2c(c1)C(C)c1cc(OC(=O)c3ccc(OC(=O)C(=C)F)c(C)c3)ccc1-2] | 197 |
| 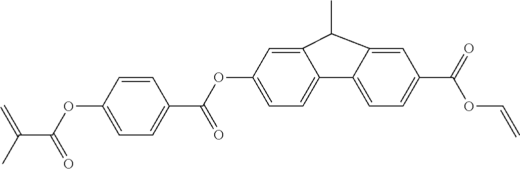 [SMILES: C=COC(=O)c1ccc2c(c1)C(C)c1cc(OC(=O)c3ccc(OC(=O)C(=C)C)cc3)ccc1-2] | 198 |
| 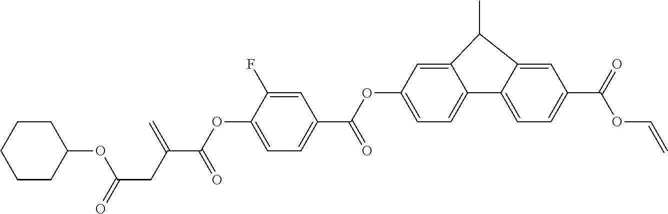 [SMILES: C=COC(=O)c1ccc2c(c1)C(C)c1cc(OC(=O)c3ccc(OC(=O)C(=C)CC(=O)OC4CCCCC4)c(F)c3)ccc1-2] | 199 |
| 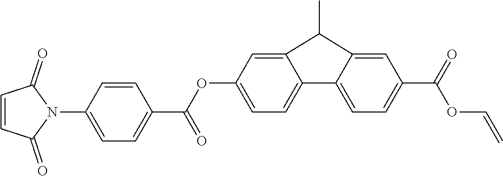 [SMILES: C=COC(=O)c1ccc2c(c1)C(C)c1cc(OC(=O)c3ccc(N4C(=O)C=CC4=O)cc3)ccc1-2] | 200 |
| 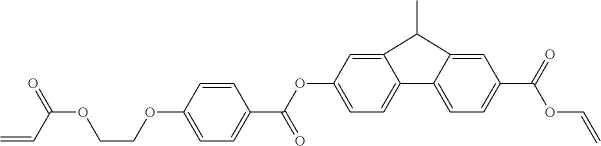 [SMILES: C=COC(=O)c1ccc2c(c1)C(C)c1cc(OC(=O)c3ccc(OCCOC(=O)C=C)cc3)ccc1-2] | 201 |
| 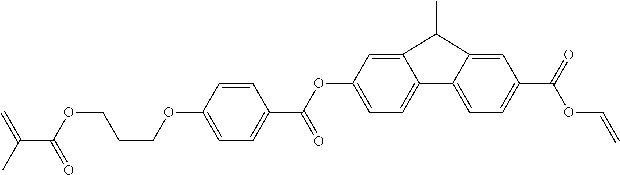 [SMILES: C=COC(=O)c1ccc2c(c1)C(C)c1cc(OC(=O)c3ccc(OCCCOC(=O)C(=C)C)cc3)ccc1-2] | 202 |
| 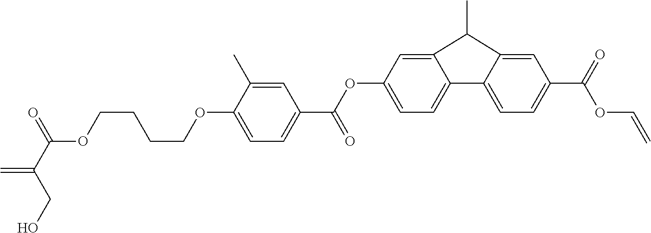 [SMILES: C=COC(=O)c1ccc2c(c1)C(C)c1cc(OC(=O)c3ccc(OCCCCOC(=O)C(=C)CO)c(C)c3)ccc1-2] | 203 |
| 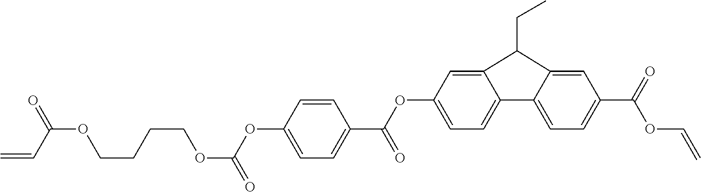 [SMILES: C=COC(=O)c1ccc2c(c1)C(CC)c1cc(OC(=O)c3ccc(OC(=O)OCCCCOC(=O)C=C)cc3)ccc1-2] | 204 |
| 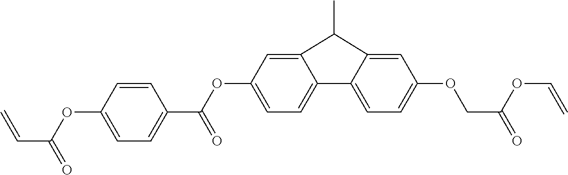 [SMILES: C=COC(=O)COc1ccc2c(c1)C(C)c1cc(OC(=O)c3ccc(OC(=O)C=C)cc3)ccc1-2] | 205 |
| 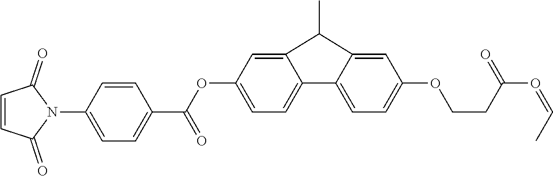 | 206 |
| 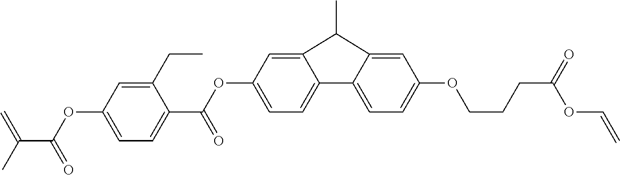 [SMILES: C=COC(=O)CCCOc1ccc2c(c1)C(C)c1cc(OC(=O)c3ccc(OC(=O)C(=C)C)cc3CC)ccc1-2] | 207 |
| 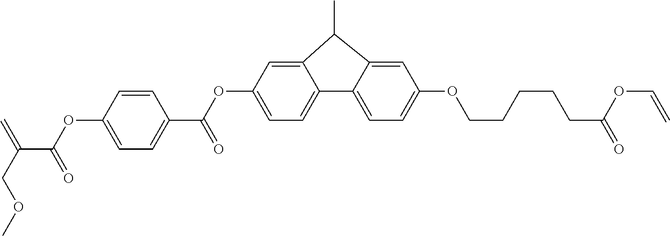 [SMILES: C=COC(=O)CCCCCOc1ccc2c(c1)C(C)c1cc(OC(=O)c3ccc(OC(=O)C(=C)COC)cc3)ccc1-2] | 208 |
| 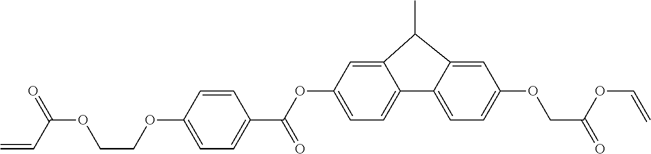 [SMILES: C=COC(=O)COc1ccc2c(c1)C(C)c1cc(OC(=O)c3ccc(OCCOC(=O)C=C)cc3)ccc1-2] | 209 |
| 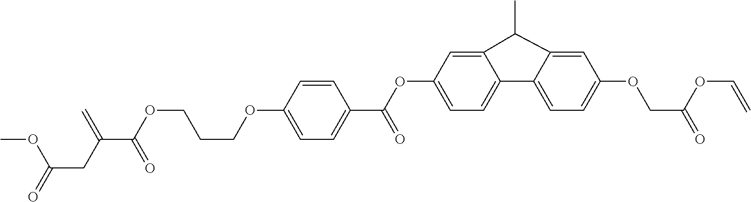 [SMILES: C=COC(=O)COc1ccc2c(c1)C(C)c1cc(OC(=O)c3ccc(OCCCOC(=O)C(=C)CC(=O)OC)cc3)ccc1-2] | 210 |
| 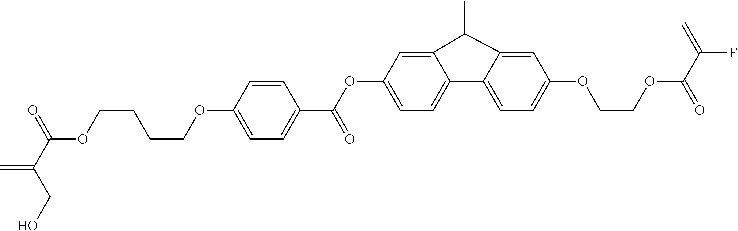 [SMILES: C=C(F)C(=O)OCCOc1ccc2c(c1)C(C)c1cc(OC(=O)c3ccc(OCCCCOC(=O)C(=C)CO)cc3)ccc1-2] | 211 |
| 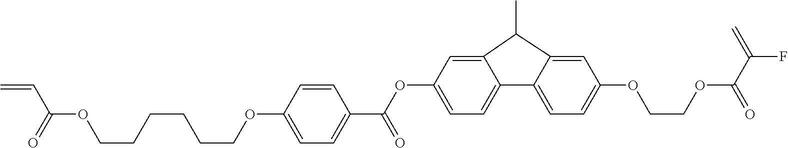 [SMILES: C=CC(=O)OCCCCCCOc1ccc(C(=O)Oc2ccc3c(c2)C(C)c2cc(OCCOC(=O)C(=C)F)ccc2-3)cc1] | 212 |
| 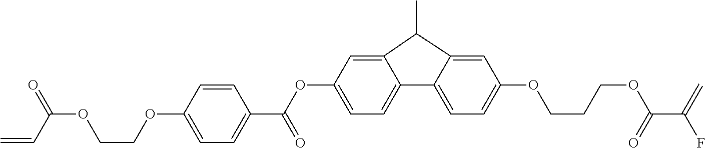 [SMILES: C=CC(=O)OCCOc1ccc(C(=O)Oc2ccc3c(c2)C(C)c2cc(OCCCOC(=O)C(=C)F)ccc2-3)cc1] | 213 |
| 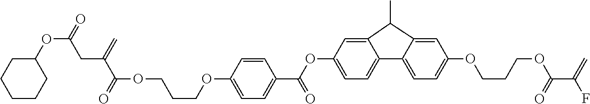 [SMILES: C=C(F)C(=O)OCCCOc1ccc2c(c1)C(C)c1cc(OC(=O)c3ccc(OCCCOC(=O)C(=C)CC(=O)OC4CCCCC4)cc3)ccc1-2] | 214 |
| 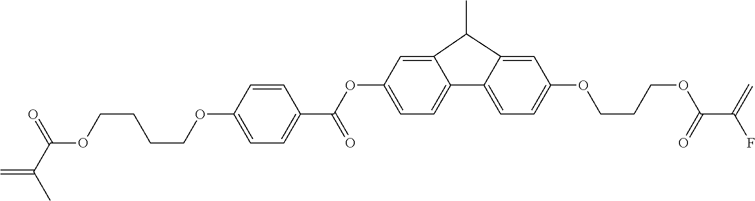 [SMILES: C=C(C)C(=O)OCCCCOc1ccc(C(=O)Oc2ccc3c(c2)C(C)c2cc(OCCCOC(=O)C(=C)F)ccc2-3)cc1] | 215 |
| 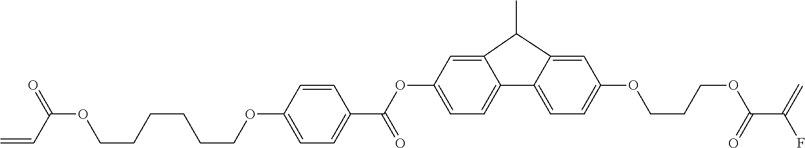 [SMILES: C=CC(=O)OCCCCCCOc1ccc(C(=O)Oc2ccc3c(c2)C(C)c2cc(OCCCOC(=O)C(=C)F)ccc2-3)cc1] | 216 |
| 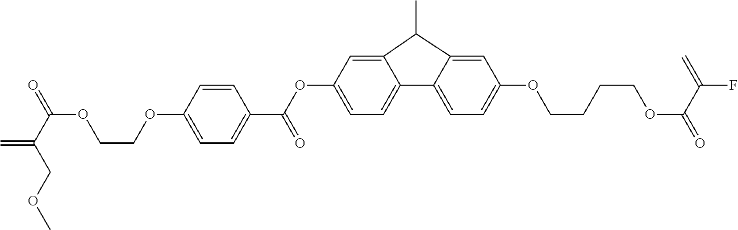 [SMILES: C=C(F)C(=O)OCCCCOc1ccc2c(c1)C(C)c1cc(OC(=O)c3ccc(OCCOC(=O)C(=C)COC)cc3)ccc1-2] | 217 |
| 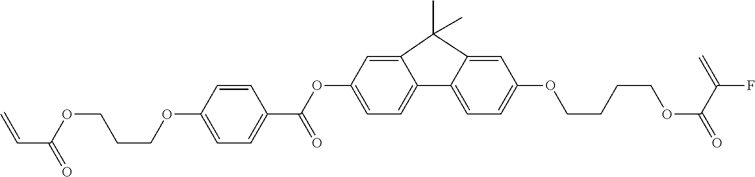 [SMILES: C=CC(=O)OCCCOc1ccc(C(=O)Oc2ccc3c(c2)C(C)(C)c2cc(OCCCCOC(=O)C(=C)F)ccc2-3)cc1] | 218 |
| 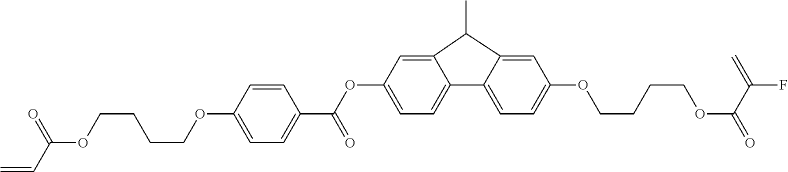 [SMILES: C=CC(=O)OCCCCOc1ccc(C(=O)Oc2ccc3c(c2)C(C)c2cc(OCCCCOC(=O)C(=C)F)ccc2-3)cc1] | 219 |
| 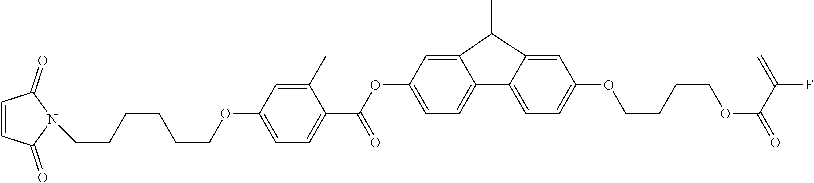 [SMILES: C=C(F)C(=O)OCCCCOc1ccc2c(c1)C(C)c1cc(OC(=O)c3ccc(OCCCCCCN4C(=O)C=CC4=O)cc3C)ccc1-2] | 220 |
| 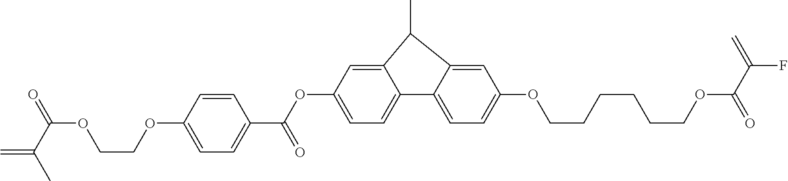 [SMILES: C=C(C)C(=O)OCCOc1ccc(C(=O)Oc2ccc3c(c2)C(C)c2cc(OCCCCCCOC(=O)C(=C)F)ccc2-3)cc1] | 221 |
| 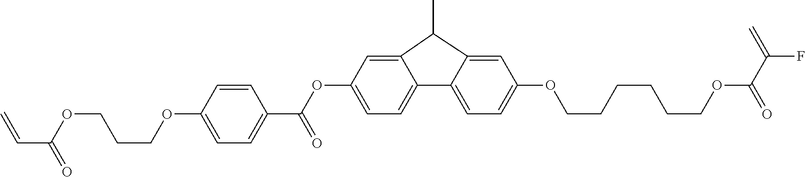 [SMILES: C=CC(=O)OCCCOc1ccc(C(=O)Oc2ccc3c(c2)C(C)c2cc(OCCCCCCOC(=O)C(=C)F)ccc2-3)cc1] | 222 |
| 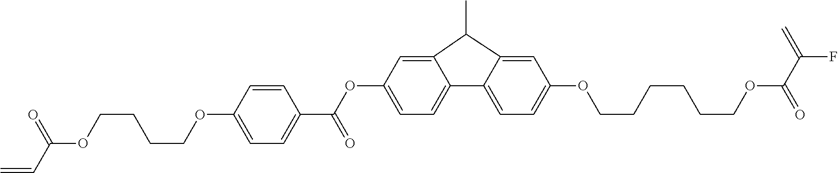 [SMILES: C=CC(=O)OCCCCOc1ccc(C(=O)Oc2ccc3c(c2)C(C)c2cc(OCCCCCCOC(=O)C(=C)F)ccc2-3)cc1] | 223 |
| 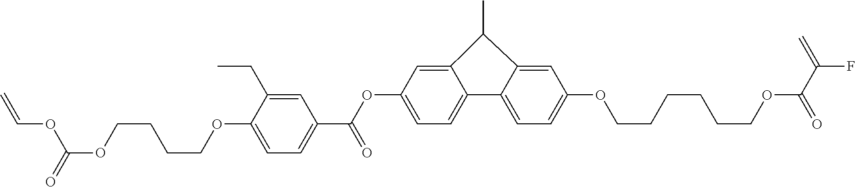 [SMILES: C=COC(=O)OCCCCOc1ccc(C(=O)Oc2ccc3c(c2)C(C)c2cc(OCCCCCCOC(=O)C(=C)F)ccc2-3)cc1CC] | 224 |

Formula 83

| | |
|---|---|
| 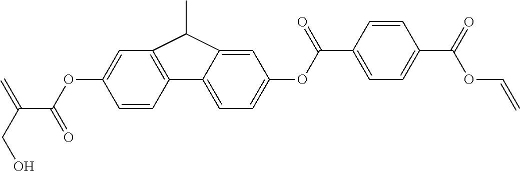 [SMILES: C=COC(=O)c1ccc(C(=O)Oc2ccc3c(c2)C(C)c2cc(OC(=O)C(=C)CO)ccc2-3)cc1] | 225 |
| 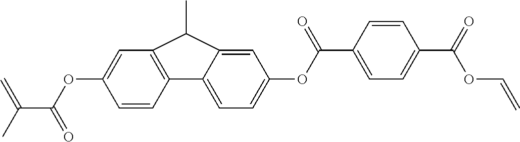 [SMILES: C=COC(=O)c1ccc(C(=O)Oc2ccc3c(c2)C(C)c2cc(OC(=O)C(=C)C)ccc2-3)cc1] | 226 |
| 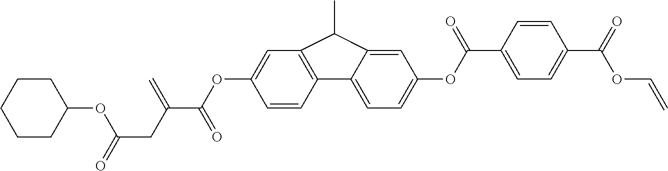 [SMILES: C=COC(=O)c1ccc(C(=O)Oc2ccc3c(c2)C(C)c2cc(OC(=O)C(=C)CC(=O)OC4CCCCC4)ccc2-3)cc1] | 227 |
| 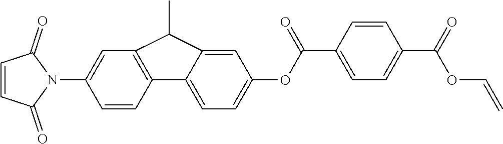 [SMILES: C=COC(=O)c1ccc(C(=O)Oc2ccc3c(c2)C(C)c2cc(N4C(=O)C=CC4=O)ccc2-3)cc1] | 228 |
| 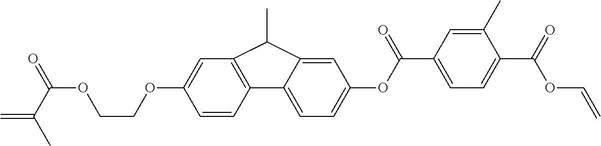 [SMILES: C=COC(=O)c1ccc(C(=O)Oc2ccc3c(c2)C(C)c2cc(OCCOC(=O)C(=C)C)ccc2-3)cc1C] | 229 |
| 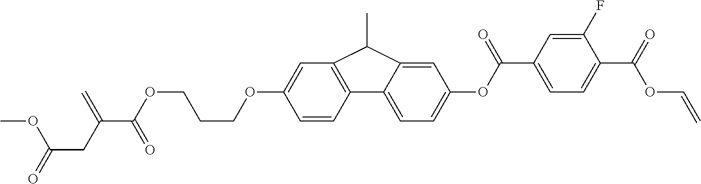 [SMILES: C=COC(=O)c1ccc(C(=O)Oc2ccc3c(c2)C(C)c2cc(OCCCOC(=O)C(=C)CC(=O)OC)ccc2-3)cc1F] | 230 |
| 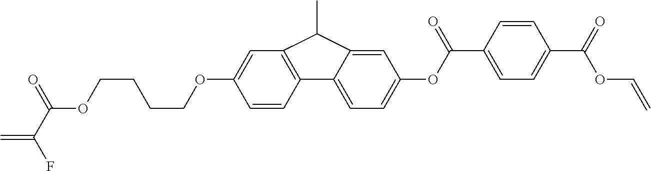 [SMILES: C=COC(=O)c1ccc(C(=O)Oc2ccc3c(c2)C(C)c2cc(OCCCCOC(=O)C(=C)F)ccc2-3)cc1] | 231 |
| 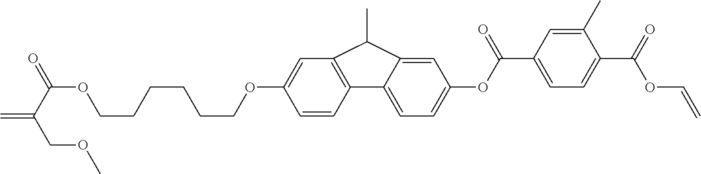 [SMILES: C=COC(=O)c1ccc(C(=O)Oc2ccc3c(c2)C(C)c2cc(OCCCCCCOC(=O)C(=C)COC)ccc2-3)cc1C] | 232 |
| 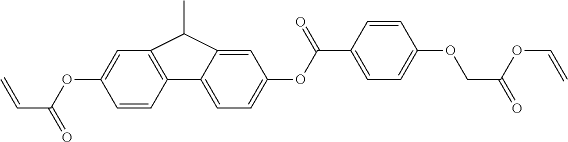 [SMILES: C=COC(=O)COc1ccc(C(=O)Oc2ccc3c(c2)C(C)c2cc(OC(=O)C=C)ccc2-3)cc1] | 233 |
| 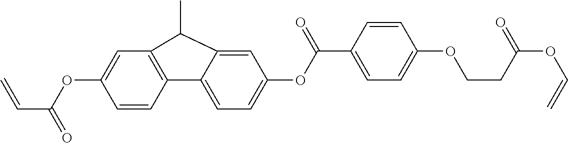 [SMILES: C=COC(=O)CCOc1ccc(C(=O)Oc2ccc3c(c2)C(C)c2cc(OC(=O)C=C)ccc2-3)cc1] | 234 |
| 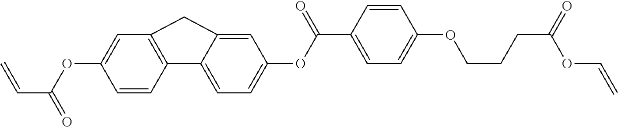 [SMILES: C=COC(=O)CCCOc1ccc(C(=O)Oc2ccc3c(c2)Cc2cc(OC(=O)C=C)ccc2-3)cc1] | 235 |
| 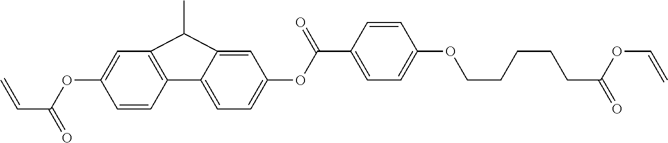 [SMILES: C=COC(=O)CCCCCOc1ccc(C(=O)Oc2ccc3c(c2)C(C)c2cc(OC(=O)C=C)ccc2-3)cc1] | 236 |
| 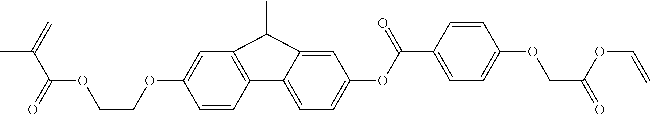 [SMILES: C=COC(=O)COc1ccc(C(=O)Oc2ccc3c(c2)C(C)c2cc(OCCOC(=O)C(=C)C)ccc2-3)cc1] | 237 |
| 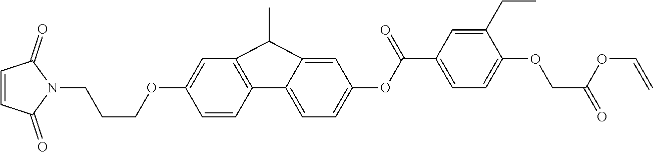 [SMILES: C=COC(=O)COc1ccc(C(=O)Oc2ccc3c(c2)C(C)c2cc(OCCCN4C(=O)C=CC4=O)ccc2-3)cc1CC] | 238 |
| 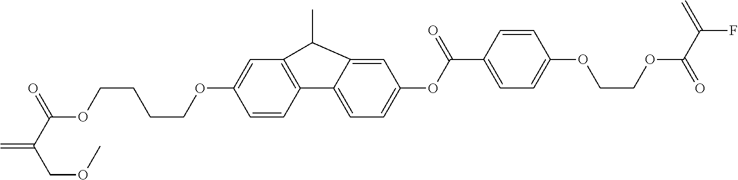 [SMILES: C=C(F)C(=O)OCCOc1ccc(C(=O)Oc2ccc3c(c2)C(C)c2cc(OCCCCOC(=O)C(=C)COC)ccc2-3)cc1] | 239 |
| 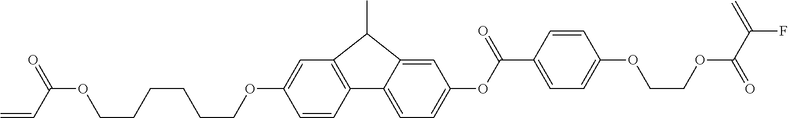 [SMILES: C=CC(=O)OCCCCCCOc1ccc2c(c1)C(C)c1cc(OC(=O)c3ccc(OCCOC(=O)C(=C)F)cc3)ccc1-2] | 240 |
| 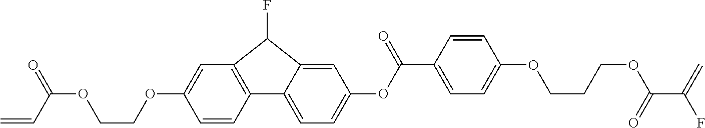 [SMILES: C=CC(=O)OCCOc1ccc2c(c1)C(F)c1cc(OC(=O)c3ccc(OCCCOC(=O)C(=C)F)cc3)ccc1-2] | 241 |
| 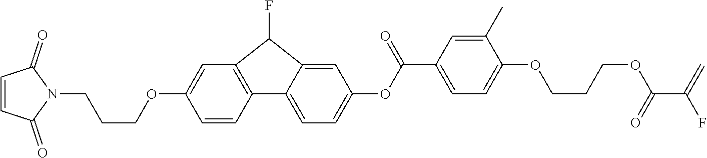 [SMILES: C=C(F)C(=O)OCCCOc1ccc(C(=O)Oc2ccc3c(c2)C(F)c2cc(OCCCN4C(=O)C=CC4=O)ccc2-3)cc1C] | 242 |
| 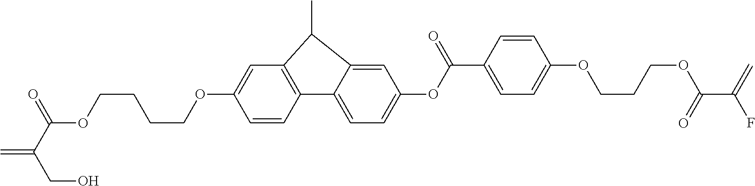 [SMILES: C=C(F)C(=O)OCCCOc1ccc(C(=O)Oc2ccc3c(c2)C(C)c2cc(OCCCCOC(=O)C(=C)CO)ccc2-3)cc1] | 243 |
| 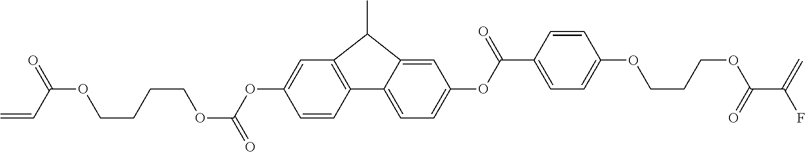 [SMILES: C=CC(=O)OCCCCOC(=O)Oc1ccc2c(c1)C(C)c1cc(OC(=O)c3ccc(OCCCOC(=O)C(=C)F)cc3)ccc1-2] | 244 |
| 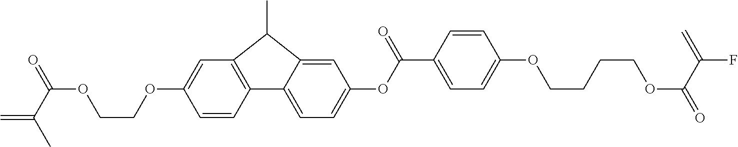 [SMILES: C=C(C)C(=O)OCCOc1ccc2c(c1)C(C)c1cc(OC(=O)c3ccc(OCCCCOC(=O)C(=C)F)cc3)ccc1-2] | 245 |
| 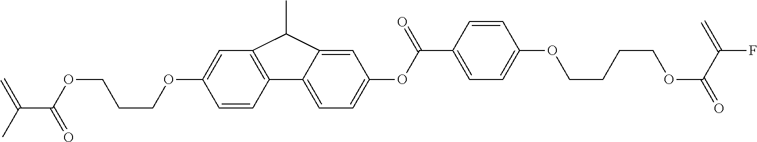 [SMILES: C=C(C)C(=O)OCCCOc1ccc2c(c1)C(C)c1cc(OC(=O)c3ccc(OCCCCOC(=O)C(=C)F)cc3)ccc1-2] | 246 |
| 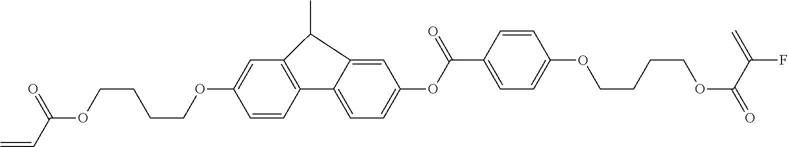 [SMILES: C=CC(=O)OCCCCOc1ccc2c(c1)C(C)c1cc(OC(=O)c3ccc(OCCCCOC(=O)C(=C)F)cc3)ccc1-2] | 247 |
| 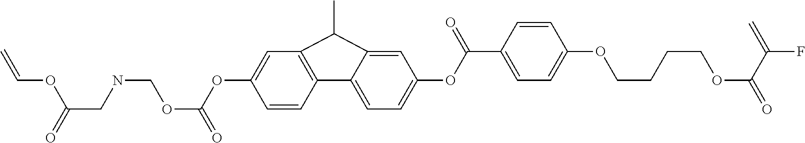 [SMILES: C=COC(=O)CNCOC(=O)Oc1ccc2c(c1)C(C)c1cc(OC(=O)c3ccc(OCCCCOC(=O)C(=C)F)cc3)ccc1-2] | 248 |
| 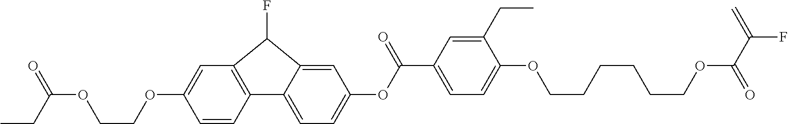 [SMILES: C=C(F)C(=O)OCCCCCCOc1ccc(C(=O)Oc2ccc3c(c2)C(F)c2cc(OCCOC(=O)CC)ccc2-3)cc1CC] | 249 |
| 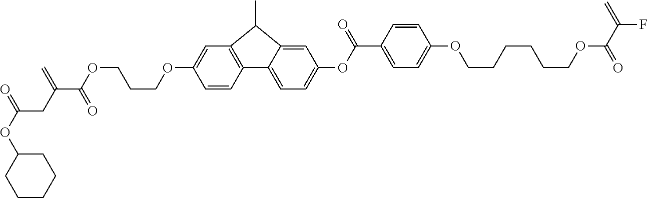 [SMILES: C=C(F)C(=O)OCCCCCCOc1ccc(C(=O)Oc2ccc3c(c2)C(C)c2cc(OCCCOC(=O)C(=C)CC(=O)OC4CCCCC4)ccc2-3)cc1] | 250 |
| 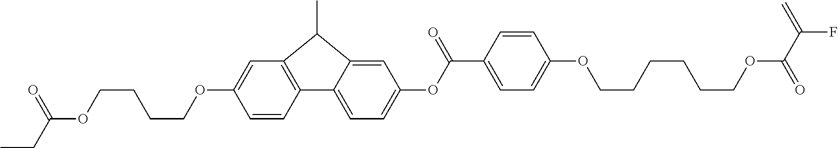 [SMILES: C=C(F)C(=O)OCCCCCCOc1ccc(C(=O)Oc2ccc3c(c2)C(C)c2cc(OCCCCOC(=O)CC)ccc2-3)cc1] | 251 |
| 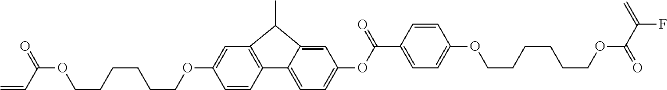 [SMILES: C=CC(=O)OCCCCCCOc1ccc2c(c1)C(C)c1cc(OC(=O)c3ccc(OCCCCCCOC(=O)C(=C)F)cc3)ccc1-2] | 252 |

Formula 84

| | |
|---|---|
| 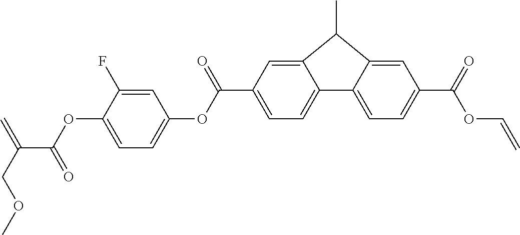 [SMILES: C=COC(=O)c1ccc2c(c1)C(C)c1cc(C(=O)Oc3ccc(OC(=O)C(=C)COC)c(F)c3)ccc1-2] | 253 |
| 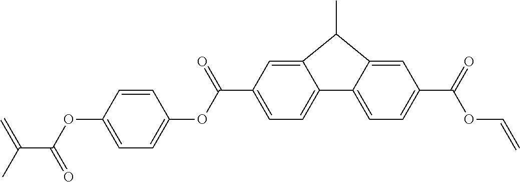 [SMILES: C=COC(=O)c1ccc2c(c1)C(C)c1cc(C(=O)Oc3ccc(OC(=O)C(=C)C)cc3)ccc1-2] | 254 |
| 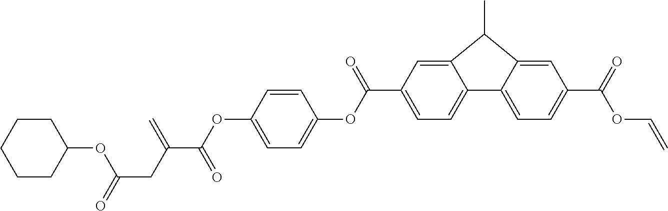 [SMILES: C=COC(=O)c1ccc2c(c1)C(C)c1cc(C(=O)Oc3ccc(OC(=O)C(=C)CC(=O)OC4CCCCC4)cc3)ccc1-2] | 255 |
| 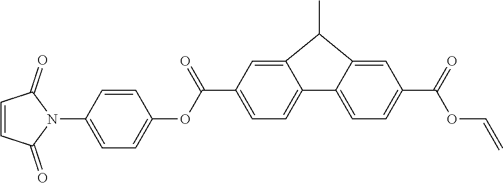 [SMILES: C=COC(=O)c1ccc2c(c1)C(C)c1cc(C(=O)Oc3ccc(N4C(=O)C=CC4=O)cc3)ccc1-2] | 256 |
| 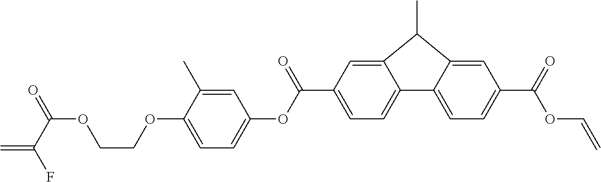 [SMILES: C=COC(=O)c1ccc2c(c1)C(C)c1cc(C(=O)Oc3ccc(OCCOC(=O)C(=C)F)c(C)c3)ccc1-2] | 257 |
| 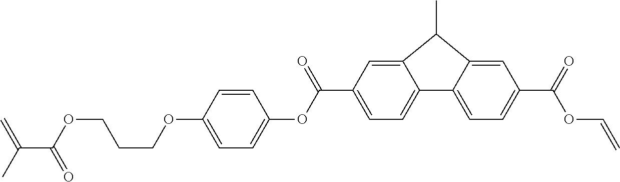 [SMILES: C=COC(=O)c1ccc2c(c1)C(C)c1cc(C(=O)Oc3ccc(OCCCOC(=O)C(=C)C)cc3)ccc1-2] | 258 |
| 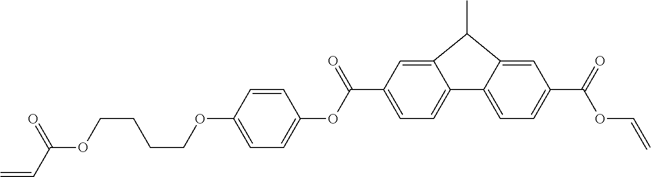 [SMILES: C=COC(=O)c1ccc2c(c1)C(C)c1cc(C(=O)Oc3ccc(OCCCCOC(=O)C=C)cc3)ccc1-2] | 259 |
| 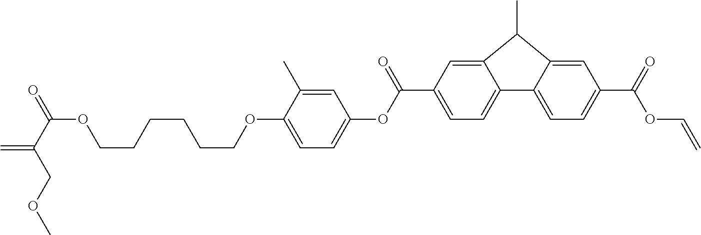 [SMILES: C=COC(=O)c1ccc2c(c1)C(C)c1cc(C(=O)Oc3ccc(OCCCCCCOC(=O)C(=C)COC)c(C)c3)ccc1-2] | 260 |
| 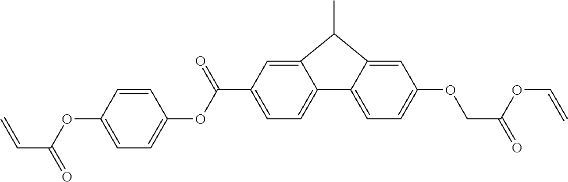 [SMILES: C=COC(=O)COc1ccc2c(c1)C(C)c1cc(C(=O)Oc3ccc(OC(=O)C=C)cc3)ccc1-2] | 261 |
| 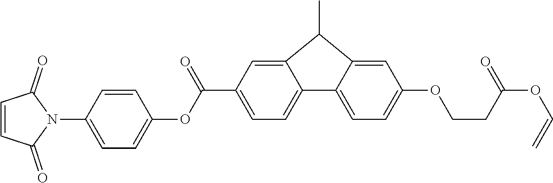 [SMILES: C=COC(=O)CCOc1ccc2c(c1)C(C)c1cc(C(=O)Oc3ccc(N4C(=O)C=CC4=O)cc3)ccc1-2] | 262 |
| 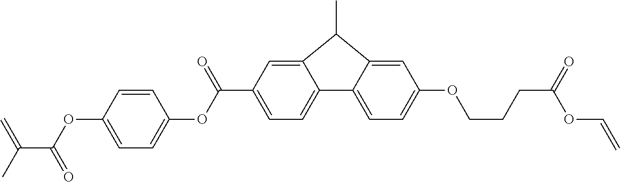 [SMILES: C=COC(=O)CCCOc1ccc2c(c1)C(C)c1cc(C(=O)Oc3ccc(OC(=O)C(=C)C)cc3)ccc1-2] | 263 |
| 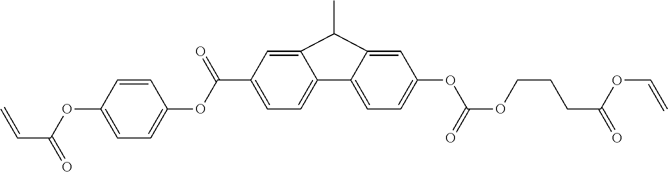 [SMILES: C=COC(=O)CCCOC(=O)Oc1ccc2c(c1)C(C)c1cc(C(=O)Oc3ccc(OC(=O)C=C)cc3)ccc1-2] | 264 |
| 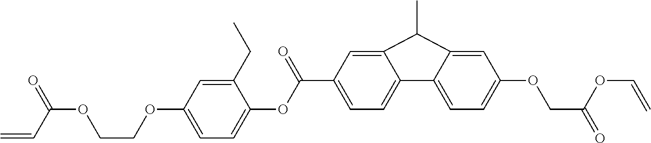 [SMILES: C=COC(=O)COc1ccc2c(c1)C(C)c1cc(C(=O)Oc3ccc(OCCOC(=O)C=C)cc3CC)ccc1-2] | 265 |
| 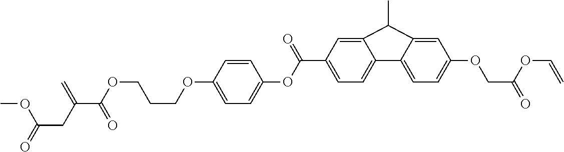 [SMILES: C=COC(=O)COc1ccc2c(c1)C(C)c1cc(C(=O)Oc3ccc(OCCCOC(=O)C(=C)CC(=O)OC)cc3)ccc1-2] | 266 |
| 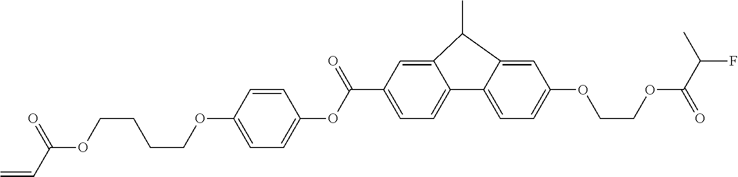 [SMILES: C=CC(=O)OCCCCOc1ccc(OC(=O)c2ccc3c(c2)C(C)c2cc(OCCOC(=O)C(C)F)ccc2-3)cc1] | 267 |
| 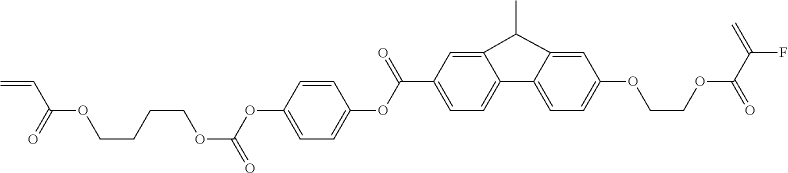 [SMILES: C=CC(=O)OCCCCOC(=O)Oc1ccc(OC(=O)c2ccc3c(c2)C(C)c2cc(OCCOC(=O)C(=C)F)ccc2-3)cc1] | 268 |
| 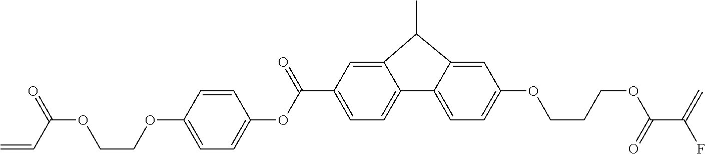 [SMILES: C=CC(=O)OCCOc1ccc(OC(=O)c2ccc3c(c2)C(C)c2cc(OCCCOC(=O)C(=C)F)ccc2-3)cc1] | 269 |
| 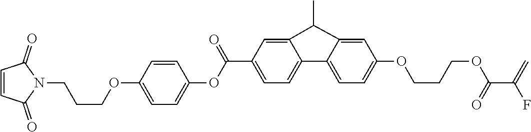 [SMILES: C=C(F)C(=O)OCCCOc1ccc2c(c1)C(C)c1cc(C(=O)Oc3ccc(OCCCN4C(=O)C=CC4=O)cc3)ccc1-2] | 270 |
| 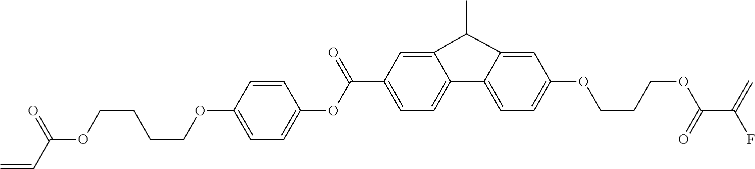 [SMILES: C=CC(=O)OCCCCOc1ccc(OC(=O)c2ccc3c(c2)C(C)c2cc(OCCCOC(=O)C(=C)F)ccc2-3)cc1] | 271 |
| 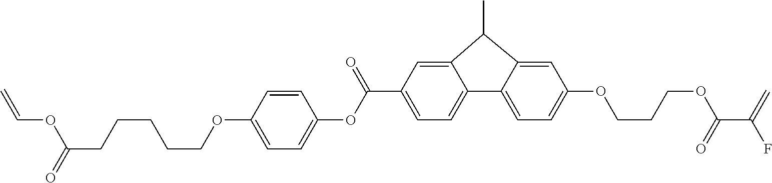 [SMILES: C=COC(=O)CCCCCOc1ccc(OC(=O)c2ccc3c(c2)C(C)c2cc(OCCCOC(=O)C(=C)F)ccc2-3)cc1] | 272 |
| 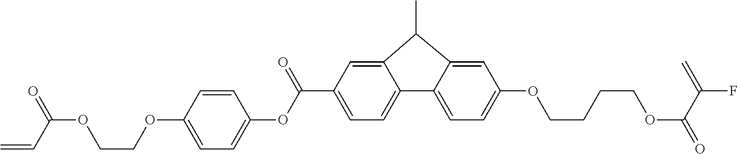 [SMILES: C=CC(=O)OCCOc1ccc(OC(=O)c2ccc3c(c2)C(C)c2cc(OCCCCOC(=O)C(=C)F)ccc2-3)cc1] | 273 |
| 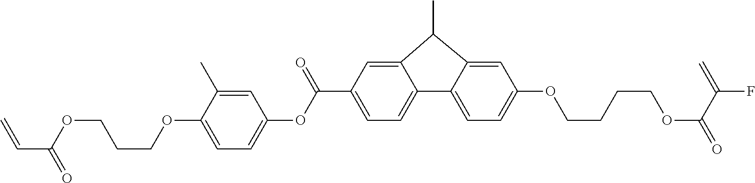 [SMILES: C=CC(=O)OCCCOc1ccc(OC(=O)c2ccc3c(c2)C(C)c2cc(OCCCCOC(=O)C(=C)F)ccc2-3)cc1C] | 274 |
| 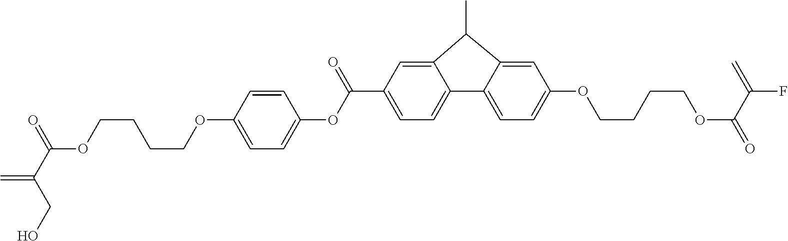 [SMILES: C=C(F)C(=O)OCCCCOc1ccc2c(c1)C(C)c1cc(C(=O)Oc3ccc(OCCCCOC(=O)C(=C)CO)cc3)ccc1-2] | 275 |
| 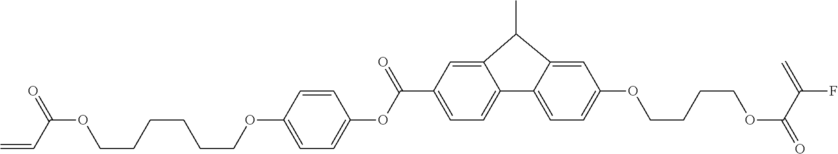 [SMILES: C=CC(=O)OCCCCCCOc1ccc(OC(=O)c2ccc3c(c2)C(C)c2cc(OCCCCOC(=O)C(=C)F)ccc2-3)cc1] | 276 |
| 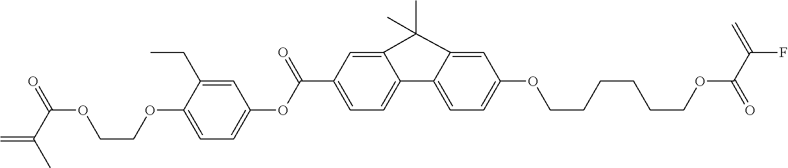 [SMILES: C=C(C)C(=O)OCCOc1ccc(OC(=O)c2ccc3c(c2)C(C)(C)c2cc(OCCCCCCOC(=O)C(=C)F)ccc2-3)cc1CC] | 277 |
| 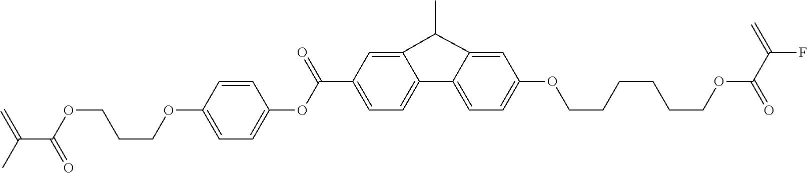 [SMILES: C=C(C)C(=O)OCCCOc1ccc(OC(=O)c2ccc3c(c2)C(C)c2cc(OCCCCCCOC(=O)C(=C)F)ccc2-3)cc1] | 278 |
| 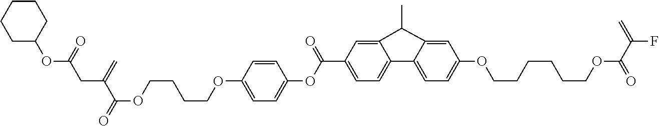 [SMILES: C=C(F)C(=O)OCCCCCCOc1ccc2c(c1)C(C)c1cc(C(=O)Oc3ccc(OCCCCOC(=O)C(=C)CC(=O)OC4CCCCC4)cc3)ccc1-2] | 279 |
| 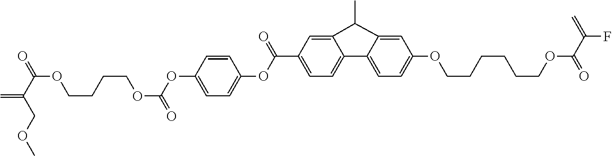 [SMILES: C=C(F)C(=O)OCCCCCCOc1ccc2c(c1)C(C)c1cc(C(=O)Oc3ccc(OC(=O)OCCCCOC(=O)C(=C)COC)cc3)ccc1-2] | 280 |

Formula 85

| | |
|---|---|
| 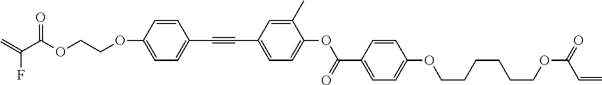 [SMILES: C=CC(=O)OCCCCCCOc1ccc(C(=O)Oc2ccc(C#Cc3ccc(OCCOC(=O)C(=C)F)cc3)cc2C)cc1] | 281 |
| 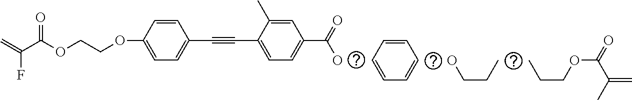 | 282 |
| 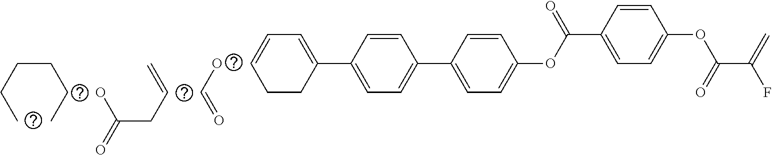 | 283 |
| 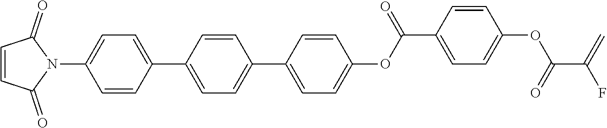 [SMILES: C=C(F)C(=O)Oc1ccc(C(=O)Oc2ccc(-c3ccc(-c4ccc(N5C(=O)C=CC5=O)cc4)cc3)cc2)cc1] | 284 |
| 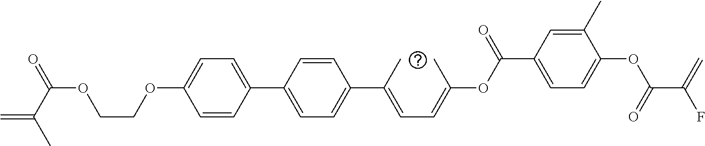 [SMILES: C=C(C)C(=O)OCCOc1ccc(-c2ccc(/C(C)=C/C=C(\C)OC(=O)c3ccc(OC(=O)C(=C)F)c(C)c3)cc2)cc1] | 285 |
| 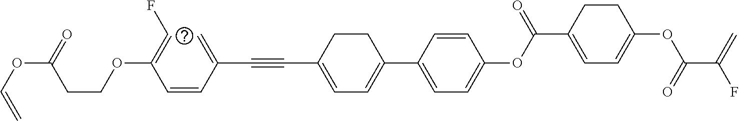 [SMILES: C=COC(=O)CCOC(/C=C\C(=C)C#CC1=CC=C(c2ccc(OC(=O)C3=CC=C(OC(=O)C(=C)F)CC3)cc2)CC1)=C\F] | 286 |
| 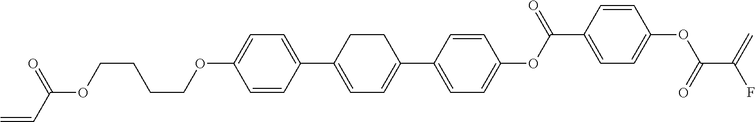 [SMILES: C=CC(=O)OCCCCOc1ccc(C2=CC=C(c3ccc(OC(=O)c4ccc(OC(=O)C(=C)F)cc4)cc3)CC2)cc1] | 287 |
| 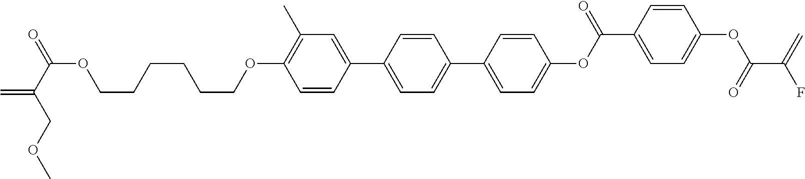 [SMILES: C=C(F)C(=O)Oc1ccc(C(=O)Oc2ccc(-c3ccc(-c4ccc(OCCCCCCOC(=O)C(=C)COC)c(C)c4)cc3)cc2)cc1] | 288 |
| 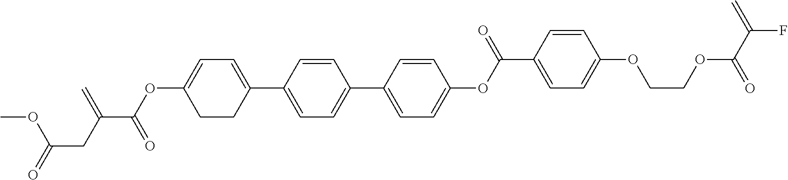 [SMILES: C=C(F)C(=O)OCCOc1ccc(C(=O)Oc2ccc(-c3ccc(C4=CC=C(OC(=O)C(=C)CC(=O)OC)CC4)cc3)cc2)cc1] | 289 |
| 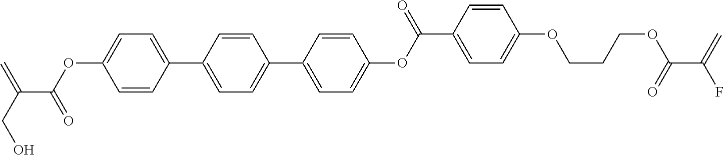 [SMILES: C=C(F)C(=O)OCCCOc1ccc(C(=O)Oc2ccc(-c3ccc(-c4ccc(OC(=O)C(=C)CO)cc4)cc3)cc2)cc1] | 290 |
| 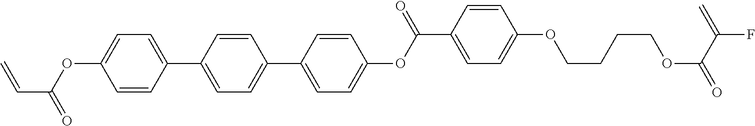 [SMILES: C=CC(=O)Oc1ccc(-c2ccc(-c3ccc(OC(=O)c4ccc(OCCCCOC(=O)C(=C)F)cc4)cc3)cc2)cc1] | 291 |
| 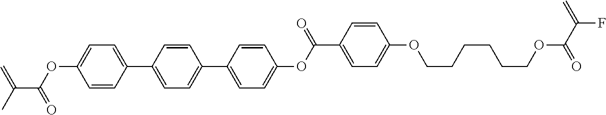 [SMILES: C=C(C)C(=O)Oc1ccc(-c2ccc(-c3ccc(OC(=O)c4ccc(OCCCCCCOC(=O)C(=C)F)cc4)cc3)cc2)cc1] | 292 |
| 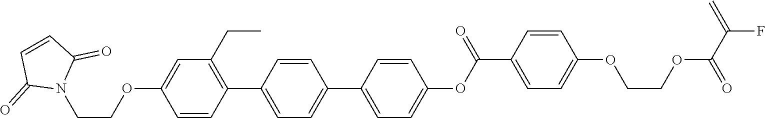 [SMILES: C=C(F)C(=O)OCCOc1ccc(C(=O)Oc2ccc(-c3ccc(-c4ccc(OCCN5C(=O)C=CC5=O)cc4CC)cc3)cc2)cc1] | 293 |
| 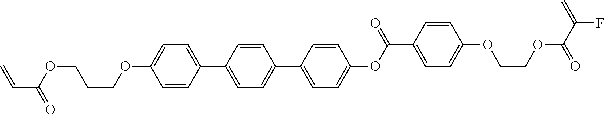 [SMILES: C=CC(=O)OCCCOc1ccc(-c2ccc(-c3ccc(OC(=O)c4ccc(OCCOC(=O)C(=C)F)cc4)cc3)cc2)cc1] | 294 |
| 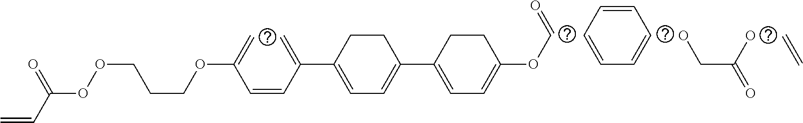 | 295 |
| 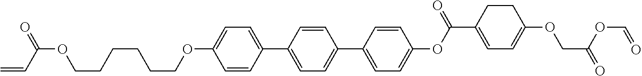 [SMILES: C=CC(=O)OCCCCCCOc1ccc(-c2ccc(-c3ccc(OC(=O)C4=CC=C(OCC(=O)OC=O)CC4)cc3)cc2)cc1] | 296 |
| 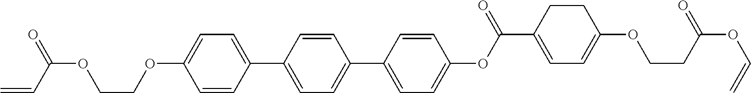 [SMILES: C=COC(=O)CCOC1=CC=C(C(=O)Oc2ccc(-c3ccc(-c4ccc(OCCOC(=O)C=C)cc4)cc3)cc2)CC1] | 297 |
| 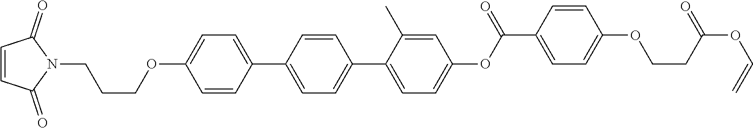 [SMILES: C=COC(=O)CCOc1ccc(C(=O)Oc2ccc(-c3ccc(-c4ccc(OCCCN5C(=O)C=CC5=O)cc4)cc3)c(C)c2)cc1] | 298 |
| 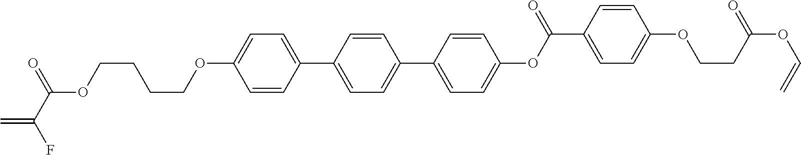 [SMILES: C=COC(=O)CCOc1ccc(C(=O)Oc2ccc(-c3ccc(-c4ccc(OCCCCOC(=O)C(=C)F)cc4)cc3)cc2)cc1] | 299 |
| 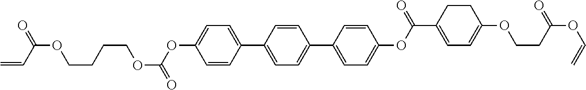 [SMILES: C=COC(=O)CCOC1=CC=C(C(=O)Oc2ccc(-c3ccc(-c4ccc(OC(=O)OCCCCOC(=O)C=C)cc4)cc3)cc2)CC1] | 300 |
| 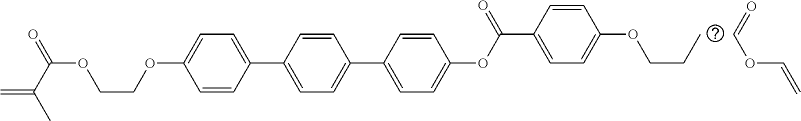 | 301 |
| 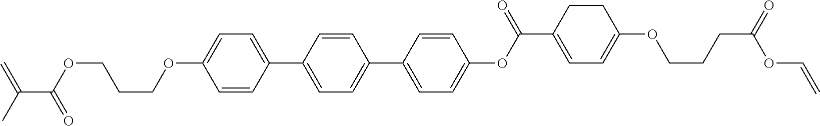 [SMILES: C=COC(=O)CCCOC1=CC=C(C(=O)Oc2ccc(-c3ccc(-c4ccc(OCCCOC(=O)C(=C)C)cc4)cc3)cc2)CC1] | 302 |
| 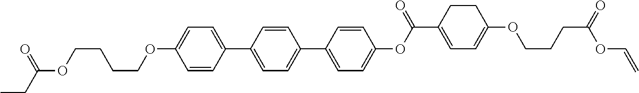 [SMILES: C=COC(=O)CCCOC1=CC=C(C(=O)Oc2ccc(-c3ccc(-c4ccc(OCCCCOC(=O)CC)cc4)cc3)cc2)CC1] | 303 |
| 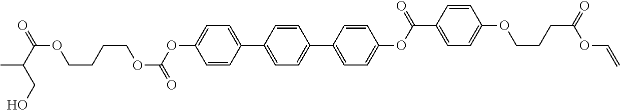 [SMILES: C=COC(=O)CCCOc1ccc(C(=O)Oc2ccc(-c3ccc(-c4ccc(OC(=O)OCCCCOC(=O)C(C)CO)cc4)cc3)cc2)cc1] | 304 |
| 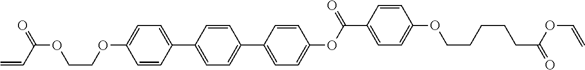 [SMILES: C=COC(=O)CCCCCOc1ccc(C(=O)Oc2ccc(-c3ccc(-c4ccc(OCCOC(=O)C=C)cc4)cc3)cc2)cc1] | 305 |
| 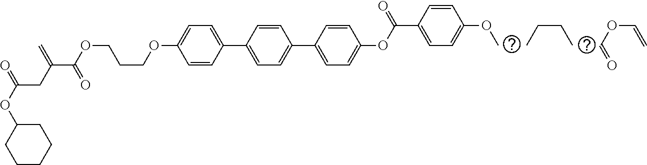 | 306 |
| 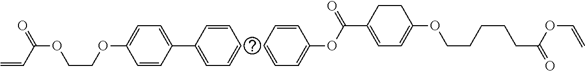 | 307 |
| 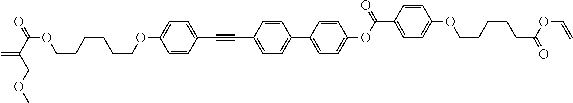 [SMILES: C=COC(=O)CCCCCOc1ccc(C(=O)Oc2ccc(-c3ccc(C#Cc4ccc(OCCCCCCOC(=O)C(=C)COC)cc4)cc3)cc2)cc1] | 308 |

Formula 86

| | |
|---|---|
| 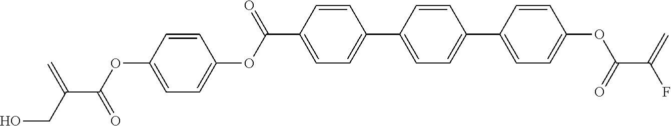 [SMILES: C=C(F)C(=O)Oc1ccc(-c2ccc(-c3ccc(C(=O)Oc4ccc(OC(=O)C(=C)CO)cc4)cc3)cc2)cc1] | 309 |
| 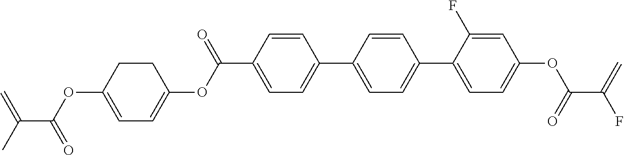 [SMILES: C=C(C)C(=O)OC1=CC=C(OC(=O)c2ccc(-c3ccc(-c4ccc(OC(=O)C(=C)F)cc4F)cc3)cc2)CC1] | 310 |
| 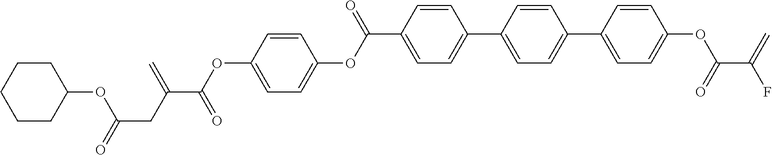 [SMILES: C=C(F)C(=O)Oc1ccc(-c2ccc(-c3ccc(C(=O)Oc4ccc(OC(=O)C(=C)CC(=O)OC5CCCCC5)cc4)cc3)cc2)cc1] | 311 |
| 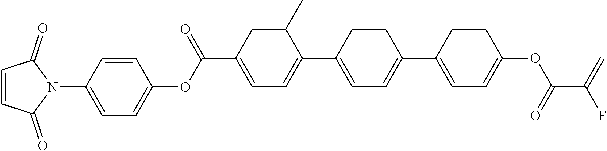 [SMILES: C=C(F)C(=O)OC1=CC=C(C2=CC=C(C3=CC=C(C(=O)Oc4ccc(N5C(=O)C=CC5=O)cc4)CC3C)CC2)CC1] | 312 |
| 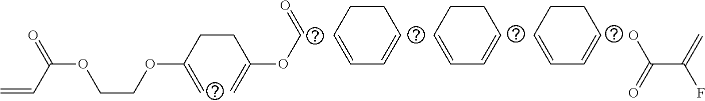 | 313 |
| 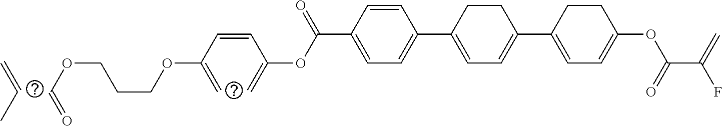 | 314 |
| 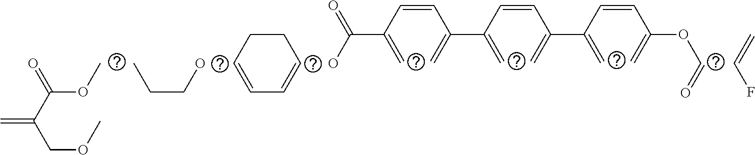 | 315 |
| 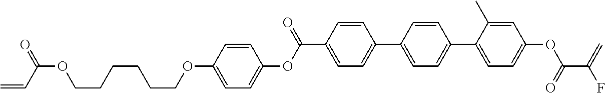 [SMILES: C=CC(=O)OCCCCCCOc1ccc(OC(=O)c2ccc(-c3ccc(-c4ccc(OC(=O)C(=C)F)cc4C)cc3)cc2)cc1] | 316 |
| 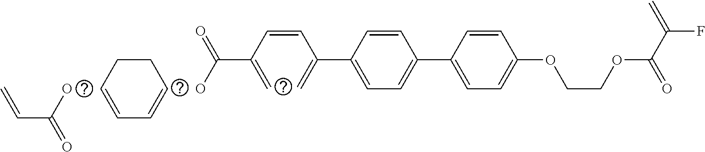 | 317 |
| 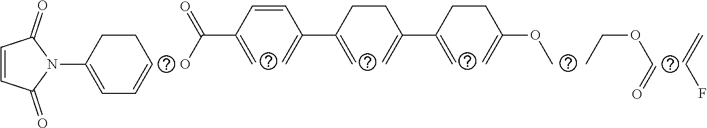 | 318 |
| 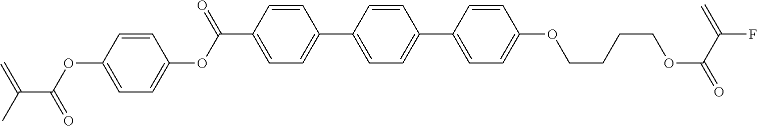 [SMILES: C=C(C)C(=O)Oc1ccc(OC(=O)c2ccc(-c3ccc(-c4ccc(OCCCCOC(=O)C(=C)F)cc4)cc3)cc2)cc1] | 319 |
| 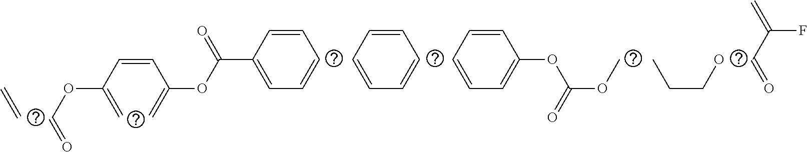 | 320 |
| 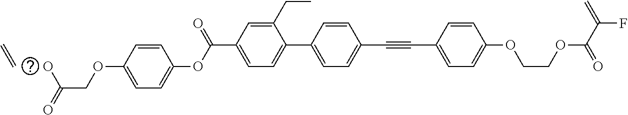 | 321 |
| 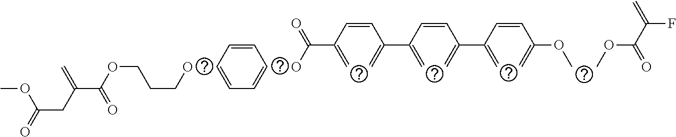 | 322 |
| 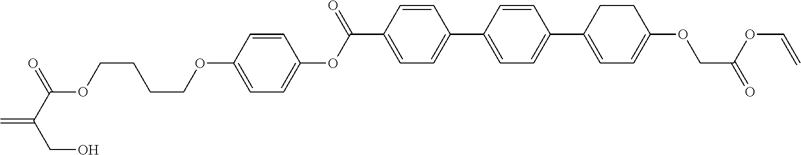 [SMILES: C=COC(=O)COC1=CC=C(c2ccc(-c3ccc(C(=O)Oc4ccc(OCCCCOC(=O)C(=C)CO)cc4)cc3)cc2)CC1] | 323 |
| 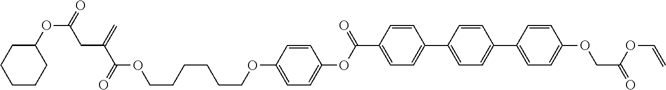 [SMILES: C=COC(=O)COc1ccc(-c2ccc(-c3ccc(C(=O)Oc4ccc(OCCCCCCOC(=O)C(=C)CC(=O)OC5CCCCC5)cc4)cc3)cc2)cc1] | 324 |
| 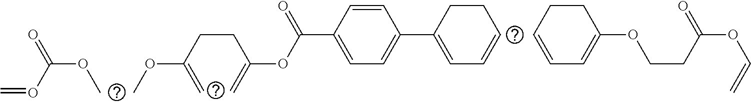 | 325 |
| 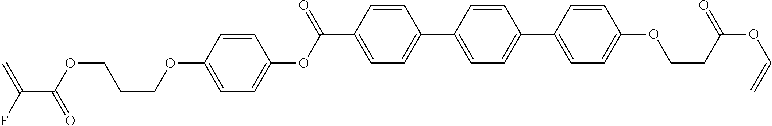 [SMILES: C=COC(=O)CCOc1ccc(-c2ccc(-c3ccc(C(=O)Oc4ccc(OCCCOC(=O)C(=C)F)cc4)cc3)cc2)cc1] | 326 |
| 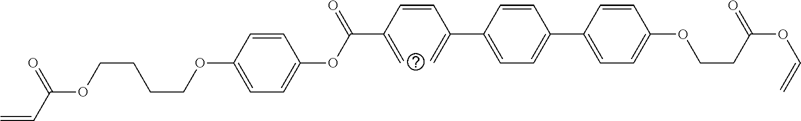 [SMILES: C=COC(=O)CCOc1ccc(-c2ccc(C(=C)/C=C\C(=C)C(=O)Oc3ccc(OCCCCOC(=O)C=C)cc3)cc2)cc1] | 327 |
| 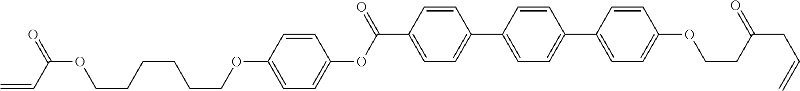 [SMILES: C=CCC(=O)CCOc1ccc(-c2ccc(-c3ccc(C(=O)Oc4ccc(OCCCCCCOC(=O)C=C)cc4)cc3)cc2)cc1] | 328 |
| 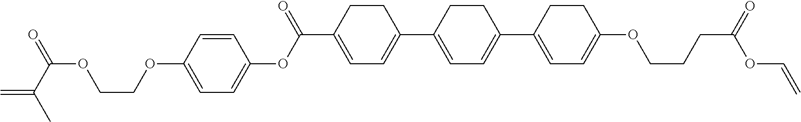 [SMILES: C=COC(=O)CCCOC1=CC=C(C2=CC=C(C3=CC=C(C(=O)Oc4ccc(OCCOC(=O)C(=C)C)cc4)CC3)CC2)CC1] | 329 |
| 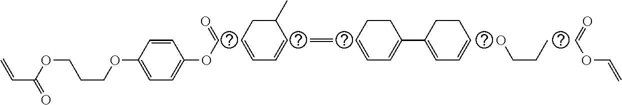 | 330 |
| 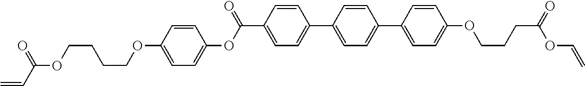 [SMILES: C=COC(=O)CCCOc1ccc(-c2ccc(-c3ccc(C(=O)Oc4ccc(OCCCCOC(=O)C=C)cc4)cc3)cc2)cc1] | 331 |
| 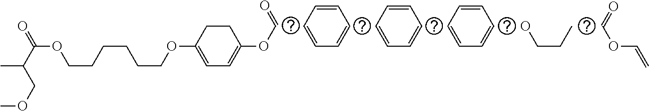 | 332 |
| 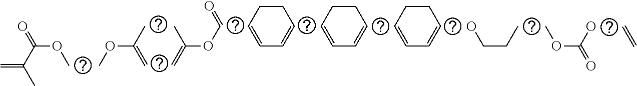 | 333 |
| 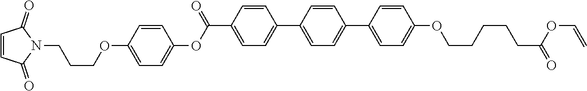 [SMILES: C=COC(=O)CCCCCOc1ccc(-c2ccc(-c3ccc(C(=O)Oc4ccc(OCCCN5C(=O)C=CC5=O)cc4)cc3)cc2)cc1] | 334 |
| 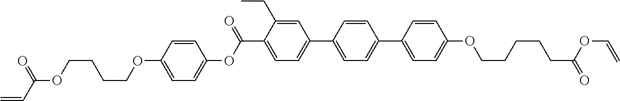 [SMILES: C=COC(=O)CCCCCOc1ccc(-c2ccc(-c3ccc(C(=O)Oc4ccc(OCCCCOC(=O)C=C)cc4)c(CC)c3)cc2)cc1] | 335 |
| 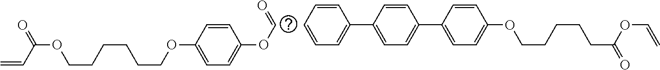 | 336 |

Formula 87

| | |
|---|---|
| 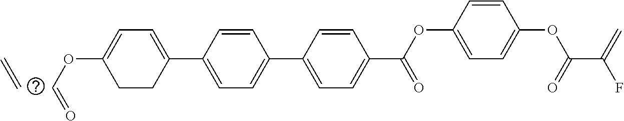 | 337 |
| 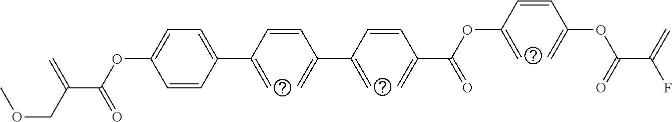 [SMILES: C=C(/C=C\C(=C)OC(=O)C(=C)/C=C\C(=C)C(=C)/C=C\C(=C)c1ccc(OC(=O)C(=C)COC)cc1)OC(=O)C(=C)F] | 338 |
| 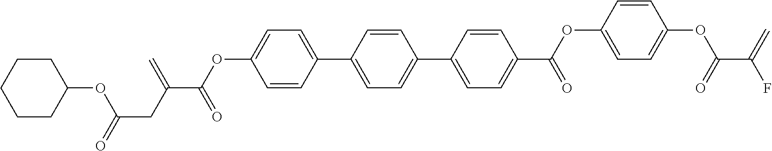 [SMILES: C=C(F)C(=O)Oc1ccc(OC(=O)c2ccc(-c3ccc(-c4ccc(OC(=O)C(=C)CC(=O)OC5CCCCC5)cc4)cc3)cc2)cc1] | 339 |
| 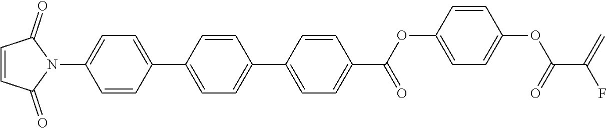 [SMILES: C=C(F)C(=O)Oc1ccc(OC(=O)c2ccc(-c3ccc(-c4ccc(N5C(=O)C=CC5=O)cc4)cc3)cc2)cc1] | 340 |
| 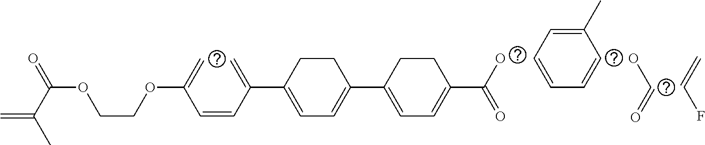 | 341 |
| 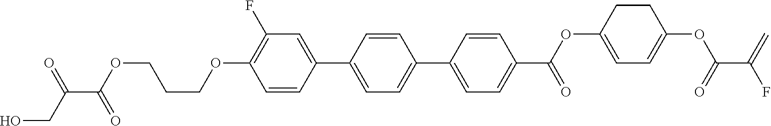 [SMILES: C=C(F)C(=O)OC1=CC=C(OC(=O)c2ccc(-c3ccc(-c4ccc(OCCCOC(=O)C(=O)CO)c(F)c4)cc3)cc2)CC1] | 342 |
| 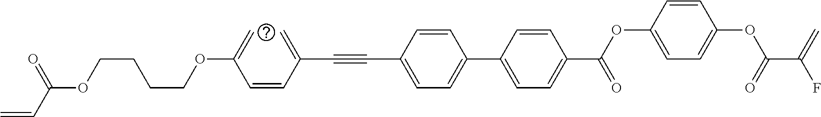 [SMILES: C=CC(=O)OCCCCOC(=C)/C=C\C(=C)C#Cc1ccc(-c2ccc(C(=O)Oc3ccc(OC(=O)C(=C)F)cc3)cc2)cc1] | 343 |
| 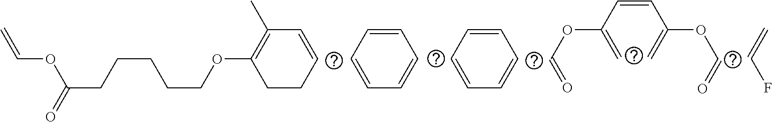 | 344 |
| 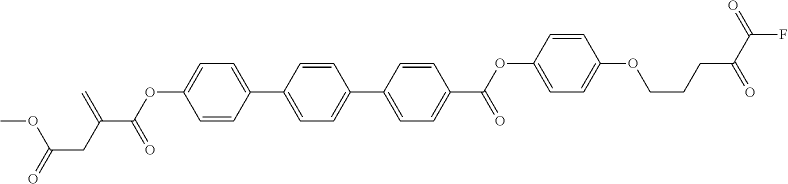 [SMILES: C=C(CC(=O)OC)C(=O)Oc1ccc(-c2ccc(-c3ccc(C(=O)Oc4ccc(OCCCC(=O)C(=O)F)cc4)cc3)cc2)cc1] | 345 |
| 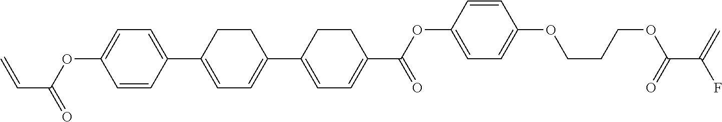 [SMILES: C=CC(=O)Oc1ccc(C2=CC=C(C3=CC=C(C(=O)Oc4ccc(OCCCOC(=O)C(=C)F)cc4)CC3)CC2)cc1] | 346 |
| 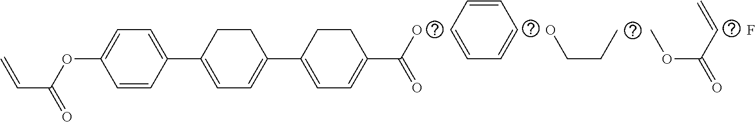 | 347 |
| 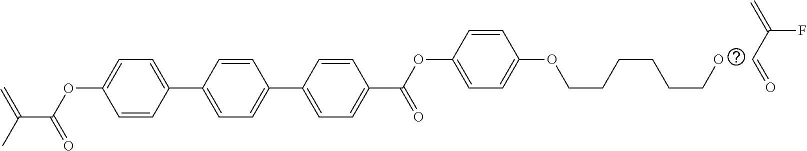 | 348 |
| 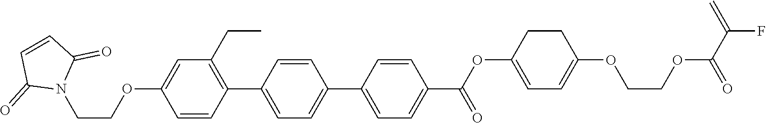 [SMILES: C=C(F)C(=O)OCCOC1=CC=C(OC(=O)c2ccc(-c3ccc(-c4ccc(OCCN5C(=O)C=CC5=O)cc4CC)cc3)cc2)CC1] | 349 |
| 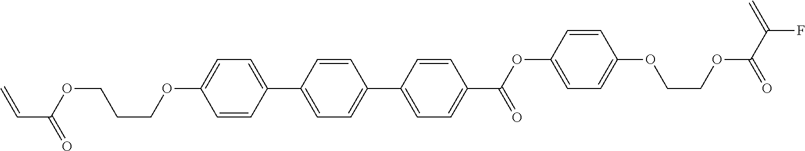 [SMILES: C=CC(=O)OCCCOc1ccc(-c2ccc(-c3ccc(C(=O)Oc4ccc(OCCOC(=O)C(=C)F)cc4)cc3)cc2)cc1] | 350 |
| 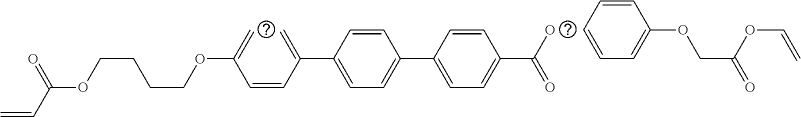 | 351 |
| 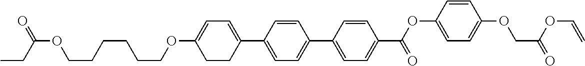 [SMILES: C=COC(=O)COc1ccc(OC(=O)c2ccc(-c3ccc(C4=CC=C(OCCCCCCOC(=O)CC)CC4)cc3)cc2)cc1] | 352 |
| 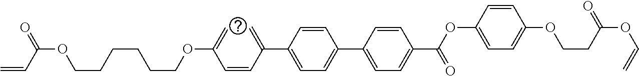 [SMILES: C=COC(=O)CCOc1ccc(OC(=O)c2ccc(-c3ccc(C(=C)/C=C\C(=C)OCCCCCCOC(=O)C=C)cc3)cc2)cc1] | 353 |
| 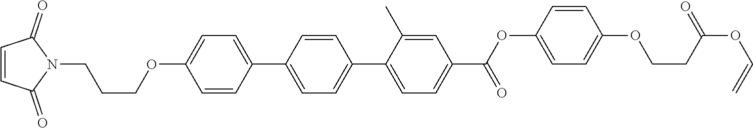 [SMILES: C=COC(=O)CCOc1ccc(OC(=O)c2ccc(-c3ccc(-c4ccc(OCCCN5C(=O)C=CC5=O)cc4)cc3)c(C)c2)cc1] | 354 |
| 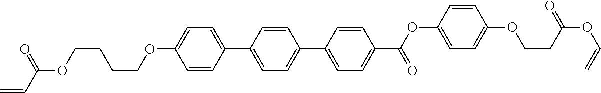 [SMILES: C=COC(=O)CCOc1ccc(OC(=O)c2ccc(-c3ccc(-c4ccc(OCCCCOC(=O)C=C)cc4)cc3)cc2)cc1] | 355 |
| 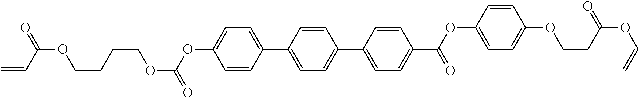 [SMILES: C=COC(=O)CCOc1ccc(OC(=O)c2ccc(-c3ccc(-c4ccc(OC(=O)OCCCCOC(=O)C=C)cc4)cc3)cc2)cc1] | 356 |
| 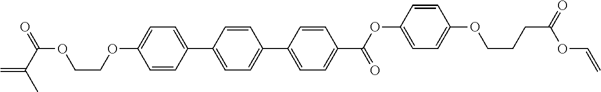 [SMILES: C=COC(=O)CCCOc1ccc(OC(=O)c2ccc(-c3ccc(-c4ccc(OCCOC(=O)C(=C)C)cc4)cc3)cc2)cc1] | 357 |
| 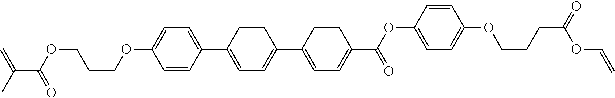 [SMILES: C=COC(=O)CCCOc1ccc(OC(=O)C2=CC=C(C3=CC=C(c4ccc(OCCCOC(=O)C(=C)C)cc4)CC3)CC2)cc1] | 358 |
| 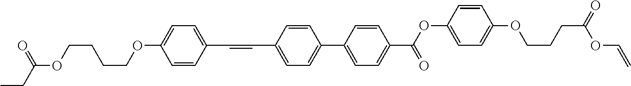 [SMILES: C=COC(=O)CCCOc1ccc(OC(=O)c2ccc(-c3ccc(C=Cc4ccc(OCCCCOC(=O)CC)cc4)cc3)cc2)cc1] | 359 |
| 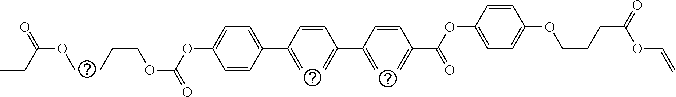 | 360 |
| 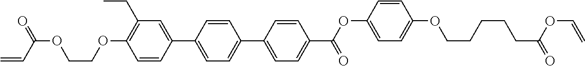 [SMILES: C=COC(=O)CCCCCOc1ccc(OC(=O)c2ccc(-c3ccc(-c4ccc(OCCOC(=O)C=C)c(CC)c4)cc3)cc2)cc1] | 361 |
| 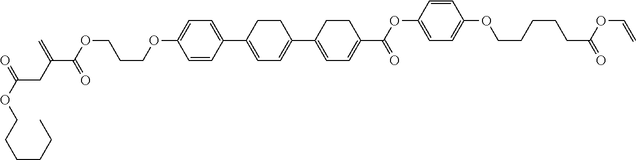 [SMILES: C=COC(=O)CCCCCOc1ccc(OC(=O)C2=CC=C(C3=CC=C(c4ccc(OCCCOC(=O)C(=C)CC(=O)OCCCCCC)cc4)CC3)CC2)cc1] | 362 |
| 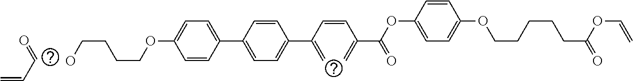 | 363 |
| 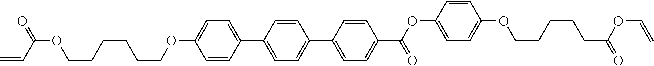 [SMILES: C=COC(=O)CCCCCOc1ccc(OC(=O)c2ccc(-c3ccc(-c4ccc(OCCCCCCOC(=O)C=C)cc4)cc3)cc2)cc1] | 364 |

Formula 88

| | |
|---|---|
| 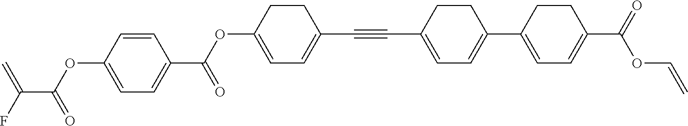 [SMILES: C=COC(=O)C1=CC=C(C2=CC=C(C#CC3=CC=C(OC(=O)c4ccc(OC(=O)C(=C)F)cc4)CC3)CC2)CC1] | 365 |
| 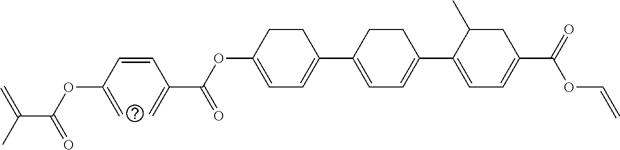 [SMILES: C=COC(=O)C1=CC=C(C2=CC=C(C3=CC=C(OC(=O)C(=C)/C=C\C(=C)OC(=O)C(=C)C)CC3)CC2)C(C)C1] | 366 |
| 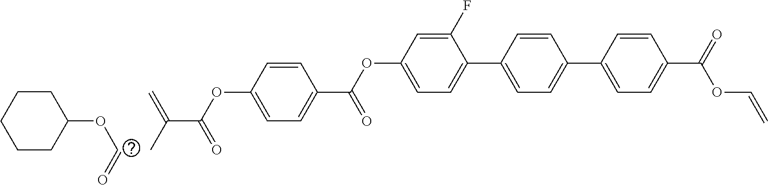 | 367 |
| 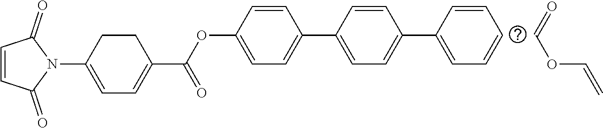 | 368 |
| 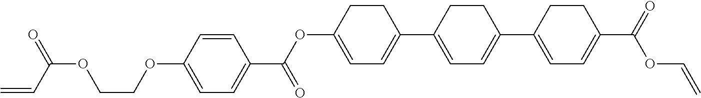 [SMILES: C=COC(=O)C1=CC=C(C2=CC=C(C3=CC=C(OC(=O)c4ccc(OCCOC(=O)C=C)cc4)CC3)CC2)CC1] | 369 |
| 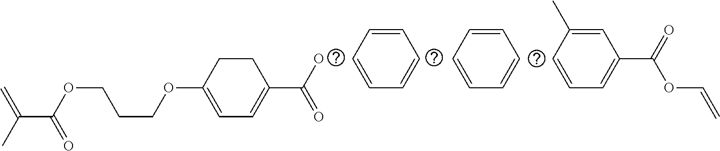 | 370 |
| 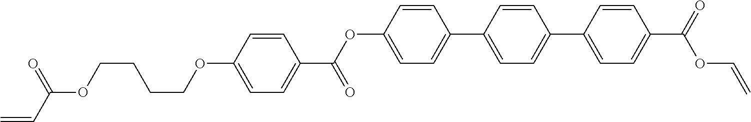 [SMILES: C=COC(=O)c1ccc(-c2ccc(-c3ccc(OC(=O)c4ccc(OCCCCOC(=O)C=C)cc4)cc3)cc2)cc1] | 371 |
| 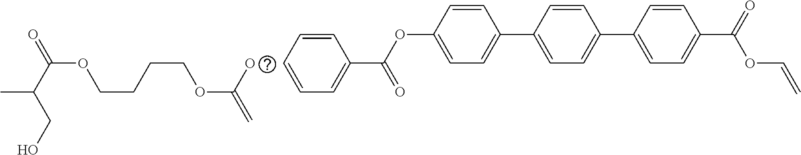 | 372 |
| 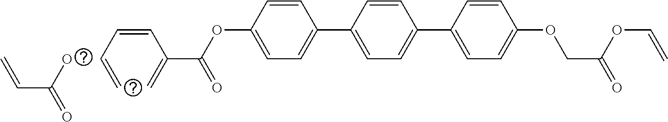 | 373 |
| 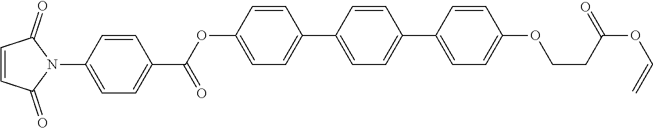 [SMILES: C=COC(=O)CCOc1ccc(-c2ccc(-c3ccc(OC(=O)c4ccc(N5C(=O)C=CC5=O)cc4)cc3)cc2)cc1] | 374 |
| 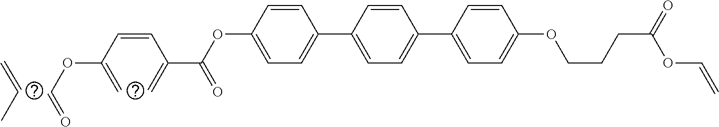 | 375 |
| 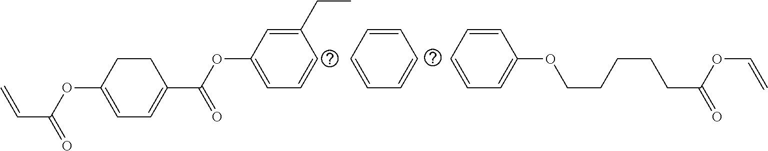 | 376 |
| 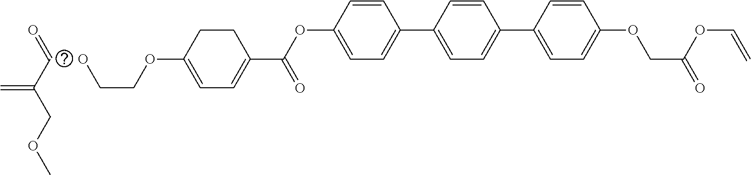 | 377 |
| 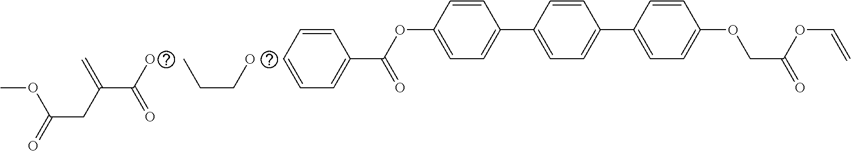 | 378 |
| 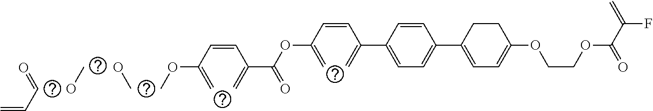 | 379 |
| 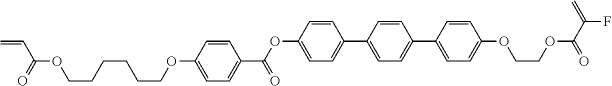 [SMILES: C=CC(=O)OCCCCCCOc1ccc(C(=O)Oc2ccc(-c3ccc(-c4ccc(OCCOC(=O)C(=C)F)cc4)cc3)cc2)cc1] | 380 |
| 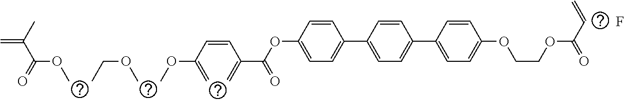 | 381 |
| 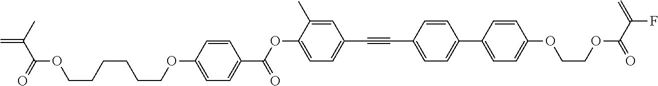 [SMILES: C=C(C)C(=O)OCCCCCCOc1ccc(C(=O)Oc2ccc(C#Cc3ccc(-c4ccc(OCCOC(=O)C(=C)F)cc4)cc3)cc2C)cc1] | 382 |
| 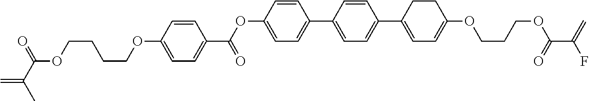 [SMILES: C=C(C)C(=O)OCCCCOc1ccc(C(=O)Oc2ccc(-c3ccc(C4=CC=C(OCCCOC(=O)C(=C)F)CC4)cc3)cc2)cc1] | 383 |
| 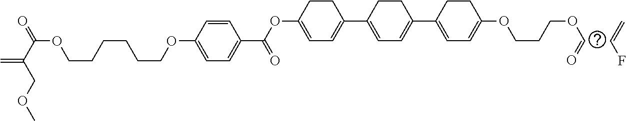 | 384 |
| 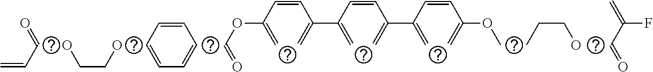 | 385 |
| 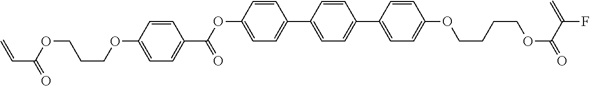 [SMILES: C=CC(=O)OCCCOc1ccc(C(=O)Oc2ccc(-c3ccc(-c4ccc(OCCCCOC(=O)C(=C)F)cc4)cc3)cc2)cc1] | 386 |
| 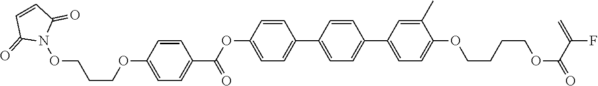 [SMILES: C=C(F)C(=O)OCCCCOc1ccc(-c2ccc(-c3ccc(OC(=O)c4ccc(OCCCON5C(=O)C=CC5=O)cc4)cc3)cc2)cc1C] | 387 |
| 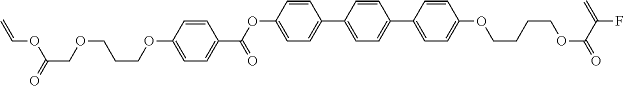 [SMILES: C=COC(=O)COCCCOc1ccc(C(=O)Oc2ccc(-c3ccc(-c4ccc(OCCCCOC(=O)C(=C)F)cc4)cc3)cc2)cc1] | 388 |
| 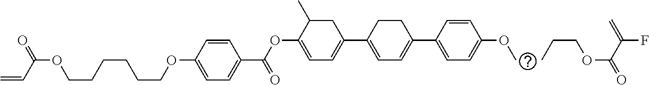 | 389 |
| 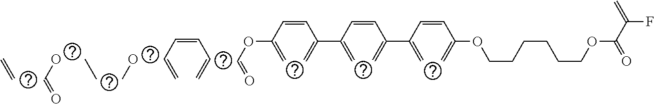 | 390 |
| 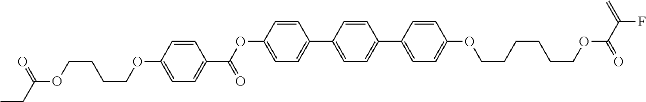 [SMILES: C=C(F)C(=O)OCCCCCCOc1ccc(-c2ccc(-c3ccc(OC(=O)c4ccc(OCCCCOC(=O)CC)cc4)cc3)cc2)cc1] | 391 |
| 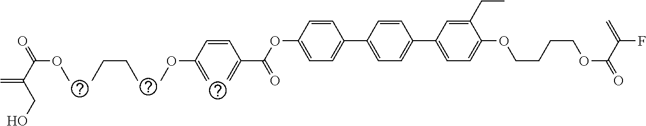 | 392 |

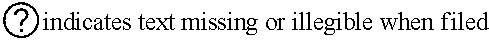

### 2. Use Examples of Devices

The compounds in Use Examples were represented using symbols based on definitions in Table 2 below. In Table 2, a configuration of 1,4-cyclohexylene is trans. A parenthesized number next to a symbolized compound corresponds to the number of the compound. A symbol (−) means any other liquid crystal compound. A proportion (percentage) of the liquid crystal compound is expressed in terms of weight percent (% by weight) based on the weight of the liquid crystal composition. Values of the characteristics of the composition are summarized in a last part.

TABLE 2
Method for description of compounds using symbols
R—(A₁)—Z₁— . . . Zₙ—(Aₙ)—R′

| 1) Left-terminal group R— | Symbol |
|---|---|
| FCₙH₂ₙ— | Fn— |
| CₙH₂ₙ₊₁— | n- |
| CₙH₂ₙ₊₁O— | nO— |
| CₘH₂ₘ₊₁OCₙH₂ₙ— | mOn— |
| CH₂═CH— | V— |
| CₙH₂ₙ₊₁—CH═CH— | nV— |
| CH₂═CH—CₙH₂ₙ— | Vn— |
| CₘH₂ₘ₊₁—CH═CH—CₙH₂ₙ— | mVn— |
| CF₂═CH— | VFF— |
| CF₂═CH—CₙH₂ₙ— | VFFn— |
| CₘH₂ₘ₊₁CF₂CₙH₂ₙ— | m(CF2)n— |
| CH₂═CHCOO— | AC— |
| CH2═C(CH [symbol] )COO— | MAC— |
| 2) Right-terminal group —R′ | Symbol |
| —CₙH₂ₙ₊₁ | -n |
| —OCₙH₂ₙ₊₁ | —On |
| —COOCH₃ | —EMe |
| —CH═CH₂ | —V |
| —CH═CH—CₙH₂ₙ₊₁ | —Vn |
| —CₙH₂ₙ—CH═CH₂ | —nV |
| —CₘH₂ₘ—CH═CH—Cₙ₂ₙ₊₁ | —mVn |
| —CH═CF₂ | —VFF |
| —F | —F |
| —Cl | —CL |
| —OCF₃ | —OCF3 |
| —OCF₂H | —OCF2H |
| —CF₃ | —CF3 |
| —CF═CH—CF₃ | —FVCF3 |
| —OCH═CH—CF₃ | —OVCF3 |
| —C≡N | —C |
| —OCOCH═CH₂ | —AC |
| —OCOC(CH₃)═CH₂ | —MAC |
| 3) Bonding group —Zₙ— | Symbol |
| —CₙH₂ₙ— | n |
| —COO— | E |
| —CH═CH— | V |
| —CH₂O— | 1O |
| —CH═CHO— | VO |
| —OCH═CH— | OV |
| —CF═CF— | VFF |
| —CH═CF— | VF |
| —OCH₂— | O1 |
| —OCF₂— | x |
| —CF₂O— | X |
| —C≡C— | T |
| 4) Ring Stucture —Aₙ— | Symbol |
| 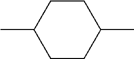 | H |
| 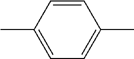 | B |
| 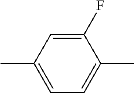 | B(F) |
| 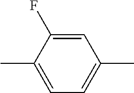 | B(2F) |
| 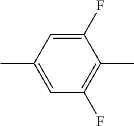 | B(F,F) |
| 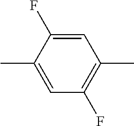 | B(2F,5F) |
| 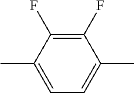 | B(2F,3F) |
| 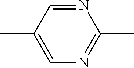 | Py |
| 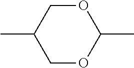 | G |
| 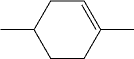 | ch |
| 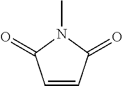 | M |
| 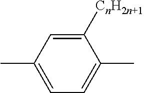 | Bm(n) |
| 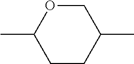 | Dh |
| 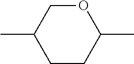 | dh |
| 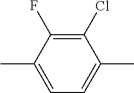 | B(2F,3Cl) |
| 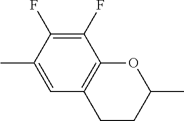 | Cro(7F,8F) |

[symbol]

indicates data missing or illegible when filed

TABLE 3
5) Examples of description
Example 1 3-HBB(2F,3F)—O2

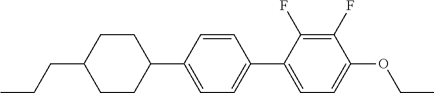 [SMILES: CCCC1CCC(c2ccc(-c3ccc(OCC)c(F)c3F)cc2)CC1]

Example 2 5-HHBB(F,F)—F

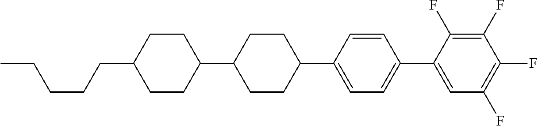 [SMILES: CCCCCC1CCC(C2CCC(c3ccc(-c4cc(F)c(F)c(F)c4F)cc3)CC2)CC1]

Example 3 3-HB—O2

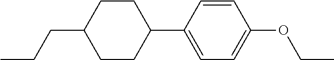 [SMILES: CCCC1CCC(c2ccc(OCC)cc2)CC1]

Example 3-HBB(F,F)—F

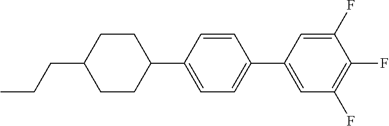 [SMILES: CCCC1CCC(c2ccc(-c3cc(F)c(F)c(F)c3)cc2)CC1]

### 1. Raw Material

A composition to which a polar compound was added was injected into a device having no alignment film. After the device was irradiated with linearly polarized light, alignment of liquid crystal molecules in the device was confirmed. First, a raw material will be described. The raw material was appropriately selected from among compositions such as composition (M1) to composition (M41), and polar compounds such as compound (No. 1) to compound (No. 230). The composition is as described below.

### Composition (M1)

| | | |
|---|---|---|
| 3-HB (2F, 3F)-O2 | (9-1) | 10% |
| 5-HB (2F, 3F)-O2 | (9-1) | 7% |
| 2-BB (2F, 3F)-O2 | (9-3) | 7% |
| 3-BB (2F, 3F)-O2 | (9-3) | 7% |
| 3-B (2F, 3F) B (2F, 3F)-O2 | (9-7) | 3% |
| 2-HHB (2F, 3F)-O2 | (10-1) | 5% |
| 3-HHB (2F, 3F)-O2 | (10-1) | 10% |
| 2-HBB (2F, 3F)-O2 | (10-7) | 8% |
| 3-HBB (2F, 3F)-O2 | (10-7) | 10% |
| 2-HH-3 | (2-1) | 14% |
| 3-HB-O1 | (2-5) | 5% |
| 3-HHB-1 | (3-1) | 3% |
| 3-HHB-O1 | (3-1) | 3% |
| 3-HHB-3 | (3-1) | 4% |
| 2-BB (F) B-3 | (3-6) | 4% |

NI=73.2° C.; Tc<−20° C.; Δn=0.113; Δε=−4.0; Vth=2.18 V; η=22.6 mPa·s.

### Composition (M2)

| | | |
|---|---|---|
| 3-HB (2F, 3F)-O4 | (9-1) | 6% |
| 3-H2B (2F, 3F)-O2 | (9-4) | 8% |
| 3-H1OB (2F, 3F)-O2 | (9-5) | 4% |
| 3-BB (2F, 3F)-O2 | (9-3) | 7% |
| 2-HHB (2F, 3F)-O2 | (10-1) | 7% |
| 3-HHB (2F, 3F)-O2 | (10-1) | 7% |
| 3-HH2B (2F, 3F)-O2 | (10-4) | 7% |
| 5-HH2B (2F, 3F)-O2 | (10-4) | 4% |
| 2-HBB (2F, 3F)-O2 | (10-7) | 5% |
| 3-HBB (2F, 3F)-O2 | (10-7) | 5% |
| 4-HBB (2F, 3F)-O2 | (10-7) | 6% |
| 2-HH-3 | (2-1) | 12% |
| 1-BB-5 | (2-8) | 12% |
| 3-HHB-1 | (3-1) | 4% |
| 3-HHB-O1 | (3-1) | 3% |
| 3-HBB-2 | (3-4) | 3% |

NI=82.8° C.; Tc<−30° C.; Δn=0.118; Δε=−4.4; Vth=2.13 V; η=22.5 mPa·s.

### Composition (M3)

| | | |
|---|---|---|
| 3-HB (2F, 3F)-O2 | (9-1) | 7% |
| 5-HB (2F, 3F)-O2 | (9-1) | 7% |
| 3-BB (2F, 3F)-O2 | (9-3) | 8% |
| 3-HHB (2F, 3F)-O2 | (10-1) | 5% |
| 5-HHB (2F, 3F)-O2 | (10-1) | 4% |
| 3-HH1OB (2F, 3F)-O2 | (10-5) | 4% |
| 2-BB (2F, 3F) B-3 | (11-1) | 5% |
| 2-HBB (2F, 3F)-O2 | (10-7) | 3% |
| 3-HBB (2F, 3F)-O2 | (10-7) | 8% |
| 4-HBB (2F, 3F)-O2 | (10-7) | 5% |
| 5-HBB (2F, 3F)-O2 | (10-7) | 8% |
| 3-HH-V | (2-1) | 27% |
| 3-HH-V1 | (2-1) | 6% |
| V-HHB-1 | (3-1) | 3% |

NI=78.1° C.; Tc<−30° C.; Δn=0.107; Δε=−3.2; Vth=2.02 V; η=15.9 mPa·s.

### Composition (M4)

| | | |
|---|---|---|
| 3-HB (2F, 3F)-O2 | (9-1) | 10% |
| 5-HB (2F, 3F)-O2 | (9-1) | 10% |
| 3-H2B (2F, 3F)-O2 | (9-4) | 8% |
| 5-H2B (2F, 3F)-O2 | (9-4) | 8% |
| 2-HBB (2F, 3F)-O2 | (10-7) | 6% |
| 3-HBB (2F, 3F)-O2 | (10-7) | 8% |
| 4-HBB (2F, 3F)-O2 | (10-7) | 7% |
| 5-HBB (2F, 3F)-O2 | (10-7) | 7% |
| 3-HDhB (2F, 3F)-O2 | (10-3) | 5% |
| 3-HH-4 | (2-1) | 14% |
| V-HHB-1 | (3-1) | 10% |
| 3-HBB-2 | (3-4) | 7% |

NI=88.5° C.; Tc<−30° C.; Δn=0.108; Δε=−3.8; Vth=2.25 V; η=24.6 mPa·s; VHR-1=99.1%; VHR-2=98.2%; VHR-3=97.8%.

### Composition (M5)

| | | |
|---|---|---|
| 3-HB (2F, 3F)-O2 | (9-1) | 7% |
| 3-HB (2F, 3F)-O4 | (9-1) | 8% |
| 3-H2B (2F, 3F)-O2 | (9-4) | 8% |
| 3-BB (2F, 3F)-O2 | (9-3) | 10% |
| 2-HHB (2F, 3F)-O2 | (10-1) | 4% |
| 3-HHB (2F, 3F)-O2 | (10-1) | 7% |
| 3-HHB (2F, 3F)-1 | (10-1) | 6% |
| 2-HBB (2F, 3F)-O2 | (10-7) | 6% |
| 3-FIBB (2F, 3F)-O2 | (10-7) | 6% |
| 4-HBB (2F, 3F)-O2 | (10-7) | 5% |
| 5-HBB (2F, 3F)-O2 | (10-7) | 4% |
| 3-HEB (2F, 3F) B (2F, 3F)-O2 | (16-1) | 3% |
| 3-H1OCro (7F, 8F)-5 | (13-2) | 3% |
| 3-HDhB (2F, 3F)-O2 | (10-3) | 5% |
| 3-HH-O1 | (2-1) | 5% |
| 1-BB-5 | (2-8) | 4% |
| V-HHB-1 | (3-1) | 4% |
| 5-HB ( F) BH-3 | (4-2) | 5% |

NI=81.1° C.; Tc<−30° C.; Δn=0.119; Δε=−4.5; Vth=1.69 V; η=31.4 mPa·s.

### Composition (M6)

| | | |
|---|---|---|
| 3-HB (2F, 3F)-O4 | (9-1) | 15% |
| 3-HBB (2F, 3F)-O2 | (10-7) | 8% |
| 4-HBB (2F, 3F)-O2 | (10-7) | 5% |
| 5-HBB (2F, 3F)-O2 | (10-7) | 7% |
| 3-dhBB (2F, 3F)-O2 | (10-9) | 5% |
| 3-chB (2F, 3F)-O2 | (16-2) | 7% |
| 2-HchB (2F, 3F)-O2 | (16-3) | 8% |
| 5-HH-V | (2-1) | 18% |
| 7-HB-1 | (2-5) | 5% |
| V-HHB-1 | (3-1) | 7% |
| V2-HHB-1 | (3-1) | 7% |
| 3-HBB (F) B-3 | (4-5) | 8% |

NI=98.8° C.; Tc<−30° C.; Δn=0.111; Δε=−3.2; Vth=2.47 V; η=23.9 mPa·s.

### Composition (M7)

| | | |
|---|---|---|
| 3-H2B (2F, 3F)-O2 | (9-4) | 18% |
| 5-H2B (2F, 3F)-O2 | (9-4) | 17% |
| 3-HHB (2F, 3Cl)-O2 | (10-12) | 5% |
| 3-FIBB (2F, 3Cl)-O2 | (10-13) | 8% |
| 5-HBB (2F, 3Cl)-O2 | (10-13) | 7% |
| 3-HDhB (2F, 3F)-O2 | (10-3) | 5% |
| 3-HH-V | (2-1) | 11% |
| 3-HH-VFF | (2-1) | 7% |
| F3-HE-V | (2-1) | 10% |
| 3-HHEH-3 | (3-13) | 4% |
| 3-HB (F) HH-2 | (4-7) | 4% |
| 3-HHEBH-3 | (4-6) | 4% |

NI=77.5° C.; Tc<−30° C.; Δn=0.084; Δε=−2.6; Vth=2.43 V; η=22.8 mPa·s.

### Composition (M8)

| | | |
|---|---|---|
| 3-HB (2F, 3F)-O2 | (9-1) | 8% |
| 3-H2B (2F, 3F)-O2 | (9-4) | 10% |
| 3-BB (2F, 3F)-O2 | (9-3) | 10% |
| 2O-BB (2F, 3F)-O2 | (9-3) | 3% |
| 2-HHB (2F, 3F)-O2 | (10-1) | 4% |
| 3-HHB (2F, 3F)-O2 | (10-1) | 7% |
| 2-HHB (2F, 3F)-1 | (10-1) | 5% |
| 2-BB (2F, 3F) B-3 | (11-1) | 6% |
| 2-BB (2F, 3F) B-4 | (11-1) | 6% |
| 2-HBB (2F, 3F)-O2 | (10-7) | 4% |
| 3-HBB (2F, 3F)-O2 | (10-7) | 7% |
| 3-HH1OCro (7F, 8F)-5 | (13-6) | 4% |
| 3-HDhB (2F, 3F)-O2 | (10-3) | 6% |
| 3-dhBB (2F, 3F)-O2 | (10-9) | 4% |
| 3-HH-V | (2-1) | 11% |
| 1-BB-5 | (2-8) | 5% |

NI=70.6° C.; Tc<−20° C.; Δn=0.129; Δε=−4.3; Vth=1.69 V; η=27.0 mPa·s.

### Composition (M9)

| | | |
|---|---|---|
| 3-HB (2F, 3F)-O4 | (9-1) | 14% |
| 3-H1OB (2F, 3F)-O2 | (9-5) | 3% |
| 3-BB (2F, 3F)-O2 | (9-3) | 10% |
| 2-HHB (2F, 3F)-O2 | (10-1) | 7% |
| 3-HHB (2F, 3F)-O2 | (10-1) | 7% |
| 3-HH1OB (2F, 3F)-O2 | (10-5) | 6% |
| 2-HBB (2F, 3F)-O2 | (10-7) | 4% |
| 3-HBB (2F, 3F)-O2 | (10-7) | 6% |
| 4-HBB (2F, 3F)-O2 | (10-7) | 4% |
| 3-HH-V | (2-1) | 14% |
| 1-BB-3 | (2-8) | 3% |
| 3-HHB-1 | (3-1) | 4% |
| 3-HHB-O1 | (3-1) | 4% |
| V-HBB-2 | (3-4) | 4% |
| 1-BB (F) B-2V | (3-6) | 6% |
| 5-HBBH-1O1 | (4-1) | 4% |

NI=93.0° C.; Tc<−30° C.; Δn=0.123; Δε=−4.0; Vth=2.27 V; η=29.6 mPa·s.

### Composition (M10)

| | | |
|---|---|---|
| 3-HB (2F, 3F)-O4 | (9-1) | 6% |
| 3-H2B (2F, 3F)-O2 | (9-4) | 8% |
| 3-H1OB (2F, 3F)-O2 | (9-5) | 5% |
| 3-BB (2F, 3F)-O2 | (9-3) | 10% |
| 2-HHB (2F, 3F)-O2 | (10-1) | 7% |
| 3-HHB (2F, 3F)-O2 | (10-1) | 7% |
| 5-HHB (2F, 3F)-O2 | (10-1) | 7% |
| 2-HBB (2F, 3F)-O2 | (10-7) | 4% |
| 3-HBB (2F, 3F)-O2 | (10-7) | 7% |
| 5-HBB (2F, 3F)-O2 | (10-7) | 6% |
| 3-HH-V | (2-1) | 11% |
| 1-BB-3 | (2-8) | 6% |
| 3-HHB-1 | (3-1) | 4% |
| 3-HHB-O1 | (3-1) | 4% |
| 3-HBB-2 | (3-4) | 4% |
| 3-B (F) BB-2 | (3-8) | 4% |

NI=87.6° C.; Tc<−30° C.; Δn=0.126; Δε=−4.5; Vth=2.21 V; η=25.3 mPa·s.

### Composition (M11)

| | | |
|---|---|---|
| 3-HB (2F, 3F)-O4 | (9-1) | 6% |
| 3-H2B (2F, 3F)-O2 | (9-4) | 8% |
| 3-H1OB (2F, 3F)-O2 | (9-5) | 4% |
| 3-BB (2F, 3F)-O2 | (9-3) | 7% |
| 2-HHB (2F, 3F)-O2 | (10-1) | 6% |
| 3-HHB (2F, 3F)-O2 | (10-1) | 10% |
| 5-HHB (2F, 3F)-O2 | (10-1) | 8% |
| 2-HBB (2F, 3F)-O2 | (10-7) | 5% |
| 3-HBB (2F, 3F)-O2 | (10-7) | 7% |
| 5-HBB (2F, 3F)-O2 | (10-7) | 5% |
| 2-HH-3 | (2-1) | 12% |
| 1-BB-3 | (2-8) | 6% |
| 3-HHB-1 | (3-1) | 3% |
| 3-HHB-O1 | (3-1) | 4% |
| 3-HBB-2 | (3-4) | 6% |
| 3-B (F) BB-2 | (3-8) | 3% |

NI=93.0° C.; Tc<−20° C.; Δn=0.124; Δε=−4.5; Vth=2.22 V; η=25.0 mPa·s.

### Composition (M12)

| | | |
|---|---|---|
| 3-HB (2F, 3F)-O2 | (9-1) | 7% |
| 5-HB (2F, 3F)-O2 | (9-1) | 7% |
| 3-BB (2F, 3F)-O2 | (9-3) | 8% |
| 3-HHB (2F, 3F)-O2 | (10-1) | 4% |
| 5-HHB (2F, 3F)-O2 | (10-1) | 5% |
| 3-HH1OB (2F, 3F)-O2 | (10-5) | 5% |
| 2-BB (2F, 3F) B-3 | (11-1) | 4% |
| 2-HBB (2F, 3F)-O2 | (10-7) | 3% |
| 3-HBB (2F, 3F)-O2 | (10-7) | 8% |
| 4-HBB (2F, 3F)-O2 | (10-7) | 5% |
| 5-HBB (2F, 3F)-O2 | (10-7) | 8% |
| 3-HH-V | (2-1) | 33% |
| V-HHB-1 | (3-1) | 3% |

NI=76.4° C.; Tc<−30° C.; Δn=0.104; Δε=−3.2; Vth=2.06 V; η=15.6 mPa·s.

### Composition (M13)

| | | |
|---|---|---|
| 2-H1OB (2F, 3F)-O2 | (9-5) | 6% |
| 3-H1OB (2F, 3F)-O2 | (9-5) | 4% |
| 3-BB (2F, 3F)-O2 | (9-3) | 3% |
| 2-HH1OB (2F, 3F)-O2 | (10-5) | 14% |
| 2-HBB (2F, 3F)-O2 | (10-7) | 7% |
| 3-HBB (2F, 3F)-O2 | (10-7) | 11% |
| 5-HBB (2F, 3F)-O2 | (10-7) | 9% |
| 2-HH-3 | (2-1) | 5% |
| 3-HH-VFF | (2-1) | 30% |
| 1-BB-3 | (2-8) | 5% |
| 3-HHB-1 | (3-1) | 3% |
| 3-HBB-2 | (3-4) | 3% |

NI=78.3° C.; Tc<−20° C.; Δn=0.103; Δε=−3.2; Vth=2.17 V; η=17.7 mPa·s.

### Composition (M14)

| | | |
|---|---|---|
| 3-HB(2F, 3F)-O2 | (9-1) | 5% |
| 5-HB(2F, 3F)-O2 | (9-1) | 7% |
| 3-BB(2F, 3F)-O2 | (9-3) | 8% |
| 3-HHB(2F, 3F)-O2 | (10-1) | 5% |
| 5-HHB(2F, 3F)-O2 | (10-1) | 4% |
| 3-HH1OB(2F, 3F)-O2 | (10-5) | 5% |
| 2-BB(2F, 3F)B-3 | (11-1) | 4% |
| 2-HBB(2F, 3F)-O2 | (10-7) | 3% |
| 3-HBB(2F, 3F)-O2 | (10-7) | 9% |
| 4-HBB(2F, 3F)-O2 | (10-7) | 4% |
| 5-HBB(2F, 3F)-O2 | (10-7) | 8% |
| 3-HH-V | (2-1) | 27% |
| 3-HH-V1 | (2-1) | 6% |
| V-HHB-1 | (3-1) | 5% |

NI=81.2° C.; Tc<−20° C.; Δn=0.107; Δε=−3.2; Vth=2.11 V; η=15.5 mPa·s.

### Composition (M15)

| | | |
|---|---|---|
| 3-H2B(2F, 3F)-O2 | (9-4) | 7% |
| 3-HHB(2F, 3F)-O2 | (10-1) | 8% |
| 3-HH1OB(2F, 3F)-O2 | (10-5) | 5% |
| 2-BB(2F, 3F)B-3 | (11-1) | 7% |
| 2-BB(2F, 3F)B-4 | (11-1) | 7% |
| 3-HDhB(2F, 3F)-O2 | (10-3) | 3% |
| 5-HDhB(2F, 3F)-O2 | (10-3) | 4% |
| 2-HchB(2F, 3F)-O2 | (16-3) | 8% |
| 4-HH-V | (2-1) | 15% |
| 3-HH-V1 | (2-1) | 6% |
| 1-HH-2V1 | (2-1) | 6% |
| 3-HH-2V1 | (2-1) | 4% |
| V2-BB-1 | (2-8) | 5% |
| 1V2-BB-1 | (2-8) | 5% |
| 3-HHB-1 | (3-1) | 6% |
| 3-HB(F)BH-3 | (4-2) | 4% |

NI=88.7° C.; Tc<−30° C.; Δn=0.115; Δε=−1.9; Vth=2.82 V; η=17.3 mPa·s.

### Composition (M16)

| | | |
|---|---|---|
| V2-H2B(2F, 3F)-O2 | (9-4) | 8% |
| V2-H1OB(2F, 3F)-O4 | (9-5) | 4% |
| 3-BB(2F, 3F)-O2 | (9-3) | 7% |
| 2-HHB(2F, 3F)-O2 | (10-1) | 7% |
| 3-HHB(2F, 3F)-O2 | (10-1) | 7% |
| 3-HH2B(2F, 3F)-O2 | (10-4) | 7% |
| 5-HH2B(2F, 3F)-O2 | (10-4) | 4% |
| V-HH2B(2F, 3F)-O2 | (10-4) | 6% |
| V2-HBB(2F, 3F)-O2 | (10-7) | 5% |
| V-HBB(2F, 3F)-O2 | (10-7) | 5% |
| V-HBB(2F, 3F)-O4 | (10-7) | 6% |
| 2-HH-3 | (2-1) | 12% |
| 1-BB-5 | (2-8) | 12% |
| 3-HHB-1 | (3-1) | 4% |
| 3-HHB-O1 | (3-1) | 3% |
| 3-HBB-2 | (3-4) | 3% |

NI=89.9° C.; Tc<−20° C.; Δn=0.122; Δε=−4.2; Vth=2.16 V; η=23.4 mPa·s.

### Composition (M17)

| | | |
|---|---|---|
| 3-HB(2F, 3F)-O2 | (9-1) | 3% |
| V-HB(2F, 3F)-O2 | (9-1) | 3% |
| V2-HB(2F, 3F)-O2 | (9-1) | 5% |
| 5-H2B(2F, 3F)-O2 | (9-4) | 5% |
| V2-BB(2F, 3F)-O2 | (9-3) | 3% |
| 1V2-BB(2F, 3F)-O2 | (9-3) | 3% |
| 3-HHB(2F, 3F)-O2 | (10-1) | 6% |
| V-HHB(2F, 3F)-O2 | (10-1) | 6% |
| V-HHB(2F, 3F)-O4 | (10-1) | 5% |
| V2-HHB(2F, 3F)-O2 | (10-1) | 4% |
| V2-BB(2F, 3F)B-1 | (11-1) | 4% |
| V2-HBB(2F, 3F)-O2 | (10-7) | 5% |
| V-HBB(2F, 3F)-O2 | (10-7) | 4% |
| V-HBB(2F, 3F)-O4 | (10-7) | 5% |
| V-HHB(2F, 3Cl)-O2 | (10-12) | 3% |
| 3-HH-V | (2-1) | 27% |
| 3-HH-V1 | (2-1) | 6% |
| V-HHB-1 | (3-1) | 3% |

NI=77.1° C.; Tc<−20° C.; Δn=0.101; Δε=−3.0; Vth=2.04 V; η=13.9 mPa·s.

### Composition (M18)

| | | |
|---|---|---|
| V-HB(2F, 3F)-O2 | (9-1) | 10% |
| V2-HB(2F, 3F)-O2 | (9-1) | 10% |
| 2-H1OB(2F, 3F)-O2 | (9-5) | 3% |
| 3-H1OB(2F, 3F)-O2 | (9-5) | 3% |
| 2O-BB(2F, 3F)-O2 | (9-3) | 3% |
| V2-BB(2F, 3F)-O2 | (9-3) | 8% |
| V2-HHB(2F, 3F)-O2 | (10-1) | 5% |
| 2-HBB(2F, 3F)-O2 | (10-7) | 3% |
| 3-HBB(2F, 3F)-O2 | (10-7) | 3% |
| V-HBB(2F, 3F)-O2 | (10-7) | 6% |
| V-HBB(2F, 3F)-O4 | (10-7) | 8% |
| V-HHB(2F, 3Cl)-O2 | (10-12) | 7% |
| 3-HH-4 | (2-1) | 14% |
| V-HHB-1 | (3-1) | 10% |
| 3-HBB-2 | (3-4) | 7% |

NI=75.9° C.; Tc<−20° C.; Δn=0.114; Δε=−3.9; Vth=2.20 V; η=24.7 mPa·s.

### Composition (M19)

| | | |
|---|---|---|
| 2-H1OB(2F, 3F)-O2 | (9-5) | 7% |
| 3-H1OB(2F, 3F)-O2 | (9-5) | 11% |
| 3-HH1OB(2F, 3F)-O2 | (10-5) | 8% |
| 2-HBB(2F, 3F)-O2 | (10-7) | 3% |
| 3-HBB(2F, 3F)-O2 | (10-7) | 9% |
| 5-HBB(2F, 3F)-O2 | (10-7) | 7% |
| V-HBB(2F, 3F)-O2 | (10-7) | 8% |
| 3-HDhB(2F, 3F)-O2 | (10-3) | 3.5% |
| 2-HH-3 | (2-1) | 21% |
| 3-HH-4 | (2-1) | 5% |
| 3-HB-O2 | (2-5) | 2.5% |
| 1-BB-3 | (2-8) | 4% |
| 3-HHB-1 | (3-1) | 1.5% |
| 3-HBB-2 | (3-4) | 9.5% |

NI=80.8° C.; Tc<−20° C.; Δn=0.108; Δε=−3.8; Vth=2.02 V; η=19.8 mPa·s,

### Composition (M20)

| | | |
|---|---|---|
| 2-H1OB(2F, 3F)-O2 | (9-5) | 5.5% |
| 2-BB(2F, 3F)-O2 | (9-3) | 11% |
| 2-HH1OB(2F, 3F)-O2 | (10-5) | 13% |
| 3-HH1OB(2F, 3F)-O2 | (10-5) | 15.5% |
| 3-HBB(2F, 3F)-O2 | (10-7) | 9% |
| 2-HH-3 | (2-1) | 25% |
| 3-HH-4 | (2-1) | 3% |
| 3-HBB-2 | (3-4) | 14% |
| 5-B(F)BB-2 | (3-8) | 4% |

NI=85.3° C.; Tc<−20° C.; Δn=0.109; Δε=−3.6; Vth=2.06 V; η=20.9 mPa·s.

### Composition (M21)

| | | |
|---|---|---|
| V-HB(2F, 3F)-O2 | (9-1) | 7% |
| V-2BB(2F, 3F)-O2 | (9-3) | 10% |
| V-HHB(2F, 3F)-O1 | (10-1) | 7% |
| V-HHB(2F, 3F)-O2 | (10-1) | 9% |
| V-2HHB(2F, 3F)-O2 | (10-1) | 8% |
| 3-HH2B(2F, 3F)-O2 | (10-4) | 9% |
| V-HBB(2F, 3F)-O2 | (10-7) | 7% |
| V-HBB(2F, 3F)-O4 | (10-7) | 7% |
| 2-HH-3 | (2-1) | 9% |
| 3-HH-4 | (2-1) | 3% |
| 3-HH-V | (2-1) | 15% |
| 3-HH-V1 | (2-1) | 6% |
| 1V2-HH-3 | (2-1) | 3% |

NI=87.5° C.; Tc<−20° C.; Δn=0.100; Δε=−3.4; Vth=2.25 V; η=16.6 mPa·s.

### Composition (M22)

| | | |
|---|---|---|
| 3-HHXB(F, F)-F | (6-100) | 6% |
| 3-BB(F, F)XB(F, F)-F | (6-97) | 13% |
| 3-HHBB(F, F)-F | (7-6) | 4% |
| 4-HHBB(F, F)-F | (7-6) | 5% |
| 3-HBBXB(F, F)-F | (7-32) | 3% |
| 3-BB(F)B(F, F)XB(F)-F | (7-46) | 2% |
| 4-BB(F)B(F, F)XB(F, F)-F | (7-47) | 8% |
| 5-BB(F)B(F, F)XB(F, F)-F | (7-47) | 7% |
| 3-HH-V | (2-1) | 44% |
| V-HHB-1 | (3-1) | 6% |
| 2-BB(F)B-3 | (3-6) | 2% |

NI=79.8° C.; Tc<−30° C.; Δn=0.106; Δε=8.5; Vth=1.45 V; η=11.6 mPa·s; γ1=60.0 mPa·s.

### Composition (M23)

| | | |
|---|---|---|
| 5-HXB(F, F)-F | (5-13) | 3% |
| 3-HHXB(F, F)-F | (6-100) | 3% |
| 3-HHXB(F, F)-CF3 | (6-100) | 3% |
| 3-HGB(F, F)-F | (6-103) | 3% |
| 3-HB(F)B(F, F)-F | (6-50) | 5% |
| 3-BB(F, F)XB(F, F)-F | (6-97) | 6% |
| 3-HHBB(F, F)-F | (7-6) | 6% |
| 5-BB(F)B(F, F)XB(F)B(F, F)-F | (-) | 2% |
| 3-BB(2F, 3F)XB(F, F)-F | (6-114) | 4% |
| 3-B(2F, 3F)BXB(F, F)-F | (6-115) | 5% |
| 3-HHB(F, F)XB(F, F)-F | (7-29) | 4% |
| 3-HB-CL | (5-2) | 3% |
| 3-HHB-OCF3 | (6-1) | 3% |
| 3-HH-V | (2-1) | 22% |
| 3-HH-V1 | (2-1) | 10% |
| 5-HB-O2 | (2-5) | 5% |
| 3-HHEH-3 | (3-13) | 3% |
| 3-HBB-2 | (3-4) | 7% |
| 5-B(F)BB-3 | (3-8) | 3% |

NI=71.2° C.; Tc<−20° C.; Δn=0.099; Δε=6.1; Vth=1.74 V; η=13.2 mPa·s; γ1=59.3 mPa·s.

### Composition (M24)

| | | |
|---|---|---|
| 5-HXB(F, F)-F | (5-13) | 6% |
| 3-HHXB(F, F)-F | (6-100) | 6% |
| V-HB(F)B(F, F)-F | (6-50) | 5% |
| 3-HHB(F)B(F, F)-F | (7-9) | 7% |
| 2-BB(F)B(F, F)XB(F)-F | (7-47) | 3% |
| 3-BB(F)B(F, F)XB(F)-F | (7-47) | 3% |
| 4-BB(F)B(F, F)XB(F)-F | (7-47) | 4% |
| 5-HB-CL | (5-2) | 5% |
| 2-HH-5 | (2-1) | 8% |
| 3-HH-V | (2-1) | 10% |
| 3-HH-V1 | (2-1) | 7% |
| 4-HH-V | (2-1) | 10% |
| 4-HH-V1 | (2-1) | 8% |
| 5-HB-O2 | (2-5) | 7% |
| 4-HHEH-3 | (3-13) | 3% |
| 1-BB(F)B-2V | (3-6) | 3% |
| 1O1-HBBH-3 | (4-6) | 5% |

NI=78.5° C.; Tc<−20° C.; Δn=0.095; Δε=3.4; Vth=1.50 V; η=8.4 mPa·s; γ1=54.2 mPa·s.

### Composition (M25)

| | | |
|---|---|---|
| 3-HHEB(F, F)-F | (6-12) | 5% |
| 3-HHXB(F, F)-F | (6-100) | 7% |
| 5-HBEB(F, F)-F | (6-39) | 5% |
| 3-BB(F, F)XB(F, F)-F | (6-97) | 10% |
| 2-HHB(F)B(F, F)-F | (7-9) | 3% |
| 3-HB(2F, 3F)BXB(F, F)-F | (7-58) | 3% |
| 3-BB(2F, 3F)BXB(F, F)-F | (7-59) | 2% |
| 5-HHB(F, F)XB(F, F)-F | (7-28) | 6% |
| 2-HH-3 | (2-1) | 8% |
| 3-HH-V | (2-1) | 20% |
| 3-HH-V1 | (2-1) | 7% |
| 4-HH-V | (2-1) | 6% |
| 5-HB-O2 | (2-5) | 5% |
| V2-B2BB-1 | (3) | 3% |
| 3-HHEBH-3 | (4-6) | 5% |
| 3-HHEBH-5 | (4-6) | 5% |

NI=90.3° C.; Tc<−20° C.; Δn=0.089; Δε=5.5; Vth=1.65 V; η=13.6 mPa·s; γ1=60.1 mPa·s.

### Composition (M26)

| | | |
|---|---|---|
| 3-BB(F, F)XB(F, F)-F | (6-97) | 12% |
| 3-HHBB(F, F)-F | (7-6) | 5% |
| 4-HHBB(F, F)-F | (7-6) | 4% |
| 3-HBBXB(F, F)-F | (7-32) | 3% |
| 3-BB(F)B(F, F)XB(F)-F | (7-46) | 3% |
| 3-BB(F)B(F, F)XB(F, F)-F | (7-47) | 3% |
| 4-BB(F)B(F, F)XB(F, F)-F | (7-47) | 5% |
| 5-BB(F)B(F, F)XB(F, F)-F | (7-47) | 4% |
| 2-HH-3 | (2-1) | 6% |
| 3-HH-5 | (2-1) | 6% |
| 3-HH-V | (2-1) | 25% |
| 3-HH-VFF | (2-1) | 6% |
| 5-HB-O2 | (2-5) | 7% |
| V-HHB-1 | (3-1) | 6% |
| V-HBB-2 | (3-4) | 5% |

NI=78.3° C.; Tc<−20° C.; Δn=0.107; Δε=7.0; Vth=1.55 V; η=11.6 mPa·s; γ1=55.6 mPa·s.

| | | |
|---|---|---|
| 3-HHXB(F, F)-F | (6-100) | 3% |
| 3-BBXB(F, F)-F | (6-91) | 3% |
| 3-BB(F, F)XB(F, F)-F | (6-97) | 8% |
| 3-HHBB(F, F)-F | (7-6) | 5% |
| 4-HHBB(F, F)-F | (7-6) | 4% |
| 3-BB(F)B(F, F)XB(F, F)-F | (7-47) | 3% |
| 4-BB(F)B(F, F)XB(F, F)-F | (7-47) | 6% |
| 5-BB(F)B(F, F)XB(F, F)-F | (7-47) | 5% |
| 3-HH-V | (2-1) | 30% |
| 3-HH-V1 | (2-1) | 5% |
| 3-HHB-O1 | (3-1) | 2% |
| V-HHB-1 | (3-1) | 5% |
| 2-BB(F)B-3 | (3-6) | 6% |
| F3-HH-V | (2-1) | 15% |

NI=80.4° C.; Tc<−20° C.; Δn=0.1.06; Δε=5.8; Vth=1.40 V; η=11.6 mPa·s; γ1=61.0 mPa·s.

### Composition (M28)

| | | |
|---|---|---|
| 3-HGB(F, F)-F | (6-103) | 3% |
| 5-GHB(F, F)-F | (6-109) | 4% |
| 3-GB(F, F)XB(F, F)-F | (6-113) | 5% |
| 3-BB(F)B(F, F)-CF3 | (6-69) | 2% |
| 3-HHBB(F, F)-F | (7-6) | 4% |
| 3-GBB(F)B(F, F)-F | (7-55) | 2% |
| 2-dhBB(F, F)XB(F, F)-F | (7-50) | 4% |
| 3-GB(F)B(F, F)XB(F, F)-F | (7-57) | 3% |
| 3-HGB(F, F)XB(F, F)-F | (-) | 5% |
| 7-HB(F, F)-F | (5-4) | 3% |
| 2-HH-3 | (2-1) | 14% |
| 2-HH-5 | (2-1) | 4% |
| 3-HH-V | (2-1) | 26% |
| 1V2-HH-3 | (2-1) | 5% |
| 1V2-BB-1 | (2-8) | 3% |
| 2-BB(F)B-3 | (3-6) | 3% |
| 3-HB(F)HH-2 | (4-7) | 4% |
| 5-HBB(F)B-2 | (4-5) | 6% |

NI=78.4° C.; Tc<−20° C.; Δn=0.094; Δε=5.6; Vth=1.45 V; η=11.5 mPa·s; γ1=61.7 mPa·s.

### Composition (M29)

| | | |
|---|---|---|
| 3-HBB (F, F)-F | (6-24) | 5% |
| 5-HBB (F, F)-F | (6-24) | 4% |
| 3-BB (F) B (F, F)-F | (6-69) | 3% |
| 3-BB (F) B (F, F) XB (F, F)-F | (7-47) | 3% |
| 4-BB (F) B (F, F) XB (F, F)-F | (7-47) | 5% |
| 3-BB (F, F) XB (F)B (F, F)-F | (7-60) | 3% |
| 5-BB (F) B (F, F) XB (F)B (F, F)-F | (—) | 4% |
| 3-HH2BB (F, F)-F | (7-15) | 3% |
| 4-HH2BB (F, F)-F | (7-15) | 3% |
| 2-HH-5 | (2-1) | 8% |
| 3-HH-V | (2-1) | 25% |
| 3-HH-V1 | (2-1) | 7% |
| 4-HH-V1 | (2-1) | 6% |
| 5-HB-O2 | (2-5) | 5% |
| 7-HB-1 | (2-5) | 5% |
| VFF-HHB-O1 | (3-1) | 8% |
| VFF-HHB-1 | (3-1) | 3% |

NI=80.0° C.; Tc<−20° C.; Δn=0.101; Δε=4.6; Vth=1.71 V; η=11.0 mPa·s; γ1=47.2 mPa·s.

### Composition (M30)

| | | |
|---|---|---|
| 3-HHB (F, F)- F | (6-3) | 8% |
| 3-GB (F) B (F)-F | (6-116) | 2% |
| 3-GB (F) B (F, F)-F | (6-117) | 3% |
| 3-BB (F, F) XB (F, F)-F | (6-97) | 8% |
| 3-GB (F) B (F, F) XB (F, F)-F | (7-57) | 6% |
| 5-GB (F) B (F, F) XB (F, F)-F | (7-57) | 5% |
| 3-HH-V | (2-1) | 30% |
| 3-HH-V1 | (2-1) | 10% |
| 1V2-HH-3 | (2-1) | 8% |
| 3-HH-VFF | (2-1) | 8% |
| V2-BB-1 | (2-8) | 2% |
| 5-HB (F) BH-3 | (4-2) | 5% |
| 5-HBBH-3 | (4-1) | 5% |

NI=78.6° C.; Tc<−20° C.; Δn=0.088; Δε=5.6; Vth=1.85 V; η=13.9 mPa·s; γ1=66.9 mPa·s.

### Composition (M31)

| | | |
|---|---|---|
| 3-HHEB (F, F)-F | (6-12) | 4% |
| 5-HHEB (F, F)-F | (6-12) | 3% |
| 3-HBEB (F, F)-F | (6-39) | 3% |
| 5-HBEB (F, F)-F | (6-39) | 3% |
| 3-BB (F) B (F, F)-F | (6-69) | 3% |
| 3-GB (F) B (F, F) XB (F, F)-F | (7-57) | 5% |
| 4-GB (F) B (F, F) XB (F, F)-F | (7-57) | 5% |
| 5-HB-CL | (2-5) | 5% |
| 3-HHB-OCF3 | (3-1) | 4% |
| 3-HHB (F, F) XB (F, F)-F | (7-29) | 5% |
| 5-HHB (F, F) XB (F, F)-F | (7-29) | 3% |
| 3-HGB (F, F) XB (F, F)-F | (—) | 5% |
| 2-HH-5 | (2-1) | 3% |
| 3-HH-5 | (2-1) | 5% |
| 3-HE-V | (2-1) | 24% |
| 4-HH-V | (2-1) | 5% |
| 1V2-HH-3 | (2-1) | 5% |
| 3-HHEH-3 | (3-13) | 5% |
| 5-B (F) BB-2 | (3-8) | 3% |
| 5-B (F) BB-3 | (3-8) | 2% |

NI=82.9° C.; Tc<−20° C.; Δn=0.093; Δε=6.9; Vth=1.50 V; η=16.3 mPa·s; γ1=65.2 mPa·s.

### Composition (M32)

| | | |
|---|---|---|
| 3-HHXB (F, F)-F | (6-100) | 9% |
| 3-HBB (F, F)-F | (6-24) | 3% |
| 3-BB (F) B (F, F)-F | (6-69) | 4% |
| 3-BB (F) B (F, F)-CF3 | (6-69) | 4% |
| 3-BB (F, F) XB (F, F)-F | (6-69) | 4% |
| 3-GBB (F) B (F, F)-F | (7-55) | 3% |
| 4-GBB (F) B (F, F)-F | (7-55) | 4% |
| 3-HH-V | (2-1) | 25% |
| 3-HH-V1 | (2-1) | 10% |
| 5-HB-O2 | (2-5) | 10% |
| 7-HB-1 | (2-5) | 5% |
| V2-BB-1 | (2-8) | 3% |
| 3-HHB-1 | (3-1) | 4% |
| 1V-HBB-2 | (3-4) | 5% |
| 5-HBB (F) B-2 | (4-5) | 6% |

NI=79.6° C.; Tc<−20° C.; Δn=0.111; Δε=4.7; Vth=1.86 V; η=9.7 mPa·s; γ1=49.9 mPa·s.

### Composition (M33)

| | | |
|---|---|---|
| 3-BB (F, F) XB (F, F)-F | (6-97) | 14% |
| 5-BB (F) B (F, F) XB (F, F)-F | (7-47) | 7% |
| 7-HB (F, F)-F | (5-4) | 6% |
| 2-HH-5 | (2-1) | 5% |
| 3-HH-V | (2-1) | 30% |
| 3-HH-V1 | (2-1) | 3% |
| 3-HH-VFF | (2-1) | 10% |
| 3-HHB-1 | (3-1) | 4% |
| 3-HHB-3 | (3-1) | 5% |
| 3-HHB-O1 | (3-1) | 3% |
| 1-BB (F) B-2V | (3-6) | 3% |
| 3-HHEBH-3 | (4-6) | 3% |
| 3-HHEBH-4 | (4-6) | 4% |
| 3-HHEBH-5 | (4-6) | 3% |

NI=83.0° C.; Tc<−20° C.; Δn=0.086; Δε=3.8; Vth=1.94 V; η=7.5 mPa·s; γ1=51.5 mPa·s.

### Composition (M34)

| | | |
|---|---|---|
| 3-HBB (F, F)-F | (6-24) | 5% |
| 5-HBB (F, F)-F | (6-24) | 4% |
| 3-BB (F) B (F, F)-F | (6-69) | 3% |
| 3-BB (F) B (F, F) XB (F, F)-F | (7-47) | 3% |
| 4-BB (F) B (F, F) XB (F, F)-F | (7-47) | 5% |
| 3-BB (F, F) XB (F) B (F, F)-F | (7-60) | 3% |
| 5-BB (F) B (F, F) XB (F) B (F, F)-F | (—) | 4% |
| 3-HH2BB (F, F)-F | (7-15) | 3% |
| 4-HH2BB (F, F)-F | (7-15) | 3% |
| 2-HH-5 | (2-1) | 8% |
| 3-HH-V | (2-1) | 28% |
| 4-HH-V1 | (2-1) | 7% |
| 5-HB-O2 | (2-5) | 2% |
| 7-HB-1 | (2-5) | 5% |
| VFF-HHB-O1 | (3-1) | 8% |
| VFF-HHB-1 | (3-1) | 3% |
| 2-BB (2F, 3F) B-3 | (11-1) | 4% |
| 3-HBB (2F, 3F)-O2 | (10-7) | 2% |

NI=81.9° C.; Tc<−20° C.; Δn=0.109; Δε=4.8; Vth=1.75 V; η=13.3 mPa·s; γ1=57.4 mPa·s.

### Composition (M35)

| | | |
|---|---|---|
| 3-HHEB (F, F)-F | (6-12) | 4% |
| 3-HBEB (F, F)-F | (6-39) | 3% |
| 5-HBEB (F, F)-F | (6-39) | 3% |
| 3-BB (F) B (F, F)-F | (6-69) | 3% |
| 3-HBBXB (F, F)-F | (7-32) | 6% |
| 4-GBB (F, F) XB (F, F)-F | (7-62) | 2% |
| 5-GBB (F, F) XB (F, F)-F | (7-62) | 2% |
| 3-GB (F) B (F, F) XB (F, F)-F | (7-57) | 5% |
| 4-GB (F) B (F, F) XB (F, F)-F | (7-57) | 5% |
| 5-HHB (F, F) XB (F, F)-F | (7-29) | 3% |
| 5-HEB (F, F)-F | (5-10) | 3% |
| 5-HB-CL | (5-2) | 2% |
| 3-HHB-OCF3 | (6-1) | 4% |
| 3-HH-5 | (2-1) | 4% |
| 3-HH-V | (2-1) | 21% |
| 3-HH-V1 | (2-1) | 3% |
| 4-HH-V | (2-1) | 4% |
| 1V2-HH-3 | (2-1) | 6% |
| 5-B (F) BB-2 | (3-8) | 3% |
| 5-B (F) BB-3 | (3-8) | 2% |
| 3-HB (2F, 3F)-O2 | (10-7) | 3% |
| 3-BB (2F, 3F)-O2 | (9-3) | 2% |
| 3-HHB (2F, 3F)-O2 | (10-1) | 4% |
| F3-HH-V | (2-1) | 3% |

NI=78.2° C.; Tc<−20° C.; Δn=0.101; Δε=6.7; Vth=1.45 V; η=17.8 mPa·s; γ1=67.8 mPa·s.

### Composition (M36)

| | | |
|---|---|---|
| 3-HHB (F, F)-F | (6-3) | 10% |
| 3-HHXB (F, F)-F | (6-100) | 2% |
| 3-GHB (F, F)-F | (6-109) | 5% |
| 3-BB (F) B (F, F)-F | (6-69) | 6% |
| 3-BB (F, F) XB (F, F)-F | (6-97) | 14% |
| 4-BB (F) B (F, F) XB (F, F)-F | (7-47) | 10% |
| 5-BB (F) B (F, F) XB (F, F)-F | (7-47) | 6% |
| 2-HH-3 | (2-1) | 5% |
| 3-HH-4 | (2-1) | 11% |
| 3-HH-O1 | (2-1) | 5% |
| 5-HB-O2 | (2-5) | 8% |
| 3-HHB-1 | (3-1) | 6% |
| 3-HHB-3 | (3-1) | 6% |
| 3-HHB-O1 | (3-1) | 6% |

NI=77.6° C.; Tc<−20° C.; Δn=0.109; Δε=10.6; Vth=1.34 V; η=22.6 mPa·s; γ1=92.4 mPa·s.

### Composition (M37)

| | | |
|---|---|---|
| 3-HBB-F | (6-22) | 3% |
| 3-BB (F, F) XB (F)-OCF3 | (6-96) | 3% |
| 3-HHB (F)-F | (6-2) | 3% |
| 3-HGB (F, F)-F | (6-103) | 3% |
| 5-GHB (F, F)-F | (6-109) | 3% |
| 3-HBB (F, F)-F | (6-24) | 4% |
| 3-BB (F, F) XB (F, F)-F | (6-97) | 5% |
| 3-HHBB (F, F)-F | (7-6) | 5% |
| 3-HBBXB (F, F)-F | (7-32) | 5% |
| 3-BBVFFXB (F, F)-F | (6-119) | 8% |
| 3-HE-V | (2-1) | 39% |
| 1-HH-V1 | (2-1) | 3% |
| 1-HH-2V1 | (2-1) | 4% |
| 3-HHEH-5 | (3-13) | 3% |
| 1-BB (F) B-2V | (3-6) | 3% |
| 3-HHEBH-3 | (4-6) | 3% |
| 5-HBB (F) B-2 | (4-5) | 3% |

NI=85.2° C.; Tc<−20° C.; Δn=0.102; Δε=4.1; Δ1=43.0 mPa·s.

### Composition (M38)

| | | |
|---|---|---|
| 3-HHBB (F)-F | (7-5) | 3% |
| 2-HHEB (F, F)-F | (6-12) | 3% |
| 5-BB (F) B (F, F)-F | (6-69) | 7% |
| 3-HHB (F) B (F, F)-F | (7-9) | 3% |
| 3-GB (F) B (F, F) XB (F, F)-F | (7-57) | 3% |
| 3-BB (F, F) XB (F) B (F, F)-F | (7-60) | 3% |
| 3-HHVFFXB (F, F)-F | (6-120) | 5% |
| 3-BBVFFXB (F, F)-F | (6-119) | 5% |
| 3-HBBVFFXB (F, F)-F | (7-61) | 3% |
| 2-HH-5 | (2-1) | 5% |
| 3-HH-V | (2-1) | 20% |
| 5-HH-V | (2-1) | 12% |
| 3-HH-V1 | (2-1) | 4% |
| 4-HH-V1 | (2-1) | 5% |
| 2-HH-2V1 | (2-1) | 3% |
| 1-BB-3 | (2-8) | 3% |
| V2-BB (F) B-1 | (3-6) | 5% |
| V2-B (F) BB-1 | (3-8) | 5% |
| 3-HB (F) HH-5 | (4-7) | 3% |

NI=85.8° C.; Tc<−20° C.; Δn=0.115; Δε=4.2; γ1=41.4 mPa·s.

### Composition (M39)

| | | |
|---|---|---|
| 3-BB (F) XB (F) B (F, F)-F | (7-60) | 5% |
| 3-HGB (F, F)-F | (6-103) | 3% |
| 5-GHB (F, F)-F | (6-109) | 4% |
| 3-GB (F, F) XB (F, F)-F | (6-113) | 5% |
| 3-HHBB (F, F)-F | (7-6) | 4% |
| 2-dhBB (F, F) XB (F, F)-F | (7-50) | 4% |
| 3-GB (F) B (F, F) XB (F, F)-F | (7-57) | 3% |
| 3-HGB (F, F) XB (F, F)-F | (7) | 5% |
| 7-HB (F, F)-F | (5-4) | 3% |
| 2-HH-3 | (2-1) | 14% |
| 2-HH-5 | (2-1) | 4% |
| 3-HH-V | (2-1) | 26% |
| 1V2-HH-3 | (2-1) | 5% |
| 1V2-BB-1 | (2-8) | 3% |
| 2-BB (F) B-3 | (3-6) | 3% |
| 3-HB (F) HH-2 | (4-7) | 4% |
| 5-HBB (F) B-2 | (4-5) | 5% |

NI=78.4° C.; Tc<−20° C.; Δn=0.094; Δε=5.6; Vth=1.45 V; η=11.5 mPa·s; γ1=61.7 mPa·s.

### Composition (M40)

| | | |
|---|---|---|
| 3-HBB (F, F)-F | (6-24) | 5% |
| 5-HBB (F, F)-F | (6-24) | 4% |
| 3-BB (F) B (F, F) XB (F, F)-F | (7-47) | 3% |
| 4-BB (F) B (F, F) XB (F, F)-F | (7-47) | 5% |
| 3-BB (F, F) XB (F)B (F, F)-F | (7-60) | 10% |
| 3-HH2BB (F, F)-F | (7-15) | 3% |
| 4-HH2BB (F, F)-F | (7-15) | 3% |
| 2-HH-5 | (2-1) | 4% |
| 3-HH-V | (2-1) | 25% |
| 3-HH-V1 | (2-1) | 10% |
| 4-HH-V1 | (2-1) | 7% |
| 5-HB-O2 | (2-5) | 5% |
| 7-HB-1 | (2-5) | 5% |
| VFF-HHB-O1 | (3-1) | 8% |
| VFF-HHB-1 | (3-1) | 3% |

NI=79.3° C.; Tc<−20° C.; Δn=0.099; Δε=5.0; Vth=1.64 V; η=10.4 mPa·s; γ1=44.7 mPa·s.

### Composition (M41)

| | | |
|---|---|---|
| 3-GBXB (F)B (F, F)-F | (7) | 5% |
| 3-HHB (F, F)-F | (6-3) | 7% |
| 3-GB (F) B (F)-F | (6-116) | 2% |
| 3-GB (F) B (F, F)-F | (6-117) | 3% |
| 3-BB (F, F) XB (F, F)-F | (6-97) | 7% |
| 3-GB (F) B (F, F) XB (F, F)-F | (7-57) | 4% |
| 5-GB (F) B (F, F) XB (F, F)-F | (7-57) | 5% |
| 3-HH-V | (2-1) | 30% |
| 3-HH-V1 | (2-1) | 10% |
| 1V2-HH-3 | (2-1) | 8% |
| 3-HH-VFF | (2-1) | 8% |
| V2-BB-1 | (2-8) | 2% |
| 5-HB (F) BH-3 | (4-2) | 4% |
| 5-HBBH-3 | (4-1) | 5% |

NI=79.7° C.; Tc<−20° C.; Δn=0.091; Δε=5.7; Vth=1.83 V; η=14.9 mPa·s; γ1=69.3 mPa·s.

### 2. Alignment of Liquid Crystal Molecules

### Use Example 1 to Use Example 7

To composition (M1), compound (No. 156) was added at a proportion of 0.1% by weight, 0.3% by weight, 0.5% by weight, 1.0% by weight, 3.0% by weight, 5.0% by weight or 10.0% by weight as a first additive, and compound (AO-1) in which R⁴⁰ is n-heptyl was added at a proportion of 150 ppm as an antioxidant. When the resulting mixture was heated and stirred at 100° C. and then returned to room temperature and allowed to stand for one week, the compounds were completely dissolved without precipitation of crystals or the like. The resulting mixture was injected into an IPS device having no alignment film at 90° C. (equal to or higher than a maximum temperature of a nematic phase). The IPS device was irradiated with linearly polarized ultraviolet light (313 nm, 2.0 J/cm²) from a direction normal to the device while heating the device at 90° C. to obtain a device subjected to alignment treatment. The resulting device was set on a polarizing microscope in which a polarizer and an analyzer were arranged perpendicularly to each other to be parallel to a polarization axis of linearly polarized light in the device. The device was irradiated with light from below, and presence or absence of light leakage was observed. A case where no light passed through the device was judged to be “Good” in alignment. A case where light passing through the device was observed was expressed by “Poor.” No light leakage was observed in the present Use Examples 1 to 7, and therefore the alignment was good.

### Use Example 8 to Use Example 28

Composition (M1) was used, compound (AO-1) in which R⁴⁰ is n-heptyl was added at a proportion of 150 ppm as an antioxidant, and a first additive was mixed thereto at a proportion shown in Table 4 below. Operation was performed in the same manner as in Use Example 1 except for the operation described above. When solubility and presence or absence of light leakage were observed in the same manner as in Use Example 1, materials were completely dissolved, and alignment was good in all.

TABLE 4

| | Liquid | | Addition | | |
|---|---|---|---|---|---|
| Use | crystal | First | concentration | | |
| Example | composition | additive | (wt %) | Solubility | Alignment |
| 1 | M1 | No. 156 | 0.1 | Soluble | Good |
| 2 | M1 | No. 156 | 0.3 | Soluble | Good |
| 3 | M1 | No. 156 | 0.5 | Soluble | Good |
| 4 | M1 | No. 156 | 1 | Soluble | Good |
| 5 | M1 | No. 156 | 3 | Soluble | Good |
| 6 | M1 | No. 156 | 5 | Soluble | Good |
| 7 | M1 | No. 156 | 10 | Soluble | Good |
| 8 | M1 | No. 157 | 0.1 | Soluble | Good |
| 9 | M1 | No. 157 | 0.3 | Soluble | Good |
| 10 | M1 | No. 157 | 0.5 | Soluble | Good |
| 11 | M1 | No. 157 | 1 | Soluble | Good |
| 12 | M1 | No. 157 | 3 | Soluble | Good |
| 13 | M1 | No. 157 | 5 | Soluble | Good |
| 14 | M1 | No. 157 | 10 | Soluble | Good |
| 15 | M1 | No. 158 | 0.1 | Soluble | Good |
| 16 | M1 | No. 158 | 0.3 | Soluble | Good |
| 17 | M1 | No. 158 | 0.5 | Soluble | Good |
| 18 | M1 | No. 158 | 1 | Soluble | Good |
| 19 | M1 | No. 158 | 3 | Soluble | Good |
| 20 | M1 | No. 158 | 5 | Soluble | Good |
| 21 | M1 | No. 158 | 10 | Soluble | Good |
| 22 | M1 | No. 81 | 0.1 | Soluble | Good |
| 23 | M1 | No. 81 | 0.3 | Soluble | Good |
| 24 | M1 | No. 81 | 0.5 | Soluble | Good |
| 25 | M1 | No. 81 | 1 | Soluble | Good |
| 26 | M1 | No. 81 | 3 | Soluble | Good |
| 27 | M1 | No. 81 | 5 | Soluble | Good |
| 28 | M1 | No. 81 | 10 | Soluble | Good |

When the same operation was performed by changing the liquid crystal compositions to be used to M2 to M41, respectively, in Use Examples 1 to 28, solubility and alignment were good also in all.

When the same operation was performed by appropriately selecting materials from among compositions from composition (M1) to composition (M41) and first additives from compound (No. 1) to compound (No. 280), solubility and alignment were good in all.

### Comparative Examples 1 to 21

Solubility and alignability were evaluated according to the same operation as in Use Examples by mixing compound [A-1-1-1] in which all polymerizable groups were an acrylate group, compound [14] according to Patent literature No. 3, and compound [Formula 2] according to Patent literature No. 2 in which all polymerizable groups were a methacrylate group, as a first additive, with liquid crystal composition (M1) at a proportion shown in Table 5 below. As a result, any compound is inferior in the solubility to the compound according to the embodiment of the invention, and the range of an addition concentration in which alignment was confirmed was limited. Moreover, when the same evaluation was performed by using liquid crystal compositions (M2) to (M41), the same tendency as in the case where composition (M1) was used was found in all.

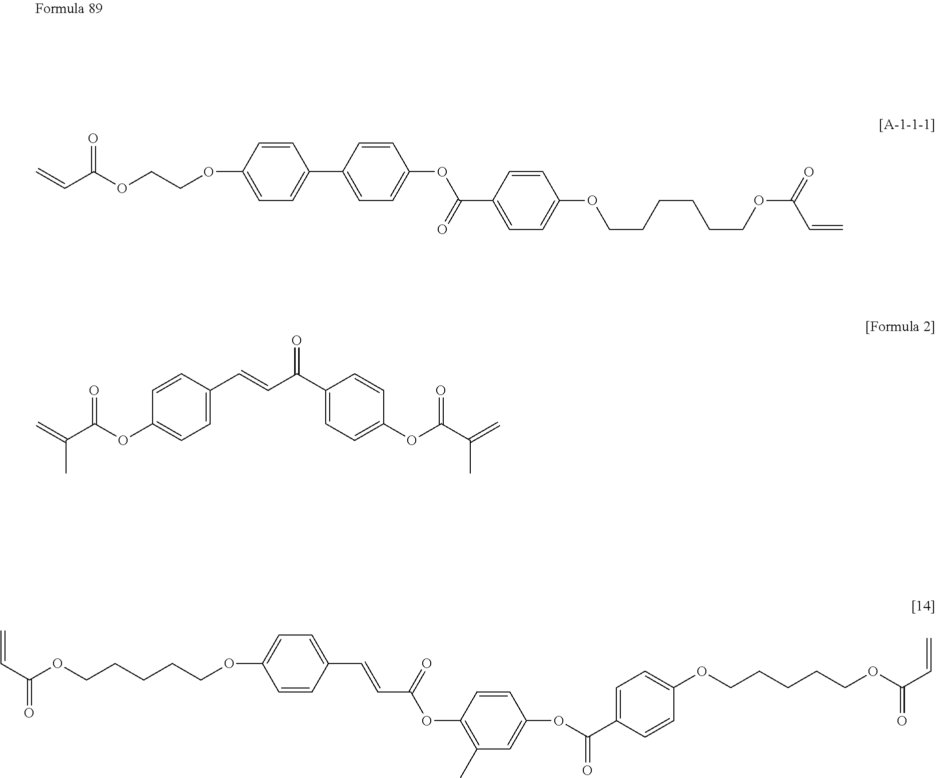

TABLE 5

| Com- | Liquid | | Addition | | |
|---|---|---|---|---|---|
| parative | crystal | First | concentration | | Align- |
| Example | composition | additive | (wt %) | Solubility | ment |
| 1 | M1 | A-1-1-1 | 0.1 | Soluble | Poor |
| 2 | M1 | A-1-1-1 | 0.3 | Soluble | Poor |
| 3 | M1 | A-1-1-1 | 0.5 | Soluble | Good |
| 4 | M1 | A-1-1-1 | 1 | Soluble | Poor |
| 5 | M1 | A-1-1-1 | 3 | Insoluble | Poor |
| 6 | M1 | A-1-1-1 | 5 | Insoluble | Poor |
| 7 | M1 | A-1-1-1 | 10 | Insoluble | Poor |
| 8 | M1 | [Formula 2] | 0.1 | Soluble | Poor |
| 9 | M1 | [Formula 2] | 0.3 | Soluble | Poor |
| 10 | M1 | [Formula 2] | 0.5 | Soluble | Good |
| 11 | M1 | [Formula 2] | 1 | Soluble | Poor |
| 12 | M1 | [Formula 2] | 3 | Insoluble | Poor |
| 13 | M1 | [Formula 2] | 5 | Insoluble | Poor |
| 14 | M1 | [Formula 2] | 10 | Insoluble | Poor |
| 15 | M1 | [14] | 0.1 | Soluble | Poor |
| 16 | M1 | [14] | 0.3 | Soluble | Poor |
| 17 | M1 | [14] | 0.5 | Soluble | Good |
| 18 | M1 | [14] | 1 | Soluble | Poor |
| 19 | M1 | [14] | 3 | Insoluble | Poor |
| 20 | M1 | [14] | 5 | Insoluble | Poor |
| 21 | M1 | [14] | 10 | Insoluble | Poor |

In Use Examples, a kind and an amount of compositions or compound (1) that is a polar compound were changed, but neither an insoluble remain nor a precipitation was found, and no light leakage from the device was observed. The results indicate that alignment is good even without an alignment film of polyimide or the like in the device, and all liquid crystal molecules are arranged in a fixed direction. On the other hand, in Comparative Examples, upon addition at high concentration, solubility was not sufficient, and an addition concentration range in which alignment is favorably observed was also limited. Accordingly, if compound (1) according to the embodiment of the invention is used, the compound can be used in a wide addition concentration range. Moreover, if the liquid crystal composition according to the embodiment of the invention is used, a liquid crystal display device having at least one of characteristics such as a wide temperature range in which the device can be used, a short response time, a high voltage holding ratio, low threshold voltage, a large contrast ratio and a long service life can be obtained. Further, a liquid crystal display device having the liquid crystal composition satisfying at least one of characteristics such as high maximum temperature of a nematic phase, low minimum temperature of the nematic phase, small viscosity, suitable optical anisotropy, large negative dielectric anisotropy, large specific resistance, high stability to ultraviolet light and high stability to heat can be obtained.

### INDUSTRIAL APPLICABILITY

A liquid crystal composition according to an embodiment of the invention can be used in a liquid crystal monitor, a liquid crystal television and so forth.

1. A compound, represented by formula (1): 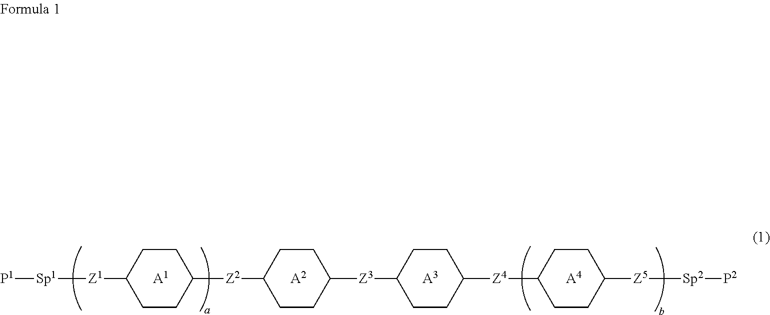
wherein, in formula (1),
a and b are independently 0, 1 or 2, and expressions: 0≤a+b≤3 hold,
ring A¹, ring A², ring A³ and ring A⁴ are independently 1,4-cyclohexylene, 1,4-cyclohexenylene, 1,4-phenylene, naphthalene-2,6-diyl, decahydronaphthalene-2,6-diyl, 1,2,3,4-tetrahydronaphthalene-2,6-diyl, tetrahydropyran-2,5-diyl, 1,3-dioxane-2,5-diyl, pyrimidine-2,5-diyl, pyridine-2,5-diyl, fluorene-2,7-diyl, phenanthrene-2,7-diyl, anthracene-2,6-diyl, perhydrocyclopenta[a]phenanthrene-3,17-diyl or 2,3,4,7,8,9,10,11,12,13,14,15,16,17-tetradecahydrocyclopenta[a]phenanthrene-3,17-diyl, and in the rings, at least one hydrogen may be replaced by fluorine, chlorine, alkyl having 1 to 12 carbons, alkenyl having 2 to 12 carbons, alkoxy having 1 to 11 carbons, alkenyloxy having 2 to 11 carbons, -Sp¹-P¹ or -Sp²-P², and in the groups, at least one hydrogen may be replaced by fluorine or chlorine, and when a is 2, two of ring A¹ may be different, and when b is 2, two of ring A⁴ may be different;
Z¹, Z², Z³, Z⁴ and Z⁵ are independently a single bond or alkylene having 1 to 10 carbons, and in the alkylene, at least one piece of —CH₂— may be replaced by —O—, —CO—, —COO—, —OCO— or —OCOO—, and at least one piece of —(CH₂)₂— may be replaced by —CH═CH— or —C≡C— and in the groups, at least one hydrogen may be replaced by halogen, in which at least one in Z², Z³ or Z⁴ is —COO—, —OCO—, —CH═CHCOO—, —OCOCH═CH—, —CH═CH—, —CH═CHCO— or —COCH═CH—, and when a is 2, two of Z¹ may be different, and two of Z⁵ may be different;
Sp¹ and Sp² are independently a single bond or alkylene having 1 to 10 carbons, and in the alkylene, at least one piece of —CH₂— may be replaced by —O—, —CO—, —COO—, —OCO— or —OCOO—, and at least one piece of —(CH₂)₂— may be replaced by —CH═CH— or —C≡C—, and in the groups, at least one hydrogen may be replaced by halogen, and when a plurality of Sp¹ or Sp² are present in a structure, the plurality each may be different; and
P¹ and P² are independently a group represented by any one of formula (1b) to formula (1h), and when a plurality of P¹ or P² are present in a structure, the plurality each may be different, in which a case where all of P¹ and P² have an identical structure is excluded; 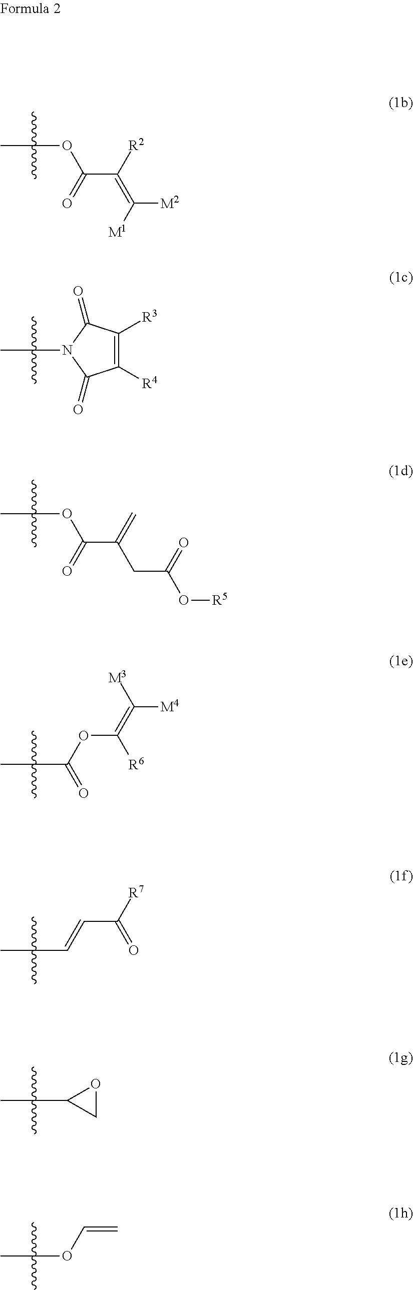
wherein, in formula (1b) to formula (1h),
M¹, M², M³ and M⁴ are independently hydrogen, halogen, alkyl having 1 to 5 carbons, or alkyl having 1 to 5 carbons in which at least one hydrogen is replaced by halogen;
R² is hydrogen, halogen or alkyl having 1 to 5 carbons, and in the alkyl, at least one hydrogen may be replaced by halogen, and at least one piece of —CH₂— may be replaced by —O—; and
R³, R⁴, R⁵, R⁶ and R⁷ are independently hydrogen or alkyl having 1 to 15 carbons, and in the alkyl, at least one piece of —CH₂— may be replaced by —O— or —S—, and at least one piece of —(CH₂)₂— may be replaced by —CH═CH— or —C≡C—, and in the groups, at least one hydrogen may be replaced by halogen.

2. The compound according to claim 1, wherein, in formula (1),
a and b are independently 0, 1 or 2, and expressions: 0≤a+b≤2 hold;
ring A¹, ring A², ring A³ and ring A⁴ are independently 1,4-cyclohexylene, 1,4-cyclohexenylene, 1,4-phenylene, naphthalene-2,6-diyl, decahydronaphthalene-2,6-diyl, 1,2,3,4-tetrahydronaphthalene-2,6-diyl, tetrahydropyran-2,5-diyl, 1,3-dioxane-2,5-diyl, pyrimidine-2,5-diyl, pyridine-2,5-diyl, fluorene-2,7-diyl, phenanthrene-2,7-diyl, anthracene-2,6-diyl, perhydrocyclopenta[a]phenanthrene-3,17-diyl or 2,3,4,7,8,9,10,11,12,13,14,15,16,17-tetradecahydrocyclopenta[a]phenanthrene-3,17-diyl, and in the rings, at least one hydrogen may be replaced by fluorine, chlorine, alkyl having 1 to 12 carbons, alkenyl having 2 to 12 carbons, alkoxy having 1 to 11 carbons, alkenyloxy having 2 to 11 carbons, -Sp¹-P¹ or -Sp²-P², and in the groups, at least one hydrogen may be replaced by fluorine or chlorine, and when a is 2, two of ring A¹ may be different, and when b is 2, two of ring A⁴ may be different;
Z¹, Z², Z³, Z⁴ and Z⁵ and are independently a single bond, —(CH₂)₂—, —CH═CH—, —C≡C—, —COO—, —OCO—, —CF₂O—, —OCF₂—, —CH₂O—, —OCH₂—, —CF═CF—, —CH═CHCOO—, —OCOCH═CH—, —CH═CHCO— or —COCH═CH—, in which at least one in Z², Z³ or Z⁴ is —COO—, —OCO—, —CH═CHCOO—, —OCOCH═CH—, —CH═CH—, —CH═CHCO— or —COCH═CH—, and when a is 2, two of Z¹ may be different, and two of Z⁵ may be different; Sp¹ and Sp² are independently a single bond or alkylene having 1 to 10 carbons, and in the alkylene, at least one piece of —CH₂— may be replaced by —O—, —COO— or —OCO—, and at least one piece of —(CH₂)₂— may be replaced by —CH═CH—, and in the groups, at least one hydrogen may be replaced by fluorine or chlorine, and when a plurality of Sp¹ or Sp² are present in a structure, the plurality each may be different; and
P¹ and P² are independently a group represented by any one of formula (1b) to formula (1h), and when a plurality of P¹ or P² are present in a structure, the plurality each may be different, in which a case where all of P¹ and P² have an identical structure is excluded; 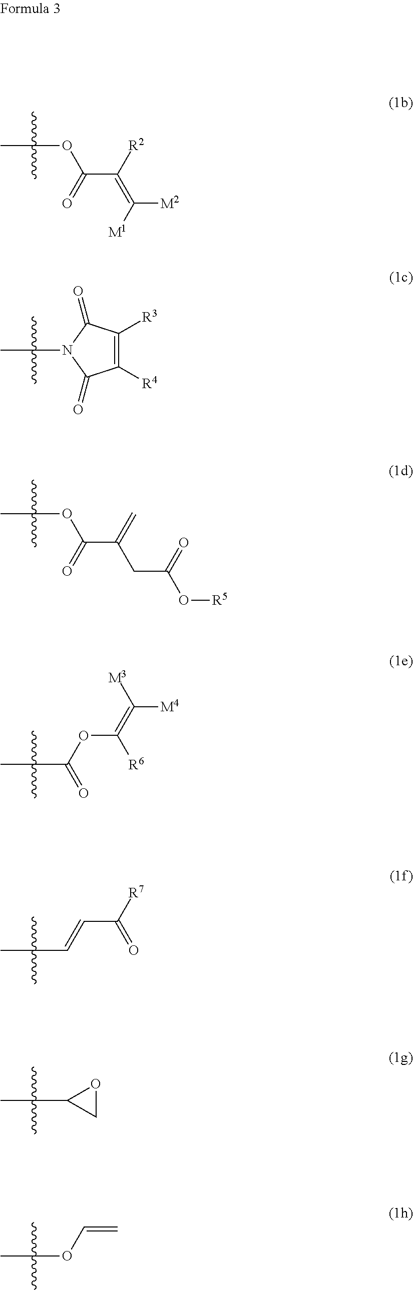
wherein, in formula (1b) to formula (1h),
M¹, M², M³ and M⁴ are independently hydrogen, halogen, alkyl having 1 to 5 carbons, or alkyl having 1 to 5 carbons in which at least one hydrogen is replaced by halogen;
R² is hydrogen, halogen or alkyl having 1 to 5 carbons, and in the alkyl, at least one hydrogen may be replaced by halogen, and at least one piece of —CH₂— may be replaced by —O—; and
R³, R⁴, R⁵, R⁶ and R⁷ are independently hydrogen or alkyl having 1 to 15 carbons, and in the alkyl, at least one piece of —CH₂— may be replaced by —O— or —S—, and at least one piece of —(CH₂)₂— may be replaced by —CH═CH— or —C≡C—, and in the groups, at least one hydrogen may be replaced by halogen.

3. The compound according to claim 1, represented by any one of formula (1-1) to formula (1-3): 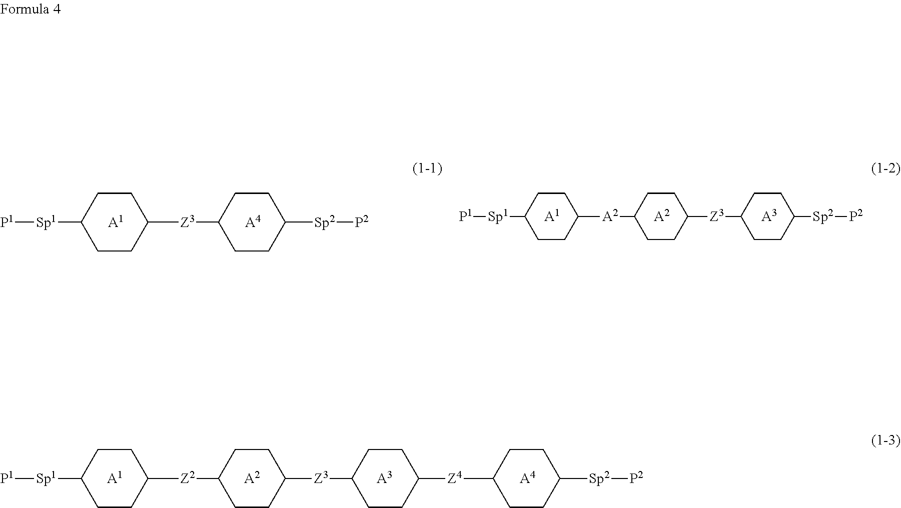
wherein, in formula (1-1) to formula (1-3),
ring A¹, ring A², ring A³ and ring A⁴ are independently 1,4-cyclohexylene, 1,4-phenylene, naphthalene-2,6-diyl, pyrimidine-2,5-diyl, pyridine-2,5-diyl, fluorene-2,7-diyl, phenanthrene-2,7-diyl or anthracene-2,6-diyl, and in the rings, at least one hydrogen may be replaced by fluorine, chlorine, alkyl having 1 to 12 carbons, alkenyl having 2 to 12 carbons, alkoxy having 1 to 11 carbons, alkenyloxy having 2 to 11 carbons, -Sp¹-P¹ or -Sp²-P², and in the groups, at least one hydrogen may be replaced by fluorine or chlorine;
Z², Z³ and Z⁴ are independently a single bond, —(CH₂)₂—, —CH═CH—, —C≡C—, —COO—, —OCO—, —CF₂O—, —OCF₂—, —CH₂O—, —OCH₂—, —CF═CF—, —CH═CHCOO—, —OCOCH═CH—, —CH═CHCO— or —COCH═CH—, in which at least one in Z², Z³ and Z⁴ is —COO—, —OCO—, —CH═CHCOO—, —OCOCH═CH—, —CH═CH—, —CH═CHCO— or —COCH═CH—;
Sp¹ and Sp² are independently a single bond or alkylene having 1 to 10 carbons, and in the alkylene, at least one piece of —CH₂— may be replaced by —O—, —COO—, —OCOO— or —OCO—, and at least one piece of —(CH₂)₂— may be replaced by —CH═CH—, and in the groups, at least one hydrogen may be replaced by fluorine or chlorine, and when a plurality of Sp¹ or Sp² are present in a structure, the plurality each may be different; and
P¹ and P² are independently a group represented by any one of formula (1b) to formula (1h), and when a plurality of P¹ or P² are present in a structure, the plurality each may be different, in which a case where all of P¹ and P² have an identical structure is excluded; 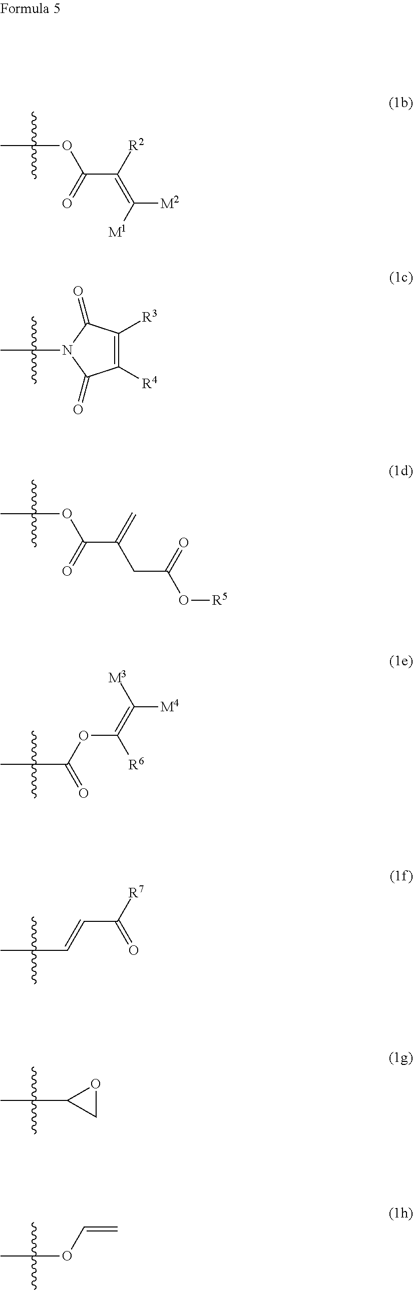
wherein
M¹, M², M³ and M⁴ are independently hydrogen, halogen, alkyl having 1 to 5 carbons, or alkyl having 1 to 5 carbons in which at least one hydrogen is replaced by halogen;
R² is hydrogen, halogen or alkyl having 1 to 5 carbons, and in the alkyl, at least one hydrogen may be replaced by halogen, and at least one piece of —CH₂— may be replaced by —O—; and
R³, R⁴, R⁵, R⁶ and R⁷ are independently hydrogen or alkyl having 1 to 15 carbons, and in the alkyl, at least one piece of —CH₂— may be replaced by —O— or —S—, and at least one piece of —(CH₂)₂— may be replaced by —CH≡CH— or —C≡C—, and in the groups, at least one hydrogen may be replaced by halogen.

4. The compound according to claim 3,
wherein, in formula (1-1), formula (1-2) and formula (1-3),
ring A¹, ring A², ring A³ and ring A⁴ are independently 1,4-cyclohexylene, 1,4-phenylene or fluorene-2,7-diyl, and in the rings, at least one hydrogen may be replaced by fluorine, chlorine, alkyl having 1 to 12 carbons, alkenyl having 2 to 12 carbons, alkoxy having 1 to 11 carbons, alkenyloxy having 2 to 11 carbons, -Sp¹-P¹ or -Sp²-P²;
Z², Z³ and Z⁴ are independently a single bond, —(CH₂)₂—, —CH═CH—, —C≡C—, —COO—, —OCO—, —CH═CHCOO—, —OCOCH═CH—, —CH═CHCO— or —COCH═CH—, in which at least one in Z², Z³ and Z⁴ is —COO—, —OCO—, —CH═CHCOO—, —OCOCH═CH—, —CH═CH—, —CH═CHCO— or —COCH═CH—;
Sp¹ and Sp² are independently a single bond or alkylene having 1 to 10 carbons, and in the alkylene, at least one piece of —CH₂— may be replaced by —O—, —COO—, —OCOO— or —OCO—, and at least one piece of —(CH₂)₂— may be replaced by —CH═CH—, and when a plurality of Sp¹ or Sp² are present in a structure, the plurality each may be different; and
P¹ and P² are independently a group represented by any one of formula (1b), formula (1c), formula (1d) or formula (1e), and when a plurality of P¹ or P² are present in a structure, the plurality each may be different, in which a case where all of P¹ and P² have an identical structure is excluded, and a case where P¹ and P² are in a combination of only acrylate or methacrylate is excluded; 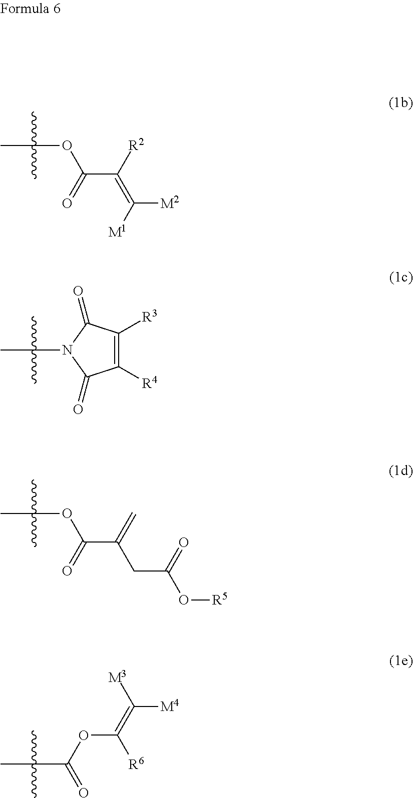
wherein
M¹, M², M³ and M⁴ are independently hydrogen, halogen, alkyl having 1 to 5 carbons, or alkyl having 1 to 5 carbons in which at least one hydrogen is replaced by halogen; and
in formula (1b) to formula (1e),
R² is hydrogen, halogen or alkyl having 1 to 5 carbons, and in the alkyl, at least one hydrogen may be replaced by halogen, and at least one piece of —CH₂— may be replaced by —O—; and
R³, R⁴, R⁵ and R⁶ are independently hydrogen or alkyl having 1 to 15 carbons, and in the alkyl, at least one piece of —CH₂— may be replaced by —O— or —S—, and at least one piece of —(CH₂)₂— may be replaced by —CH═CH— or —C≡C—, and in the groups, at least one hydrogen may be replaced by halogen.

5. The compound according to claim 4, wherein, in the compound represented by formula (1-1), formula (1-2) or formula (1-3), any one of Z², Z³ or Z⁴ is —COO— or —OCO—.

6. The compound according to claim 4, wherein, in the compound represented by formula (1-1), formula (1-2) or formula (1-3), any one of Z², Z³ or Z⁴ is —CH═CHCOO—, —OCOCH═CH—, —CH═CH—, —CH═CHCO— or —COCH═CH—.

7. The compound according to claim 1, represented by formula (1-A):
Formula 7
P¹-Sp¹-Y-Sp²-P² (1-A)
wherein
P¹ and P² are independently a group represented by formula (1b-1), (1b-2), (1b-3), (1b-4), (1b-5), (1c-1), (1d-1), (1d-2) or (1e-1), in which a case where all of P¹ and P² have an identical structure is excluded, and a case where P¹ and P² are in a combination of only formulas (1b-1) and (1b-2) is excluded;
Sp¹ and Sp² are independently a single bond or alkylene having 1 to 10 carbons, and in the alkylene, at least one piece of —CH₂— may be replaced by —O—, —COO—, —OCOO— or —OCO—, and at least one piece of —(CH₂)₂— may be replaced by —CH═CH—; 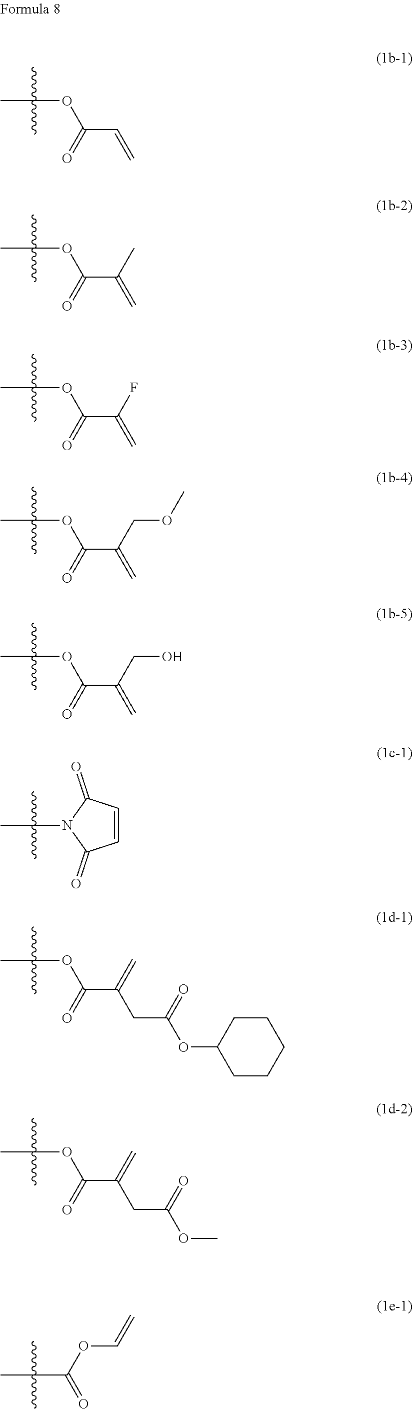
wherein
Y is a group represented by any one of formulas (MES-1-01) to (MES-1-10); 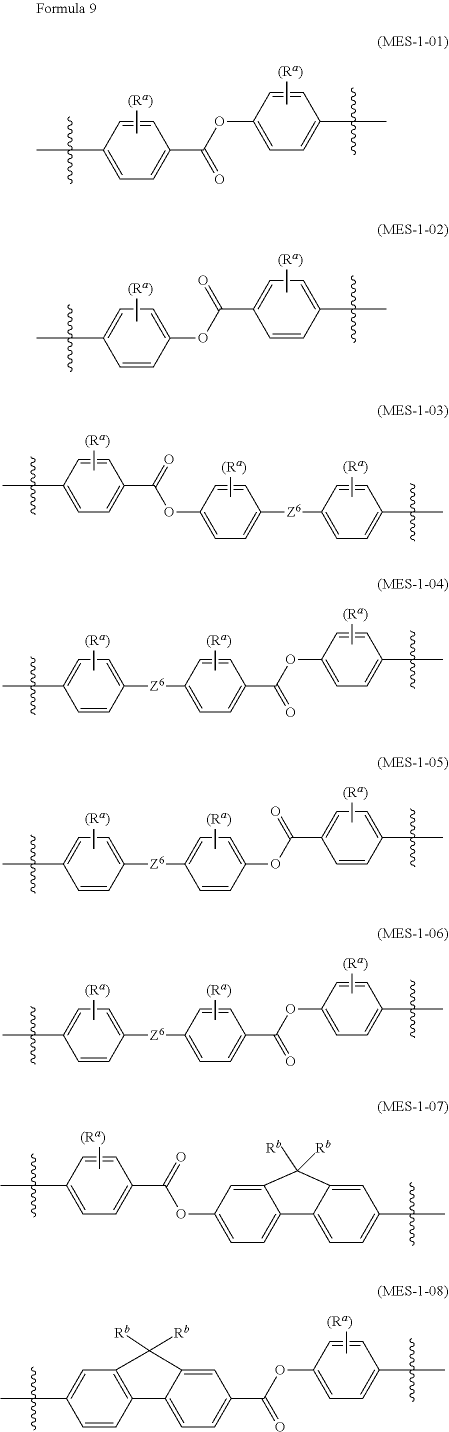 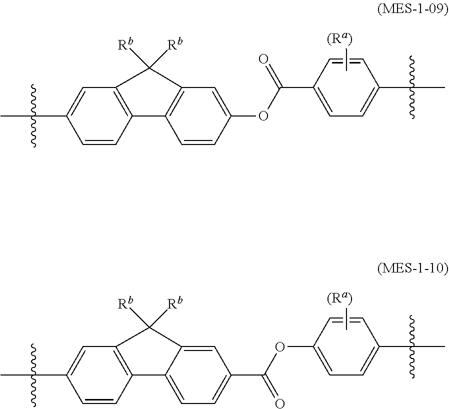
wherein
R^{a} is independently fluorine, chlorine, methyl or ethyl;
R^{b} is independently hydrogen, fluorine, methyl or ethyl;
Z⁶ is independently a single bond or —C≡C—; and
a representation formed by connecting 1,4-phenylene with (R^{a}) by a straight line as shown below in the formulas indicates 1,4-phenylene in which one or two hydrogens may be replaced by R^{a}:

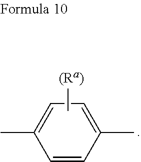

8. The compound according to claim 1, represented by formula (1-A):
Formula 11
P¹-Sp¹-Y-Sp²-P² (1-A)
wherein
P¹ and P² are independently a group represented by formula (1b-1), (1b-2), (1b-3), (1b-4), (1b-5), (1c-1), (1d-1), (1d-2) or (1e-1), in which a case where P¹ and P² have an identical structure is excluded, and a case where P¹ and P² are in a combination of only formulas (1b-1) and (1b-2) is excluded; 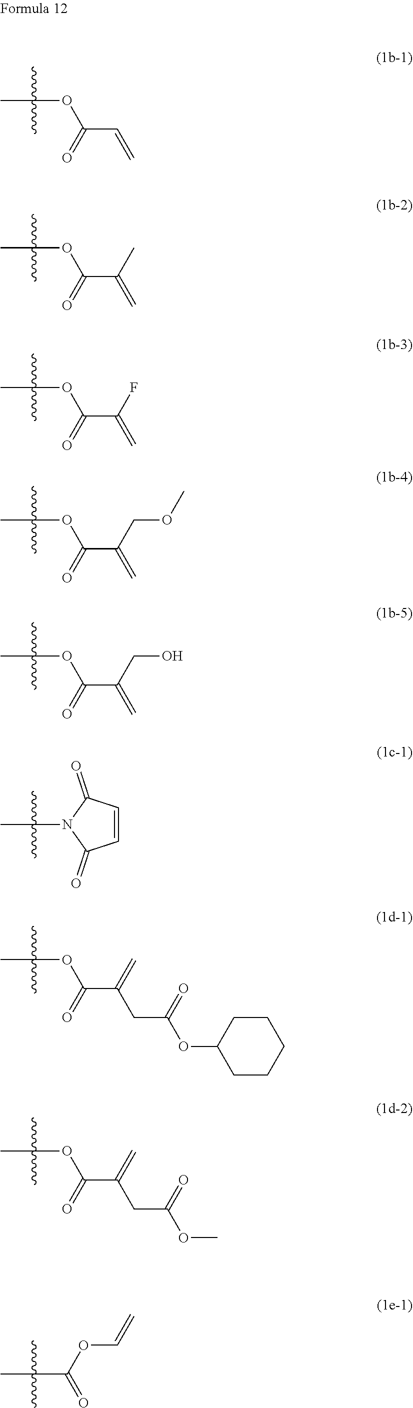
wherein
Sp¹ and Sp² are independently a single bond or alkylene having 1 to 10 carbons, and in the alkylene, at least one piece of —CH₂— may be replaced by —O—, —COO—, —OCOO— or —OCO—, and at least one piece of —(CH₂)₂— may be replaced by —CH═CH—; and
Y is a group represented by any one of formulas (MES-2-01) to (MES-2-15); 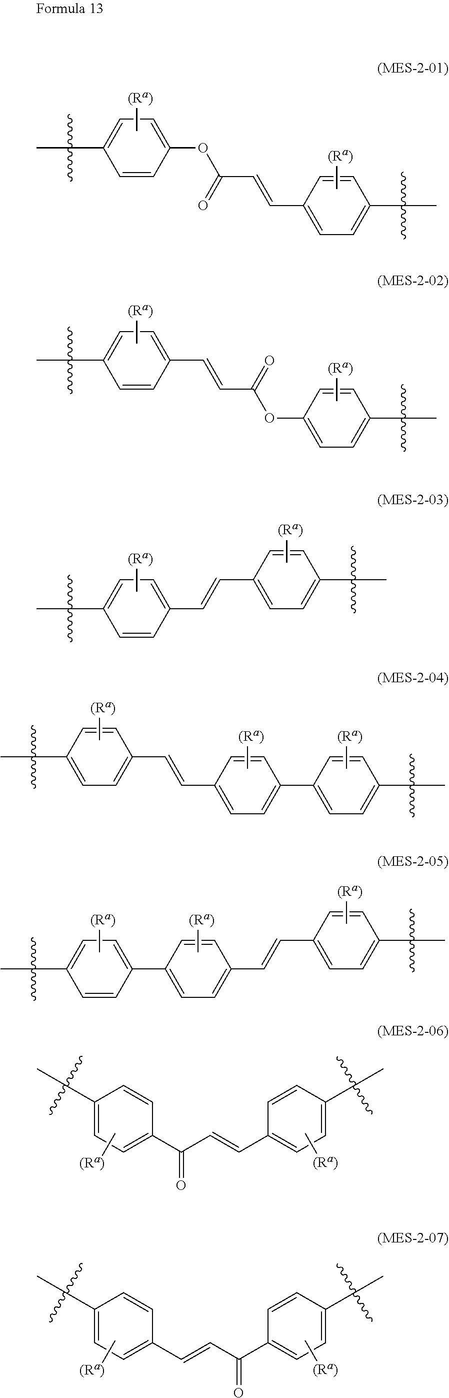 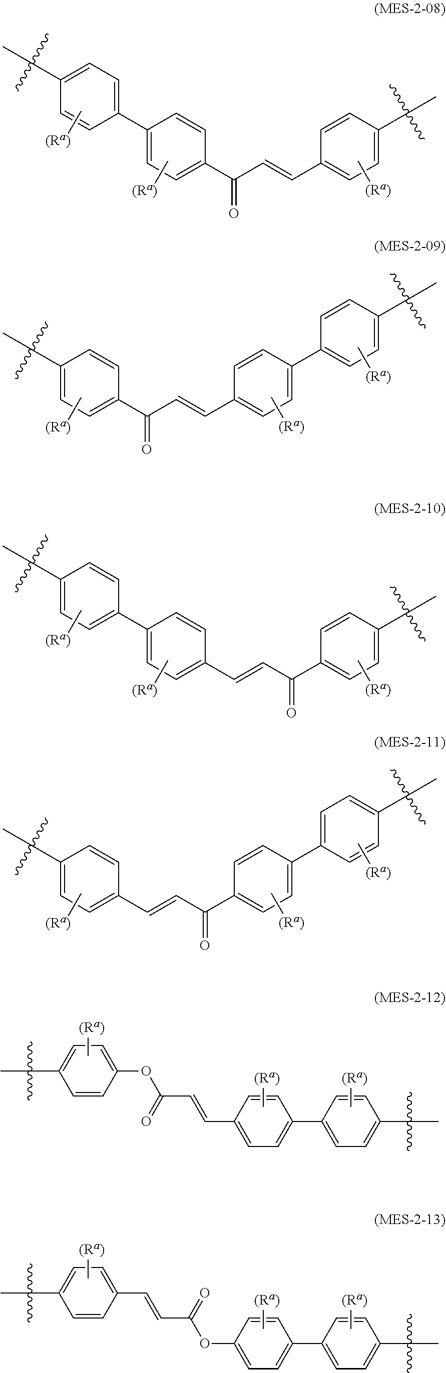 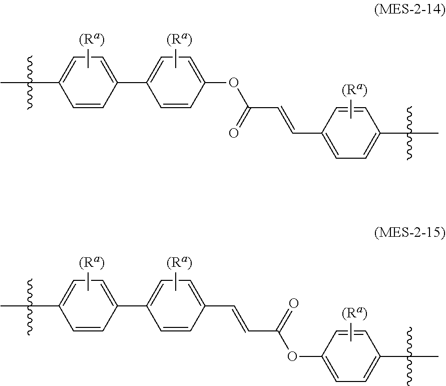
wherein
R^{a} is independently fluorine, chlorine, methyl or ethyl; and
a representation formed by connecting 1,4-phenylene with (R^{a}) by a straight line as shown below in the formulas indicates 1,4-phenylene in which one or two hydrogens may be replaced by R^{a}:

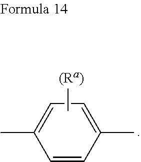

9. A liquid crystal composition, containing at least one compound according to claim 1.

10. The liquid crystal composition according to claim 9, further containing at least one compound selected from the group of compounds represented by formula (2) to formula (4): 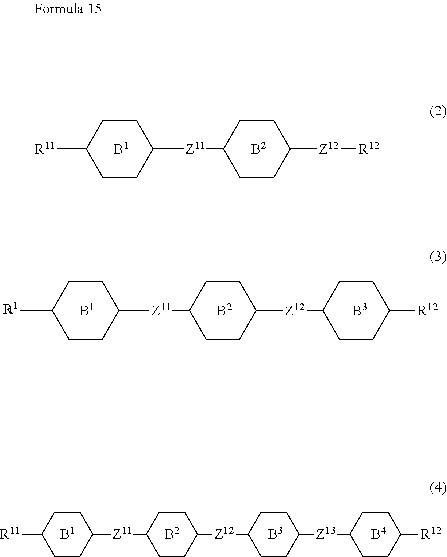
wherein, in formula (2) to formula (4),
R¹¹ and R¹² are independently alkyl having 1 to 10 carbons or alkenyl having 2 to 10 carbons, and in the alkyl and the alkenyl, at least one piece of —CH₂— may be replaced by —O—, and at least one hydrogen may be replaced by fluorine;
ring B¹, ring B², ring B³ and ring B⁴ are independently 1,4-cyclohexylene, 1,4-phenylene, 2-fluoro-1,4-phenylene, 2,5-difluoro-1,4-phenylene or pyrimidine-2,5-diyl; and
Z¹¹, Z¹² and Z¹³ are independently a single bond, —CH₂CH₂—, —CH═CH—, —C≡C— or —COO—.

11. The liquid crystal composition according to claim 9, further containing at least one compound selected from the group of compounds represented by formula (5) to formula (7): 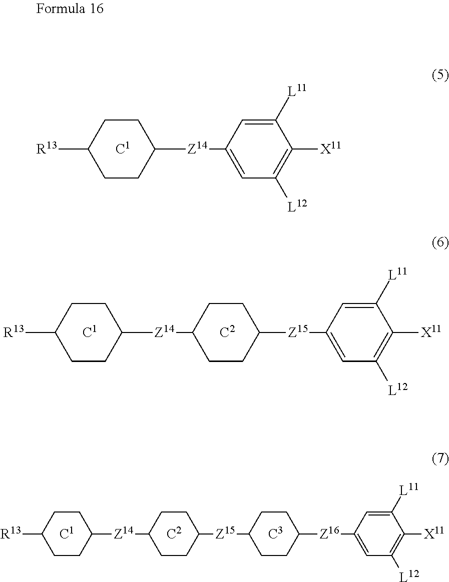
wherein, in formula (5) to formula (7),
R¹³ is alkyl having 1 to 10 carbons or alkenyl having 2 to 10 carbons, and in the alkyl and the alkenyl, at least one piece of —CH₂— may be replaced by —O—, and at least one hydrogen may be replaced by fluorine;
X¹¹ is fluorine, chlorine, —OCF₃, —OCHF₂, —CF₃, —CHF₂, —CH₂F, —OCF₂CHF₂ or —OCF₂CHFCF₃;
ring C¹, ring C² and ring C³ are independently 1,4-cyclohexylene, 1,4-phenylene in which at least one hydrogen may be replaced by fluorine, tetrahydropyran-2,5-diyl, 1,3-dioxane-2,5-diyl or pyrimidine-2,5-diyl;
Z¹⁴, Z¹⁵ and Z¹⁶ are independently a single bond, —CH₂CH₂—, —CH═CH—, —C≡C—, —COO—, —CF₂O—, —OCF₂—, —CH₂O—, —CF═CF—, —CH═CF— or —(CH₂)₄—; and
L¹¹ and L¹² are independently hydrogen or fluorine.

12. The liquid crystal composition according to claim 9, further containing at least one compound of compounds represented by formula (8): 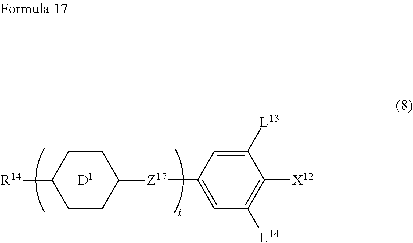
wherein, in formula (8),
R¹⁴ is alkyl having 1 to 10 carbons or alkenyl having 2 to 10 carbons, and in the alkyl and the alkenyl, at least one piece of —CH₂— may be replaced by —O—, and at least one hydrogen may be replaced by fluorine;
X¹² is —C≡N or —C≡C—C≡N;
ring D¹ is 1,4-cyclohexylene, 1,4-phenylene in which at least one hydrogen may be replaced by fluorine, tetrahydropyran-2,5-diyl, 1,3-dioxane-2,5-diyl or pyrimidine-2,5-diyl;
Z¹⁷ is a single bond, —CH₂CH₂—, —C≡C—, —COO—, —CF₂O—, —OCF₂— or —CH₂O—;
L¹³ and L¹⁴ are independently hydrogen or fluorine; and
i is 1, 2, 3 or 4.

13. The liquid crystal composition according to claim 9, further containing at least one compound selected from the group of compounds represented by formula (9) to formula (15): 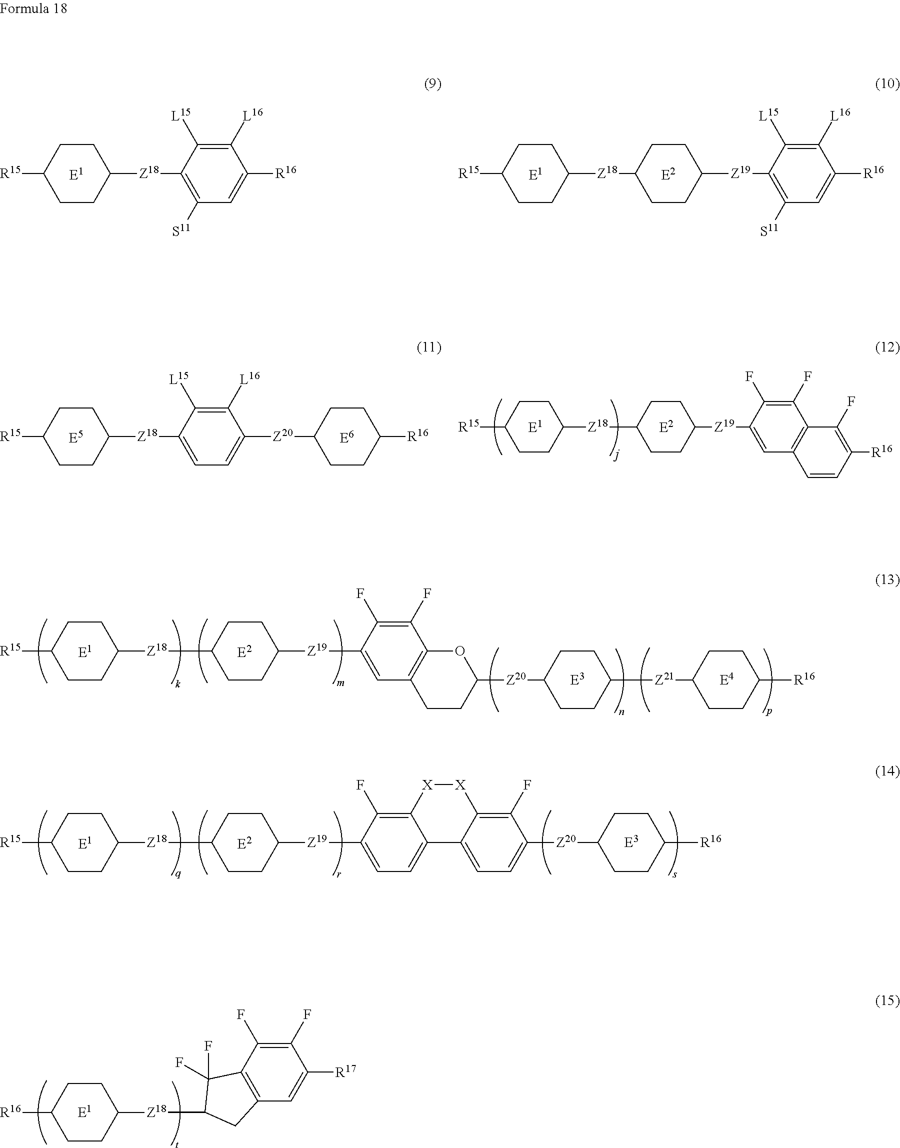
wherein, in formula (9) to formula (15),
R¹⁵ and R¹⁶ are independently alkyl having 1 to 10 carbons or alkenyl having 2 to 10 carbons, and in the alkyl and the alkenyl, at least one piece of —CH₂— may be replaced by —O—, and at least one hydrogen may be replaced by fluorine;
R¹⁷ is hydrogen, fluorine, alkyl having 1 to 10 carbons or alkenyl having 2 to 10 carbons, and in the alkyl and the alkenyl, at least one piece of —CH₂— may be replaced by —O—, and at least one hydrogen may be replaced by fluorine;
ring E¹, ring E², ring E³ and ring E⁴ are independently 1,4-cyclohexylene, 1,4-cyclohexenylene, 1,4-phenylene in which at least one hydrogen may be replaced by fluorine, tetrahydropyran-2,5-diyl or decahydronaphthalene-2,6-diyl;
ring E⁵ and ring E⁶ are independently 1,4-cyclohexylene, 1,4-cyclohexenylene, 1,4-phenylene, tetrahydropyran-2,5-diyl or decahydronaphthalene-2,6-diyl;
Z¹⁸, Z¹⁹, Z²⁰ and Z²¹ are independently a single bond, —CH₂CH₂—, —COO—, —CH₂O—, —OCF₂— or —OCF₂CH₂CH₂—;
L¹⁵ and L¹⁶ are independently fluorine or chlorine;
S¹¹ is hydrogen or methyl;
X is —CHF— or —CF₂—; and
j, k, m, n, p, q, r and s are independently 0 or 1, a sum of k, m, n and p is 1 or 2, a sum of q, r and s is 0, 1,2 or 3, and t is 1,2 or 3.

14. The liquid crystal composition according to claim 9, containing at least one polymerizable compound of compounds represented by formula (16): 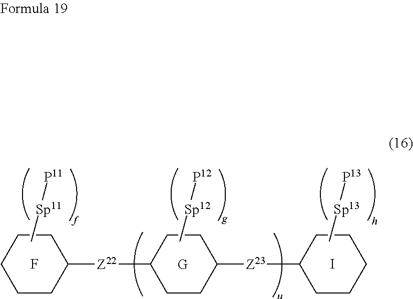
wherein, in formula (16),
ring F and ring I are independently cyclohexyl, cyclohexenyl, phenyl, 1-naphthyl, 2-naphthyl, tetrahydropyran-2-yl, 1,3-dioxane-2-yl, pyrimidine-2-yl or pyridine-2-yl, and in the rings, at least one hydrogen may be replaced by halogen, alkyl having 1 to 12 carbons, or alkyl having 1 to 12 carbons in which at least one hydrogen is replaced by halogen;
ring G is 1,4-cyclohexylene, 1,4-cyclohexenylene, 1,4-phenylene, naphthalene-1,2-diyl, naphthalene-1,3-diyl, naphthalene-1,4-diyl, naphthalene-1,5-diyl, naphthalene-1,6-diyl, naphthalene-1,7-diyl, naphthalene-1,8-diyl, naphthalene-2,3-diyl, naphthalene-2,6-diyl, naphthalene-2,7-diyl, tetrahydropyran-2,5-diyl, 1,3-dioxane-2,5-diyl, pyrimidine-2,5-diyl or pyridine-2,5-diyl, and in the rings, at least one hydrogen may be replaced by halogen, alkyl having 1 to 12 carbons, alkoxy having 1 to 12 carbons, or alkyl having 1 to 12 carbons in which at least one hydrogen is replaced by halogen;
Z²² and Z²³ are independently a single bond or alkylene having 1 to 10 carbons, and in the alkylene, at least one piece of —CH₂— may be replaced by —O—, —CO—, —COO— or —OCO—, and at least one piece of —CH₂CH₂— may be replaced by —CH═CH—, —C(CH₃)═CH—, —CH═C(CH₃)— or —C(CH₃)═C(CH₃)—, and in the groups, at least one hydrogen may be replaced by fluorine or chlorine; and
P¹¹, P¹² and P¹³ are independently a polymerizable group selected from the group of groups represented by formula (P-1) to formula (P-5); 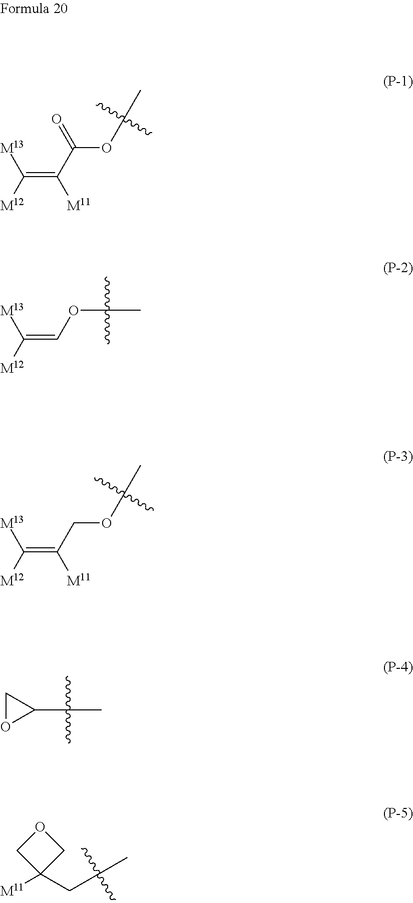
wherein
M¹¹, M¹² and M¹³ are independently hydrogen, fluorine, alkyl having 1 to 5 carbons, or alkyl having 1 to 5 carbons in which at least one hydrogen is replaced by fluorine or chlorine;
Sp¹¹, Sp¹² and Sp¹³ are independently a single bond or alkylene having 1 to 10 carbons, and in the alkylene, at least one piece of —CH₂— may be replaced by —O—, —COO—, —OCO— or —OCOO—, and at least one piece of —CH₂CH₂— may be replaced by —CH═CH— or —C≡C— and in the groups, at least one hydrogen may be replaced by fluorine or chlorine;
u is 0, 1 or 2; and
f, g and h are independently 0, 1, 2, 3 or 4, and a sum of f, g and h is 2 or more.

15. The liquid crystal composition according to claim 9, containing at least one polymerizable compound selected from the group of compounds represented by formula (16-1) to formula (16-27): 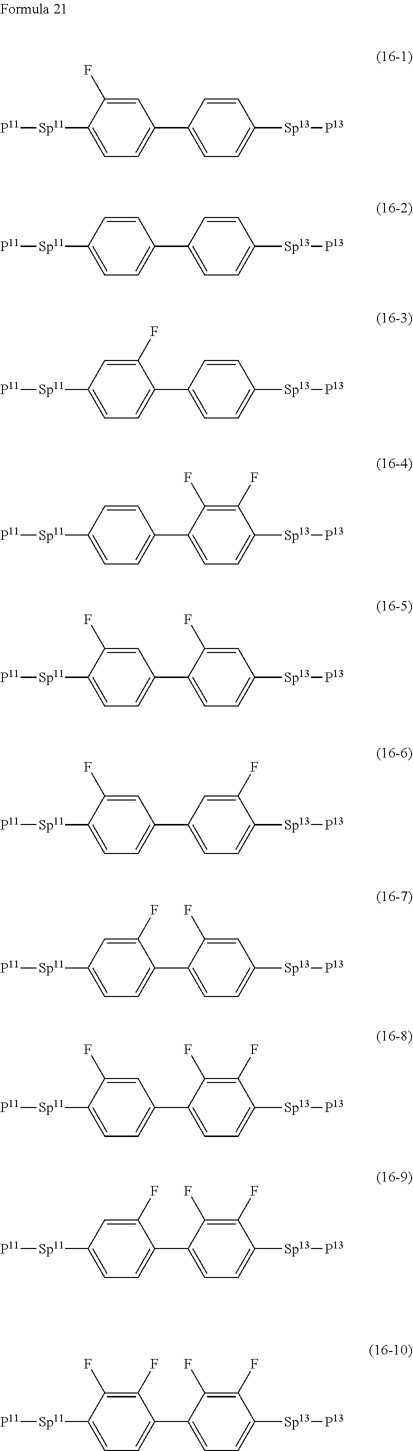 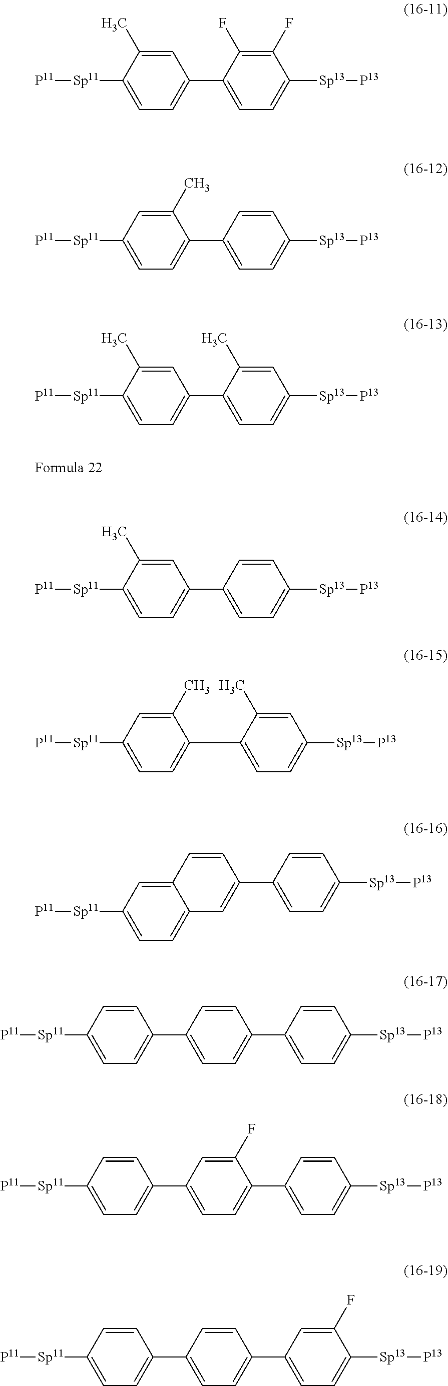 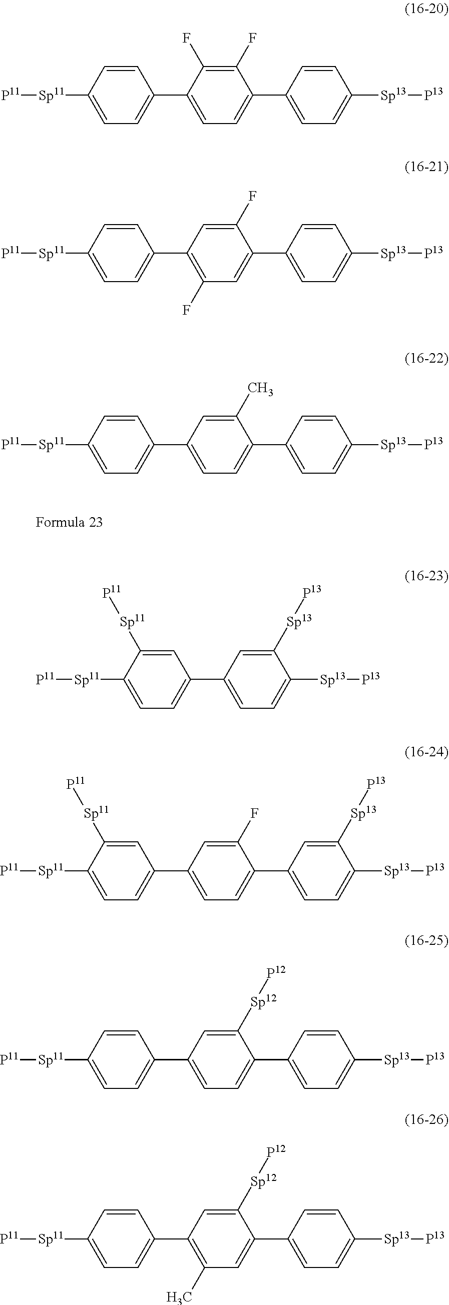 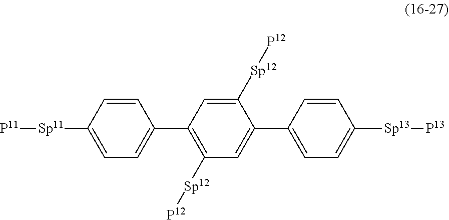
wherein, in formula (16-1) to formula (16-27),
P¹¹, P¹² and P¹³ are independently a polymerizable group selected from the group of groups represented by formula (P-1) to formula (P-3), in which M¹¹, M¹² and M¹³ are independently hydrogen, fluorine, alkyl having 1 to 5 carbons, or alkyl having 1 to 5 carbons in which at least one hydrogen is replaced by halogen; 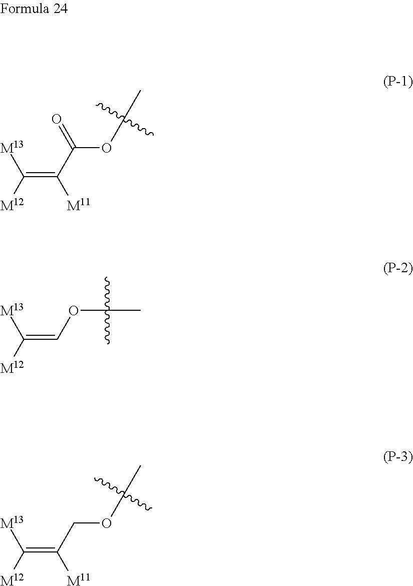
wherein
Sp¹¹, Sp¹² and Sp¹³ are independently a single bond or alkylene having 1 to 10 carbons, and in the alkylene, at least one piece of —CH₂— may be replaced by —O—, —COO—, —OCO— or —OCOO—, and at least one piece of —CH₂CH₂— may be replaced by —CH═CH— or —C≡C—, and in the groups, at least one hydrogen may be replaced by fluorine or chlorine.

16. The liquid crystal composition according to claim 9, further containing at least one of a polymerizable compound other than formula (1) and formula (16), a polymerization initiator, a polymerization inhibitor, an optically active compound, an antioxidant, an ultraviolet light absorber, a light stabilizer, a heat stabilizer and an antifoaming agent.

17. A liquid crystal display device, including the liquid crystal composition according to claim 9.
